(19) United States
(12) Patent Application Publication
(10) Pub. No.: US 2003/0092003 A1
(43) Pub. Date: May 15, 2003

(54) Method and reagent for the treatment of Alzheimer's disease

(75) Inventors: Lawrence Blatt, Boulder, CO (US); James McSwiggen, Boulder, CO (US)

Correspondence Address: MCDONNELL BOEHNEN HULBERT & BERGHOFF, 300 SOUTH WACKER DRIVE, SUITE 3200, CHICAGO, IL, 60606, US

(73) Assignee: Ribozyme Pharmaceuticals, Inc.

(21) Appl. No.: 09/930,423

(22) Filed: Aug. 15, 2001

Related U.S. Application Data
(63) Continuation-in-part of application No. 09/745,237, filed on Dec. 20, 2000.
(60) Provisional application No. 60/173,612, filed on Dec. 29, 1999.

(51) Int. Cl. C12Q 1/68; C07H 21/02; C12N 9/22

(52) U.S. Cl. 435/6; 435/199; 536/23.1

(57) ABSTRACT

Nucleic acid molecules, including antisense and enzymatic nucleic acid molecules, such as hammerhead ribozymes, DNAzymes, allozymes (allosteric ribozymes, aptazymes) and antisense, which modulate and/or detect the expression of molecular targets impacting the development and progression of Alzheimer's disease, in particular, the expression of beta secretase (BACE), presenilin-2 (ps-2), and amyloid precursor protein (APP) genes.

This patent application is a continuation-in-part of Blatt et al., U.S. Ser. No. 09/745,237, filed Dec. 20, 2000, entitled “METHOD AND REAGENT FOR THE TREATMENT OF ALZHEIMER'S DISEASE” which claims the benefit of Blatt et al., U.S. provisional application serial No. 60/173,612, filed Dec. 29, 1999, entitled “METHOD AND REAGENT FOR THE TREATMENT OF ALZHEIMER'S DISEASE” These applications are hereby incorporated by reference herein in their entirety including the drawings.

### BACKGROUND OF THE INVENTION

The present invention concerns compounds, compositions, and methods for the study, diagnosis, and treatment of Alzheimer's disease (AD).

The following is a brief description of the current understanding of Alzheimer's disease. The discussion is not meant to be complete and is provided only to assist understanding the invention that follows. The summary is not an admission that any of the work described below is prior art to the claimed invention.

Alzheimer's disease (AD) is a progressive, degenerative disease of the brain which affects approximately 4 million people in the United States alone. An estimated 14 million Americans will have Alzheimer's disease by the middle of the next century if no cure or definitive prevention of the disease is found. Nearly one out of ten people over age 65 and nearly half of those over 85 have Alzheimer's disease. Alzheimer's disease is not confined to the elderly, a small percentage of people in their 30's and 40's are afflicted with early onset AD. Alzheimer's disease is the most common form of dementia, and amounts to the third most expensive disease in the US following heart disease and cancer. An estimated 100 billion dollars are spent annually on Alzheimer's disease (National Alzheimer's Association, 1999).

Alzheimer's disease is characterized by the progressive formation of insoluble plaques and vascular deposits in the brain consisting of the 4 kD amyloid P peptide (Aβ). These plaques are characterized by dystrophic neurites that show profound synaptic loss, neurofibrillary tangle formation, and gliosis. Aβ arises from the proteolytic cleavage of the large type I transmembrane protein, β-amyloid precursor protein (APP) (Kang et al., 1987, Nature, 325, 733). Processing of APP to generate Aβ requires two sites of cleavage by a β-secretase and a γ-secretase. β-secretase cleavage of APP results in the cytoplasmic release of a 100 kD soluble amino-terminal fragment, APPsβ, leaving behind a 12 kD transmembrane carboxy-terminal fragment, C99. Alternately, APP can be cleaved by a α-secretase to generate cytoplasmic APPsα and transmembrane C83 fragments. Both remaining transmembrane fragments, C99 and C83, can be further cleaved by a γ-secretase, leading to the release and secretion of Alzheimer's related Aβ and a non-pathogenic peptide, p3, respectively (Vassar et al., 1999, Science, 286, 735-741). Early onset familial Alzheimer's disease is characterized by mutant APP protein with a Met to Leu substitution at position P1, characterized as the “Swedish” familial mutation (Mullan et al., 1992, Nature Genet., 1, 345). This APP mutation is characterized by a dramatic enhancement in β-secretase cleavage (Citron et al., 1992, Nature, 360, 672).

The identification of β-secretase, and γ-secretase constituents involved in the release of β-amyloid protein is of primary importance in the development of treatment strategies for Alzheimer's disease. Characterization of α-secretase is also important in this regard since α-secretase cleavage may compete with β-secretase cleavage resulting in non-pathogenic vs. pathogenic protein production. Involvement of the two metalloproteases, ADAM 10, and TACE has been demonstrated in α-cleavage of AAP (Buxbaum et al., 1999, J Biol. Chem., 273, 27765, and Lammich et al., 1999, Proc. Natl. Acad. Sci. U.S.A., 96, 3922). Studies of γ-secretase activity have demonstrated presenilin dependence (De Stooper et al., 1998, Nature, 391, 387, and De Stooper et al., 1999, Nature, 398, 518), and as such, presenilins have been proposed as γ-secretase even though presenilin does not present proteolytic activity (Wolfe et al., 1999, Nature, 398, 513).

Recently, Vassar et al., 1999, supra reported β-secretase cleavage of AAP by the transmembrane aspartic protease beta site APP cleaving enzyme, BACE. While other potential candidates for β-secretase have been proposed (for review see Evin et al., 1999, Proc. Natl. Acad. Sci. U.S.A., 96, 3922), none have demonstrated the full range of characteristics expected from this enzyme. Vassar et al, supra, demonstrate that BACE expression and localization are as expected for β-secretase, that BACE overexpression in cells results in increased β-secretase cleavage of APP and Swedish APP, that isolated BACE demonstrates site specific proteolytic activity on APP derived peptide substrates, and that antisense mediated endogenous BACE inhibition results in dramatically reduced β-secretase activity.

Current treatment strategies for Alzheimer's disease rely on either the prevention or the alleviation of symptoms and/or the slowing down of disease progression. Two drugs approved in the treatment of Alzheimer's, donepezil (Aricept®) and tacrine (Cognex®), both cholinomimetics, attempt to slow the loss of cognitive ability by increasing the amount of acetylcholine available to the brain. Antioxidant therapy through the use of antioxidant compounds such as alpha-tocopherol (vitamin E), melatonin, and selegeline (Eldepryl®) attempt to slow disease progression by minimizing free radical damage. Estrogen replacement therapy is thought to incur a possible preventative benefit in the development of Alzheimer's disease based on limited data. The use of anti-inflammatory drugs may be associated with a reduced risk of Alzheimer's as well. Calcium channel blockers such as Nimodipine® are considered to have a potential benefit in treating Alzheimer's disease due to protection of nerve cells from calcium overload, thereby prolonging nerve cell survival. Nootropic compounds, such as acetyl-L-carnitine (Alcar®) and insulin, have been proposed to have some benefit in treating Alzheimer's due to enhancement of cognitive and memory function based on cellular metabolism.

Whereby the above treatment strategies may all improve quality of life in Alzheimer's patients, there exists an unmet need in the comprehensive treatment and prevention of this disease. As such, there exists the need for therapeutics effective in reversing the physiological changes associated with Alzheimer's disease, specifically, therapeutics that can eliminate and/or reverse the deposition of amyloid β peptide. The use of compounds to modulate the expression of proteases that are instrumental in the release of amyloid β peptide, namely β-secretase (BACE), and γ-secretase (presenilin), is of therapeutic significance.

Tsai et al., 1999, Book of Abstrasts, 218^{th} ACS National Meeting, New Orleans, August 22-26, describes substrate-based alpha-aminoisobutyric acid derivatives of difluoro ketone peptidomimetic inhibitors of amyloid β peptide through γ-secretase inhibition.

Czech et al., International PCT publication No. WO 99/21886, describes peptides capable of inhibiting the interaction between presenilins and the β-amyloid peptide or its precursor for therapeutic use.

Fournier et al., International PCT publication No. WO 99/16874, describes human brain proteins capable of interacting with presenilins and cDNAs encoding them toward therapeutic use.

St. George-Hyslop et al., International PCT publication No. WO 97/27296, describes genes for proteins that interact with presenilins and their role in Alzheimer's disease toward therapeutic use.

Vassar et al., 1999, Science, 286, 735-741, describes specific antisense oligonucleotides targeting BACE, used for inhibition studies of endogenous BACE expression in 101 cells and APPsw cells via lipid mediated transfection.

Fechteler et al., International PCT publication No. WO 00/03004, describes specific hammerhead ribozymes targeting presenilin-2 RNA.

Denman, 2000, Mol. Cell Biol. Res. Commun., 4, 239-247; and Van Leeuwen, International PCT publication No. WO 98/45322, describe specific ribozymes targeting beta-amyloid precursor protein.

### SUMMARY OF THE INVENTION

The invention features novel nucleic acid-based techniques (e.g., enzymatic nucleic acid molecules, for example ribozymes, decoys, and RNA interference, for example double stranded RNA “dsRNA” such as short interfering RNA, “siRNA”), and methods for their use to modulate the expression of molecular targets impacting the development and progression of Alzheimer's disease. The invention also features nucleic acid sensor molecules whose activity is modulated by Alzheimer's related proteins, peptides, RNA or DNA, for example beta-amyloid proteins or peptides; beta-secretase (BACE) proteins, peptides, RNA or DNA; presenilin-2 (ps-2) proteins, peptides, RNA or DNA; or amyloid precursor protein (APP) proteins, peptides, RNA or DNA. Specifically, the present invention features nucleic acid sensor molecules for the diagnosis and treatment of Alzheimer's disease.

In one embodiment, the invention features use of such novel nucleic acid-based techniques, independently or in combination, to modulate, down regulate, or inhibit the expression of beta secretase, such as beta-site APP-cleaving enzyme (BACE, also known as Asp-2) (GenBank accession AF190725), and gamma secretase, such as presenilin 2 (ps-2) (e.g., GenBank accession L43964) involved in cleaving beta-amyloid precursor protein to yield amyloid β peptide.

In another embodiment, the invention features use of such novel nucleic acid based techniques, independently or in combination, to modulate, down regulate, or inhibit the expression of presenilin 1 (ps-1), for example GenBank accession No. L76517.

In another embodiment, the invention features use of such novel nucleic acid-based techniques, independently or in combination, to modulate, down regulate, or inhibit the expression of amyloid precursor protein, for example GenBank accession No. M33112.

In one embodiment, the invention features a nucleic acid sensor molecule capable of modulating the expression of beta-secretase, for example BACE, in the presence of beta-amyloid protein.

In another embodiment, the invention features a nucleic acid sensor molecule capable of modulating the expression of gamma-secretase, for example presenilin-2, in the presence of beta-amyloid protein.

In another embodiment, the invention features a nucleic acid sensor molecule capable of modulating the expression of amyloid precursor protein (APP), for example GenBank accession No. M33112, in the presence of beta-amyloid protein.

In one embodiment, the invention features a nucleic acid sensor molecule capable of modulating the expression of beta-secretase, for example BACE, in the presence of amyloid precursor protein.

In another embodiment, the invention features a nucleic acid sensor molecule capable of modulating the expression of gamma-secretase, for example presenilin-2, in the presence of amyloid precursor protein.

In another embodiment, the invention features a nucleic acid sensor molecule capable of modulating the expression of amyloid precursor protein (APP), for example GenBank accession No. M33112, in the presence of amyloid precursor protein.

In one embodiment, the invention features a nucleic acid sensor molecule capable of modulating the expression of beta-secretase, for example BACE, in the presence of beta-secretase RNA.

In another embodiment, the invention features a nucleic acid sensor molecule capable of modulating the expression of gamma-secretase, for example presenilin-2, in the presence of beta-secretase RNA.

In another embodiment, the invention features a nucleic acid sensor molecule capable of modulating the expression of amyloid precursor protein (APP), for example GenBank accession No. M33112, in the presence of beta-secretase RNA.

In one embodiment, the invention features a nucleic acid sensor molecule capable of modulating the expression of beta-secretase, for example BACE, in the presence of gamma-secretase RNA.

In another embodiment, the invention features a nucleic acid sensor molecule capable of modulating the expression of gamma-secretase, for example presenilin-2, in the presence of gamma-secretase RNA.

In another embodiment, the invention features a nucleic acid sensor molecule capable of modulating the expression of amyloid precursor protein (APP), for example GenBank accession No. M33112, in the presence of gamma-secretase RNA.

In one embodiment, the invention features a nucleic acid sensor molecule capable of modulating the expression of beta-secretase, for example BACE, in the presence of amyloid precursor protein (APP) RNA.

In another embodiment, the invention features a nucleic acid sensor molecule capable of modulating the expression of gamma-secretase, for example presenilin-2, in the presence of amyloid precursor protein (APP) RNA.

In another embodiment, the invention features a nucleic acid sensor molecule capable of modulating the expression of amyloid precursor protein (APP), for example GenBank accession No. M33112, in the presence of amyloid precursor protein (APP) RNA.

In one embodiment, the nucleic acid sensor molecule of the invention is a half zyme.

In another embodiment, the invention features the use of an enzymatic nucleic acid molecule, preferably in the hammerhead, NCH, G-cleaver, hairpin, Zinzyme, Amberzyme and/or DNAzyme motif, to inhibit the expression of beta-site APP-cleaving enzyme (BACE) gene, amyloid precursor protein (APP) gene, and/or the presenilin-2 (ps-2) gene.

The present invention also relates to nucleic acid-based molecular sensors whose activity can be modulated by the presence or absence of various signaling agents, ligands, and/or target signaling molecules associated with Alzheimer's disease. The invention further relates to a method for the detection of specific target signaling molecules such as nucleic acid molecules, proteins, peptides, antibodies, polysaccharides, lipids, sugars, metals, microbial or cellular metabolites, analytes, pharmaceuticals, and other organic and inorganic molecules related to Alzheimer's disease, by using nucleic acid sensor molecules of the invention in a variety of settings, including clinical, genomic, and research applications. The invention further relates to the use of the nucleic acid sensor molecule as molecular sensors capable of modulating the activity, function, or physical properties of other molecules, for example molecules associated with Alzheimer's disease. The present invention also contemplates the use of the nucleic acid sensor molecule constructs as molecular switches, capable of inducing or negating a response to or against Alzheimer's disease in a system, for example in biological system.

The invention further relates to the use of nucleic acid sensor molecules in a diagnostic application to identify the presence of a target signaling molecule such as a gene and/or gene products which are indicative of a particular genotype and/or phenotype, for example, the presence or absence of gene expression associated with Alzheimer's disease, within patients or patient samples. The invention also relates to a method for the diagnosis of disease states or physiological abnormalities related to the expression of RNA and DNA related to the maintenance or progression of Alzheimer's disease.

Diagnostic applications of the nucleic acid sensor molecules include the use of the nucleic acid sensor molecules for prospective diagnosis of neurological diseases including Alzheimer's disease, prognosis of therapeutic effect and/or dosing of a drug or class of drugs related to the treatment of neurological diseases, prognosis and monitoring of neurological disease outcome, monitoring of patient progress as a function of an approved drug or a drug under development for the treatment of neurological diseases such as Alzheimer's disease, patient surveillance and screening for drug and/or drug treatments for neurological diseases. Diagnostic applications include the use of nucleic acid sensors for research, development and commercialization of products for the rapid detection of macromolecules, such as molecules related to the maintenance or progression of neurological diseases such as Alzheimer's disease.

Nucleic acid sensor molecules can also be used in assays to assess the specificity, toxicity and effectiveness of various small molecules, nucleoside analogs, or non-nucleic acid drugs, or doses of a specific small molecules, nucleoside analogs or nucleic acid and non-nucleic acid drugs, against validated targets or biochemical pathways associated with neurological diseases such as Alzheimer's disease, and include the use of nucleic acid sensors in assays involved in high-throughput screening, biochemical assays, including cellular assays, in vivo animal models, clinical trial management, and for mechanistic studies in human clinical studies related to neurological diseases such as Alzheimer's disease.

In one embodiment, the nucleic acid sensor molecule of the invention comprises an enzymatic nucleic acid component and one or more sensor components, wherein, in response to an interaction of a target signaling molecule, for example BACE, ps-2, or APP, with the nucleic acid sensor molecule, the enzymatic nucleic acid component catalyzes a chemical reaction; such as covalent attachment of at least a portion of a reporter molecule to the nucleic acid sensor molecule, or cleavage of a reporter molecule.

In another embodiment, the invention features a method, comprising the steps of: (a) contacting a nucleic acid sensor molecule comprising an enzymatic nucleic acid component and one or more sensor components, in which the enzymatic nucleic acid component catalyzes a chemical reaction in response to an interaction between a target signaling molecule, for example BACE, ps-2, or APP, and the nucleic acid sensor molecule, with a system under conditions suitable for the enzymatic nucleic acid component to catalyze a chemical reaction involving the attachment of at least a portion of a reporter molecule to the nucleic acid sensor molecule in the presence of a target signaling agent; and (b) assaying for the attachment of the reporter molecule to the nucleic acid sensor molecule.

In another embodiment, the invention features a method, comprising the steps of: (a) contacting a nucleic acid sensor molecule comprising an enzymatic nucleic acid component and one or more sensor components, in which the enzymatic nucleic acid component catalyzes a chemical reaction in response to an interaction between a target signaling molecule, for example BACE, ps-2, or APP, and the nucleic acid sensor molecule, with a system under conditions suitable for the enzymatic nucleic acid component to catalyze a chemical reaction involving the cleavage of at least a portion of a reporter molecule, for example a molecular beacon, in the presence of a target signaling agent; and (b) assaying for the cleavage of the reporter molecule.

In another embodiment, the invention features a method, comprising the steps of: (a) contacting a nucleic acid sensor molecule comprising an enzymatic nucleic acid component and one or more sensor components, in which the enzymatic nucleic acid component catalyzes a chemical reaction in response to an interaction between a target signaling molecule, for example BACE, ps-2, or APP, and the nucleic acid sensor molecule, with a system under conditions suitable for the enzymatic nucleic acid component to catalyze a chemical reaction involving the cleavage of at least a portion of a reporter molecule from the nucleic acid sensor molecule in the presence of a target signaling agent; and (b) assaying for the cleavage of the reporter molecule from the nucleic acid sensor molecule.

In another embodiment, the invention features a method, comprising the steps of: (a) contacting a nucleic acid sensor molecule comprising an enzymatic nucleic acid component and one or more sensor components, in which the enzymatic nucleic acid component catalyzes a chemical reaction in response to an interaction between a target signaling molecule, for example BACE, ps-2, or APP, and the nucleic acid sensor molecule, with a system under conditions suitable for the enzymatic nucleic acid component to catalyze a chemical reaction involving the cleavage of at least a portion of a reporter molecule that is attached to a solid support in the presence of a target signaling agent; and (b) assaying for the cleavage of the reporter molecule from the solid support.

In one embodiment, the nucleic acid sensor molecule of the instant invention features an enzymatic component and a sensor component that are distinct moieties.

In another embodiment, the nucleic acid sensor molecule of the instant invention features a linker region that joins a sensor component to an enzymatic nucleic acid component.

In another embodiment, the invention features a nucleic acid sensor molecule comprising an enzymatic nucleic acid component and one or more sensor components, wherein, in response to an interaction of a target signaling molecule with the nucleic acid sensor molecule, the enzymatic nucleic acid component catalyzes a chemical reaction involving covalent attachment of at least a portion of a reporter molecule to at least a portion of the nucleic acid sensor molecule, wherein the reporter molecule comprises the formula:

R₁—L—R₂

wherein R1 is selected from the group consisting of alkyl, alkoxy, hydrogen, hydroxy, sulfhydryl, ester, anhydride, acid halide, amide, nitrile, phosphate, phosphonate, nucleoside, nucleotide, oligonucleotide; R2 is selected from the group consisting of molecular beacons, small molecules, fluorophores, chemophores, ionophores, radio-isotopes, photophores, peptides, proteins, enzymes, antibodies, nucleic acids, and enzymatic nucleic acids; L represents a linker which can be present or absent, and “—” represents a chemical bond.

In another embodiment, the invention features a nucleic acid sensor molecule comprising an enzymatic nucleic acid component and one or more sensor components, wherein, in response to an interaction of a target signaling molecule with the nucleic acid sensor molecule, the enzymatic nucleic acid component catalyzes a chemical reaction involving cleavage of at least a portion of a reporter molecule, wherein the reporter molecule comprises the formula:

R₁—L—R₂

wherein R1 is selected from the group consisting of alkyl, alkoxy, hydrogen, hydroxy, sulfhydryl, ester, anhydride, acid halide, amide, nitrile, phosphate, phosphonate, nucleoside, nucleotide, oligonucleotide; R2 is selected from the group consisting of molecular beacons, small molecules, fluorophores, chemophores, ionophores, radio-isotopes, photophores, peptides, proteins, enzymes, antibodies, nucleic acids, and enzymatic nucleic acids; L represents a linker which can be present or absent, and “—” represents a chemical bond.

In one embodiment, the detection of a chemical reaction catalyzed by a nucleic acid sensor molecule of the instant invention is indicative of the presence of the target signaling molecule in a system.

In another embodiment, the absence of a chemical reaction catalyzed by a nucleic acid sensor molecule of the instant invention is indicative of a system lacking the target signaling molecule.

In another embodiment, the invention features a method comprising the steps of: (a) contacting a nucleic acid sensor molecule which comprises (i) an enzymatic nucleic acid component comprising a substrate binding region and a catalytic region; and (ii) a sensor component comprising a nucleic acid sequence that upon interacting with a complementary sequence in the enzymatic nucleic acid component, inhibits the activity of the enzymatic nucleic acid component, and a reporter molecule comprising a nucleic acid sequence complementary to the substrate binding region of the enzymatic nucleic acid component of the nucleic acid sensor molecule, with a system under conditions suitable for the enzymatic nucleic acid component of the nucleic acid sensor molecule to catalyze cleavage of the reporter molecule in the presence of a target signaling molecule; and (b) assaying for the cleavage reaction of step (a).

In another embodiment, the invention features a method comprising the steps of: (a) contacting a nucleic acid sensor molecule which comprises (i) an enzymatic nucleic acid component comprising a substrate binding region and a catalytic region; and (ii) a sensor component comprising a nucleic acid sequence that upon interacting with a complementary sequence in the enzymatic nucleic acid component inhibits the activity of the enzymatic nucleic acid component and a reporter molecule comprising a nucleic acid sequence complementary to the substrate binding region of the enzymatic nucleic acid component of the nucleic acid sensor molecule, with a system under conditions suitable for the enzymatic nucleic acid component of the nucleic acid sensor molecule to catalyze a ligation reaction involving the reporter molecule in the presence of a target signaling molecule, and (b) assaying for the ligation reaction in step (a).

In one embodiment of the inventive method, the ligation reaction catalyzed by the nucleic acid sensor molecule causes at least a portion of a reporter molecule to be attached to the nucleic acid sensor molecule.

In another embodiment, the ligation reaction catalyzed by the nucleic acid sensor molecule causes at least a portion of a reporter molecule to be attached to a separate molecule. Suitable molecules include, for example, a separate nucleic acid molecule, peptide, protein, small molecule, biotin, or surface.

Also, in one embodiment of the inventive method, the cleavage of a reporter molecule catalyzed by the nucleic acid sensor molecule is indicative of the presence of the target signaling molecule in the system. In another embodiment, the absence of cleavage of a reporter molecule catalyzed by the nucleic acid sensor molecule is indicative of the system lacking the target signaling molecule.

In another embodiment of the inventive method, the ligation of a reporter molecule catalyzed by the nucleic acid sensor molecule is indicative of the presence of the target signaling molecule in the system. In another embodiment, the absence of ligation of a reporter molecule catalyzed by the nucleic acid sensor molecule is indicative of the system lacking the target signaling molecule.

In one embodiment, the system of the instant invention is an in vitro system. Preferably, the in vitro system is a sample derived from the group consisting of a patient, plant, water, beverage, and food preparation.

In one embodiment, the target signaling molecule of the instant invention is an RNA, DNA, analog of RNA or analog of DNA. Preferably, the target signaling molecule of the instant invention is an RNA derived from a bacteria, virus, fungi, plant or mammalian genome.

In one embodiment, the enzymatic nucleic acid component of the nucleic acid sensor molecule is selected from the group consisting of hammerhead, hairpin, inozyme, G-cleaver, Zinzyme, RNase P EGS nucleic acid and Amberzyme motif. In another embodiment, the enzymatic nucleic acid component of the nucleic acid sensor molecule is a DNAzyme.

In one embodiment, the reporter molecule of the instant invention comprises a detectable label selected from the group consisting of chromogenic substrate, fluorescent labels, chemiluminescent labels, and radioactive labels and enzymes. Suitable enzymes include, for example, luciferase, horseradish peroxidase, and alkaline phosphatase.

In another embodiment, the reporter molecule of the instant invention is immobilized on a solid support. Suitable solid supports include silicon-based chips, silicon-based beads, controlled pore glass, polystyrene, cross-linked polystyrene, nitrocellulose, biotin, plastics, metals and polyethylene films.

In one embodiment the sensor component of the nucleic acid sensor molecule is RNA, DNA, analog of RNA or analog of DNA.

In another embodiment, the sensor component of the nucleic acid sensor molecule is covalently linked to the nucleic acid sensor molecule by a linker. Suitable linkers include one or more nucleotides, abasic moieties, polyethers, polyamines, polyamides, peptides, carbohydrates, lipids, and polyhydrocarbon compounds, and any combination thereof.

In another embodiment, the sensor component of the nucleic acid sensor molecule is not covalently linked to the nucleic acid sensor molecule.

In another embodiment, the reporter molecule of the instant invention is RNA, DNA, RNA analog, or DNA analog.

In another embodiment, the invention features a kit comprising: (a) a nucleic acid sensor molecule which comprises (i) an enzymatic nucleic acid component comprising a substrate binding region and a catalytic region; and (ii) a sensor component comprising a nucleic acid which interacts with a complementary sequence in the enzymatic nucleic acid component to inhibit the activity of the enzymatic nucleic acid component; and (b) a reporter molecule that can be modified, i.e., cleaved, ligated, polymerized, isomerized, phorphorylated, and/or dephosphorylated by the enzymatic nucleic acid component of the nucleic acid sensor molecule in the presence of a target signaling molecule, for example BACE, ps-2, or APP, wherein the reporter molecule comprises a chemical moiety capable of emitting a detectable signal upon its modification.

In another embodiment, the invention features a kit which comprises: (a) a nucleic acid sensor molecule comprising an enzymatic nucleic acid component and one or more sensor components; and (b) a reporter molecule, wherein, in response to an interaction of a target signaling molecule, for example BACE, ps-2, or APP, with the nucleic acid sensor molecule, the enzymatic nucleic acid component catalyzes a chemical reaction involving covalent attachment of at least a portion of a reporter molecule to the nucleic acid sensor molecule.

In another embodiment, the invention features a kit which comprises: (a) a nucleic acid sensor molecule comprising, an enzymatic nucleic acid component and one or more sensor components; and (b) a reporter molecule, wherein in response to an interaction of a target signaling molecule, for example BACE, ps-2, or APP, with the nucleic acid sensor molecule, the enzymatic nucleic acid component is capable of carrying out a chemical reaction involving isomerization of at least a portion of a reporter molecule.

In another embodiment, the invention features a kit which comprises: (a) a nucleic acid sensor molecule comprising an enzymatic nucleic acid component and one or more sensor components; and (b) a reporter molecule having a non-oligonucleotide-based portion, wherein, in response to an interaction of a target signaling molecule, for example BACE, ps-2, or APP, with the nucleic acid sensor molecule, the enzymatic component catalyses a chemical reaction involving phosphorylation of a non-oligonucleotide-based portion of a reporter molecule.

In another embodiment, the invention features a kit which comprises: (a) a nucleic acid sensor molecule comprising an enzymatic nucleic acid component and one or more sensor components; and (b) a reporter molecule having a non-oligonucleotide-based portion, wherein, in response to an interaction of a target signaling molecule, for example BACE, ps-2, or APP, with the nucleic acid sensor molecule, the enzymatic component catalyses a chemical reaction involving dephosphorylation of a non-oligonucleotide-based portion of a reporter molecule.

In another embodiment, the invention features a method comprising the step of contacting one or more components of a kit of the instant invention with a system under conditions suitable for the reporter molecule in the kit to be cleaved by the nucleic acid sensor molecule in the kit in the presence of a target signaling molecule, for example BACE, ps-2, or APP.

In another embodiment, the invention features a method comprising the step of contacting one or more components of a kit of the instant invention with a system under conditions suitable for at least a portion of the reporter molecule in the kit to be covalently attached to the nucleic acid sensor molecule in the kit in the presence of a target signaling molecule, for example BACE, ps-2, or APP.

In one embodiment, the invention features a method for isolating a nucleic acid sensor molecule of the instant invention, comprising the steps of: (a) contacting a random pool of nucleic acids with a target signaling molecule and a reporter molecule, and (b) isolating a nucleic acid sensor molecule that can catalyze a chemical reaction involving covalent attachment of at least a portion of the reporter molecule to the nucleic acid sensor molecule in the presence of the target signaling molecule, for example BACE, ps-2, or APP.

In another embodiment, the invention features a method for isolating a nucleic acid sensor molecule of the instant invention comprising the steps of: (a) contacting a random pool of nucleic acids with a target signaling molecule and a reporter molecule, and (b) isolating a nucleic acid sensor molecule that can catalyze a chemical reaction involving ligation of at least a portion of the reporter molecule to the nucleic acid sensor molecule in the presence of the target signaling molecule, for example BACE, ps-2, or APP.

In another embodiment, the invention features a method for isolating a nucleic acid sensor molecule of the instant invention comprising the steps of: (a) contacting a random pool of nucleic acids with a target signaling molecule and a non-oligonucleotide-based reporter molecule, and (b) isolating a nucleic acid sensor molecule that can catalyze a chemical reaction involving phosphorylation a non-oligonucleotide-based portion of the reporter molecule by the nucleic acid sensor molecule in the presence of the target signaling molecule, for example BACE, ps-2, or APP.

In another embodiment, the invention features a method for isolating a nucleic acid sensor molecule of the instant invention, comprising the steps of: (a) contacting a random pool of nucleic acids with a target signaling molecule and a non-oligonucleotide-based reporter molecule, and (b) isolating a nucleic acid sensor molecule that can catalyze a chemical reaction involving dephosphorylation of a non-oligonucleotide-based portion of the reporter molecule by the nucleic acid sensor molecule in the presence of the target signaling molecule, for example BACE, ps-2, or APP.

In one embodiment, the invention features a nucleic acid sensor molecule comprising an enzymatic nucleic acid component and one or more sensor components, wherein, in response to an interaction of a single stranded RNA (ssRNA) having a SNP, for example a ssRNA related to the maintenance or progression of Alzheimer's disease, with the nucleic acid sensor molecule in a system, the enzymatic nucleic acid component catalyzes a chemical reaction resulting in a detectable response.

In another embodiment, the invention features a nucleic acid sensor molecule comprising an enzymatic nucleic acid component and one or more sensor components, wherein, in response to an interaction of a single stranded DNA (ssDNA) having a SNP, for example a ssDNA related to the maintenance or progression of Alzheimer's disease, with the nucleic acid sensor molecule in a system, the enzymatic nucleic acid component catalyzes a chemical reaction resulting in a detectable response.

In yet another embodiment, the invention features a nucleic acid sensor molecule comprising an enzymatic nucleic acid component and one or more sensor components, wherein, in response to an interaction of a peptide, for example a peptide related to the maintenance or progression of Alzheimer's disease, with the nucleic acid sensor molecule in a system, the enzymatic nucleic acid component catalyzes a chemical reaction resulting in a detectable response.

In another embodiment, the invention features a nucleic acid sensor molecule comprising an enzymatic nucleic acid component and one or more sensor components, wherein, in response to an interaction of a protein, for example a protein related to the maintenance or progression of Alzheimer's disease, with the nucleic acid sensor molecule in a system, the enzymatic nucleic acid component catalyzes a chemical reaction resulting in a detectable response.

In one embodiment, the invention features a nucleic acid sensor molecule comprising an enzymatic nucleic acid component and one or more sensor components, wherein, in response to an interaction of a single stranded RNA (ssRNA), for example a ssRNA related to the maintenance or progression of Alzheimer's disease, with the nucleic acid sensor molecule in a system, the enzymatic nucleic acid component catalyzes a chemical reaction resulting in the cleavage of a predetermined RNA molecule associated with a disease.

In another embodiment, the invention features a nucleic acid sensor molecule comprising an enzymatic nucleic acid component and one or more sensor components, wherein, in response to an interaction of a single stranded DNA (ssDNA), for example a ssDNA related to the maintenance or progression of Alzheimer's disease, with the nucleic acid sensor molecule in a system, the enzymatic nucleic acid component catalyzes a chemical reaction resulting in the cleavage of a predetermined RNA molecule associated with a disease.

In yet another embodiment, the invention features a nucleic acid sensor molecule comprising an enzymatic nucleic acid component and one or more sensor components, wherein, in response to an interaction of a peptide, for example a peptide related to the maintenance or progression of Alzheimer's disease, with the nucleic acid sensor molecule in a system, the enzymatic nucleic acid component catalyzes a chemical reaction resulting in the cleavage of a predetermined RNA molecule associated with a disease.

In another embodiment, the invention features a nucleic acid sensor molecule comprising an enzymatic nucleic acid component and one or more sensor components, wherein, in response to an interaction of a protein, for example a protein related to the maintenance or progression of Alzheimer's disease, with the nucleic acid sensor molecule in a system, the enzymatic nucleic acid component catalyzes a chemical reaction resulting in the cleavage of a predetermined RNA molecule associated with a disease.

In one embodiment, the invention features a nucleic acid sensor molecule comprising an enzymatic nucleic acid component and one or more sensor components, wherein, in response to an interaction of a single stranded RNA (ssRNA), for example a ssRNA related to the maintenance or progression of Alzheimer's disease, with the nucleic acid sensor molecule in a system, the enzymatic nucleic acid component catalyzes a chemical reaction resulting in ligation of a predetermined RNA molecule to another predetermined RNA molecule.

In another embodiment, the invention features a nucleic acid sensor molecule comprising an enzymatic nucleic acid component and one or more sensor components, wherein, in response to an interaction of a single stranded DNA (ssDNA), for example a dsDNA related to the maintenance or progression of Alzheimer's disease, with the nucleic acid sensor molecule in a system, the enzymatic nucleic acid component catalyzes a chemical reaction resulting in ligation of a predetermined RNA molecule to another predetermined RNA molecule.

In yet another embodiment, the invention features a nucleic acid sensor molecule comprising an enzymatic nucleic acid component and one or more sensor components, wherein, in response to an interaction of a peptide, for example a peptide related to the maintenance or progression of Alzheimer's disease, with the nucleic acid sensor molecule in a system, the enzymatic nucleic acid component catalyzes a chemical reaction resulting in ligation of a predetermined RNA molecule to another predetermined RNA molecule.

In still another embodiment, the invention features a nucleic acid sensor molecule comprising an enzymatic nucleic acid component and one or more sensor components, wherein, in response to an interaction of a protein, for example a protein related to the maintenance or progression of Alzheimer's disease, with the nucleic acid sensor molecule in a system, the enzymatic nucleic acid component catalyzes a chemical reaction resulting in ligation of a predetermined RNA molecule to another predetermined RNA molecule.

In one embodiment, the invention features a method comprising: (a) contacting a nucleic acid sensor molecule of the invention with a system comprising at least one ssRNA having a SNP related to the maintenance or progression of Alzheimer's disease, under conditions suitable for the enzymatic nucleic acid component of the nucleic acid sensor molecule to catalyze a chemical reaction resulting in a detectable response; and (b) assaying for the detectable response.

In another embodiment, the invention features a method comprising: (a) contacting a nucleic acid sensor molecule of the invention with a system comprising at least one ssDNA having a SNP related to the maintenance or progression of Alzheimer's disease, under conditions suitable for the enzymatic nucleic acid component of the nucleic acid sensor molecule to catalyze a chemical reaction resulting in a detectable response; and (b) assaying for the detectable response.

In another embodiment, the invention features a method comprising: (a) contacting a nucleic acid sensor molecule of the invention with a system comprising at least one peptide related to the maintenance or progression of Alzheimer's disease, under conditions suitable for the enzymatic nucleic acid component of the nucleic acid sensor molecule to catalyze a chemical reaction resulting in a detectable response; and (b) assaying for the detectable response.

In yet another embodiment, the invention features a method comprising: (a) contacting a nucleic acid sensor molecule of the invention with a system comprising at least one protein related to the maintenance or progression of Alzheimer's disease, under conditions suitable for the enzymatic nucleic acid component of the nucleic acid sensor molecule to catalyze a chemical reaction resulting in a detectable response; and (b) assaying for the detectable response.

In one embodiment, the invention features a method comprising contacting a nucleic acid sensor molecule of the invention with a system comprising at least one ssRNA related to the maintenance or progression of Alzheimer's disease, under conditions suitable for the enzymatic nucleic acid component of the nucleic acid sensor molecule to cleave a predetermined RNA molecule.

In another embodiment, the invention features a method comprising the steps of contacting a nucleic acid sensor molecule of the invention with a system comprising at least one ssDNA related to the maintenance or progression of Alzheimer's disease, under conditions suitable for the enzymatic nucleic acid component of the nucleic acid sensor molecule to cleave a predetermined RNA molecule In yet another embodiment, the invention features a method comprising the steps of contacting a nucleic acid sensor molecule of the invention with a system comprising at least one peptide related to the maintenance or progression of Alzheimer's disease, under conditions suitable for the enzymatic nucleic acid component of the nucleic acid sensor molecule to cleave a predetermined RNA molecule.

In another embodiment, the invention features a method comprising the steps of contacting a nucleic acid sensor molecule of the invention with a system comprising at least one protein related to the maintenance or progression of Alzheimer's disease, under conditions suitable for the enzymatic nucleic acid component of the nucleic acid sensor molecule to cleave a predetermined RNA molecule.

In one embodiment, the invention features a method comprising contacting a nucleic acid sensor molecule of the invention with a system comprising at least one ssRNA having a SNP related to the maintenance or progression of Alzheimer's disease, under conditions suitable for the enzymatic nucleic acid component of the nucleic acid sensor molecule to ligate a predetermined RNA molecule to another predetermined RNA molecule.

In another embodiment, the invention features a method comprising the steps of contacting a nucleic acid sensor molecule of the invention with a system comprising at least one ssDNA having a SNP related to the maintenance or progression of Alzheimer's disease, under conditions suitable for the enzymatic nucleic acid component of the nucleic acid sensor molecule to ligate a predetermined RNA molecule to another predetermined RNA molecule.

In yet another embodiment, the invention features a method comprising the steps of contacting a nucleic acid sensor molecule of the invention with a system comprising at least one peptide related to the maintenance or progression of Alzheimer's disease, under conditions suitable for the enzymatic nucleic acid component of the nucleic acid sensor molecule to ligate a predetermined RNA molecule to another predetermined RNA molecule.

In another embodiment, the invention features a method comprising the steps of contacting a nucleic acid sensor molecule of the invention with a system comprising at least one protein related to the maintenance or progression of Alzheimer's disease, under conditions suitable for the enzymatic nucleic acid component of the nucleic acid sensor molecule to ligate a predetermined RNA molecule to another predetermined RNA molecule.

In one embodiment, the invention features a method of using the nucleic acid sensor molecules of the invention to determine the function or validate a predetermined gene target, a predetermined RNA target, a predetermined peptide target, or a predetermined protein target related to the maintenance or progression of Alzheimer's disease.

In another embodiment, the invention features a method of using the nucleic acid sensor molecules of the invention to determine a genotype or to characterize single nucleotide polymorphisms SNPs in a gene or genome related to the maintenance or progression of Alzheimer's disease. In another embodiment, the invention features a method of using the nucleic acid sensor molecules of the invention to determine SNP scoring related to the maintenance or progression of Alzheimer's disease.

In another embodiment, the invention features a method of using the nucleic acid sensor molecules of the invention to determine a proteome, for example a disease specific proteome or treatment specific proteome related to the maintenance or progression of Alzheimer's disease. In yet another embodiment, the invention features a method of using the nucleic acid sensor molecules of the invention to determine a proteome map or to determine proteome scoring related to the maintenance or progression of Alzheimer's disease.

In one embodiment, the invention features a method of using the nucleic acid sensor molecules of the invention to determine the dosage of a therapy used in treating a patient, to determine susceptibility of a patient to disease, to determine drug metabolism in a patient, to select a patient for a clinical trail, to determine a choice of therapy in a patient, or to treat a patient.

In another embodiment, the detection of a chemical reaction in a method of the invention is indicative of the presence of the target signaling agent in the system.

In another embodiment, the absence of a chemical reaction in a method of the invention is indicative of the system lacking the target signaling agent.

In one embodiment, a system of the invention is an in vitro system, for example a sample derived from a patient.

In another embodiment, a system of the invention is an in vivo system, for example a mammal, mammalian cell, human, or human cell. In another embodiment, a component of a nucleic acid sensor molecule of the invention comprises a hammerhead, hairpin, inozyme, G-cleaver, Zinzyme, RNase P EGS nucleic acid, DNAzyme or Amberzyme motif.

In one embodiment, a chemical reaction of a nucleic sensor molecule of the invention comprises cleavage of a phosphodiester internucleotide linkage, ligation of a predetermined nucleic acid molecule to the nucleic acid sensor molecule, ligation of a predetermined nucleic acid molecule to another predetermined nucleic acid molecule, isomerization, phosphorylation of a peptide or protein, dephosphorylation of a peptide or protein, RNA polymerase activity, an increase or decrease in fluorescence, an increase or decrease in enzymatic activity, an increase or decrease in the production of a precipitate, an increase or decrease in chemoluminescence, or an increase or decrease in radioactive emission.

In another embodiment, the invention features a kit comprising a nucleic acid sensor molecule of the invention.

In another embodiment, the invention features an array of nucleic acid sensor molecules comprising a predetermined number of nucleic acid sensor molecules of the invention. In one embodiment, a nucleic acid sensor molecule of the instant invention is attached to a solid surface. Preferably, the surface of the instant invention comprises silicon-based chips, silicon-based beads, controlled pore glass, polystyrene, cross-linked polystyrene, nitrocellulose, biotin, plastics, metals and polyethylene films.

In one embodiment, the target signaling molecule of the invention comprises BACE, ps-2, and/or APP protein.

In another embodiment, the invention features a nucleic acid sensor molecule comprising an enzymatic nucleic acid component and one or more sensor components, wherein, in response to an interaction of a BACE, ps-2, and/or APP peptide with the nucleic acid sensor molecule in a system, the enzymatic nucleic acid component catalyzes a chemical reaction resulting in the cleavage of a predetermined RNA molecule associated with a disease, for example BACE, ps-2, and/or APP RNA.

In yet another embodiment, the invention features a nucleic acid sensor molecule comprising an enzymatic nucleic acid component and one or more sensor components, wherein, in response to an interaction of a BACE, ps-2, and/or APP protein, with the nucleic acid sensor molecule in a system, the enzymatic nucleic acid component catalyzes a chemical reaction resulting in the cleavage of a predetermined RNA molecule associated with a disease, for example BACE, ps-2, and/or APP RNA.

In another embodiment, the invention features a pharmaceutical composition comprising a nucleic acid sensor molecule in a pharmaceutically acceptable carrier.

In one embodiment, the invention features a method of administering to a cell, for example a mammalian cell or human cell, a nucleic acid sensor molecule of the invention comprising contacting the cell with the nucleic acid sensor molecule under conditions suitable for the administration. The method of administration can be in the presence of a delivery reagent, for example a lipid, cationic lipid, phospholipid, or liposome.

In another embodiment, the invention features a cell, for example a human cell, plant cell, bacterial cell, or fungal cell, including a nucleic acid sensor molecule of the invention.

In another embodiment, the invention features an expression vector comprising a nucleic acid sequence encoding at least one nucleic acid sensor molecule of the invention in a manner which allows expression of the nucleic acid sensor molecule.

In yet another embodiment, the invention features a mammalian cell, for example a human cell, including an expression vector of the invention.

In one embodiment, an expression vector of the invention further comprises a sequence for a nucleic acid sensor molecule complementary to an RNA having BACE, ps-2, and/or APP sequence.

In another embodiment, an expression vector of the invention comprises a nucleic acid sequence encoding two or more nucleic acid sensor molecules, which can be the same or different.

In another embodiment, a predetermined RNA of the invention is associated with Alzheimer's disease.

In another embodiment, the method of the instant invention is carried out more than once.

By “inhibit”, “down-regulate”, or “reduce”, it is meant that the expression of the gene, or level of RNAs or equivalent RNAs encoding one or more protein subunits, or activity of one or more protein subunits, such as BACE, ps-2, or APP, is reduced below that observed in the absence of the nucleic acid molecules of the invention. In one embodiment, inhibition, down-regulation or reduction with a nucleic acid molecule preferably is below that level observed in the presence of an enzymatically inactive or attenuated molecule that is able to bind to the same site on the target RNA, but is unable to cleave that RNA. In another embodiment, inhibition, down-regulation, or reduction with antisense oligonucleotides is preferably below that level observed in the presence of, for example, an oligonucleotide with scrambled sequence or with mismatches. In another embodiment, inhibition, down-regulation, or reduction of BACE, ps-2, or APP with the nucleic acid molecule of the instant invention is greater in the presence of the nucleic acid molecule than in its absence.

By “modulate” is meant that the expression of the gene, or level of RNAs or equivalent RNAs encoding one or more protein subunits, or activity of one or more protein subunit(s) is up-regulated or down-regulated, such that the expression, level, or activity is greater than or less than that observed in the absence of the nucleic acid molecules of the invention.

By “up-regulate” is meant that the expression of the gene, or level of RNAs or equivalent RNAs encoding one or more protein subunits, or activity of one or more protein subunits, such as BACE, PS-2, or APP subunit(s), is greater than that observed in the absence of the nucleic acid molecules of the invention. For example, the expression of a gene, such as BACE, PS-2, or APP gene, can be increased in order to treat, prevent, ameliorate, or modulate a pathological condition caused or exacerbated by an absence or low level of gene expression.

By “enzymatic nucleic acid molecule” it is meant a nucleic acid molecule that has complementarity in a substrate binding region to a specified gene target, and also has an enzymatic activity which is active to specifically cleave target RNA. That is, the enzymatic nucleic acid molecule is able to intermolecularly cleave RNA and thereby inactivate a target RNA molecule. These complementary regions allow sufficient hybridization of the enzymatic nucleic acid molecule to the target RNA and thus permit cleavage. One hundred percent complementarity is preferred, but complementarity as low as 50-75% may also be useful in this invention. The nucleic acids may be modified at the base, sugar, and/or phosphate groups. The term enzymatic nucleic acid is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not meant to be limiting and those skilled in the art will recognize that all that is important in an enzymatic nucleic acid molecule of this invention is that it have a specific substrate binding site which is complementary to one or more of the target nucleic acid regions, and that it have nucleotide sequences within or surrounding that substrate binding site which impart a nucleic acid cleaving activity to the molecule (Cech et al., U.S. Pat. No. 4,987,071; Cech et al., 1988, JAMA).

The term “nucleic acid sensor molecule” “aptazyme” or “allozyme” as used herein refers to an allosteric enzymatic nucleic acid molecule such as the nucleic acid sensor molecules described herein or other allosteric nucleic acid molecules, for example as described by George et al., U.S. Pat. No. 5,834,186 and U.S. Pat. No. 5,741,679, Shih et al., U.S. Pat. No. 5,589,332, Nathan et al., U.S. Pat. No 5,871,914, Nathan and Ellington, International PCT publication No. WO 00/24931, Breaker et al., International PCT Publication Nos. WO 00/26226 and 98/27104, Sullenger et al., International PCT publication No. WO 99/29842, Usman et al., U.S. Ser. No. 09/800,594 and U.S. Ser. No. 09/877,526.

By “halfzyme” is meant an enzymatic nucleic acid molecule assembled from two or more nucleic acid components. The enzymatic nucleic acid in the halfzyme configuration is active when all the necessary components interact with each other. The halfzyme construct can be engineered to have a component lacking from the structure or sequence of the enzymatic nucleic acid molecule such that enzymatic activity is inhibited. In the presence of the target signaling agent, the required component for enzymatic activity is provided such that the halfzyme is catalytically active (see for example FIG. 6).By “enzymatic portion” or “catalytic domain” is meant that portion/region of the enzymatic nucleic acid molecule essential for cleavage of a nucleic acid substrate (for example see FIGS. 1-5).

By “substrate binding arm” or “substrate binding domain” is meant that portion/region of a ribozyme which is complementary to (i.e., able to base-pair with) a portion of its substrate. Generally, such complementarity is 100%, but can be less if desired. For example, as few as 10 bases out of 14 may be base-paired. Such arms are shown generally in FIGS. 1-5. That is, these arms contain sequences within a ribozyme which are intended to bring ribozyme and target RNA together through complementary base-pairing interactions. The ribozyme of the invention may have binding arms that are contiguous or non-contiguous and may be of varying lengths. The length of the binding arm(s) are preferably greater than or equal to four nucleotides and of sufficient length to stably interact with the target RNA; a specific embodiment 12-100 nucleotides; more preferably 14-24 nucleotides long. If two binding arms are chosen, the design is such that the length of the binding arms are symmetrical (i.e., each of the binding arms is of the same length; e.g., five and five nucleotides, six and six nucleotides or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like).

The term “Inozyme” or “NCH” motif as used herein, refers to an enzymatic nucleic acid molecule comprising a motif as is generally described as NCH Rz in FIG. 2. Inozymes possess endonuclease activity to cleave RNA substrates having a cleavage triplet NCH/, where N is a nucleotide, C is cytidine and H is adenosine, uridine or cytidine, and / represents the cleavage site. H is used interchangeably with X. Inozymes can also possess endonuclease activity to cleave RNA substrates having a cleavage triplet NCN/, where N is a nucleotide, C is cytidine, and / represents the cleavage site. “I” in FIG. 2 represents an Inosine nucleotide, preferably a ribo-Inosine or xylo-Inosine nucleoside. See for example, Ludwig et al., U.S. Serial No. 60/156,236, filed Sep. 27, 1999, entitled “COMPOSITIONS HAVING RNA CLEAVING ACTIVITY”, and Ludwig et al., International PCT Publication No. WO 98/58058.

The term “G-cleaver” motif as used herein, refers to an enzymatic nucleic acid molecule comprising a motif as is generally described as G-cleaver Rz in FIG. 2. G-cleavers possess endonuclease activity to cleave RNA substrates having a cleavage triplet NYN/, where N is a nucleotide, Y is uridine or cytidine and / represents the cleavage site. G-cleavers can be chemically modified as is generally shown in FIG. 2. See for example Eckstein et al., U.S. Ser. No. 09/444,209, entitled “NUCLEIC ACID CATALYSTS WITH ENDONUCLEASE ACTIVITY,” which was filed on Nov. 19, 1999 and which is a continuation-in-part of U.S. Ser. No. 09/159,274, and Eckstein et al., International PCT Publication No. WO 99/16871.

The term “amberzyme” motif as used herein, refers to an enzymatic nucleic acid molecule comprising a motif as is generally described in FIG. 3. Amberzymes possess endonuclease activity to cleave RNA substrates having a cleavage triplet NG/N, where N is a nucleotide, G is guanosine, and / represents the cleavage site. Amberzymes can be chemically modified to increase nuclease stability through substitutions as are generally shown in FIG. 3. In addition, differing nucleoside and/or non-nucleoside linkers can be used to substitute the 5′-gaaa-3′ loops shown in the figure. Amberzymes represent a non-limiting example of an enzymatic nucleic acid molecule that does not require a ribonucleotide (2′-OH) group within its own nucleic acid sequence for activity. See for example Beigelman et al., U.S. Ser. No. 09/301,511 filed Apr. 28, 1999, entitled “NUCLEOTIDE TRIPHOSPHATES AND THEIR INCORPORATION INTO OLIGONUCLEOTIDES” and Beigelman et al., International PCT Publication No. WO 99/55857.

The term “zinzyme” motif as used herein, refers to an enzymatic nucleic acid molecule comprising a motif as is generally described in FIG. 4. Zinzymes possess endonuclease activity to cleave RNA substrates having a cleavage triplet including but not limited to YG/Y, where Y is uridine or cytidine, and G is guanosine and / represents the cleavage site. Zinzymes can be chemically modified to increase nuclease stability through substitutions as are generally shown in FIG. 4, including substituting 2′-O-methyl guanosine nucleotides for guanosine nucleotides. In addition, differing nucleotide and/or non-nucleotide linkers can be used to substitute the 5′-gaaa-2′ loop shown in the figure. Zinzymes represent a non-limiting example of an enzymatic nucleic acid molecule that does not require a ribonucleotide (2′-OH) group within its own nucleic acid sequence for activity. See for example Beigelman et al., U.S. Ser. No. 09/301,511 filed Apr. 28, 1999, entitled “NUCLEOTIDE TRIPHOSPHATES AND THEIR INCORPORATION INTO OLIGONUCLEOTIDES” and Beigelman et a., International PCT Publication No. WO 99/55857.

The term ‘DNAzyme’ as used herein, refers to an enzymatic nucleic acid molecule that does not require the presence of a 2′-OH group for its activity. In particular embodiments the enzymatic nucleic acid molecule can have an attached linker(s) or other attached or associated groups, moieties, or chains containing one or more nucleotides with 2′-OH groups. DNAzymes can be synthesized chemically or expressed endogenously in vivo, by means of a single stranded DNA vector or equivalent thereof. An example of a DNAzyme is shown in FIG. 5 and is generally reviewed in Usman et al., International PCT Publication No. WO 95/11304; Chartrand et al., 1995, NAR 23, 4092; Breaker et al., 1995, Chem. Bio. 2, 655; Santoro et al., 1997, PNAS 94, 4262; Breaker, 1999, Nature Biotechnology, 17, 422423; and Santoro et. al., 2000, J. Am. Chem. Soc., 122, 2433-39. Additional DNAzyme motifs can be selected for using techniques similar to those described in these references, and hence, are within the scope of the present invention.

By “decoy” is meant a nucleic acid molecule or aptamer that is designed to preferentially bind to a predetermined ligand. Such binding can result in the inhibition or activation of a target molecule. The decoy RNA or aptamer can compete with a naturally occurring binding target for the binding of a specific ligand. For example, it has been shown that over-expression of HIV trans-activation response (TAR) RNA can act as a “decoy” and efficiently binds HIV tat protein, thereby preventing it from binding to TAR sequences encoded in the HIV RNA (Sullenger et al., 1990, Cell, 63, 601-608). This is but a specific example and those in the art will recognize that other embodiments can be readily generated using techniques generally known in the art, see for example Gold et al., 1995, Annu. Rev. Biochem., 64, 763; Brody and Gold, 2000, J. Biotechnol., 74, 5; Sun, 2000, Curr. Opin. Mol. Ther., 2, 100; Kusser, 2000, J Biotechnol., 74, 27; Hermann and Patel, 2000, Science, 287, 820; and Jayasena, 1999, Clinical Chemistry, 45, 1628. Similarly, a decoy RNA can be designed to bind to a EGFR receptor and block the binding of EGFR or a decoy RNA can be designed to bind to EGFR and prevent interaction with the EGFR receptor.

By “RNA interference” as used herein refers to the degradation of target RNA molecules mediated by double stranded RNA molecules in which one strand of the double stranded RNA is complementary to the target RNA. RNA interference is mediated by short interfering RNA “siRNA”, see for example Bass, 2001, Nature, 411, 428429; Elbashir et al., 2001, Nature, 411, 494-498).

The term “double stranded RNA” or “dsRNA” as used herein refers to a double stranded RNA molecule capable of RNA interference, including short interfering RNA “siRNA” see for example Bass, 2001, Nature, 411, 428-429; Elbashir et al., 2001, Nature, 411, 494-498).

By “sufficient length” is meant an oligonucleotide of greater than or equal to 3 nucleotides. In connection with the binding arms of an enzymatic nucleic acid molecule, “sufficient length” means that the binding arms are long enough to provide a stable interaction with a target RNA under the expected conditions. Preferably the binding arms are not so long as to prevent a useful level of turnover.

By “stably interact” is meant, interaction of the oligonucleotides with target nucleic acid (e.g., by forming hydrogen bonds with complementary nucleotides in the target under physiological conditions).

By “equivalent” RNA to BACE is meant to include those naturally occurring RNA molecules having homology (partial or complete) to RNA encoding BACE proteins or encoding for proteins with similar function as BACE in various organisms, including human, rodent, primate, rabbit, pig, protozoans, fungi, plants, and other microorganisms and parasites. The equivalent RNA sequence also includes in addition to the coding region, regions such as 5′-untranslated region, 3′-untranslated region, introns, intron-exon junction and the like.

By “equivalent” RNA to ps-2 is meant to include those naturally occurring RNA molecules having homology (partial or complete) to RNA encoding ps-2 proteins or encoding for proteins with similar function as ps-2 in various organisms, including human, rodent, primate, rabbit, pig, protozoans, fungi, plants, and other microorganisms and parasites. The equivalent RNA sequence also includes in addition to the coding region, regions such as 5′-untranslated region, 3′-untranslated region, introns, intron-exon junction and the like.

By “equivalent” RNA to APP is meant to include those naturally occurring RNA molecules having homology (partial or complete) to RNA encoding APP proteins or encoding for proteins with similar function as APP in various organisms, including human, rodent, primate, rabbit, pig, protozoans, fungi, plants, and other microorganisms and parasites. The equivalent RNA sequence also includes in addition to the coding region, regions such as 5′-untranslated region, 3′-untranslated region, introns, intron-exon junction and the like.

By “homology” is meant the nucleotide sequence of two or more nucleic acid molecules is partially or completely identical. Preferably, the sequences are at least 70%, 80%, 90%, or 95% identical over an analysis window of at least 50 or 100 contiguous nucleotides.

By “antisense nucleic acid” it is meant a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review see Stein and Cheng, 1993 Science 261, 1004). Typically, antisense molecules will be complementary to a target sequence along a single contiguous sequence of the antisense molecule. However, in certain embodiments, an antisense molecule may bind to substrate such that the substrate molecule forms a loop, and/or an antisense molecule may bind such that the antisense molecule forms a loop. Thus, the antisense molecule may be complementary to two (or even more) non-contiguous substrate sequences or two (or even more) non-contiguous sequence portions of an antisense molecule may be complementary to a target sequence or both.

By “2-5A antisense chimera” it is meant, an antisense oligonucleotide containing a 5′ phosphorylated 2′-5′-linked adenylate residues. These chimeras bind to target RNA in a sequence-specific manner and activate a cellular 2-5A-dependent ribonuclease which, in turn, cleaves the target RNA (Torrence et al., 1993 Proc. Natl. Acad. Sci. USA 90, 1300).

By “triplex DNA” it is meant an oligonucleotide that can bind to a double-stranded DNA in a sequence-specific manner to form a triple-strand helix. Formation of such triple helix structure has been shown to inhibit transcription of the targeted gene (Duval-Valentin et al., 1992 Proc. Natl. Acad. Sci. USA 89, 504).

By “gene” it is meant a nucleic acid that encodes an RNA.

By “amyloid precursor protein” or “APP” is meant, a protein, protein fragment, or peptide comprising the type I transmembrane protein, β-amyloid precursor protein (see for example Kang et al., 1987, Nature, 325, 733). The terms amyloid precursor protein and APP also refer to mutant proteins, protein fragments, or peptides comprising the type I transmembrane protein, β-amyloid precursor protein, such as proteins encoded by Swedish mutant APP, where a Lys to Asn or Met to Leu substitution at the P1 position of APP is thought to result in early onset Alzheimer's disease (see for example Mullan et al., 1992, Nat. Genet., 1, 345-7).

By “signaling agent” or “target signaling agent” is meant a chemical or physical entity capable of interacting with a nucleic acid sensor molecule, specifically a sensor component of a nucleic acid sensor molecule, resulting in modification of the enzymatic nucleic acid component of the nucleic acid sensor molecule via chemical, physical, topological, or conformational changes to the structure of the molecule such that the activity of the enzymatic nucleic acid component is modulated, for example is activated or deactivated. Signaling agents can comprise target signaling molecules such as macromolecules, ligands, small molecules, metals and ions, nucleic acid molecules including but not limited to RNA and DNA or analogs thereof, proteins, peptides, antibodies, polysaccharides, lipids, sugars, microbial or cellular metabolites, pharmaceuticals, and organic and inorganic molecules in a purified or unpurified form, or physical signals including magnetism, temperature, light, sound, shock, pH, capacitance, voltage, and ionic conditions. Exemplary signaling agents of the instant invention include molecules associated with the progression and/or maintenance of Alzheimer's disease.

By “system” is meant, material, in a purified or unpurified form, from biological or non-biological sources, including but not limited to human, animal, plant, bacteria, virus, fungi, soil, water, mechanical devices, circuits, networks, computers, or others that comprises the target signaling agent or target signaling molecule to be detected or amplified. System also refers to a group of substances or components that can be collectively combined or identified. A system can comprise a biological system, for example an organism, cell, or components, extracts, and samples thereof. A system can further comprise an experimental or artificial system, where various substances or components are intentionally combined together.

The “biological system” as used herein can be a eukaryotic system or a prokaryotic system, for example a bacterial cell, plant cell or a mammalian cell, or of plant origin, mammalian origin, yeast origin, Drosophila origin, or archebacterial origin.

By “reporter molecule” is meant a molecule, such as a nucleic acid sequence (e.g., RNA or DNA or analogs thereof) or peptides and/or other chemical moieties, able to stably interact with the nucleic acid sensor molecule and function as a substrate for the nucleic acid sensor molecule.

The reporter molecule can also contain chemical moieties capable of generating a detectable response, including but not limited to, fluorescent, chromogenic, radioactive, enzymatic and/or chemiluminescent or other detectable labels that can then be detected using standard assays known in the art. The reporter molecule can also act as an intermediate in a chain of events, for example, by acting as an amplicon, inducer, promoter, or inhibitor of other events that can act as second messengers in a system.

In one embodiment, the reporter molecule of the invention is an oligonucleotide primer, template, or probe, which can be used to modulate the amplification of additional nucleic acid sequences, for example, sequences comprising reporter molecules, target signaling molecules, effector molecules, inhibitor molecules, and/or additional nucleic acid sensor molecules of the instant invention.

By “sensor component” of the nucleic acid sensor molecule is meant, a molecule such as a nucleic acid sequence (e.g., RNA or DNA or analogs thereof), peptide, or other chemical moiety which can interact with one or more regions of the enzymatic nucleic acid component of the nucleic acid sensor molecule to modulate, such as inhibit or activate, the catalytic activity of the nucleic acid sensor molecule. In the presence of a signaling agent, the ability of the sensor component, for example, to modulate the catalytic activity of the enzymatic nucleic acid component is inhibited or diminished. The sensor component can comprise recognition properties relating to chemical or physical signals capable of modulating the enzymatic nucleic acid component via chemical or physical changes to the structure of the nucleic acid sensor molecule. The sensor component can be derived from a naturally occurring nucleic acid protein binding sequence, for example RNAs that bind to proteins and/or nucleic acid molecules associated with nuerodegenerative diseases such as Alzheimer's disease, for example APP, BACE, or ps-2 peptides, proteins, DNA, or RNA. The sensor component can also be derived from a nucleic acid sequence that is obtained through in vitro or in vivo selection techniques as are know in the art. Such sequences or “aptamers” can be designed to bind a specific protein, peptide, nucleic acid, co-factor, metabolite, drug, or other small molecule with varying affinity. The sensor component can be covalently linked to the nucleic acid sensor molecule, or can be non-covalently associated. A person skilled in the art will recognize that all that is required is that the sensor component is able to selectively inhibit the activity of the nucleic acid sensor molecule.

In a preferred embodiment the linker region, when present in the nucleic acid sensor molecule and/or reporter molecule is further comprised of nucleotide, non-nucleotide chemical moieties or combinations thereof. Non-limiting examples of non-nucleotide chemical moieties can include ester, anhydride, amide, nitrile, and/or phosphate groups.

By “nucleic acid circuit” or “nucleic acid-based circuit” is meant an electronic circuit comprising one or more nucleic acids or oligonucleotides.

By “nucleic acid computer” or “nucleic acid-based computer” is meant a computing device or system comprising one or more nucleic acids or oligonucleotides. The nucleic acid computer can be used to interface biological systems, control other devices, or can be utilized to solve problems and/or manipulate data. Furthermore, the nucleic acid computer can comprise nucleic acid circuits.

By “predetermined RNA molecule” is meant a particular RNA molecule of known sequence, such as a cellular RNA, viral RNA, messenger RNA, transfer RNA, ribosomal RNA etc.

By “detectable response” is meant a chemical or physical property that can be measured, including, but not limited to changes in temperature, pH, frequency, charge, capacitance, or changes in fluorescent, chromogenic, radioactive, enzymatic and/or chemiluminescent levels or properties that can then be detected using standard methods known in the art.

By “single stranded RNA” (ssRNA) is meant a naturally occurring or synthetic ribonucleic acid molecule comprising a linear single strand, for example a ssRNA can be a messenger RNA (mRNA), transfer RNA (tRNA), ribosomal RNA (rRNA) etc. of a gene associated with a neurodegenerative disease, such as BACE, APP, or ps-2.

By “single stranded DNA” (ssDNA) is meant a naturally occurring or synthetic deoxyribonucleic acid molecule comprising a linear single strand, for example, a ssDNA can be a sense or antisense gene sequence, single nucleotide polymorphism (SNP), or EST (Expressed Sequence Tag) associated with a neurodegenerative disease, such as Alzheimer's disease, such as an EST or SNP associated with familial early onset Alzheimer's disease.

By “predetermined target” is meant a signaling agent or target signaling agent that is chosen to interact with a nucleic acid sensor molecule to generate a detectable response, for example a protein, peptide, RNA or DNA associated with a neurodegenerative disease such as Alzheimer's disease.

By “validate a predetermined gene target” is meant to confirm that a particular gene is associated with a specific phenotype, disease, or biological function in a system. Once the relationship between a gene and its function or resulting phenotype is determined, the gene can be targeted to modulate the activity of the gene.

By “validate a predetermined RNA target” is meant to confirm that a particular RNA transcript of a gene or other RNA is associated with a specific phenotype, disease, or biological function in a system, for example Alzheimer's disease or the presence of beta-amyloid protein. Once the relationship between the RNA and its function or resulting phenotype is determined, the RNA can be targeted to modulate the activity of the RNA or the gene encoding the RNA.

By “validate a predetermined peptide target” is meant to confirm that a particular peptide is associated with a specific phenotype, disease, or biological function in a system, for example Alzheimer's disease or the presence of beta-amyloid protein. Once the relationship between the peptide and its function or resulting phenotype is determined, the peptide or RNA encoding the peptide can be targeted to modulate the activity of the peptide or the gene encoding the peptide.

By “validate a predetermined protein target” is meant to confirm that a particular protein is associated with a specific phenotype, disease, or biological function in a system, for example Alzheimer's disease or the presence of beta-amyloid protein. Once the relationship between the protein and its function or resulting phenotype is determined, the protein or RNA encoding the protein can be targeted to modulate the activity of the protein or the gene encoding the protein.

By “validate a predetermined SNP target” is meant to confirm that a particular SNP of a gene is associated with a specific phenotype, disease, or biological function in a system, for example Alzheimer's disease or the presence of beta-amyloid protein. Once the relationship between the SNP and its function, associated gene function, or resulting phenotype is determined, the SNP can be targeted to modulate the activity of the SNP or the gene associated with the SNP.

By “SNP scoring” is meant a process of identifying and measuring the presence of SNPs in a genome, for example SNPs associated with neurodegenerative disease, such as Alzheimer's disease. SNP scoring can also refer to a system of ranking single nucleotide polymorphisms in terms of the relationship between a particular SNP and a certain disease state such as Alzheimer's disease or drug response in an organism, for example a human. SNP scoring can be used in determining the genotype of an organism.

By “SNP” is meant a single nucleotide polymorphism as is known in the art to include single nucleotide substitutions or mismatches in a genome (see Brookes, 1999, Gene, 234, 177-186; Stephens, 1999, Molecular Diagnosis, 4, 309-317). SNPs can be used to identify genes and gene functions as well as to characterize a genotype.

By “proteome” is meant the complete set of proteins found in a particular system, such as a cell or organism, for example a human cell or human.

By “proteome map” is meant the functional relationship between different protein constituents of a proteome.

By “proteome scoring” is meant a process of identifying and measuring the presence of proteins in a proteome. Proteome scoring can also refer to a system of ranking protiens in terms of the relationship between a particular protein and a certain disease state or drug response in an organism, for example a human. Proteome scoring can be used in determining the phenotype of an organism.

By “disease specific proteome” is meant a proteome associated with a particular disease or condition.

By “treatment specific proteome” is meant a proteome associated with a particular treatment or therapy.

By “complementarity” is meant that a nucleic acid can form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., ribozyme cleavage, antisense or triple helix inhibition. Determination of binding free energies for nucleic acid molecules is well known in the art (see, e.g., Turner et al., 1987, CSH Symp. Quant. Biol. LII pp. 123-133; Frier et al., 1986, Proc. Nat. Acad. Sci. USA 83:9373-9377; Turner et al., 1987, J. Am. Chem. Soc. 109:3783-3785.) A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

At least seven basic varieties of naturally-occurring enzymatic RNAs are known presently. Each can catalyze the hydrolysis of RNA phosphodiester bonds in trans (and thus can cleave other RNA molecules) under physiological conditions. Table I summarizes some of the characteristics of these ribozymes. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets. Thus, a single ribozyme molecule is able to cleave many molecules of target RNA. In addition, the ribozyme is a highly specific inhibitor of gene expression, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of a ribozyme.

The enzymatic nucleic acid molecules that cleave the specified sites in BACE-specific RNAs and/or ps-2-specific RNAs represent a novel therapeutic approach to treat a variety of pathologic indications, including Alzheimer's disease and dementia.

In one of the embodiments of the inventions described herein, the enzymatic nucleic acid molecule is formed in a hammerhead or hairpin motif, but may also be formed in the motif of a hepatitis delta virus, group I intron, group II intron or RNase P RNA (in association with an RNA guide sequence), Neurospora VS RNA, DNAzymes, NCH cleaving motifs, or G-cleavers. Examples of such hammerhead motifs are described by Dreyfus, supra, Rossi et al., 1992, AIDS Research and Human Retroviruses 8, 183. Examples of hairpin motifs by Hampel et al., EP0360257; Hampel and Tritz, 1989 Biochemistry 28, 4929; Feldstein et al., 1989, Gene 82, 53; Haseloff and Gerlach, 1989, Gene, 82, 43; Hampel et al., 1990 Nucleic Acids Res. 18, 299; Chowrira & McSwiggen, U.S. Pat. No. 5,631,359. Examples of the hepatitis delta virus motif is described by Perrotta and Been, 1992 Biochemistry 31, 16. The RNase P motif is described by Guerrier-Takada et al., 1983 Cell 35, 849; Forster and Altman, 1990, Science 249, 783; Li and Altman, 1996, Nucleic Acids Res. 24, 835. Neurospora VS RNA ribozyme motif is described by Collins (Saville and Collins, 1990 Cell 61, 685-696; Saville and Collins, 1991 Proc. Natl. Acad. Sci. USA 88, 8826-8830; Collins and Olive, 1993 Biochemistry 32, 2795-2799; Guo and Collins, 1995, EMBO. J. 14, 363). Group II introns are described by Griffin et al., 1995, Chem. Biol. 2, 761; Michels and Pyle, 1995, Biochemistry 34, 2965; Pyle et al., International PCT Publication No. WO 96/22689. The Group I intron motif is described by Cech et al., U.S. Pat. No. 4,987,071 and of DNAzymes by Usman et al., International PCT Publication No. WO 95/11304; Chartrand et al., 1995, NAR 23, 4092; Breaker et al., 1995, Chem. Bio. 2, 655; Santoro et al., 1997, PNAS 94, 4262. NCH cleaving motifs are described in Ludwig & Sproat, International PCT Publication No. WO 98/58058; and G-cleavers are described in Kore et al., 1998, Nucleic Acids Research 26, 4116-4120 and Eckstein et al., International PCT Publication No. WO 99/16871. Additional motifs such as the Aptazyme (Breaker et al., WO 98/43993), Amberzyme (FIG. 3; Beigelman et al., U.S. Ser. No. 09/301,511) and Zinzyme (Beigelman et al., U.S. Ser. No. 09/301,511). All these references are incorporated by reference herein, including drawings. Any of these motifs can be used in the present invention. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule (Cech et al., U.S. Pat. No. 4,987,071).

In certain embodiments of the present invention, a nucleic acid molecule, e.g., an enzymatic nucleic acid molecule, allozyme, antisense molecule, or triplex DNA, is 13 to 100 nucleotides in length, e.g., in specific embodiments 35, 36, 37, or 38 nucleotides in length (e.g., for particular ribozymes or antisense). In particular embodiments, the nucleic acid molecule is 15-100, 17-100, 20-100, 21-100, 23-100, 25-100, 27-100, 30-100, 32-100, 35-100, 40-100, 50-100, 60-100, 70-100, or 80-100 nucleotides in length. Instead of 100 nucleotides being the upper limit on the length ranges specified above, the upper limit of the length range can be, for example, 30, 40, 50, 60, 70, or 80 nucleotides. Thus, for any of the length ranges, the length range for particular embodiments has lower limit as specified, with an upper limit as specified which is greater than the lower limit. For example, in a particular embodiment, the length range can be 35-50 nucleotides in length. All such ranges are expressly included. Also in particular embodiments, a nucleic acid molecule can have a length which is any of the lengths specified above, for example, 21 nucleotides in length.

In one embodiment, the invention provides a method for producing a class of nucleic acid-based gene-inhibiting agents which exhibit a high degree of specificity for the RNA of a desired target. For example, the enzymatic nucleic acid molecule is preferably targeted to a highly conserved sequence region of target RNAs encoding BACE proteins (specifically BACE gene) such that specific treatment of a disease or condition can be provided with either one or several nucleic acid molecules of the invention. Such nucleic acid molecules can be delivered exogenously to specific tissue or cellular targets as required. Alternatively, the nucleic acid molecules (e.g., ribozymes and antisense) can be expressed from DNA and/or RNA vectors that are delivered to specific cells.

By “BACE proteins” is meant, a protein or a mutant protein derivative thereof, comprising secretase associated proteolytic cleavage activity of APP. In particular embodiments, the BACE protein can be referred to by other names used to describe a β-secretase, such as Asp2 (Gurney, 1999, Nature, 402, 533-537).

By “highly conserved sequence region” is meant, a nucleotide sequence of one or more regions in a target gene does not vary significantly from one generation to the other or from one biological system to the other as understood by those skilled in the art.

The nucleic acid-based inhibitors of BACE, ps-2, or APP expression are useful for the prevention of the diseases and conditions Alzheimer's disease, dementia, and any other diseases or conditions that are related to the levels of BACE, ps-2, or APP in a cell or tissue. Thus, the reduction of BACE, ps-2, or APP expression (specifically BACE, ps-2, or APP gene RNA levels} and thus reduction in the level of the respective protein relieves, to some extent, the symptoms of the disease or condition.

The nucleic acid-based inhibitors of the invention can be added directly, or can be complexed with cationic lipids, packaged within liposomes, or otherwise delivered to target cells or tissues. The nucleic acid or nucleic acid complexes can be locally administered to relevant tissues ex vivo, or in vivo through injection, infusion pump or stent, with or without their incorporation in biopolymers. In preferred embodiments, the enzymatic nucleic acid inhibitors comprise sequences, which are complementary to the substrate sequences in Tables III to VIII. Examples of such enzymatic nucleic acid molecules also are shown in Tables III to VIII. Examples of such enzymatic nucleic acid molecules consist essentially of sequences defined in these Tables.

In yet another embodiment, the invention features antisense nucleic acid molecules and 2-5A chimera including sequences complementary to the substrate sequences shown in Tables III to VIII. Such nucleic acid molecules can include sequences as shown for the binding arms of the enzymatic nucleic acid molecules in Tables III to VIII. Similarly, triplex molecules can be provided targeted to the corresponding DNA target regions, and containing the DNA equivalent of a target sequence or a sequence complementary to the specified target (substrate) sequence. Typically, antisense molecules will be complementary to a target sequence along a single contiguous sequence of the antisense molecule. However, in certain embodiments, an antisense molecule may bind to substrate such that the substrate molecule forms a loop, and/or an antisense molecule may bind such that the antisense molecule forms a loop. Thus, the antisense molecule may be complementary to two (or even more) non-contiguous substrate sequences or two (or even more) non-contiguous sequence portions of an antisense molecule may be complementary to a target sequence or both.

By “consists essentially of” is meant that the active enzymatic nucleic acid molecule of the invention contains an enzymatic center or core equivalent to those in the examples, and binding arms able to bind RNA such that cleavage at the target site occurs. Other sequences may be present which do not interfere with such cleavage. Thus, a core region may, for example, include one or more loop or stem-loop structures, which do not prevent enzymatic activity. Such sequences can be designated as “X”, for example, as in a loop or stem/loop structure. For example, a core sequence for a hammerhead enzymatic nucleic acid can be 5′-CUGAUGAG-3′ and 5′-CGAA-3′ connected by “X”, where X=5′-GCCGUUAGGC-3′ (SEQ ID NO: 4550), or any other stem II region known in the art. Similarly, for other enzymatic nucleic acid molecules of the instant invention, additional sequences may be present that do not interfere with the function of the nucleic acid molecule.

In another embodiment of the invention, ribozymes or antisense molecules that cleave target RNA molecules or inhibit the Alzheimer's disease related genes identified above are expressed from transcription units inserted into DNA or RNA vectors. Preferably, ribozymes or antisense molecules that cleave BACE, ps-2, or APP (preferably BACE, ps-2, or APP gene) activity are expressed from transcription units inserted into DNA or RNA vectors. The recombinant vectors are preferably DNA plasmids or viral vectors. Ribozyme or antisense expressing viral vectors can be constructed based on, but not limited to, adeno-associated virus, retrovirus, adenovirus, or alphavirus. Preferably, the recombinant vectors capable of expressing the ribozymes or antisense are delivered as described above, and persist in target cells. Alternatively, viral vectors may be used that provide for transient expression of ribozymes or antisense. Such vectors can be repeatedly administered as necessary. Once expressed, the ribozymes or antisense bind to the target RNA and inhibit its function or expression. Delivery of ribozyme or antisense expressing vectors can be systemic, such as by intravenous or intramuscular administration, by administration to target cells ex-planted from the patient followed by reintroduction into the patient, or by any other means that would allow for introduction into the desired target cell.

By “vectors” is meant any nucleic acid- and/or viral-based technique used to deliver a desired nucleic acid.

By “patient” is meant an organism, which is a donor or recipient of explanted cells or the cells themselves. “Patient” also refers to an organism to which the nucleic acid molecules of the invention can be administered. Preferably, a patient is a mammal or mammalian cells. More preferably, a patient is a human or human cells.

The nucleic acid molecules of the instant invention, individually, or in combination or in conjunction with other drugs, can be used to treat diseases or conditions discussed above. For example, to treat a disease or condition associated with the levels of BACE, the patient may be treated, or other appropriate cells may be treated, as is evident to those skilled in the art, individually or in combination with one or more drugs under conditions suitable for the treatment.

In a further embodiment, the described molecules, such as antisense or ribozymes, can be used in combination with other known treatments to treat conditions or diseases discussed above. For example, the described molecules could be used in combination with one or more known therapeutic agents to treat Alzheimer's disease and dementia.

In another embodiment, the invention features nucleic acid-based inhibitors (e.g., enzymatic nucleic acid molecules (ribozymes), antisense nucleic acids, 2-5A antisense chimeras, triplex DNA, antisense nucleic acids containing RNA cleaving chemical groups) and methods for their use to down regulate or inhibit the expression of genes (e.g., BACE) capable of progression and/or maintenance of Alzheimer's disease.

In one embodiment, the invention features nucleic acid-based techniques (e.g., enzymatic nucleic acid molecules (ribozymes), antisense nucleic acids, 2-5A antisense chimeras, triplex DNA, antisense nucleic acids containing RNA cleaving chemical groups) and methods for their use to down regulate or inhibit the expression of BACE, ps-2, or APP gene expression.

By “comprising” is meant including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Other features and advantages of the invention will be apparent from the following description of the preferred embodiments thereof, and from the claims.

### BRIEF DESCRIPTION OF THE DRAWINGS

FIG. 1 shows the secondary structure model for seven different classes of enzymatic nucleic acid molecules. Arrow indicates the site of cleavage. --------- indicate the target sequence. Lines interspersed with dots are meant to indicate tertiary interactions. - is meant to indicate base-paired interaction. Group I Intron: P1-P9.0 represent various stem-loop structures (Cech et al., 1994, Nature Struc. Bio., 1, 273). RNase P (M1RNA): EGS represents external guide sequence (Forster et al., 1990, Science, 249, 783; Pace et al., 1990, J Biol. Chem., 265, 3587). Group II Intron: 5′SS means 5′ splice site; 3′SS means 3′-splice site; IBS means intron binding site; EBS means exon binding site (Pyle et al., 1994, Biochemistry, 33, 2716). VS RNA: I-VI are meant to indicate six stem-loop structures; shaded regions are meant to indicate tertiary interaction (Collins, International PCT Publication No. WO 96/19577). HDV Ribozyme: : I-IV are meant to indicate four stem-loop structures (Been et al., U.S. Pat. No. 5,625,047). Hammerhead Ribozyme: I-III are meant to indicate three stem-loop structures; stems I-III can be of any length and may be symmetrical or asymmetrical (Usman et al., 1996, Curr. Op. Struct. Bio., 1, 527). Hairpin Ribozyme: Helix 1, 4 and 5 can be of any length; Helix 2 is between 3 and 8 base-pairs long; Y is a pyrimidine; Helix 2 (H2) is provided with a least 4 base pairs (i.e., n is 1, 2, 3 or 4) and helix 5 can be optionally provided of length 2 or more bases (preferably 3-20 bases, i.e., m is from 1-20 or more). Helix 2 and helix 5 may be covalently linked by one or more bases (i.e., r is ≧1 base). Helix 1, 4 or 5 may also be extended by 2 or more base pairs (e.g., 4-20 base pairs) to stabilize the ribozyme structure, and preferably is a protein binding site. In each instance, each N and N′ independently is any normal or modified base and each dash represents a potential base-pairing interaction. These nucleotides may be modified at the sugar, base or phosphate. Complete base-pairing is not required in the helices, but is preferred. Helix 1 and 4 can be of any size (i.e., o and p is each independently from 0 to any number, e.g., 20) as long as some base-pairing is maintained. Essential bases are shown as specific bases in the structure, but those in the art will recognize that one or more may be modified chemically (abasic, base, sugar and/or phosphate modifications) or replaced with another base without significant effect. Helix 4 can be formed from two separate molecules, i.e., without a connecting loop. The connecting loop when present may be a ribonucleotide with or without modifications to its base, sugar or phosphate. “q” ≧is 2 bases. The connecting loop can also be replaced with a non-nucleotide linker molecule. H refers to bases A, U, or C. Y refers to pyrimidine bases. (Burke et al., 1996, Nucleic Acids & Mol. Biol., 10, 129; Chowrira et al., U.S. Pat. No. 5,631,359).

FIG. 2 shows examples of chemically stabilized ribozyme motifs. HH Rz, represents hammerhead ribozyme motif (Usman et al., 1996, Curr. Op. Struct. Bio., 1, 527); NCH Rz represents the NCH ribozyme motif (Ludwig & Sproat, International PCT Publication No. WO 98/58058); G-Cleaver, represents G-cleaver ribozyme motif (Kore et al., 1998, Nucleic Acids Research 26, 4116-4120). N or n, represent independently a nucleotide which may be same or different and have complementarity to each other; rI, represents ribo-Inosine nucleotide; arrow indicates the site of cleavage within the target. Position 4 of the HH Rz and the NCH Rz is shown as having 2′-C-allyl modification, but those skilled in the art will recognize that this position can be modified with other modifications well known in the art, so long as such modifications do not significantly inhibit the activity of the ribozyme.

FIG. 3 shows an example of the Amberzyme ribozyme motif that is chemically stabilized (see for example Beigelman et al., U.S. Ser. No. 09/301,511, incorporated by reference herein; also referred to as Class I Motif).

FIG. 4 shows an example of the Zinzyme ribozyme motif that is chemically stabilized (see for example Beigelman et al., U.S. Ser. No. 09/301,511, incorporated by reference herein; also referred to as Class A or Class II Motif).

FIG. 5 shows an example of a DNAzyme motif described by Santoro et al., 1997, PNAS, 94, 4262.

FIG. 6 shows a non-limiting example of a halfzyme enzymatic nucleic acid molecule of the invention. (a) The halfzyme is engineered by removing a portion of the enzymatic nucleic acid molecule (in this case Zinzyme) required for the activity of the enzymatic nucleic acid molecule. (b) A target molecule is used which allows the halfzyme to become active.

FIG. 7 shows a non-limiting example of a nucleic acid sensor molecule assay of the invention. Interaction of the target molecule with the sensor portion of the nucleic acid sensor molecule results in the activation of the nucleic acid sensor molecule. Detection of the chemical reaction catalyzed by the nucleic acid sensor molecule, for example cleavage of a reporter molecule, provides a signal that can be assayed.

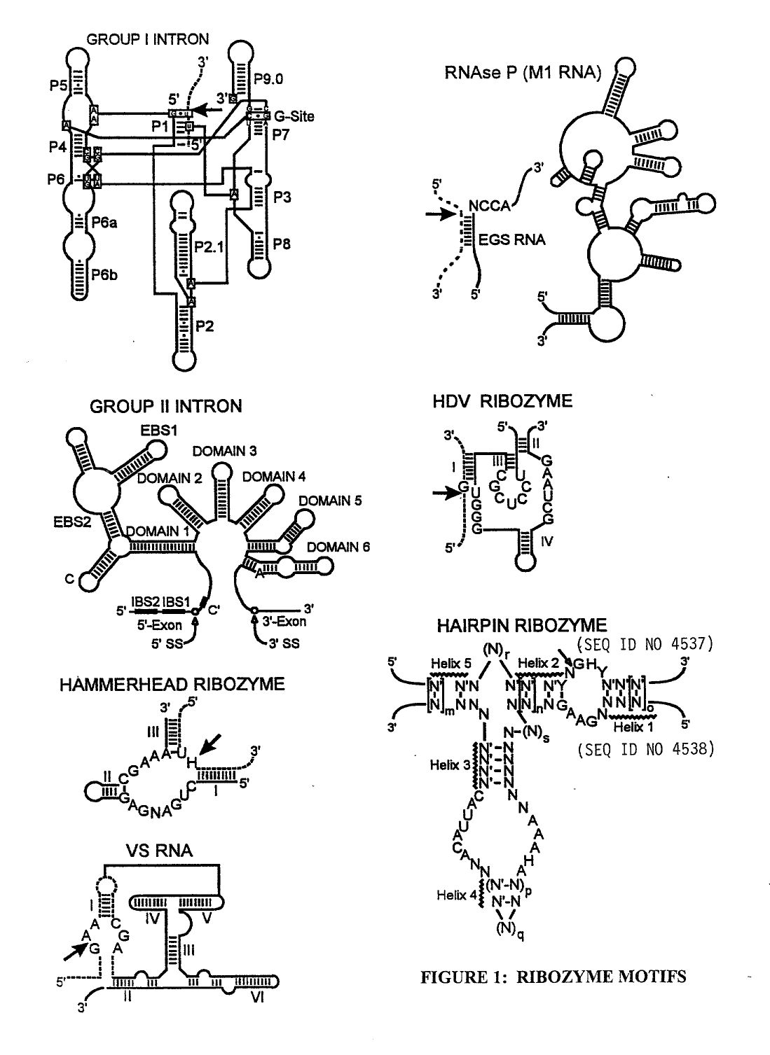

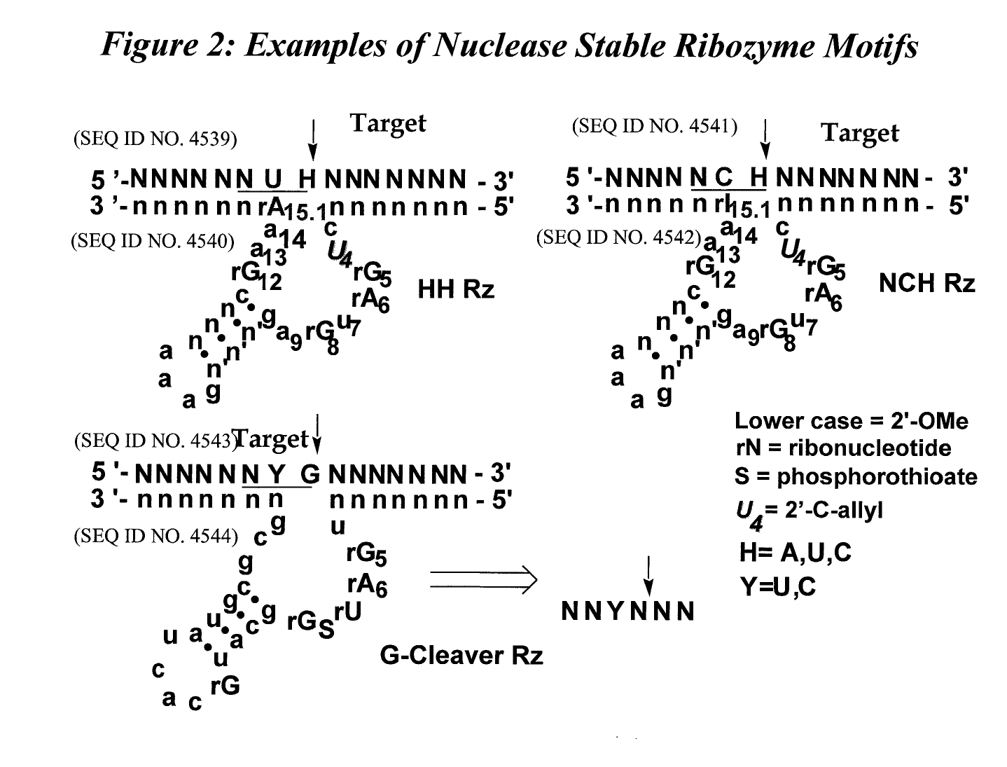

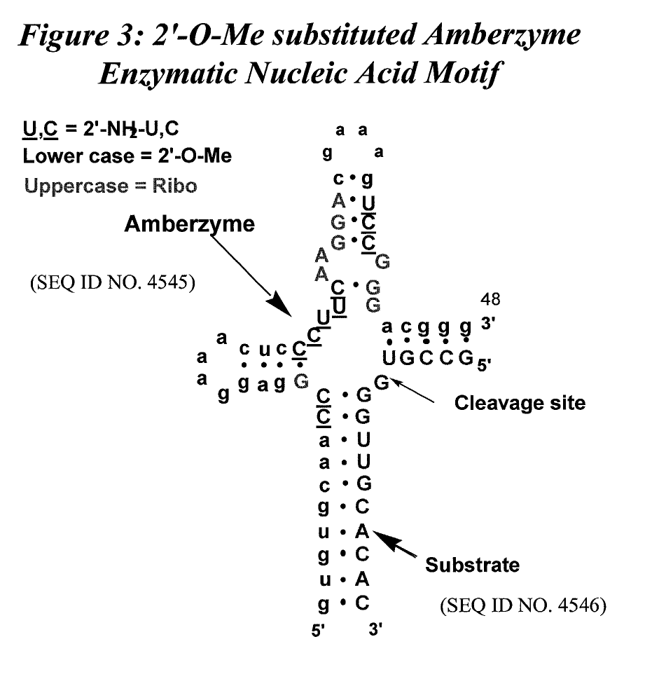

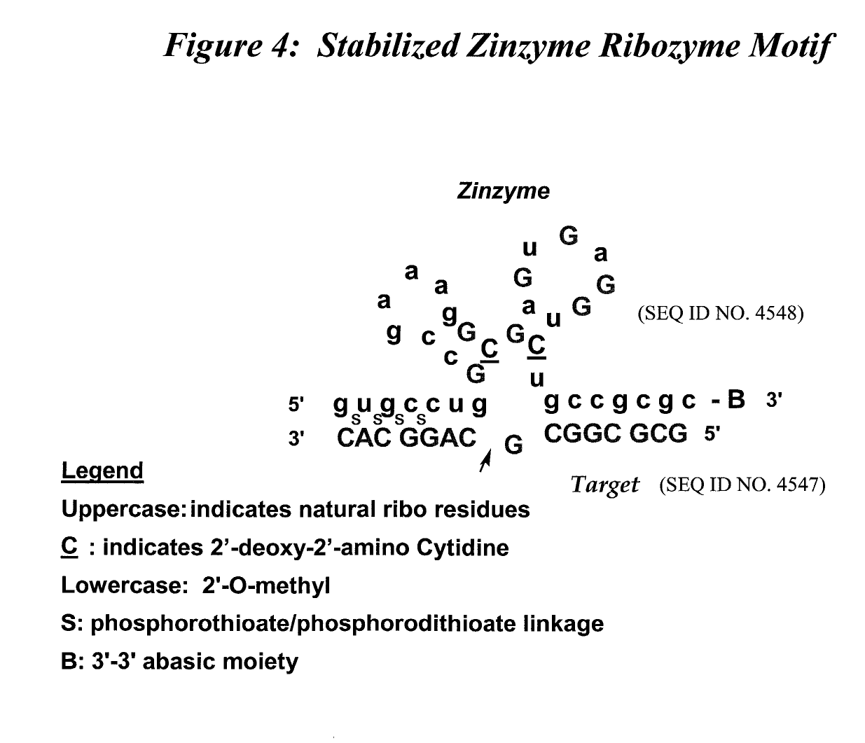

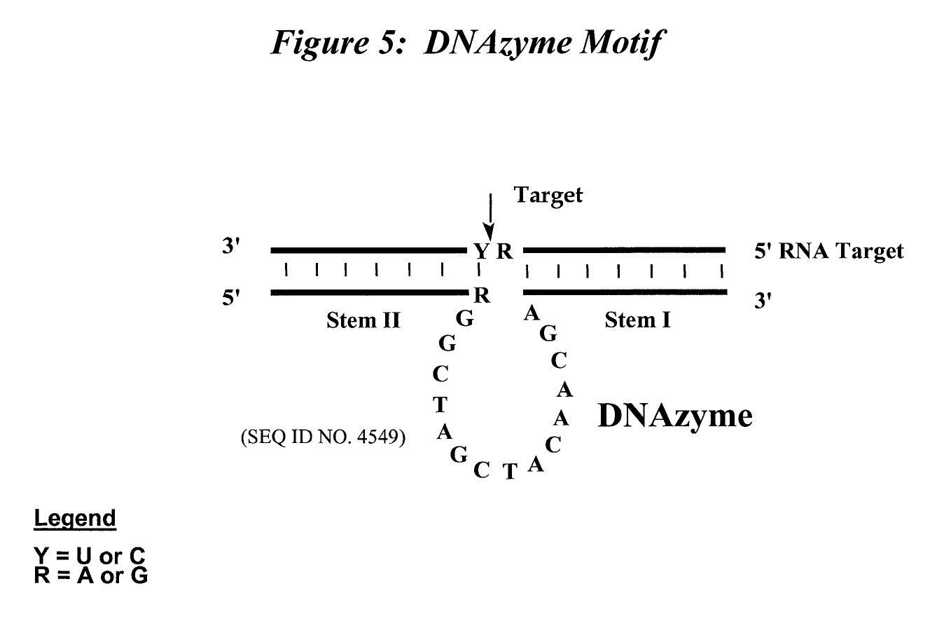

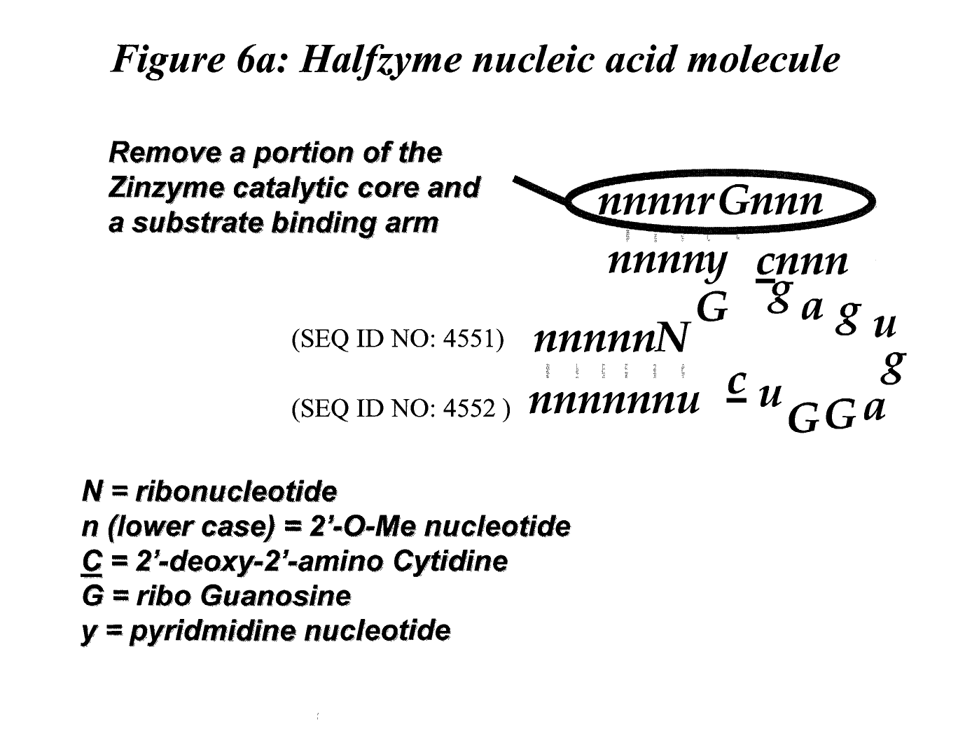

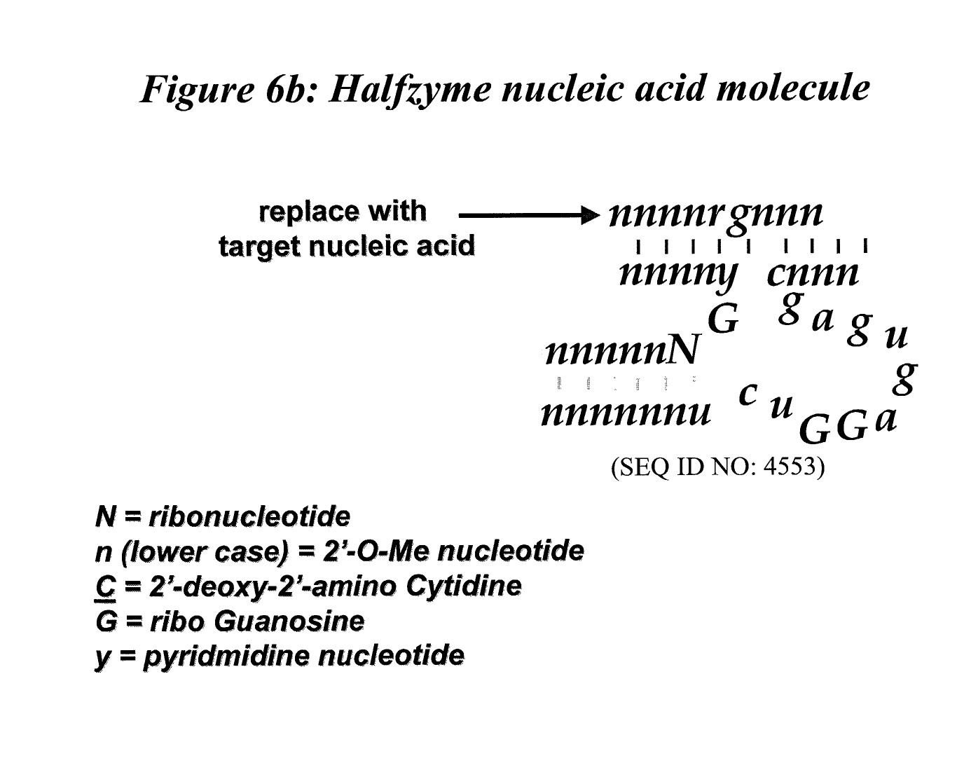

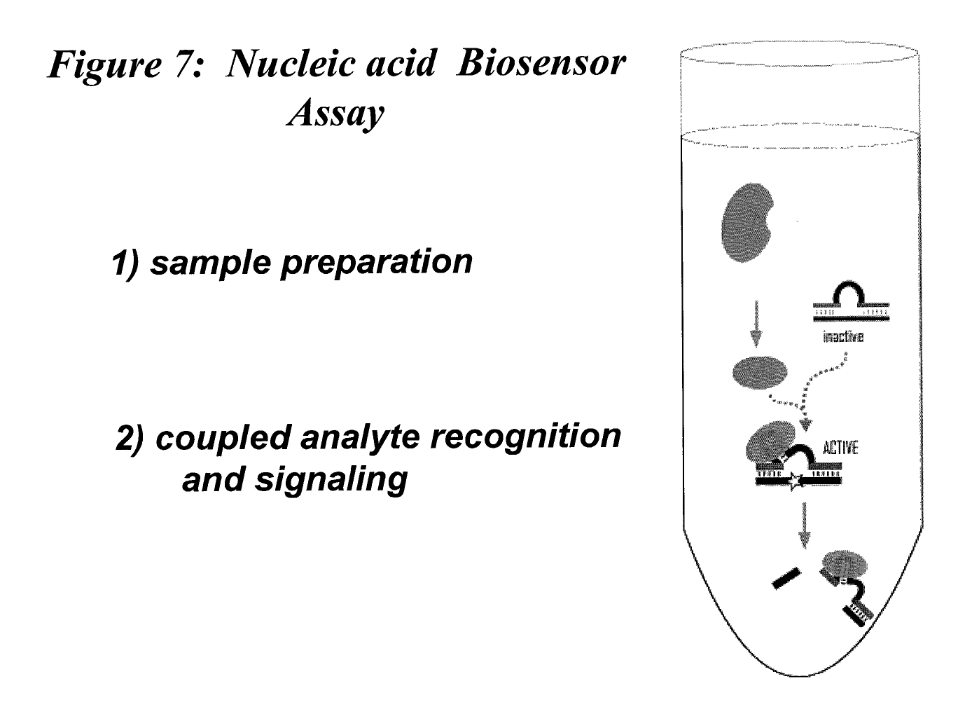

### DESCRIPTION OF THE PREFERRED EMBODIMENTS

Mechanism of Action of Nucleic Acid Molecules of the Invention

Antisense:

Antisense molecules may be modified or unmodified RNA, DNA, or mixed polymer oligonucleotides and primarily function by specifically binding to matching sequences resulting in inhibition of peptide synthesis (Wu-Pong, Nov 1994, BioPharm, 20-33). The antisense oligonucleotide binds to target RNA by Watson Crick base-pairing and blocks gene expression by preventing ribosomal translation of the bound sequences either by steric blocking or by activating RNase H enzyme. Antisense molecules may also alter protein synthesis by interfering with RNA processing or transport from the nucleus into the cytoplasm (Mukhopadhyay & Roth, 1996, Crit. Rev. in Oncogenesis 7, 151-190).

In addition, binding of single stranded DNA to RNA may result in nuclease degradation of the heteroduplex (Wu-Pong, supra; Crooke, supra). To date, the only backbone modified DNA chemistry which will act as substrates for RNase H are phosphorothioates, phosphorodithioates, and borontrifluoridates. Recently it has been reported that 2′-arabino and 2′-fluoro arabino-containing oligos can also activate RNase H activity.

A number of antisense molecules have been described that utilize novel configurations of chemically modified nucleotides, secondary structure, and/or RNase H substrate domains (Woolf et al., International PCT Publication No. WO 98/13526; Thompson et al., U.S. Ser. No. 60/082,404 which was filed on Apr. 20, 1998; Hartmann et al., U.S. Ser. No. 60/101,174 which was filed on Sep. 21, 1998) all of these are incorporated by reference herein in their entirety.

Triplex Forming Oligonucleotides (TFO):

Single stranded DNA may be designed to bind to genomic DNA in a sequence specific manner. TFOs are comprised of pyrimidine-rich oligonucleotides which bind DNA helices through Hoogsteen Base-pairing (Wu-Pong, supra). The resulting triple helix composed of the DNA sense, DNA antisense, and TFO disrupts RNA synthesis by RNA polymerase. The TFO mechanism may result in gene expression or cell death since binding may be irreversible (Mukhopadhyay & Roth, supra)

2-5A Antisense Chimera:

The 2-5A system is an interferon-mediated mechanism for RNA degradation found in higher vertebrates (Mitra et al., 1996, Proc Nat Acad Sci USA 93, 6780-6785). Two types of enzymes, 2-5A synthetase and RNase L, are required for RNA cleavage. The 2-5A synthetases require double stranded RNA to form 2′-5′ oligoadenylates (2-5A). 2-5A then acts as an allosteric effector for utilizing RNase L which has the ability to cleave single stranded RNA. The ability to form 2-5A structures with double stranded RNA makes this system particularly useful for inhibition of viral replication.

(2′-5′) oligoadenylate structures may be covalently linked to antisense molecules to form chimeric oligonucleotides capable of RNA cleavage (Torrence, supra). These molecules putatively bind and activate a 2-5A dependent RNase, the oligonucleotide/enzyme complex then binds to a target RNA molecule which can then be cleaved by the RNase enzyme.

Enzymatic Nucleic Acid:

Seven basic varieties of naturally-occurring enzymatic RNAs are presently known. In addition, several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al., 1993, Science 261:1411-1418; Szostak, 1993, TIBS 17, 89-93; Kumar et al., 1995, FASEB J, 9, 1183; Breaker, 1996, Curr. Op. Biotech., 7, 442; Santoro et al., 1997, Proc. Natl. Acad. Sci., 94, 4262; Tang et al., 1997, RNA 3, 914; Nakamaye & Eckstein, 1994, supra; Long & Uhlenbeck, 1994, supra; Ishizaka et al., 1995, supra; Vaish et al., 1997, Biochemistry 36, 6495; all of these are incorporated by reference herein). Each can catalyze a series of reactions including the hydrolysis of phosphodiester bonds in trans (and thus can cleave other RNA molecules) under physiological conditions.

Nucleic acid molecules of this invention will block to some extent BACE protein expression and can be used to treat disease or diagnose disease associated with the levels of BACE.

The enzymatic nature of a ribozyme has significant advantages, such as the concentration of ribozyme necessary to affect a therapeutic treatment is lower. This advantage reflects the ability of the ribozyme to act enzymatically. Thus, a single ribozyme molecule is able to cleave many molecules of target RNA. In addition, the ribozyme is a highly specific inhibitor, with the specificity of inhibition depending not only on the base-pairing mechanism of binding to the target RNA, but also on the mechanism of target RNA cleavage. Single mismatches, or base-substitutions, near the site of cleavage can be chosen to completely eliminate catalytic activity of a ribozyme.

Nucleic acid molecules having an endonuclease enzymatic activity are able to repeatedly cleave other separate RNA molecules in a nucleotide base sequence-specific manner. With the proper design, such enzymatic nucleic acid molecules can be targeted to RNA transcripts, and achieve efficient cleavage in vitro (Zaug et al., 324, Nature 429 1986; Uhlenbeck, 1987 Nature 328, 596; Kim et al., 84 Proc. Natl. Acad. Sci. USA 8788, 1987; Dreyfus, 1988, Einstein Quart. J Bio. Med., 6, 92; Haseloff and Gerlach, 334 Nature 585, 1988; Cech, 260 JAMA 3030, 1988; and Jefferies et al., 17 Nucleic Acids Research 1371, 1989; Santoro et al., 1997 supra).

Because of their sequence specificity, trans-cleaving ribozymes show promise as therapeutic agents for human disease (Usman & McSwiggen, 1995 Ann. Rep. Med. Chem. 30, 285-294; Christoffersen and Marr, 1995 J Med. Chem. 38, 2023-2037). Ribozymes can be designed to cleave specific RNA targets within the background of cellular RNA. Such a cleavage event renders the RNA non-functional and abrogates protein expression from that RNA. In this manner, synthesis of a protein associated with a disease state can be selectively inhibited (Warashina et al., 1999, Chemistry and Biology, 6, 237-250.

Enzymatic nucleic acid molecules of the invention that are allosterically regulated (“allozymes”) can be used to down-regulate the expression of genes associated with the maintenance and/or progression of Alzheimer's disease, for example BACE, presenilin-2 (ps-2), or amyloid precursor protein (APP) expression. These allosteric enzymatic nucleic acids or allozymes (see for example George et al., U.S. Pat. Nos. 5,834,186 and 5,741,679, Shih et al., U.S. Pat. No. 5,589,332, Nathan et al, U.S. Pat. No 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842) are designed to respond to a signaling agent, for example, mutant BACE, ps-2, or APP protein, wild-type BACE, ps-2, or APP protein, mutant BACE, ps-2, or APP RNA, wild-type BACE, ps-2, or APP RNA, other proteins and/or RNAs involved in BACE, ps-2, or APP activity, compounds, metals, polymers, molecules and/or drugs that are targeted to BACE, ps-2, or APP expressing cells etc., which in turn modulates the activity of the enzymatic nucleic acid molecule. In response to interaction with a predetermined signaling agent, the allosteric enzymatic nucleic acid molecule's activity is activated or inhibited such that the expression of a particular target is selectively down-regulated. The target can comprise wild-type BACE, ps-2, or APP, mutant BACE, ps-2, or APP, a component of BACE, ps-2, or APP, and/or a predetermined cellular component that modulates BACE, ps-2, or APP activity. In a specific example, allosteric enzymatic nucleic acid molecules that are activated by interaction with a RNA encoding BACE, ps-2, or APP protein are used as therapeutic agents in vivo. The presence of RNA encoding the BACE, ps-2, or APP protein activates the allosteric enzymatic nucleic acid molecule that subsequently cleaves the RNA encoding BACE, ps-2, or APP protein resulting in the inhibition of BACE, ps-2, or APP protein expression. In this manner, cells that express the BACE, ps-2, or APP protein are selectively targeted.

In another non-limiting example, an allozyme can be activated by a BACE, ps-2, or APP protein, peptide, or mutant polypeptide that caused the allozyme to inhibit the expression of BACE, ps-2, or APP gene, by, for example, cleaving RNA encoded by BACE, ps-2, or APP gene. In this non-limiting example, the allozyme acts as a decoy to inhibit the function of BACE, ps-2, or APP and also inhibit the expression of BACE, ps-2, or APP once activated by the BACE, ps-2, or APP protein.

Target Sites

Targets for useful ribozymes and antisense nucleic acids can be determined as disclosed in Draper et al., WO 93/23569; Sullivan et al., WO 93/23057; Thompson et al., WO 94/02595; Draper et al., WO 95/04818; McSwiggen et al., U.S. Pat. No. 5,525,468, all are hereby incorporated by reference herein in their totality. Other examples include the following PCT applications, which concern inactivation of expression of disease-related genes: WO 95/23225, WO 95/13380, WO 94/02595, all incorporated by reference herein. Rather than repeat the guidance provided in those documents here, specific examples of such methods are provided below, not limiting to those in the art. Ribozymes and antisense to such targets are designed as described in those applications and synthesized, to be tested in vitro and in vivo, as also described. The sequences of human BACE RNAs were screened for optimal enzymatic nucleic acid and antisense target sites using a computer-folding algorithm. Antisense, hammerhead, DNAzyme, NCH, amberzyme, zinzyme, or G-Cleaver ribozyme binding/cleavage sites were identified. These sites are shown in Tables III to VIII (all sequences are 5′ to 3′ in the tables; X can be any base-paired sequence, the actual sequence is not relevant here). The nucleotide base position is noted in the Tables as that site to be cleaved by the designated type of enzymatic nucleic acid molecule. Thus, the position that is cleaved is following the substrate nucleotide that is written separated from the sequences on either side. For example, in Table m, for Seq. ID No. 1, nucleotide position 9 is the central “C”, and cleavage occurs at or following that nucleotide. While human sequences can be screened and enzymatic nucleic acid molecule and/or antisense thereafter designed, as discussed in Stinchcomb et al., WO 95/23225, mouse targeted ribozymes may be useful to test efficacy of action of the enzymatic nucleic acid molecule and/or antisense prior to testing in humans.

Antisense, hammerhead, DNAzyme, NCH, amberzyme, zinzyme or G-Cleaver ribozyme binding/cleavage sites were identified. The nucleic acid molecules were individually analyzed by computer folding (Jaeger et al., 1989 Proc. Natl. Acad. Sci. USA, 86, 7706) to assess whether the sequences fold into the appropriate secondary structure. Those nucleic acid molecules with unfavorable intramolecular interactions such as between the binding arms and the catalytic core were eliminated from consideration. Varying binding arm lengths can be chosen to optimize activity.

Antisense, hammerhead, DNAzyme, NCH, amberzyme, zinzyme or G-Cleaver ribozyme binding/cleavage sites were identified and were designed to anneal to various sites in the RNA target. The binding arms are complementary to the target site sequences described above. The nucleic acid molecules were chemically synthesized. The method of synthesis used follows the procedure for normal DNA/RNA synthesis as described below and in Usman et al., 1987 J Am. Chem. Soc., 109, 7845; Scaringe et al., 1990 Nucleic Acids Res., 18, 5433; and Wincott et al., 1995 Nucleic Acids Res. 23, 2677-2684; Caruthers et al., 1992, Methods in Enzymology 211,3-19.

Synthesis of Nucleic Acid Molecules

Synthesis of nucleic acids greater than 100 nucleotides in length is difficult using automated methods, and the therapeutic cost of such molecules is prohibitive. In this invention, small nucleic acid motifs (“small refers to nucleic acid motifs no more than 100 nucleotides in length, preferably no more than 80 nucleotides in length, and most preferably no more than 50 nucleotides in length; e.g., antisense oligonucleotides, hammerhead or the hairpin ribozymes) are preferably used for exogenous delivery. The simple structure of these molecules increases the ability of the nucleic acid to invade targeted regions of RNA structure. Exemplary molecules of the instant invention were chemically synthesized, and others can similarly be synthesized. Oligodeoxyribonucleotides were synthesized using standard protocols as described in Caruthers et al., 1992, Methods in Enzymology 211, 3-19, and is incorporated herein by reference.

The method of synthesis used for normal RNA, including certain enzymatic nucleic acid molecules, follows the procedure as described in Usman et al., 1987, J Am. Chem. Soc., 109, 7845; Scaringe et al., 1990, Nucleic Acids Res., 18, 5433; Wincott et al., 1995, Nucleic Acids Res. 23, 2677-2684; and Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses were conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table II outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by calorimetric quantitation of the trityl fractions, were 97.5-99%. Other oligonucleotide synthesis reagents for the 394 Applied Biosystems, Inc. synthesizer; detritylation solution was 3% TCA in methylene chloride (ABI); capping was performed with 16% N-methyl imidazole in THF (ABI) and 10% acetic anhydride/10% 2,6-lutidine in THF (ABI); oxidation solution was 16.9 mM I₂, 49 mM pyridine, 9% water in THF (PERSEPTIVETM). Burdick & Jackson Synthesis Grade acetonitrile was used directly from the reagent bottle. S-Ethyltetrazole solution (0.25 M in acetonitrile) was made up from the solid obtained from American International Chemical, Inc.

Deprotection of the RNA was performed using either a two-pot or one-pot protocol. For the two-pot protocol, the polymer-bound trityl-on oligoribonucleotide was transferred to a 4 mL glass screw top vial and suspended in a solution of 40% aq. methylamine (1 mL) at 65° C. for 10 min. After cooling to −20° C., the supernatant was removed from the polymer support. The support was washed three times with 1.0 mL of EtOH:MeCN:H2O/3:1:1, vortexed and the supernatant was then added to the first supernatant. The combined supernatants, containing the oligoribonucleotide, were dried to a white powder. The base deprotected oligoribonucleotide was resuspended in anhydrous TEA/HF/NMP solution (300 μL of a solution of 1.5 mL N-methylpyrrolidinone, 750 μL TEA and 1 mL TEA.3HF to provide a 1.4 M HF concentration) and heated to 65° C. After 1.5 h, the oligomer was quenched with 1.5 M NH₄HCO₃.

Alternatively, for the one-pot protocol, the polymer-bound trityl-on oligoribonucleotide was transferred to a 4 mL glass screw top vial and suspended in a solution of 33% ethanolic methylamine/DMSO: 1/1 (0.8 mL) at 65° C. for 15 min. The vial was brought to r.t. TEA.3HF (0.1 mL) was added and the vial was heated at 65° C. for 15 min. The sample was cooled at −20° C. and then quenched with 1.5 M NH₄HCO₃.

For purification of the trityl-on oligomers, the quenched NH₄HCO₃ solution was loaded onto a C-18 containing cartridge that had been prewashed with acetonitrile followed by 50 mM TEAA. After washing the loaded cartridge with water, the RNA was detritylated with 0.5% TFA for 13 min. The cartridge was then washed again with water, salt exchanged with 1 M NaCl and washed with water again. The oligonucleotide was then eluted with 30% acetonitrile.

Inactive hammerhead ribozymes or binding attenuated control (BAC) oligonucleotides) were synthesized by substituting a U for G5 and a U for A₁₄ (numbering from Hertel, K. J., et al., 1992, Nucleic Acids Res., 20, 3252). Similarly, one or more nucleotide substitutions can be introduced in other enzymatic nucleic acid molecules to inactivate the molecule and such molecules can serve as a negative control.

The average stepwise coupling yields were >98% (Wincott et al., 1995 Nucleic Acids Res. 23, 2677-2684). Those of ordinary skill in the art will recognize that the scale of synthesis can be adapted to be larger or smaller than the example described above, including but not limited to 96 well format. All that is important is the ratio of chemicals used in the reaction.

Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention can be modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Ribozymes are purified by gel electrophoresis using general methods or are purified by high pressure liquid chromatography (HPLC; See Wincott et al., supra, the totality of which is hereby incorporated herein by reference) and are re-suspended in water.

The sequences of the ribozymes and antisense constructs that are chemically synthesized, useful in this study, are shown in Tables III to VIII. Those in the art will recognize that these sequences are representative only of many more such sequences where the enzymatic portion of the ribozyme (all but the binding arms) is altered to affect activity. The ribozyme and antisense construct sequences listed in Tables III to VIII may be formed of ribonucleotides or other nucleotides or non-nucleotides. Such ribozymes with enzymatic activity are equivalent to the ribozymes described specifically in the Tables. Optimizing Activity of the nucleic acid molecule of the invention.

Chemically synthesizing nucleic acid molecules with modifications (base, sugar and/or phosphate) that prevent their degradation by serum ribonucleases may increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules herein). All these publications are hereby incorporated by reference herein. Modifications which enhance their efficacy in cells, and removal of bases from nucleic acid molecules to shorten oligonucleotide synthesis times and reduce chemical requirements are desired.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al, 1996, Biochemistry, 35, 14090). Sugar modification of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Earnshaw and Gait, 1998, Biopolymers (Nucleic acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated in their totality by reference herein). Such publications describe general methods and strategies to determine the location of incorporation of sugar, base and/or phosphate modifications and the like into ribozymes without inhibiting catalysis, and are incorporated by reference herein. In view of such teachings, similar modifications can be used as described herein to modify the nucleic acid molecules of the instant invention.

While chemical modification of oligonucleotide internucleotide linkages with phosphorothioate, phosphorodithioate, and/or 5′-methylphosphonate linkages improves stability, too many of these modifications may cause some toxicity. Therefore, when designing nucleic acid molecules, the amount of these internucleotide linkages should be minimized, but can be balanced to provide acceptable stability while reducing potential toxicity. The reduction in the concentration of these linkages should lower toxicity resulting in increased efficacy and higher specificity of these molecules.

Nucleic acid molecules having chemical modifications which maintain or enhance activity are provided. Such nucleic acid is also generally more resistant to nucleases than unmodified nucleic acid. Thus, in a cell and/or in vivo the activity may not be significantly lowered. Therapeutic nucleic acid molecules delivered exogenously must optimally be stable within cells until translation of the target RNA has been inhibited long enough to reduce the levels of the undesirable protein. This period of time varies between hours to days depending upon the disease state. Clearly, exogenously delivered nucleic acid molecules should be resistant to nucleases in order to function as effective intracellular therapeutic agents. Improvements in the chemical synthesis of RNA and DNA (see, e.g., Wincott et al., 1995 Nucleic Acids Res. 23, 2677; Caruthers et al., 1992, Methods in Enzymology 211,3-19 (all incorporated by reference herein) have expanded the ability to modify nucleic acid molecules by introducing nucleotide modifications to enhance their nuclease stability as described above.

Use of the nucleic acid-based molecules of the invention will lead to better treatment of disease progression by affording the possibility of combination therapies (e.g., multiple antisense or enzymatic nucleic acid molecules targeted to different genes, nucleic acid molecules coupled with known small molecule inhibitors, or intermittent treatment with combinations of molecules (including different motifs) and/or other chemical or biological molecules). The treatment of patients with nucleic acid molecules may also include combinations of different types of nucleic acid molecules.

By “enhanced enzymatic activity” is meant to include activity measured in cells and/or in vivo where the activity is a reflection of both catalytic activity and ribozyme stability. In this invention, the product of these properties is increased or not significantly (less than 10-fold) decreased in vivo compared to an all RNA ribozyme or all DNA enzyme.

In yet another preferred embodiment, nucleic acid catalysts having chemical modifications which maintain or enhance enzymatic activity are provided. Such nucleic acid is also generally more resistant to nucleases than unmodified nucleic acid. Thus, in a cell and/or in vivo the activity may not be significantly lowered. As exemplified herein, such ribozymes are useful in a cell and/or in vivo, even if activity over all is reduced 10-fold (Burgin et al., 1996, Biochemistry, 35, 14090). Such ribozymes herein are said to “maintain” the enzymatic activity on all RNA ribozyme.

In another aspect, the nucleic acid molecules comprise a 5′ and/or a 3′-cap structure.

By “cap structure” is meant chemical modifications, which have been incorporated at the terminus of the oligonucleotide (see for example Wincott et al., WO 97/26270, incorporated by reference herein). These terminal modifications protect the nucleic acid molecule from exonuclease degradation, and may help in delivery and/or localization within a cell. The cap may be present at the 5′-terminus (5′-cap) or at the 3′-terminus (3′-cap) or may be present on both termini. In non-limiting examples: the 5′-cap is selected from the group comprising inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Beigelman et al., International PCT publication No. WO 97/26270, incorporated by reference herein). In yet another preferred embodiment, the 3′-cap is selected from a group comprising, 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine.

An “alkyl” group refers to a saturated aliphatic hydrocarbon, including straight-chain, branched-chain, and cyclic alkyl groups. Preferably, the alkyl group has 1 to 12 carbons. More preferably it is a lower alkyl of from 1 to 7 carbons, still more preferably 1 to 4 carbons. The alkyl group may be substituted or unsubstituted. When substituted the substituted group(s) is preferably, hydroxyl, cyano, alkoxy, ═O, ═S, NO₂ or N(CH₃)₂, amino, or SH. The term also includes alkenyl groups which are unsaturated hydrocarbon groups containing at least one carbon-carbon double bond, including straight-chain, branched-chain, and cyclic groups. Preferably, the alkenyl group has 1 to 12 carbons. More preferably it is a lower alkenyl of from 1 to 7 carbons, still more preferably 1 to 4 carbons. The alkenyl group may be substituted or unsubstituted. When substituted the substituted group(s) is preferably, hydroxyl, cyano, alkoxy, ═O, ═S, NO₂, halogen, N(CH₃)₂, amino, or SH. The term “alkyl” also includes alkynyl groups which have an unsaturated hydrocarbon group containing at least one carbon-carbon triple bond, including straight-chain, branched-chain, and cyclic groups. Preferably, the alkynyl group has 1 to 12 carbons. More preferably it is a lower alkynyl of from 1 to 7 carbons, more preferably 1 to 4 carbons. The alkynyl group may be substituted or unsubstituted. When substituted the substituted group(s) is preferably, hydroxyl, cyano, alkoxy, ═O, ═S, NO₂ or N(CH₃)₂, amino or SH.

Such alkyl groups may also include aryl, alkylaryl, carbocyclic aryl, heterocyclic aryl, amide and ester groups. An “aryl” group refers to an aromatic group which has at least one ring having a conjugated p electron system and includes carbocyclic aryl, heterocyclic aryl and biaryl groups, all of which may be optionally substituted. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

By “nucleotide” as used herein is as recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra; all are hereby incorporated by reference herein). There are several examples of modified nucleic acid bases known in the art. These have been recently summarized by Limbach et al., 1994, Nucleic Acids Res. 22, 2183. Some of the non-limiting examples of base modifications that can be introduced into nucleic acid molecules include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases may be used at any position, for example, within the catalytic core of an enzymatic nucleic acid molecule and/or in the substrate-binding regions of the nucleic acid molecule.

By “abasic” is meant sugar moieties lacking a base or having other chemical groups in place of a base at the 1′ position.

By “ribonucleotide” is meant a nucleotide with a hydroxyl group at the 2′ position of a D-ribo-furanose moiety.

By “unmodified nucleoside” is meant one of the bases adenine, cytosine, guanine, uracil joined to the 1′ carbon of β-D-ribo-furanose and without substitutions on either moiety.

By “modified nucleoside” is meant any nucleotide base which contains a modification in the chemical structure of an unmodified nucleotide base, sugar and/or phosphate.

In connection with 2′-modified nucleotides as described for the present invention, by “amino” is meant 2′-NH₂ or 2′-O-NH₂, which may be modified or unmodified. Such modified groups are described, for example, in Eckstein et al., U.S. Pat. No. 5,672,695 and Matulic-Adamic et al., WO 98/28317, respectively, which are both incorporated by reference in their entireties.

Various modifications to nucleic acid (e.g., antisense and ribozyme) structure can be made to enhance the utility of these molecules. Such modifications will enhance shelf-life, half-life in vitro, stability, and ease of introduction of such oligonucleotides to the target site, e.g., to enhance penetration of cellular membranes, and confer the ability to recognize and bind to targeted cells.

Use of these molecules will lead to better treatment of disease progression by affording the possibility of combination therapies (e.g., multiple ribozymes targeted to different genes, ribozymes coupled with known small molecule inhibitors, or intermittent treatment with combinations of ribozymes (including different ribozyme motifs) and/or other chemical or biological molecules). The treatment of patients with nucleic acid molecules may also include combinations of different types of nucleic acid molecules. Therapies may be devised which include a mixture of ribozymes (including different ribozyme motifs), antisense and/or 2-5A chimera molecules to one or more targets to alleviate symptoms of a disease.

Administration of Nucleic Acid Molecules

Methods for the delivery of nucleic acid molecules are described in Akhtar et al., 1992, Trends Cell Bio., 2, 139; and Delivery Strategies for Antisense Oligonucleotide Therapeutics, ed. Akhtar, 1995, which are both incorporated herein by reference. Sullivan et al., PCT WO 94/02595, further describes the general methods for delivery of enzymatic RNA molecules. These protocols may be utilized for the delivery of virtually any nucleic acid molecule. Nucleic acid molecules may be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, nucleic acid molecules may be directly delivered ex vivo to cells or tissues with or without the aforementioned vehicles. Alternatively, the nucleic acid/vehicle combination is locally delivered by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of nucleic acid delivery and administration are provided in Sullivan et al., supra, Draper et al., PCT WO93/23569, Beigelman et al., PCT WO99/05094, and Klimuk et al., PCT WO99/04819 all of which have been incorporated by reference herein.

The molecules of the instant invention can be used as pharmaceutical agents. Pharmaceutical agents prevent, inhibit the occurrence, or treat (alleviate a symptom to some extent, preferably all of the symptoms) of a disease state in a patient.

The negatively charged polynucleotides of the invention can be administered (e.g., RNA, DNA or protein) and introduced into a patient by any standard means, with or without stabilizers, buffers, and the like, to form a pharmaceutical composition. When it is desired to use a liposome delivery mechanism, standard protocols for formation of liposomes can be followed. The compositions of the present invention may also be formulated and used as tablets, capsules or elixirs for oral administration; suppositories for rectal administration; sterile solutions; suspensions for injectable administration; and the like.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or patient, preferably a human. Suitable forms, in part, depend upon the use or the route of entry, for example oral, transdermal, or by injection. Such forms should not prevent the composition or formulation to reach a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By pharmaceutically acceptable formulation is meant, a composition or formulation that allows for the effective distribution of the nucleic acid molecules of the instant invention in the physical location most suitable for their desired activity. Nonlimiting examples of agents suitable for formulation with the nucleic acid molecules of the instant invention include: P-glycoprotein inhibitors (such as Pluronic P85) which can enhance entry of drugs into the CNS (Jolliet-Riant and Tillement, 1999, Fundam. Clin. Pharmacol., 13, 16-26); biodegradable polymers, such as poly (DL-lactide-coglycolide) microspheres for sustained release delivery after intracerebral implantation (Emerich, D F et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies for the nucleic acid molecules of the instant invention include materials described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058.

The invention also features the use compositions comprising surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392; all of these are incorporated by reference herein). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents may be provided. These include sodium benzoate, sorbic acid and esters of p-hydroxybenzoic acid. In addition, antioxidants and suspending agents may be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

The nucleic acid molecules of the present invention may also be administered to a patient in combination with other therapeutic compounds to increase the overall therapeutic effect. The use of multiple compounds to treat an indication may increase the beneficial effects while reducing the presence of side effects.

Alternatively, certain of the nucleic acid molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; Dropulic et al., 1992, J. Virol., 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al., 1995, Nucleic Acids Res., 23, 2259; Good et al., 1997, Gene Therapy, 4, 45; all which are hereby incorporated by reference herein in their totalities). Those skilled in the art realize that any nucleic acid can be expressed in eukaryotic cells from the appropriate DNA/RNA vector. The activity of such nucleic acids can be augmented by their release from the primary transcript by a ribozyme (Draper et al., PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856; all which are hereby incorporated by reference herein in their totalities).

In another aspect of the invention, RNA molecules of the present invention are preferably expressed from transcription units (see, for example, Couture et al., 1996, TIG., 12, 510) inserted into DNA or RNA vectors. The recombinant vectors are preferably DNA plasmids or viral vectors. Ribozyme expressing viral vectors could be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors may be used that provide for transient expression of nucleic acid molecules. Such vectors might be repeatedly administered as necessary. Once expressed, the nucleic acid molecule binds to the target mRNA. Delivery of nucleic acid molecule expressing vectors could be systemic, such as by intravenous or intramuscular administration, by administration to target cells ex-planted from the patient followed by reintroduction into the patient, or by any other means that would allow for introduction into the desired target cell (for a review see Couture et al., 1996, TIG., 12, 510).

In one aspect the invention features an expression vector comprising nucleic acid sequence encoding at least one of the nucleic acid molecules of the instant invention. The nucleic acid sequence encoding the nucleic acid molecule of the instant invention is operable linked in a manner which allows expression of that nucleic acid molecule.

In another aspect the invention features, an expression vector comprising: a transcription initiation region (e.g., eukaryotic pol I, II or III initiation region); b) a transcription termination region (e.g. eukaryotic pol I, II or III termination region); c) a nucleic acid sequence encoding at least one of the nucleic acid catalyst of the instant invention; and wherein said sequence is operably linked to said initiation region and said termination region, in a manner which allows expression and/or delivery of said nucleic acid molecule. The vector may optionally include an open reading frame (ORF) for a protein operably linked on the 5′ side or the 3′-side of the gene encoding the nucleic acid catalyst of the invention; and/or an intron (intervening sequences).

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters will be expressed at high levels in all cells; the levels of a given pol II promoter in a given cell type will depend on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. U S A, 87, 6743-7; Gao and Huang 1993, Nucleic Acids Res., 21, 2867-72; Lieber et al., 1993, Methods Enzymol., 217, 47-66; Zhou et al., 1990, Mol. Cell. Biol., 10, 4529-37). Several investigators have demonstrated that nucleic acid molecules, such as ribozymes expressed from such promoters can function in mammalian cells (e.g., Kashani-Sabet et al., 1992, Antisense Res. Dev., 2, 3-15; Ojwang et al., 1992, Proc. Natl. Acad. Sci. USA, 89, 10802-6; Chen et al., 1992, Nucleic Acids Res., 20, 4581-9; Yu et al., 1993, Proc. Natl. Acad. Sci. U S A, 90, 6340-4; L'Huillier et al., 1992, EMBO J, 11, 4411-8; Lisziewicz et al., 1993, Proc. Natl. Acad. Sci. U. S. A, 90, 8000-4; Thompson et al, 1995, Nucleic Acids Res., 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as ribozymes in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; Beigelman et al., International PCT Publication No. WO 96/18736; all of these publications are incorporated by reference herein. The above ribozyme transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In yet another aspect, the invention features an expression vector comprising nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner which allows expression and/or delivery of said nucleic acid molecule. In another preferred embodiment, the expression vector comprises: a) a transcription initiation region; b) a transcription termination region; c) an open reading frame; d) a nucleic acid sequence encoding at least one said nucleic acid molecule, wherein said sequence is operably linked to the 3′-end of said open reading frame; and wherein said sequence is operably linked to said initiation region, said open reading frame and said termination region, in a manner which allows expression and/or delivery of said nucleic acid molecule. In yet another embodiment, the expression vector comprises: a) a transcription initiation region; b) a transcription termination region; c) an intron; d) a gene encoding at least one said nucleic acid molecule; and wherein said gene is operably linked to said initiation region, said intron and said termination region, in a manner which allows expression and/or delivery of said nucleic acid molecule. In another embodiment, the expression vector comprises: a) a transcription initiation region; b) a transcription termination region; c) an intron; d) an open reading frame; e) a nucleic acid sequence encoding at least one said nucleic acid molecule, wherein said sequence is operably linked to the 3′-end of said open reading frame; and wherein said sequence is operably linked to said initiation region, said intron, said open reading frame and said termination region, in a manner which allows expression and/or delivery of said nucleic acid molecule.

### EXAMPLES

The following are non-limiting examples showing the selection, isolation, synthesis and activity of nucleic acids of the instant invention.

The following examples demonstrate the selection and design of antisense, hammerhead, DNAzyme, NCH, or G-Cleaver ribozyme molecules and binding/cleavage sites within BACE RNA.

### Example 1

### Identification of Potential Target Sites in Human BACE RNA

The sequence of human BACE was screened for accessible sites using a computer-folding algorithm. Regions of the RNA that did not form secondary folding structures and contained potential ribozyme and/or antisense binding/cleavage sites were identified. The sequences of these cleavage sites are shown in Tables III-VIII.

### Example 2

### Selection of Enzymatic Nucleic Acid Cleavage Sites in Human BACE RNA

Ribozyme target sites were chosen by analyzing sequences of Human BACE (Genbank sequence accession number: AF190725) and prioritizing the sites on the basis of folding. Ribozymes were designed that could bind each target and were individually analyzed by computer folding (Christoffersen et al., 1994 J. Mol. Struc. Theochem, 311, 273; Jaeger et al., 1989, Proc. Natl. Acad. Sci. USA, 86, 7706) to assess whether the ribozyme sequences fold into the appropriate secondary structure. Those ribozymes with unfavorable intramolecular interactions between the binding arms and the catalytic core were eliminated from consideration. As noted below, varying binding arm lengths can be chosen to optimize activity. Generally, at least 5 bases on each arm are able to bind to, or otherwise interact with, the target RNA.

### Example 3

### Chemical Synthesis and Purification of Ribozymes and Antisense for Efficient Cleavage and/or Blocking of BACE RNA

Ribozymes and antisense constructs were designed to anneal to various sites in the RNA message. The binding arms of the ribozymes are complementary to the target site sequences described above, while the antisense constructs are filly complimentary to the target site sequences described above. The ribozymes and antisense constructs were chemically synthesized. The method of synthesis used followed the procedure for normal RNA synthesis as described above and in Usman et al., (1987 J. Am. Chem. Soc., 109, 7845), Scaringe et al., (1990 Nucleic Acids Res., 18, 5433) and Wincott et al., supra, and made use of common nucleic acid protecting and coupling groups, such as dimethoxytrityl at the 5′-end, and phosphoramidites at the 3′-end. The average stepwise coupling yields were >98%.

Ribozymes and antisense constructs were also synthesized from DNA templates using bacteriophage T7 RNA polymerase (Milligan and Uhlenbeck, 1989, Methods Enzymol. 180, 51). Ribozymes and antisense constructs were purified by gel electrophoresis using general methods or were purified by high pressure liquid chromatography (HPLC; See Wincott et al., supra; the totality of which is hereby incorporated herein by reference) and were resuspended in water. The sequences of the chemically synthesized ribozymes and antisense constructs used in this study are shown below in Table III-VIII.

### Example 4

### Ribozyme Cleavage of BACE RNA Target in vitro

Ribozymes targeted to the human BACE RNA are designed and synthesized as described above. These ribozymes can be tested for cleavage activity in vitro, for example, using the following procedure. The target sequences and the nucleotide location within the BACE RNA are given in Tables III-VIII.

Cleavage Reactions:

Full-length or partially full-length, internally-labeled target RNA for ribozyme cleavage assay is prepared by in vitro transcription in the presence of [a-³²P] CTP, passed over a G 50 Sephadex column by spin chromatography and used as substrate RNA without further purification. Alternately, substrates are 5′-³²P-end labeled using T4 polynucleotide kinase enzyme. Assays are performed by pre-warming a 2× concentration of purified ribozyme in ribozyme cleavage buffer (50 mM Tris-HCl, pH 7.5 at 37° C., 10 mM MgCl₂) and the cleavage reaction was initiated by adding the 2× ribozyme mix to an equal volume of substrate RNA (maximum of 1-5 nM) that was also pre-warmed in cleavage buffer. As an initial screen, assays are carried out for 1 hour at 37° C. using a final concentration of either 40 nM or 1 mM ribozyme, i.e., ribozyme excess. The reaction is quenched by the addition of an equal volume of 95% formamide, 20 mM EDTA, 0.05% bromophenol blue and 0.05% xylene cyanol after which the sample is heated to 95° C. for 2 minutes, quick chilled and loaded onto a denaturing polyacrylamide gel. Substrate RNA and the specific RNA cleavage products generated by ribozyme cleavage are visualized on an autoradiograph of the gel. The percentage of cleavage is determined by Phosphor Imager® quantitation of bands representing the intact substrate and the cleavage products.

### Example 5

### Protein (APP/BACE/ps-2) Target Activation of Nucleic Acid Sensor Molecule

One method for protein detection contemplated by the invention utilizes a catalytically attenuated enzymatic nucleic acid molecule that is fused to a high affinity RNA ligand for a target protein in such a way that target association induces catalytic activity. A variation of combinatorial selection methods can be easily and quickly used to create high affinity RNA ligands (RNA sensor domains) for specific proteins. Combinatorial selection of RNA aptamers has been automated and multiplexed, providing a high throughput method for their production. As with antibodies, RNA aptamers display picomolar affinities for their targets and can discriminate between protein homologs, isoforms, and even different activation states of the same protein. Alternately, RNA sensor domains can be obtained from natural sources, such as the RNA binding domains of a virus (e.g. rev response elements and TAR elements of HIV) or eukaryotic RNA binding proteins (e.g. protein kinase PKR, promoters, RNA polymerase, ribosomal RNA binding domains etc). In addition, a random sequence can be attached to an attenuated enzymatic nucleic acid molecule and through the use of combinatorial selection, allosteric nucleic acid molecules can be isolated that are modulated in the presence of a target signaling agent or molecule.

This approach relies upon binding of a protein target to an RNA aptamer domain in the nucleic acid sensor molecule to induce catalytic activity. To accomplish this activation, the sensor and enzymatic nucleic acid molecule domains are fused via a third element, a communication module, that is responsible promoting enzymatic nucleic acid molecule catalysis upon target binding. The communication module is a nucleic acid sequence or sequences that promote a conformational rearrangement of the enzymatic nucleic acid molecule domain into its active structure upon target binding. Two routes exist for the production of communication modules: rational design or combinatorial selection. One approach utilizes rational design where pre-made communication module or modules are fused to preexisting enzymatic nucleic acid molecule and aptamer domains in a modular strategy.

An RNA sensor domain that binds to BACE, ps-2, or APP protein is appended to a variant of the hammerhead enzymatic nucleic acid molecule through a communication module developed through rational design. The salient feature of this design strategy is that substrate-binding elements in the enzymatic nucleic acid molecule domain are sequestered by complementary allosteric effector sequences present in the communication module in the absence of target. Target association with the sensor domain forces an alternative RNA conformation in which the substrate binding elements become available for interaction with cleavage substrate, thus promoting catalysis.

This nucleic acid sensor displays little catalytic activity in the absence of the BACE, ps-2, or APP protein but is activated in the presence of recombinant protien. No nucleic acid sensor activation is observed if another protein, for example bovine serum albumin (BSA), replaces BACE, ps-2, or APP in the reaction, indicating that activation specifically requires BACE, ps-2, or APP. An enzymatic nucleic acid molecule that does not contain the BACE, ps-2, or APP sensor component displays nearly identical activity in the presence or absence of the protein target. To examine the dependence of activation on the concentration of BACE, ps-2, or APP, various amounts of BACE, ps-2, or APP are added to different reactions.

Cell Culture Models

Vassar et al., 1999, Science, 286, 735-741, describe a cell culture model for studying BACE inhibition. Specific antisense nucleic acid molecules targeting BACE mRNA were used for inhibition studies of endogenous BACE expression in 101 cells and APPsw (Swedish type amyloid precursor protein expressing) cells via lipid mediated transfection. Antisense treatment resulted in dramatic reduction of both BACE mRNA by Northern blot analysis, and APPsβsw (“Swedish” type β-secretase cleavage product) by ELISA, with maximum inhibition of both parameters at 75-80%. This model was also used to study the effect of BACE inhibition on amyloid β-peptide production in APPsw cells.

Animal Models

Games et al., 1995, Nature, 373, 523-527, describe a transgenic mouse model in which mutant human familial type APP (Phe 717 instead of Val) is overexpressed. This model results in mice that progressively develop many of the pathological hallmarks of Alzheimer's disease, and as such, provides a model for testing therapeutic drugs.

Indications

Particular degenerative and disease states that can be associated with BACE, APP, as ps-2 expression modulation include but are not limited to neurodegenerative diseases such as Alzheimer's disease and dementia.

The present body of knowledge in BACE, APP, as ps-2 research indicates the need for methods to assay BACE activity and for compounds that can regulate BACE, APP, as ps-2 expression for research, diagnostic, and therapeutic use.

Donepezil, tacrine, selegeline, and acetyl-L-camitine are non-limiting examples of pharmaceutical agents that can be combined with or used in conjunction with the nucleic acid molecules (e.g. ribozymes and antisense molecules) of the instant invention. Those skilled in the art will recognize that other drugs such as diuretic and antihypertensive compounds and therapies can be similarly be readily combined with the nucleic acid molecules of the instant invention (e.g. ribozymes and antisense molecules) are hence within the scope of the instant invention.

Diagnostic Uses

The nucleic acid molecules of this invention (e.g., enzymatic nucleic acid molecules) can be used as diagnostic tools to examine genetic drift and mutations within diseased cells or to detect the presence of BACE, PS-2, or APP RNA in a cell. The close relationship between enzymatic nucleic acid molecule activity and the structure of the target RNA allows the detection of mutations in any region of the molecule which alters the base-pairing and three-dimensional structure of the target RNA. By using multiple enzymatic nucleic acid molecules described in this invention, one can map nucleotide changes which are important to RNA structure and function in vitro, as well as in cells and tissues. Cleavage of target RNAs with enzymatic nucleic acid molecules can be used to inhibit gene expression and define the role (essentially) of specified gene products in the progression of disease. In this manner, other genetic targets can be defined as important mediators of the disease. These experiments can lead to better treatment of the disease progression by affording the possibility of combinational therapies (e.g., multiple enzymatic nucleic acid molecules targeted to different genes, enzymatic nucleic acid molecules coupled with known small molecule inhibitors, or intermittent treatment with combinations of enzymatic nucleic acid molecules and/or other chemical or biological molecules). Other in vitro uses of enzymatic nucleic acid molecules of this invention are well known in the art, and include detection of the presence of mRNAs associated with BACE, PS-2, or APP-related condition.

Such RNA is detected by determining the presence of a cleavage product after treatment with an enzymatic nucleic acid molecule using standard methodology.

In a specific example, enzymatic nucleic acid molecules which cleave only wild-type or mutant forms of the target RNA are used for the assay. The first enzymatic nucleic acid molecule is used to identify wild-type RNA present in the sample and the second enzymatic nucleic acid molecule is used to identify mutant RNA in the sample. As reaction controls, synthetic substrates of both wild-type and mutant RNA are cleaved by both enzymatic nucleic acid molecules to demonstrate the relative enzymatic nucleic acid molecule efficiencies in the reactions and the absence of cleavage of the “non-targeted” RNA species. The cleavage products from the synthetic substrates also serve to generate size markers for the analysis of wild-type and mutant RNAs in the sample population. Thus each analysis requires two enzymatic nucleic acid molecules, two substrates and one unknown sample which is combined into six reactions. The presence of cleavage products is determined using an RNAse protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (i.e., BACE, PS-2, or APP) is adequate to establish risk. If probes of comparable specific activity are used for both transcripts, then a qualitative comparison of RNA levels will be adequate and will decrease the cost of the initial diagnosis. Higher mutant form to wild-type ratios are correlated with higher risk whether RNA levels are compared qualitatively or quantitatively. The use of enzymatic nucleic acid molecules in diagnostic applications contemplated by the instant invention is described, for example, in George et al, U.S. Pat. Nos. 5,834,186 and 5,741,679, Shih et al., U.S. Pat. No. 5,589,332, Nathan et al., U.S. Pat. No 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842.

Additional Uses

Potential usefulness of sequence-specific enzymatic nucleic acid molecules of the instant invention might have many of the same applications for the study of RNA that DNA restriction endonucleases have for the study of DNA (Nathans et al., 1975 Ann. Rev. Biochem. 44:273). For example, the pattern of restriction fragments could be used to establish sequence relationships between two related RNAs, and large RNAs could be specifically cleaved to fragments of a size more useful for study. The ability to engineer sequence specificity of the enzymatic nucleic acid molecule is ideal for cleavage of RNAs of unknown sequence. Applicant describes the use of nucleic acid molecules to down-regulate gene expression of target genes in bacterial, microbial, fungal, viral, and eukaryotic systems including plant, or mammalian cells.

TABLE III
Human BACE Hammerhead Ribozyme and Target Sequence

| Pos | Substrate | Seq ID | Ribozyme | Seq ID |
|---|---|---|---|---|
| 9 | CCACGCGU C CGCAGCCC | 1 | GGGCUGCG CUGAUGAG GCCGUUAGGC CGAA ACGCGUGG | 1776 |
| 47 | AGCUGGAU U AUGCUGGC | 2 | GCCACCAU CUGAUGAG GCCGUUAGGC CGAA AUCCAGCU | 1777 |
| 48 | GCUGGAUU A UGGUGGCC | 3 | GGCCACCA CUGAUGAG GCCGUUAGGC CGAA AAUCCAGC | 1778 |
| 93 | GGAGCCCU U GCCCCUGC | 4 | GCAGGGGC CUGAUGAG GCCGUUAGGC CGAA AGGGCUCC | 1779 |
| 163 | CCGCCCCU C CCAGCCCC | 5 | GGGGCUGG CUGAUGAG GCCGUUAGGC CGAA AGGGGCGG | 1780 |
| 221 | GCCGAUGU A GCGGGCUC | 6 | GAGCCCGC CUGAUGAG GCCGUUAGGC CGAA ACAUCGGC | 1781 |
| 229 | AGCGGGCU C CGGAUCCC | 7 | GGGAUCCG CUGAUGAG GCCGUUAGGC CGAA AGCCCGCU | 1782 |
| 235 | CUCCGGAU C CCAGCCUC | 8 | GAGGCUGG CUGAUGAG GCCGUUAGGC CGAA AUCCGGAG | 1783 |
| 243 | CCCAGCCU C UCCCCUGC | 9 | GCAGGGGA CUGAUGAG GCCGUUAGGC CGAA AGGCUGGG | 1784 |
| 245 | CAGCCUCU C CCCUGCUC | 10 | GAUCAGUG CUGAUGAG GCCGUUAGGC CGAA AGAGGCUG | 1785 |
| 253 | CCCCUGCU C CCGUGCUC | 11 | GAGCACGG CUGAUGAG GCCGUUAGGC CGAA AGCAGGGG | 1786 |
| 261 | CCCGUGCU C UGCGGAUC | 12 | GAUCCGCA CUGAUGAG GCCGUUAGGC CGAA AGCACGGG | 1787 |
| 269 | CUGCGGAU C UCCCCUGA | 13 | UCAGGUGA CUGAUGAG GCCGUUAGGC CGAA AUCCGCAG | 1788 |
| 271 | GCGGAUCU C CCCUGACC | 14 | UGUCAGUG CUGAUGAG GCCGUUAGGC CGAA AGAUCCGC | 1789 |
| 283 | UGACCGCU C UCCACAUC | 15 | UCUGUGGA CUGAUGAG GCCGUUAGGC CGAA AGCGGUCA | 1790 |
| 285 | ACCGCUCU C CACAGCCC | 16 | GGGCUGUG CUGAUGAG GCCGUUAGGC CGAA AGAGCGGU | 1791 |
| 334 | CCUGGCGU C CUGAUGCC | 17 | GUCAUCAG CUGAUGAG GCCGUUAGGC CGAA ACGCCAGG | 1792 |
| 351 | CCCAAGCU C CCUCUCCU | 18 | AGGAGAUG CUGAUGAG GCCGUUAGGC CGAA AGCUUGGG | 1793 |
| 355 | AGCUCCCU C UCCUGAGA | 19 | UCUCAGGA CUGAUGAG GCCGUUAGGC CGAA AGGGAGCU | 1794 |
| 357 | CUCCCUCU C CUGAGAAG | 20 | CUUCUCAG CUGAUGAG GCCGUUAGGC CGAA AGAGGGAG | 1795 |
| 386 | CCCAGACU U GUGGUCAG | 21 | CUGCCCCC CUGAUGAG GCCGUUAGGC CGAA AGUCUGGG | 1796 |
| 477 | CCCUGUCU C CUGCUGUG | 22 | CACAGCAG CUGAUGAG GCCGUUAGGC CGAA AGCCAGGG | 1797 |
| 531 | CACGGCAU C CGGCUGCC | 23 | GGCAGCCG CUGAUGAG GCCGUUAGGC CGAA AUGCCGUG | 1798 |
| 632 | GGGCAGCU U UGUGGAGA | 24 | UCUCCACA CUGAUGAG GCCGUUAGGC CGAA AGCUGCCC | 1799 |
| 633 | GGCAGCUU U GUGGAGAU | 25 | AUCUCCAC CUGAUGAG GCCGUUAGGC CGAA AAGCUGCC | 1800 |
| 665 | GGGCAAGU C UGGUCAUG | 26 | CCUGCCCC CUGAUGAG GCCGUUAGGC CGAA ACUUGCCC | 1801 |
| 677 | GCAGUUCU A CUACGUGU | 27 | CCACUUAG CUGAUGAG GCCGUUAGGC CGAA AGCCCUGC | 1802 |
| 680 | GGGCUACU A CGUGGAGA | 28 | UCUCCACG CUGAUGAG GCCGUUAGGC CGAA AGUAGCCC | 1803 |
| 717 | CAGACGCU C AACAUCCU | 29 | AGGAUGUU CUGAUGAG GCCGUUAGGC CGAA AGCGUCUG | 1804 |
| 723 | CUCAACAU C CUGGUGGA | 30 | UCCACCAG CUGAUGAG GCCGUUAGGC CGAA AUGUUGAG | 1805 |
| 733 | UGGUGGAU A CAGGCAGC | 31 | GCUGCCUG CUGAUGAG GCCGUUAGGC CGAA AUCCACCA | 1806 |
| 745 | GCAGCAGU A ACUUUGCA | 32 | UGCAAAGU CUGAUGAG GCCGUUAGGC CGAA ACUGCUGC | 1807 |
| 749 | CAGUAACU U UGCAGUGG | 33 | CCACUGCA CUGAUGAG GCCGUUAGGC CGAA AGUUACUG | 1808 |
| 750 | AGUAACUU U GCAGUGGG | 34 | CCCACUGC CUGAUGAG GCCGUUAGGC CGAA AAGUUACU | 1809 |
| 776 | CCACCCCU U CCUGCAUC | 35 | GAUGCAGG CUGAUGAG GCCGUUAGGC CGAA AGGGGUGG | 1810 |
| 777 | CACCCCUU C CUGCAUCG | 36 | CGAUGCAG CUGAUGAG GCCGUUAGGC CGAA AAGGGGUG | 1811 |
| 784 | UCCUGCAU C GCUACUAC | 37 | GUAGUAGC CUGAUGAG GCCGUUAGGC CGAA AUGCAGGA | 1812 |
| 788 | GCAUCGCU A CUACCAGA | 38 | UCUGGUAG CUGAUGAG GCCGUUAGGC CGAA AGCGAUGC | 1813 |
| 791 | UCGCUACU A CCAGAGGC | 39 | GCCUCUGG CUGAUGAG GCCGUUAGGC CGAA AGUAGCGA | 1814 |
| 806 | GCAGCUGU C CAGCACAU | 40 | AUGUGCUG CUGAUGAG GCCGUUAGGC CGAA ACAGCUGC | 1815 |
| 815 | CAGCACAU A CCGGGACC | 41 | GGUCCCGG CUGAUGAG GCCGUUAGGC CGAA AUGUGCUG | 1816 |
| 825 | CGGGACCU C CGGAAGGG | 42 | CCCUUCCG CUGAUGAG GCCGUUAGGC CGAA AGGUCCCG | 1817 |
| 839 | GGGUGUGU A UGUGCCCU | 43 | AGGGCACA CUGAUGAG GCCGUUAGGC CGAA ACACACCC | 1818 |
| 848 | UGUGCCCU A CACCCAGG | 44 | CCUGGGUG CUGAUGAG GCCGUUAGGC CGAA AGGGCACA | 1819 |
| 891 | GACCUGGU A AGCAUCCC | 45 | GGGAUGCU CUGAUGAG GCCGUUAGGC CGAA ACCAGGUC | 1820 |
| 897 | GUAAGCAU C CCCCAUGG | 46 | CCAUGGGG CUGAUGAG GCCGUUAGGC CGAA AUGCUUAC | 1821 |
| 915 | CCCAACGU C ACUGUGCG | 47 | CGCACAGU CUGAUGAG GCCGUUAGGC CGAA ACGUUGGG | 1822 |
| 933 | GCCAACAU U GCUGCCAU | 48 | AUGGCAGC CUGAUGAG GCCGUUAGGC CGAA AUGUUGGC | 1823 |
| 942 | GCUGCCAU C ACUGAAUC | 49 | GAUUCAGU CUGAUGAG GCCGUUAGGC CGAA AUGGCAGC | 1824 |
| 950 | CACUGAAU C AGACAAGU | 50 | ACUUGUCU CUGAUGAG GCCGUUAGGC CGAA AUUCAGUG | 1825 |
| 959 | AGACAAGU U CUUCAUCA | 51 | UGAUGAAG CUGAUGAG GCCGUUAGGC CGAA ACUUGUCU | 1826 |
| 960 | GACAAGUU C UUCAUCAA | 52 | UUGAUGAA CUGAUGAG GCCGUUAGGC CGAA AACUUGUC | 1827 |
| 962 | CAAGUUCU U CAUCAACG | 53 | CGUUGAUG CUGAUGAG GCCGUUAGGC CGAA AGAACUUG | 1828 |
| 963 | AAGUUCUU C AUCAACGG | 54 | CCGUUGAU CUGAUGAG GCCGUUAGGC CGAA AAGAACUU | 1829 |
| 966 | UUCUUCAU C AACGGCUC | 55 | GAGCCGUU CUGAUGAG GCCGUUAGGC CGAA AUGAAGAA | 1830 |
| 974 | CAACGGCU C CAACUGGG | 56 | CCCAGUUG CUGAUGAG GCCGUUAGGC CGAA AGCCGUUG | 1831 |
| 990 | GAAGGCAU C CUGGGGCU | 57 | AGCCCCAG CUGAUGAG GCCGUUAGGC CGAA AUGCCUUC | 1832 |
| 1004 | GCUGGCCU A UGCUGAGA | 58 | UCUCAGCA CUGAUGAG GCCGUUAGGC CGAA AGGCCAGC | 1833 |
| 1014 | GCUGAGAU U GCCAGGCC | 59 | GGCCUGGC CUGAUGAG GCCGUUAGGC CGAA AUCUCAGC | 1834 |
| 1031 | UGACGACU C CCUGGAGC | 60 | GCUCCAGG CUGAUGAG GCCGUUAGGC CGAA AGUCGUCA | 1835 |
| 1042 | UGGAGCCU U UCUUGGAC | 61 | GUCAAAGA CUGAUGAG GCCGUUAGGC CGAA AGGCUCCA | 1836 |
| 1043 | GGAGCCUU U CUUUGACU | 62 | AGUCAAAG CUGAUGAG GCCGUUAGGC CGAA AAGGCUCC | 1837 |
| 1044 | GAGCCUUU C UUUGACUC | 63 | GAGUCAAA CUGAUGAG GCCGUUAGGC CGAA AAAGGCUC | 1838 |
| 1046 | GCCUUUCU U UGACUCUC | 64 | GAGAGUCA CUGAUGAG GCCGUUAGGC CGAA AGAAAGGC | 1839 |
| 1047 | CCUUUCUU U GACUCUCU | 65 | AGAGAGUC CUGAUGAG GCCGUUAGGC CGAA AAGAAGG | 1840 |
| 1052 | CUUUGACU C UCUGGUAA | 66 | UUACCAGA CUGAUGAG GCCGUUAGGC CGAA AGUCAAAG | 1841 |
| 1054 | UUGACUCU C UGGUAAAG | 67 | CUUUACCA CUGAUGAG GCCGUUAGGC CGAA AGAGUCAA | 1842 |
| 1059 | UCUCUGGU A AAGCAGAC | 68 | GUCUGCUU CUGAUGAG GCCGUUAGGC CGAA ACCAGAGA | 1843 |
| 1074 | ACCCACGU U CCCAACCU | 69 | AGGUUGGG CUGAUGAG GCCGUUAGGC CGAA ACGUGGGU | 1844 |
| 1075 | CCCACGUU C CCAACCUC | 70 | GAGGUUGG CUGAUGAG GCCGUUAGGC CGAA AACGUGGG | 1845 |
| 1083 | CCCAACCU C UUCUCCCU | 71 | AGGGAGAA CUGAUGAG GCCGUUAGGC CGAA AGGUUGGG | 1846 |
| 1085 | CAACCUCU U CUCCCUGC | 72 | GCAGGGAG CUGAUGAG GCCGUUAGGC CGAA AGAGGUUG | 1847 |
| 1086 | AACCUCUU C UCCCUGCA | 73 | UGCAGGGA CUGAUGAG GCCGUUAGGC CGAA AAGAGGUU | 1848 |
| 1088 | CCUCUUCU C CCUGCAGC | 74 | GCUGCAGG CUGAUGAG GCCGUUAGGC CGAA AGAAGAGG | 1849 |
| 1098 | CUGCAGCU U UGUGGUGC | 75 | GCACCACA CUGAUGAG GCCGUUAGGC CGAA AGCUGCAG | 1850 |
| 1099 | UGCAGCUU U GUGGUGCU | 76 | AGCACCAC CUGAUGAG GCCGUUAGGC CGAA AAGCUGCA | 1851 |
| 1112 | UGCUGGCU U CCCCCUCA | 77 | UGAGGGGG CUGAUGAG GCCGUUAGGC CGAA AGCCAGCA | 1852 |
| 1113 | GCUGGCUU C CCCCUCAA | 78 | UUGAGGGG CUGAUGAG GCCGUUAGGC CGAA AAGCCAGC | 1853 |
| 1119 | UUCCCCCU C AACCAGUC | 79 | GACUGGUU CUGAUGAG GCCGUUAGGC CGAA AGGGGGAA | 1854 |
| 1127 | CAACCAGU C UGAAGUGC | 80 | GCACUUCA CUGAUGAG GCCGUUAGGC CGAA ACUGGUUG | 1855 |
| 1142 | GCUGGCCU C UGUCGGAG | 81 | CUCCGACA CUGAUGAG GCCGUUAGGC CGAA AGGCCAGC | 1856 |
| 1146 | GCCUCUGU C GGAGGGAG | 82 | CUCCCUCC CUGAUGAG GCCGUUAGGC CGAA ACAGAGGC | 1857 |
| 1161 | AGCAUGAU C AUUGGAGG | 83 | CCUCCAAU CUGAUGAG GCCGUUAGGC CGAA AUCAUGCU | 1858 |
| 1164 | AUGAUCAU U GGAGGUAU | 84 | AUACCUCC CUGAUGAG GCCGUUAGGC CGAA AUGAUCAU | 1859 |
| 1171 | UUGGAGGU A UCGACCAC | 85 | GUGGUCCA CUGAUGAG GCCGUUAGGC CGAA ACCUCCAA | 1860 |
| 1173 | GGAGGUAU C GACCACUC | 86 | GAGUGGUC CUGAUGAG GCCGUUAGGC CGAA AUACCUCC | 1861 |
| 1181 | CGACCACU C GCUGUACA | 87 | UGUACAGC CUGAUGAG GCCGUUAGGC CGAA AGUGGUCG | 1862 |
| 1187 | CUCGCUGU A CACAGGCA | 88 | UGCCUGUG CUGAUGAG GCCGUUAGGC CGAA ACAGCGAG | 1863 |
| 1198 | CAGGCAGU C UCUGGUAU | 89 | AUACCAGA CUGAUGAG GCCGUUAGGC CGAA ACUGCCUG | 1864 |
| 1200 | GGCAGUCU C UGGUAUAC | 90 | GUAUACCA CUGAUGAG GCCGUUAGGC CGAA AGACUGCC | 1865 |
| 1205 | UCUCUGGU A UACACCCA | 91 | UGGGUGUA CUGAUGAG GCCGUUAGGC CGAA ACCAGAGA | 1866 |
| 1207 | UCUGGUAU A CACCCAUC | 92 | GAUGGGUG CUGAUGAG GCCGUUAGGC CGAA AUACCAGA | 1867 |
| 1215 | ACACCCAU C CGGCGGGA | 93 | UCCCGCCG CUGAUGAG GCCGUUAGGC CGAA AUGGGUGU | 1868 |
| 1229 | GGAGUGGU A UUAUGAGG | 94 | CCUCAUAA CUGAUGAG GCCGUUAGGC CGAA ACCACUCC | 1869 |
| 1231 | AGUGGUAU U AUGAGGUG | 95 | CACCUCAU CUGAUGAG GCCGUUAGGC CGAA AUACCACU | 1870 |
| 1232 | GUGGUAUU A UGAGGUGA | 96 | UCACCUCA CUGAUGAG GCCGUUAGGC CGAA AAUACCAC | 1871 |
| 1242 | GAGGUGAU C AUUGUGCG | 97 | CGCACAAU CUGAUGAG GCCGUUAGGC CGAA AUCACCUC | 1872 |
| 1245 | GUGAUCAU U GUGCGGGU | 98 | ACCCGCAC CUGAUGAG GCCGUUAGGC CGAA AUGAUCAC | 1873 |
| 1260 | GUGGAGAU C AAUGGACA | 99 | UGUCCAUU CUGAUGAG GCCGUUAGGC CGAA AUCUCCAC | 1874 |
| 1273 | GACAGGAU C UGAAAAUG | 100 | CAUUUUCA CUGAUGAG GCCGUUAGGC CGAA AUCCUGUC | 1875 |
| 1295 | CAAGGAGU A CAACUAUG | 101 | CAUAGUUG CUGAUGAG GCCGUUAGGC CGAA ACUCCUUG | 1876 |
| 1301 | GUACAACU A UGACAAGA | 102 | UCUUGUCA CUGAUGAG GCCGUUAGGC CGAA AGUUGUAC | 1877 |
| 1314 | AAGAGCAU U GUGGACAG | 103 | CUGUCCAC CUGAUGAG GCCGUUAGGC CGAA AUGCUCUU | 1878 |
| 1338 | ACCAACCU U CGUUUGCC | 104 | GGCAAACG CUGAUGAG GCCGUUAGGC CGAA AGGUUGGU | 1879 |
| 1339 | CCAACCUU C GUUUGCCC | 105 | GGGCAAAC CUGAUGAG GCCGUUAGGC CGAA AAGGUUGG | 1880 |
| 1342 | ACCUUCGU U UGCCCAAG | 106 | CUUGGGCA CUGAUGAG GCCGUUAGGC CGAA ACGAAGGU | 1881 |
| 1343 | CCUUCGUU U GCCCAAGA | 107 | UCUUGGGC CUGAUGAG GCCGUUAGGC CGAA AACGAAGG | 1882 |
| 1358 | GAAAGUGU U UGAAGCUG | 108 | CAGCUUCA CUGAUGAG GCCGUUAGGC CGAA ACACUUUC | 1883 |
| 1359 | AAAGUGUU U GAAGCUGC | 109 | GCAGCUUC CUGAUGAG GCCGUUAGGC CGAA AACACUUU | 1884 |
| 1371 | GCUGCAGU C AAAUCCAU | 110 | AUGGAUUU CUGAUGAG GCCGUUAGGC CGAA ACUGCAGC | 1885 |
| 1376 | AGUCAAAU C CAUCAAGG | 111 | CCUUGAUG CUGAUGAG GCCGUUAGGC CGAA AUUUGACU | 1886 |
| 1380 | AAAUCCAU C AAGGCAGC | 112 | GCUGCCUU CUGAUGAG GCCGUUAGGC CGAA AUGGAUUU | 1887 |
| 1391 | GGCAGCCU C CUCCACGG | 113 | CCGUGGAG CUGAUGAG GCCGUUAGGC CGAA AGGCUGCC | 1888 |
| 1394 | AGCCUCCU C CACGGAGA | 114 | UCUCCGUG CUGAUGAG GCCGUUAGGC CGAA AGGAGGCU | 1889 |
| 1406 | GGAGAAGU U CCCUGAUG | 115 | CAUCAGGG CUGAUGAG GCCGUUAGGC CGAA ACUUCUCC | 1890 |
| 1407 | GAGAAGUU C CCUGAUGG | 116 | CCAUCAGG CUGAUGAG GCCGUUAGGC CGAA AACUUCUC | 1891 |
| 1417 | CUGAUGGU U UCUGGCUA | 117 | UAGCCAGA CUGAUGAG GCCGUUAGGC CGAA ACCAUCAG | 1892 |
| 1418 | UGAUGGUU U CUGGCUAG | 118 | CUAGCCAG CUGAUGAG GCCGUUAGGC CGAA AACCAUCA | 1893 |
| 1419 | GAUGGUUU C UGGCUAGG | 119 | CCUAGCCA CUGAUGAG GCCGUUAGGC CGAA AAACCAUC | 1894 |
| 1425 | UUCUGGCU A GGAGAGCA | 120 | UGCUCUCC CUGAUGAG GCCGUUAGGC CGAA AGCCAGAA | 1895 |
| 1465 | CCACCCCU U GGAACAUU | 121 | AAUGUUCC CUGAUGAG GCCGUUAGGC CGAA AGGGGUGG | 1896 |
| 1473 | UGGAACAU U UUCCCAGU | 122 | ACUGGGAA CUGAUGAG GCCGUUAGGC CGAA AUGUUCCA | 1897 |
| 1474 | GGAACAUU U UCCCAGUC | 123 | GACUGGGA CUGAUGAG GCCGUUAGGC CGAA AAUGUUCC | 1898 |
| 1475 | GAACAUUU U CCCAGUCA | 124 | UGACUGGG CUGAUGAG GCCGUUAGGC CGAA AAAUGUUC | 1899 |
| 1476 | AACAUUUU C CCAGUCAU | 125 | AUGACUGG CUGAUGAG GCCGUUAGGC CGAA AAAAUGUU | 1900 |
| 1482 | UUCCCAGU C AUCUCACU | 126 | AGUGAGAU CUGAUGAG GCCGUUAGGC CGAA ACUGGGAA | 1901 |
| 1485 | CCAGUCAU C UCACUCUA | 127 | UAGAGUGA CUGAUGAG GCCGUUAGGC CGAA AUGACUGG | 1902 |
| 1487 | AGUCAUCU C ACUCUACC | 128 | GGUAGAGU CUGAUGAG GCCGUUAGGC CGAA AGAUGACU | 1903 |
| 1491 | AUCUCACU C UACCUAAU | 129 | AUUAGGUA CUGAUGAG GCCGUUAGGC CGAA AGUGAGAU | 1904 |
| 1493 | CUCACUCU A CCUAAUGG | 130 | CCAUUAGG CUGAUGAG GCCGUUAGGC CGAA AGAGUGAG | 1905 |
| 1497 | CUCUACCU A AUGGGUGA | 131 | UCACCCAU CUGAUGAG GCCGUUAGGC CGAA AGGUAGAG | 1906 |
| 1509 | GGUGAGGU U ACCAACCA | 132 | UGGUUGGU CUGAUGAG GCCGUUAGGC CGAA ACCUCACC | 1907 |
| 1510 | GUGAGGUU A CCAACCAG | 133 | CUGGUUGG CUGAUGAG GCCGUUAGGC CGAA AACCUCAC | 1908 |
| 1520 | CAACCAGU C CUUCCGCA | 134 | UGCGGAAG CUGAUGAG GCCGUUAGGC CGAA ACUGGUUG | 1909 |
| 1523 | CCAGUCCU U CCGCAUCA | 135 | UGAUGCGG CUGAUGAG GCCGUUAGGC CGAA AGGACUGG | 1910 |
| 1524 | CAGUCCUU C CGCAUCAC | 136 | GUGAUGCG CUGAUGAG GCCGUUAGGC CGAA AAGGACUG | 1911 |
| 1530 | UUCCGCAU C ACCAUCCU | 137 | AGGAUGGU CUGAUGAG GCCGUUAGGC CGAA AUGCGGAA | 1912 |
| 1536 | AUCACCAU C CUUCCGCA | 138 | UGCGGAAG CUGAUGAG GCCGUUAGGC CGAA AUGGUGAU | 1913 |
| 1539 | ACCAUCCU U CCGCAGCA | 139 | UGCUGCGG CUGAUGAG GCCGUUAGGC CGAA AGGAUGGU | 1914 |
| 1540 | CCAUCCUU C CGCAGCAA | 140 | UUGCUGCG CUGAUGAG GCCGUUAGGC CGAA AAGGAUGG | 1915 |
| 1550 | GCAGCAAU A CCUGCGGC | 141 | GCCGCAGG CUGAUGAG GCCGUUAGGC CGAA AUUGCUGC | 1916 |
| 1580 | GGCCACGU C CCAAGACG | 142 | CGUCUUGG CUGAUGAG GCCGUUAGGC CGAA ACGUGGCC | 1917 |
| 1594 | ACGACUGU U ACAAGUUU | 143 | AAACUUGU CUGAUGAG GCCGUUAGGC CGAA ACAGUCGU | 1918 |
| 1595 | CGACUGUU A CAAGUUUG | 144 | CAAACUUG CUGAUGAG GCCGUUAGGC CGAA AACAGUCG | 1919 |
| 1601 | UUACAAGU U UGCCAUCU | 145 | AGAUGGCA CUGAUGAG GCCGUUAGGC CGAA ACUUGUAA | 1920 |
| 1602 | UACAAGUU U GCCAUCUC | 146 | GAGAUGGC CUGAUGAG GCCGUUAGGC CGAA AACUUGUA | 1921 |
| 1608 | UUUGCCAU C UCACAGUC | 147 | GACUGUGA CUGAUGAG GCCGUUAGGC CGAA AUGGCAAA | 1922 |
| 1610 | UGCCAUCU C ACAGUCAU | 148 | AUGACUGU CUGAUGAG GCCGUUAGGC CGAA AGAUGGCA | 1923 |
| 1616 | CUCACAGU C AUCCACGG | 149 | CCGUGGAU CUGAUGAG GCCGUUAGGC CGAA ACUGUGAG | 1924 |
| 1619 | ACAGUCAU C CACGGGCA | 150 | UGCCCGUG CUGAUGAG GCCGUUAGGC CGAA AUGACUGU | 1925 |
| 1632 | GGCACUGU U AUGGGAGC | 151 | GCUCCCAU CUGAUGAG GCCGUUAGGC CGAA ACAGUGCC | 1926 |
| 1633 | GCACUGUU A UGGGAGCU | 152 | AGCUCCCA CUGAUGAG GCCGUUAGGC CGAA AACAGUGC | 1927 |
| 1644 | GGAGCUGU U AUCAUGGA | 153 | UCCAUGAU CUGAUGAG GCCGUUAGGC CGAA ACAGCUCC | 1928 |
| 1645 | GAGCUGUU A UCAUGGAG | 154 | CUCCAUGA CUGAUGAG GCCGUUAGGC CGAA AACAGCIC | 1929 |
| 1647 | GCUGUUAU C AUGGAGGG | 155 | CCCUCCAU CUGAUGAG GCCGUUAGGC CGAA AUAACAGC | 1930 |
| 1658 | GGAGGGCU U CUACGUUG | 156 | CAACGUAG CUGAUGAG GCCGUUAGGC CGAA AGCCCUCC | 1931 |
| 1659 | GAGGGCUU C UACGUUGU | 157 | ACAACGUA CUGAUGAG GCCGUUAGGC CGAA AAGCCCUC | 1932 |
| 1661 | GGGCUUCU A CGUUGUCU | 158 | AGACAACG CUGAUGAG GCCGUUAGGC CGAA AGAAGCCC | 1933 |
| 1665 | UUCUACGU U GUCUUUGA | 159 | UCAAAGAC CUGAUGAG GCCGUUAGGC CGAA ACGUAGAA | 1934 |
| 1668 | UACGUUGU C UUUGAUCG | 160 | CGAUCAAA CUGAUGAG GCCGUUAGGC CGAA ACAACGUA | 1935 |
| 1670 | CGUUGUCU U UGAUCGGG | 161 | CCCGAUCA CUGAUGAG GCCGUUAGGC CGAA AGACAACG | 1936 |
| 1671 | GUUGUCUU U GAUCGGGC | 162 | GCCCGAUC CUGAUGAG GCCGUUAGGC CGAA AAGACAAC | 1937 |
| 1675 | UCUUUGAU C GGGCCCGA | 163 | UCGGGCCC CUGAUGAG GCCGUUAGGC CGAA AUCAAAGA | 1938 |
| 1692 | AAACGAAU U GGCUUUGC | 164 | GCAAAGCC CUGAUGAG GCCGUUAGGC CGAA AUUCGUUU | 1939 |
| 1697 | AAUUGGCU U UGCUGUCA | 165 | UGACAGCA CUGAUGAG GCCGUUAGGC CGAA AGCCAAUU | 1940 |
| 1698 | AUUGGCUU U GCUGUCAG | 166 | CUGACAGC CUGAUGAG GCCGUUAGGC CGAA AAGCCAAU | 1941 |
| 1704 | UUUGCUGU C AGCGCUUG | 167 | CAAGCGCU CUGAUGAG GCCGUUAGGC CGAA ACAGCAAA | 1942 |
| 1711 | UCAGCGCU U GCCAUGUG | 168 | CACAUGGC CUGAUGAG GCCGUUAGGC CGAA AGCGCUGA | 1943 |
| 1730 | CGAUGAGU U CAGGACGG | 169 | CCGUCCUG CUGAUGAG GCCGUUAGGC CGAA ACUCAUCG | 1944 |
| 1731 | GAUGAGUU C AGGACGGC | 170 | GCCGUCCU CUGAUGAG GCCGUUAGGC CGAA AACUCAUC | 1945 |
| 1756 | AAGGCCCU U UUGUCACC | 171 | GGUGACAA CUGAUGAG GCCGUUAGGC CGAA AGGGCCUU | 1946 |
| 1757 | AGGCCCUU U UGUCACCU | 172 | AGGUGACA CUGAUGAG GCCGUUAGGC CGAA AAGGGCU | 1947 |
| 1758 | GGCCCUUU U GUCACCUU | 173 | AAGGUGAC CUGAUGAG GCCGUUAGGC CGAA AAAGGGCC | 1948 |
| 1761 | CCUUUUGU C ACCUUGGA | 174 | UCCAAGGU CUGAUGAG GCCGUUAGGC CGAA ACAAAAGG | 1949 |
| 1766 | UGUCACCU U GGACAUGG | 175 | CCAUGUCC CUGAUGAG GCCGUUAGGC CGAA AGGUGACA | 1950 |
| 1787 | CUGUGGCU A CAACAUUC | 176 | GAAUGUUG CUGAUGAG GCCGUUAGGC CGAA AGCCACAG | 1951 |
| 1794 | UACAACAU U CCACAGAC | 177 | GUCUGUGG CUGAUGAG GCCGUUAGGC CGAA AUGUUGUA | 1952 |
| 1795 | ACAACAUU C CACAGACA | 178 | UGUCUGUG CUGAUGAG GCCGUUAGGC CGAA AAUGUUGU | 1953 |
| 1811 | AGAUGAGU C AACCCUCA | 179 | UGAGGGUU CUGAUGAG GCCGUUAGGC CGAA ACUCAUCU | 1954 |
| 1818 | UCAACCCU C AUGACCAU | 180 | AUGGUCAU CUGAUGAG GCCGUUAGGC CGAA AGGGUUGA | 1955 |
| 1827 | AUGACCAU A GCCUAUGU | 181 | ACAUAGGC CUGAUGAG GCCGUUAGGC CGAA AUGGUCAU | 1956 |
| 1832 | CAUAGCCU A UGUCAUGG | 182 | CCAUGACA CUGAUGAG GCCGUUAGGC CGAA AGGCUAUG | 1957 |
| 1836 | GCCUAUGU C AUGGCUGC | 183 | GCAGCCAU CUGAUGAG GCCGUUAGGC CGAA ACAUAGGC | 1958 |
| 1848 | GCUGCCAU C UGCGCCCU | 184 | AGGGCGCA CUGAUGAG GCCGUUAGGC CGAA AUGGCAGC | 1959 |
| 1857 | UGCGCCCU C UUCAUGCU | 185 | AGCAUGAA CUGAUGAG GCCGUUAGGC CGAA AGGGCGCA | 1960 |
| 1859 | CGCCCUCU U CAUGCUGC | 186 | GCAGCAUG CUGAUGAG GCCGUUAGGC CGAA AGAGGGCG | 1961 |
| 1860 | GCCCUCUU C AUGCUGCC | 187 | GGCAGCAU CUGAUGAG GCCGUUAGGC CGAA AAGAGGGC | 1962 |
| 1872 | CUGCCACU C UGCCUCAU | 188 | AUGAGGCA CUGAUGAG GCCGUUAGGC CGAA AGUGGCAG | 1963 |
| 1878 | CUCUGCCU C AUGGUGUG | 189 | CACACCAU CUGAUGAG GCCGUUAGGC CGAA AGGCAGAG | 1964 |
| 1888 | UGGUGUGU C AGUGGCGC | 190 | GCGCCACU CUGAUGAG GCCGUUAGGC CGAA ACACACCA | 1965 |
| 1902 | CGCUGCCU C CGCUGCCU | 191 | AGGCAGCG CUGAUGAG GCCGUUAGGC CGAA AGGCAGCG | 1966 |
| 1931 | UGAUGACU U UGCUGAUG | 192 | CAUCAGCA CUGAUGAG GCCGUUAGGC CGAA AGUCAUCA | 1967 |
| 1932 | GAUGACUU U GCUGAUGA | 193 | UCAUCAGC CUGAUGAG GCCGUUAGGC CGAA AAGUCAUC | 1968 |
| 1944 | GAUGACAU C UCCCUGCU | 194 | AGCAGGGA CUGAUGAG GCCGUUAGGC CGAA AUGUCAUC | 1969 |
| 1946 | UGACAUCU C CCUGCUGA | 195 | UCAGCAGG CUGAUGAG GCCGUUAGGC CGAA AGAUGUCA | 1970 |
| 1981 | CAGAAGAU A GAGAUUCC | 196 | GGAAUCUC CUGAUGAG GCCGUUAGGC CGAA AUCUUCUG | 1971 |
| 1987 | AUAGAGAU U CCCCUGGA | 197 | UCCAGGGG CUGAUGAG GCCGUUAGGC CGAA AUCUCUAU | 1972 |
| 1988 | UAGAGAUU C CCCUGGAC | 198 | GUCCAGGG CUGAUGAG GCCGUUAGGC CGAA AAUCUCUA | 1973 |
| 2004 | CCACACCU C CGUGGUUC | 199 | GAACCACG CUGAUGAG GCCGUUAGGC CGAA AGGUGUGG | 1974 |
| 2011 | UCCGUGGU U CACUUUGG | 200 | CCAAAGUG CUGAUGAG GCCGUUAGGC CGAA ACCACGGA | 1975 |
| 2012 | CCGUGGUU C ACUUUGGU | 201 | ACCAAAGU CUGAUGAG GCCGUUAGGC CGAA AACCACGG | 1976 |
| 2016 | GGUUCACU U UGGUCACA | 202 | UGUGACCA CUGAUGAG GCCGUUAGGC CGAA AGUGAACC | 1977 |
| 2017 | GUUCACUU U GGUCACAA | 203 | UUGUGACC CUGAUGAG GCCGUUAGGC CGAA AAGUGAAC | 1978 |
| 2021 | ACUUUGGU C ACAAGUAG | 204 | CUACUUGU CUGAUGAG GCCGUUAGGC CGAA ACCAAAGU | 1979 |
| 2028 | UCACAAGU A GGAGACAC | 205 | GUGUCUCC CUGAUGAG GCCGUUAGGC CGAA ACUUGUGA | 1980 |
| 2063 | GAGCACCU C AGGACCCU | 206 | AGGGUCCU CUGAUGAG GCCGUUAGGC CGAA AGGUGCUC | 1981 |
| 2072 | AGGACCCU C CCCACCCA | 207 | UGGGUGGG CUGAUGAG GCCGUUAGGC CGAA AGGGUCCU | 1982 |
| 2091 | AAAUGCCU C UGCCUUGA | 208 | UCAAGGCA CUGAUGAG GCCGUUAGGC CGAA AGGCAUUU | 1983 |
| 2097 | CUCUGCCU U GAUGGAGA | 209 | UCUCCAUC CUGAUGAG GCCGUUAGGC CGAA AGGCAGAG | 1984 |
| 2129 | AGGUGGGU U CCAGGGAC | 210 | GUCCCUGG CUGAUGAG GCCGUUAGGC CGAA ACCCACCU | 1985 |
| 2130 | GGUGGGUU C CAGGGACU | 211 | AGUCCCUG CUGAUGAG GCCGUUAGGC CGAA AACCCACC | 1986 |
| 2141 | GGGACUGU A CCUGUAGG | 212 | CCUACAGG CUGAUGAG GCCGUUAGGC CGAA ACAGUCCC | 1987 |
| 2147 | GUACCUGU A GGAAACAG | 213 | CUGUUUCC CUGAUGAG GCCGUUAGGC CGAA ACAGGUAC | 1988 |
| 2177 | GAAGCACU C UGCUGGCG | 214 | CGCCAGCA CUGAUGAG GCCGUUAGGC CGAA AGUGCUUC | 1989 |
| 2191 | GCGGGAAU A CUCUUGGU | 215 | ACCAAGAG CUGAUGAG GCCGUUAGGC CGAA AUUCCCGC | 1990 |
| 2194 | GGAAUACU C UUGGUCAC | 216 | GUGACCAA CUGAUGAG GCCGUUAGGC CGAA AGUAUUCC | 1991 |
| 2196 | AAUACUCU U GGUCACCU | 217 | AGGUGACC CUGAUGAG GCCGUUAGGC CGAA AGAGUAUU | 1992 |
| 2200 | CUCUUGGU C ACCUCAAA | 218 | UUUGAGGU CUGAUGAG GCCGUUAGGC CGAA ACCAAGAG | 1993 |
| 2205 | GGUCACCU C AAAUUUAA | 219 | UUAAAUUU CUGAUGAG GCCGUUAGGC CGAA AGGUGACC | 1994 |
| 2210 | CCUCAAAU U UAAGUCGG | 220 | CCGACUUA CUGAUGAG GCCGUUAGGC CGAA AUUUGAGG | 1995 |
| 2211 | CUCAAAUU U AAGUCGGG | 221 | CCCGACUU CUGAUGAG GCCGUUAGGC CGAA AAUUUGAG | 1996 |
| 2212 | UCAAAUUU A AGUCUGGA | 222 | UCCCGACU CUGAUGAG GCCGUUAGGC CGAA AAAUUUGA | 1997 |
| 2216 | AUUUAAGU C GGGAAAUU | 223 | AAUUUCCC CUGAUGAG GCCGUUAGGC CGAA ACUUAAAU | 1998 |
| 2224 | CGGGAAAU U CUGCUGCU | 224 | AGCAGCAG CUGAUGAG GCCGUUAGGC CGAA AUUUCCCG | 1999 |
| 2225 | GGGAAAUU C UGCUGCUU | 225 | AAGCAGCA CUGAUGAG GCCGUUAGGC CGAA AAUUUCCC | 2000 |
| 2233 | CUGCUGCU U GAAACUUC | 226 | GAAGUUUC CUGAUGAG GCCGUUAGGC CGAA AGCAGCAG | 2001 |
| 2240 | UUGAAACU U CAGCCCUG | 227 | CAGGGCUG CUGAUGAG GCCGUUAGGC CGAA AGUUUCAA | 2002 |
| 2241 | UGAAACUU C AGCCCUGA | 228 | UCAGGGCU CUGAUGAG GCCGUUAGGC CGAA AAGUUUCA | 2003 |
| 2254 | CUGAACCU U UGUCCACC | 229 | GGUGGACA CUGAUGAG GCCGUUAGGC CGAA AGGUUCAG | 2004 |
| 2255 | UGAACCUU U GUCCACCA | 230 | UGGUGGAC CUGAUGAG GCCGUUAGGC CGAA AAGGUUCA | 2005 |
| 2258 | ACCUUUGU C CACCAUUC | 231 | GAAUGGUG CUGAUGAG GCCGUUAGGC CGAA ACAAAGGU | 2006 |
| 2265 | UCCACCAU U CCUUUAAA | 232 | UUUAAAGG CUGAUGAG GCCGUUAGGC CGAA AUGGUGGA | 2007 |
| 2266 | CCACCAUU C CUUUAAAU | 233 | AUUUAAAG CUGAUGAG GCCGUUAGGC CGAA AAUGGUGG | 2008 |
| 2269 | CCAUUCCU U UAAAUUCU | 234 | AGAAUUUA CUGAUGAG GCCGUUAGGC CGAA AGGAAUGG | 2009 |
| 2270 | CAUUCCUU U AAAUUCUC | 235 | GAGAAUUU CUGAUGAG GCCGUUAGGC CGAA AAGGAAUG | 2010 |
| 2271 | AUUCCUUU A AAUUCUCC | 236 | GGAGAAUU CUGAUGAG GCCGUUAGGC CGAA AAAGGAAU | 2011 |
| 2275 | CUUUAAAU U CUCCAACC | 237 | GGUUGGAG CUGAUGAG GCCGUUAGGC CGAA AUUUAAAG | 2012 |
| 2276 | UUUAAAUU C UCCAACCC | 238 | GGGUUGGA CUGAUGAG GCCGUUAGGC CGAA AAUUUAAA | 2013 |
| 2278 | UAAAUUCU C CAACCCAA | 239 | UUGGGUUG CUGAUGAG GCCGUUAGGC CGAA AGAAUUUA | 2014 |
| 2290 | CCCAAAGU A UUCUUCUU | 240 | AAGAAGAA CUGAUGAG GCCGUUAGGC CGAA ACUUUGGG | 2015 |
| 2292 | CAAAGUAU U CUUCUUUU | 241 | AAAAGAAG CUGAUGAG GCCGUUAGGC CGAA AUACUUUG | 2016 |
| 2293 | AAAGUAUU C UUCUUUUC | 242 | GAAAAGAA CUGAUGAG GCCGUUAGGC CGAA AAUACUUU | 2017 |
| 2295 | AGUAUUCU U CUUUUCUU | 243 | AAGAAAAG CUGAUGAG GCCGUUAGGC CGAA AGAAUACU | 2018 |
| 2296 | GUAUUCUU C UUUUCUUA | 244 | UAAGAAAA CUGAUGAG GCCGUUAGGC CGAA AAGAAUAC | 2019 |
| 2298 | AUUCUUCU U UUCUUAGU | 245 | ACUAAGAA CUGAUGAG GCCGUUAGGC CGAA AGAAGAAU | 2020 |
| 2299 | UUCUUCUU U UCUUAGUU | 246 | AACUAAGA CUGAUGAG GCCGUUAGGC CGAA AAGAAGAA | 2021 |
| 2300 | UCUUCUUU U CUUAGUUU | 247 | AAACUAAG CUGAUGAG GCCGUUAGGC CGAA AAAGAAGA | 2022 |
| 2301 | CUUCUUUU C UUAGUUUC | 248 | GAAACUAA CUGAUGAG GCCGUUAGGC CGAA AAAAGAAG | 2023 |
| 2303 | UCUUUUCU U AGUUUCAG | 249 | CUGAAACU CUGAUGAG GCCGUUAGGC CGAA AGAAAAGA | 2024 |
| 2304 | CUUUUCUU A GUUUCAGA | 250 | UCUGAAAC CUGAUGAG GCCGUUAGGC CGAA AAGAAAAG | 2025 |
| 2307 | UUCUUAGU U UCAGAAGU | 251 | ACUUCUGA CUGAUGAG GCCGUUAGGC CGAA ACUAAGAA | 2026 |
| 2308 | UCUUAGUU U CAGAAGUA | 252 | UACUUCUG CUGAUGAG GCCGUUAGGC CGAA AACUAAGA | 2027 |
| 2309 | CUUAGUUU C AGAAGUAC | 253 | GUACUUCU CUGAUGAG GCCGUUAGGC CGAA AAACUAAG | 2028 |
| 2316 | UCAGAAGU A CUGGCAUC | 254 | GAUGCCAG CUGAUGAG GCCGUUAGGC CGAA ACUUCUGA | 2029 |
| 2324 | ACUGGCAU C ACACGCAG | 255 | CUGCGUGU CUGAUGAG GCCGUUAGGC CGAA AUGCCAGU | 2030 |
| 2335 | ACGCAGGU U ACCUUGGC | 256 | GCCAAGGU CUGAUGAG GCCGUUAGGC CGAA ACCUGCGU | 2031 |
| 2336 | CGCAGGUU A CCUUGGCG | 257 | CGCCAAGG CUGAUGAG GCCGUUAGGC CGAA AACCUGCG | 2032 |
| 2340 | GGUUACCU U GGCGUGUG | 258 | CACACGCC CUGAUGAG GCCGUUAGGC CGAA AGGUAACC | 2033 |
| 2350 | GCGUGUGU C CCUGUGGU | 259 | ACCACAGG CUGAUGAG GCCGUUAGGC CGAA ACACACGC | 2034 |
| 2359 | CCUGUGGU A CCCUGGCA | 260 | UGCCAGGG CUGAUGAG GCCGUUAGGC CGAA ACCACAGG | 2035 |
| 2384 | ACCAAGCU U GUUUCCCU | 261 | AGGGAAAC CUGAUGAG GCCGUUAGGC CGAA AGCUUGGU | 2036 |
| 2387 | AAGCUUGU U UCCCUGCU | 262 | AGCAGGGA CUGAUGAG GCCGUUAGGC CGAA ACAAGCUU | 2037 |
| 2388 | AGCUUGUU U CCCUGCUG | 263 | CAGCAGGG CUGAUGAG GCCGUUAGGC CGAA AACAAGCU | 2038 |
| 2389 | GCUUGUUU C CCUGCUGG | 264 | CCAGCAGG CUGAUGAG GCCGUUAGGC CGAA AAACAAGC | 2039 |
| 2405 | GCCAAAGU C AGUAGGAG | 265 | CUCCUACU CUGAUGAG GCCGUUAGGC CGAA ACUUUGGC | 2040 |
| 2409 | AAGUCAGU A GGAGAGGA | 266 | UCCUCUCC CUGAUGAG GCCGUUAGGC CGAA ACUGACUU | 2041 |
| 2426 | UGCACAGU U UGCUAUUU | 267 | AAAUAGCA CUGAUGAG GCCGUUAGGC CGAA ACUGUGCA | 2042 |
| 2427 | GCACAGUU U GCUAUUUG | 268 | CAAAUAGC CUGAUGAG GCCGUUAGGC CGAA AACUGUGC | 2043 |
| 2431 | AGUUUGCU A UUUGCUUU | 269 | AAAGCAAA CUGAUGAG GCCGUUAGGC CGAA AGCAAACU | 2044 |
| 2433 | UUUGCUAU U UGCUUUAG | 270 | CUAAAGCA CUGAUGAG GCCGUUAGGC CGAA AUAGCAAA | 2045 |
| 2434 | UUGCUAUU U GCUUUAGA | 271 | UCUAAAGC CUGAUGAG GCCGUUAGGC CGAA AAUAGCAA | 2046 |
| 2438 | UAUUUGCU U UAGAGACA | 272 | UGUCUCUA CUGAUGAG GCCGUUAGGC CGAA AGCAAAUA | 2047 |
| 2439 | AUUUGCUU U AGAGACAG | 273 | CUGUCUCU CUGAUGAG GCCGUUAGGC CGAA AAGCAAAU | 2048 |
| 2440 | UUUGCUUU A GAGACAGG | 274 | CCUGUCUC CUGAUGAG GCCGUUAGGC CGAA AAAGCAAA | 2049 |
| 2455 | GGGACUGU A UAAACAAG | 275 | CUUGUUUA CUGAUGAG GCCGUUAGGC CGAA ACAGUCCC | 2050 |
| 2457 | GACUGUAU A AACAAGCC | 276 | GGCUUGUU CUGAUGAG GCCGUUAGGC CGAA AUACAGUC | 2051 |
| 2467 | ACAAGCCU A ACAUUGGU | 277 | ACCAAUGU CUGAUGAG GCCGUUAGGC CGAA AGGCUUGU | 2052 |
| 2472 | CCUAACAU U GGUGCAAA | 278 | UUUGCACC CUGAUGAG GCCGUUAGGC CGAA AUGUUAGG | 2053 |
| 2484 | GCAAAGAU U GCCUCUUG | 279 | CAAGAGGC CUGAUGAG GCCGUUAGGC CGAA AUCUUUGC | 2054 |
| 2489 | GAUUGCCU C UUGAAUUA | 280 | UAAUUCAA CUGAUGAG GCCGUUAGGC CGAA AGGCAAUC | 2055 |
| 2491 | UUGCCUCU U GAAUUAAA | 281 | UUUAAUUC CUGAUGAG GCCGUUAGGC CGAA AGAGGCAA | 2056 |
| 2496 | UCUUGAAU U AAAAAAAA | 282 | UUUUUUUU CUGAUGAG GCCGUUAGGC CGAA AUUCAAGA | 2057 |
| 2497 | CUUGAAUU A AAAAAAAA | 283 | UUUUUUUU CUGAUGAG GCCGUUAGGC CGAA AAUUCAAG | 2058 |
| 2510 | AAAAAACU A GAAAAAAA | 284 | UUUUUUUC CUGAUGAG GCCGUUAGGC CGAA AGUUUUUU | 2059 |

Input Sequence = AF100308.
Cut Site = UH/.
Stem Length = 8.
Core Sequence = CUGAUGAG GCCGUUAGGC CGAA AF100308 (Hepatitis B virus strain 2-18, 3215 bp)
Underlined region can be any X sequence or linker, as described herein.

TABLE IV
Human BACE Inozyme and Target Sequence

| Pos | Substrate | Seq ID | Inozyme | Seq ID |
|---|---|---|---|---|
| 10 | CACGCGYC C GCAGCCCG | 285 | CGGGCUGC CUGAUGAG GCCGUUAGGC CGAA IACGCGUG | 2060 |
| 13 | GCGUCCGC A GCCCGCCC | 286 | GGGCGGGC CUGAUGAG GCCGUUAGGC CGAA ICGGACGC | 2061 |
| 16 | UCCGCAGC C CGCCCGGG | 287 | CCCGGGCG CUGAUGAG GCCGUUAGGC CGAA ICUGCGGA | 2062 |
| 17 | CCGCAGCC C GCCCGGGA | 288 | UCCCGGGC CUGAUGAG GCCGUUAGGC CGAA IGCUGCGG | 2063 |
| 20 | CAGCCCGC C CGGGAGCU | 289 | AGCUCCCG CUGAUGAG GCCGUUAGGC CGAA ICGGGCUG | 2064 |
| 21 | AGCCCGCC C GGGAGCUG | 290 | CAGCUCCC CUGAUGAG GCCGUUAGGC CGAA IGCGGGCU | 2065 |
| 28 | CCGGGAGC U GCGAGCCG | 291 | CGGCUCGC CUGAUGAG GCCGUUAGGC CGAA ICUCCCGG | 2066 |
| 35 | CUGCGAGC C GCGAGCUG | 292 | CAGCUCGC CUGAUGAG GCCGUUAGGC CGAA ICUCGCAG | 2067 |
| 42 | CCGCGAGC U GGAUUAUG | 293 | CAUAAUCC CUGAUGAG GCCGUUAGGC CGAA ICUCGCGG | 2068 |
| 56 | AUGGUGGC C UGAGCAGC | 294 | GCUGCUCA CUGAUGAG GCCGUUAGGC CGAA ICCACCAU | 2069 |
| 57 | UGGUGGCC U GAGCAGCC | 295 | GGCUGCUC CUGAUGAG GCCGUUAGGC CGAA IGCCACCA | 2070 |
| 62 | GCCUGAGC A GCCAACGC | 296 | GCGUUGGC CUGAUGAG GCCGUUAGGC CGAA ICUCAGGC | 2071 |
| 65 | UGAGCAGC C AACGCAGC | 297 | GCUGCGUU CUGAUGAG GCCGUUAGGC CGAA ICUGCUCA | 2072 |
| 66 | GAGCAGCC A ACGCAGCC | 298 | GGCUGCGU CUGAUGAG GCCGUUAGGC CGAA IGCUGCUC | 2073 |
| 71 | GCCAACGC A GCCGCAGG | 299 | CCUGCGGC CUGAUGAG GCCGUUAGGC CGAA ICGUUGGC | 2074 |
| 74 | AACGCAGC C GCAGGAGC | 300 | GCUCCUGC CUGAUGAG GCCGUUAGGC CGAA ICUGCGUU | 2075 |
| 77 | GCAGCCGC A GGAGCCCG | 301 | CGGGCUCC CUGAUGAG GCCGUUAGGC CGAA ICGGCUGC | 2076 |
| 83 | GCAGGAGC C CGGAGCCC | 302 | GGGCUCCG CUGAUGAG GCCGUUAGGC CGAA ICUCCUGC | 2077 |
| 84 | CAGGAGCC C GGAGCCCU | 303 | AGGGCUCC CUGAUGAG GCCGUUAGGC CGAA IGCUCCUG | 2078 |
| 90 | CCCGGAGC C CUUGCCCC | 304 | GGGGCAAG CUGAUGAG GCCGUUAGGC CGAA ICUCCGGG | 2079 |
| 91 | CCGGAGCC C UUGCCCCU | 305 | AGGGGCAA CUGAUGAG GCCGUUAGGC CGAA IGCUCCGG | 2080 |
| 92 | CGGAGCCC U UGCCCCUG | 306 | CAGGGGCA CUGAUGAG GCCGUUAGGC CGAA IGGCUCCG | 2081 |
| 96 | GCCCUUGC C CCUGCCCG | 307 | CGGGCAGG CUGAUGAG GCCGUUAGGC CGAA ICAAGGGC | 2082 |
| 97 | CCCUUGCC C CUGCCCGC | 308 | GCGGGCAG CUGAUGAG GCCGUUAGGC CGAA IGCAAGGG | 2083 |
| 98 | CCUUGCCC C UGCCCGCG | 309 | CGCGGGCA CUGAUGAG GCCGUUAGGC CGAA IGGCAAGG | 2084 |
| 99 | CUUGCCCC U GCCCGCGC | 310 | GCGCGGGC CUGAUGAG GCCGUUAGGC CGAA IGGGCAAG | 2085 |
| 102 | GCCCCUGC C CGCGCCGC | 311 | GCGGCGCG CUGAUGAG GCCGUUAGGC CGAA ICAGGGGC | 2086 |
| 103 | CCCCUGCC C GCGCCGCC | 312 | GGCGGCGC CUGAUGAG GCCGUUAGGC CGAA IGCAGGGG | 2087 |
| 108 | GCCCGCGC C GCCGCCCG | 313 | CGGGCGGC CUGAUGAG GCCGUUAGGC CGAA ICGCGGGC | 2088 |
| 111 | CGCGCCGC C GCCCGCCG | 314 | CGGCGGGC CUGAUGAG GCCGUUAGGC CGAA ICGGCGCG | 2089 |
| 114 | GCCGCCGC C CGCCGGGG | 315 | CCCCGGCG CUGAUGAG GCCGUUAGGC CGAA ICGGCGGC | 2090 |
| 115 | CCGCCGCC C GCCGGGGG | 316 | CCCCCGGC CUGAUGAG GCCGUUAGGC CGAA IGCGGCGG | 2091 |
| 118 | CCGCCCGC C GGGGGGAC | 317 | GUCCCCCC CUGAUGAG GCCGUUAGGC CGAA ICGGGCGG | 2092 |
| 127 | GGGGGGAC C AGGGAAGC | 318 | GCUUCCCU CUGAUGAG GCCGUUAGGC CGAA IUCCCCCC | 2093 |
| 128 | GGGGGACC A GGGAAGCC | 319 | GGCUUCCC CUGAUGAG GCCGUUAGGC CGAA IGUCCCCC | 2094 |
| 136 | AGGGAAGC C GCCACCGG | 320 | CCGGUGGC CUGAUGAG GCCGUUAGGC CGAA ICUUCCCU | 2095 |
| 139 | GAAGCCGC C ACCGGCCC | 321 | GGGCCGGU CUGAUGAG GCCGUUAGGC CGAA ICGGCUUC | 2096 |
| 140 | AAGCCGCC A CCGGCCCG | 322 | CGGGCCGG CUGAUGAG GCCGUUAGGC CGAA IGCGGCUU | 2097 |
| 142 | GCCGCCAC C GGCCCGCC | 323 | GGCGGGCC CUGAUGAG GCCGUUAGGC CGAA IUGGCGGC | 2098 |
| 146 | CCACCGGC C CGCCAUGC | 324 | GCAUGGCG CUGAUGAG GCCGUUAGGC CGAA ICCGGUGG | 2099 |
| 147 | CACCGGCC C GCCAUGCC | 325 | GGCAUGGC CUGAUGAG GCCGUUAGGC CGAA IGCCGGUG | 2100 |
| 150 | CGGCCCGC C AUGCCCGC | 326 | GCGGGCAU CUGAUGAG GCCGUUAGGC CGAA ICGGGCCG | 2101 |
| 151 | GGCCCGCC A UGCCCGCC | 327 | GGCGGGCA CUGAUGAG GCCGUUAGGC CGAA IGCGGGCC | 2102 |
| 155 | CGCCAUGC C CGCCCCUC | 328 | GAGGGGCG CUGAUGAG GCCGUUAGGC CGAA ICAUGGCG | 2103 |
| 156 | GCCAUGCC C GCCCCUCC | 329 | GGAGGGGC CUGAUGAG GCCGUUAGGC CGAA IGCAUGGC | 2104 |
| 159 | AUGCCCGC C CCUCCCAG | 330 | CUGGGAGG CUGAUGAG GCCGUUAGGC CGAA ICGGGCAU | 2105 |
| 160 | UGCCCGCC C CUCCCAGC | 331 | GCUGGGAG CUGAUGAG GCCGUUAGGC CGAA IGCGGGCA | 2106 |
| 161 | GCCCGCCC C UCCCAGCC | 332 | GGCUGGGA CUGAUGAG GCCGUUAGGC CGAA IGGCGGGC | 2107 |
| 162 | CCCGCCCC U CCCAGCCC | 333 | GGGCUGGG CUGAUGAG GCCGUUAGGC CGAA IGGGCGGG | 2108 |
| 164 | CGCCCCUC C CAGCCCCG | 334 | CGGGGCUG CUGAUGAG GCCGUUAGGC CGAA IAGGGGCG | 2109 |
| 165 | GCCCCUCC C AGCCCCGC | 335 | GCGGGGCU CUGAUGAG GCCGUUAGGC CGAA IGAGGGGC | 2110 |
| 166 | CCCCUCCC A GCCCCGCC | 336 | GGCGGGGC CUGAUGAG GCCGUUAGGC CGAA IGGAGGGG | 2111 |
| 169 | CUCCCAGC C CCGCCGGG | 337 | CCCGGCGG CUGAUGAG GCCGUUAGGC CGAA ICUGGGAG | 2112 |
| 170 | UCCCAGCC C CGCCGGGA | 338 | UCCCGGCG CUGAUGAG GCCGUUAGGC CGAA IGCUGGGA | 2113 |
| 171 | CCCAGCCC C GCCGGGAG | 339 | CUCCCGGC CUGAUGAG GCCGUUAGGC CGAA IGGCUGGG | 2114 |
| 174 | AGCCCCGC C GGGAGCCC | 340 | GGGCUCCC CUGAUGAG GCCGUUAGGC CGAA ICGGGGCU | 2115 |
| 181 | CCGGGAGC C CGCGCCCG | 341 | CGGGCGCG CUGAUGAG GCCGUUAGGC CGAA ICUCCCGG | 2116 |
| 182 | CGGGAGCC C GCGCCCGC | 342 | GCGGGCGC CUGAUGAG GCCGUUAGGC CGAA IGCUCCCG | 2117 |
| 187 | GCCCGCGC C CGCUGCCC | 343 | GGGCAGCG CUGAUGAG GCCGUUAGGC CGAA ICGCGGGC | 2118 |
| 188 | CCCGCGCC C GCUGCCCA | 344 | UGGGCAGC CUGAUGAG GCCGUUAGGC CGAA IGCGCGGG | 2119 |
| 191 | GCGCCCGC U GCCCAGGC | 345 | GCCUGGGC CUGAUGAG GCCGUUAGGC CGAA ICGGGCGC | 2120 |
| 194 | CCCGCUGC C CAGGCUGG | 346 | CCAGCCUG CUGAUGAG GCCGUUAGGC CGAA ICAGCGGG | 2121 |
| 195 | CCGCUGCC C AGGCUGGC | 347 | GCCAGCCU CUGAUGAG GCCGUUAGGC CGAA IGCAGCGG | 2122 |
| 196 | CGCUGCCC A GGCUGGCC | 348 | GGCCAGCC CUGAUGAG GCCGUUAGGC CGAA IGGCAGCG | 2123 |
| 200 | GCCCAGGC U GGCCGCCG | 349 | CGGCGGCC CUGAUGAG GCCGUUAGGC CGAA ICCUGGGC | 2124 |
| 204 | AGGCUGGC C GCCGCCGU | 350 | ACGGCGGC CUGAUGAG GCCGUUAGGC CGAA ICCAGCCU | 2125 |
| 207 | CUGGCCGC C GCCGUGCC | 351 | GGCACGGC CUGAUGAG GCCGUUAGGC CGAA ICGGCCAG | 2126 |
| 210 | GCCGCCGC C GUGCCGAU | 352 | AUCGGCAC CUGAUGAG GCCGUUAGGC CGAA ICGGCGGC | 2127 |
| 215 | CGCCGUGC C GAUGUAGC | 353 | GCUACAUC CUGAUGAG GCCGUUAGGC CGAA ICACGGCG | 2128 |
| 228 | UAGCGGGC U CCGGAUCC | 354 | GGAUCCGG CUGAUGAG GCCGUUAGGC CGAA ICCCGCUA | 2129 |
| 230 | GCGGGCUC C GGAUCCCA | 355 | UGGGAUCC CUGAUGAG GCCGUUAGGC CGAA IAGCCCGC | 2130 |
| 236 | UCCGGAUC C CAGCCUCU | 356 | AGAGGCUG CUGAUGAG GCCGUUAGGC CGAA IAUCCGGA | 2131 |
| 237 | CCGGAUCC C AGCCUCUC | 357 | GAGAGGCU CUGAUGAG GCCGUUAGGC CGAA IGAUCCGG | 2132 |
| 238 | CGGAUCCC A GCCUCUCC | 358 | GGAGAGGC CUGAUGAG GCCGUUAGGC CGAA IGGAUCCG | 2133 |
| 241 | AUCCCAGC C UCUCCCCU | 359 | AGGGGAGA CUGAUGAG GCCGUUAGGC CGAA ICUGGGAU | 2134 |
| 242 | UCCCAGCC U CUCCCCUG | 360 | CAGGGGAG CUGAUGAG GCCGUUAGGC CGAA IGCUGGGA | 2135 |
| 244 | CCAGCCUC U CCCCUGCU | 361 | AGCAGGGG CUGAUGAG GCCGUUAGGC CGAA IAGGCUGG | 2136 |
| 246 | AGCCUCUC C CCUGCUCC | 362 | GGAGCAGG CUGAUGAG GCCGUUAGGC CGAA IAGAGGCU | 2137 |
| 247 | GCCUCUCC C CUGCUCCC | 363 | GGGAGCAG CUGAUGAG GCCGUUAGGC CGAA IGAGAGGC | 2138 |
| 248 | CCUCUCCC C UGCUCCCG | 364 | CGGGAGCA CUGAUGAG GCCGUUAGGC CGAA IGGAGAGG | 2139 |
| 249 | CUCUCCCC U GCUCCCGU | 365 | ACGGGAGC CUGAUGAG GCCGUUAGGC CGAA IGGGAGAG | 2140 |
| 252 | UCCCCUGC U CCCGUGCU | 366 | AGCACGGG CUGAUGAG GCCGUUAGGC CGAA ICAGGGGA | 2141 |
| 254 | CCCUGCUC C CGUGCUCU | 367 | AGAGCACG CUGAUGAG GCCGUUAGGC CGAA IAGCAGGG | 2142 |
| 255 | CCUGCUCC C GUGCUCUG | 368 | CAGAGCAC CUGAUGAG GCCGUUAGGC CGAA IGAGCAGG | 2143 |
| 260 | UCCCGUGC U CUGCGGAU | 369 | AUCCGCAG CUGAUGAG GCCGUUAGGC CGAA ICACGGGA | 2144 |
| 262 | CCGUGCUC U GCGGAUCU | 370 | AGAUCCGC CUGAUGAG GCCGUUAGGC CGAA IAGCACGG | 2145 |
| 270 | UGCGGAUC U CCCCUGAC | 371 | GUCAGGGG CUGAUGAG GCCGUUAGGC CGAA IAUCCGCA | 2146 |
| 272 | CGGAUCUC C CCUGACCG | 372 | CGGUCAGG CUGAUGAG GCCGUUAGGC CGAA IAGAUCCG | 2147 |
| 273 | GGAUCUCC C CUGACCGC | 373 | GCGGUCAG CUGAUGAG GCCGUUAGGC CGAA IGAGAUCC | 2148 |
| 274 | GAUCUCCC C UGACCGCU | 374 | AGCGGUCA CUGAUGAG GCCGUUAGGC CGAA IGGAGAUC | 2149 |
| 275 | AUCUCCCC U GACCGCUC | 375 | GAGCGGUC CUGAUGAG GCCGUUAGGC CGAA IGGGAGAU | 2150 |
| 279 | CCCCUGAC C GCUCUCCA | 376 | UGGAGAGC CUGAUGAG GCCGUUAGGC CGAA IUCAGGGG | 2151 |
| 282 | CUGACCGC U CUCCACAG | 377 | CUGUGGAG CUGAUGAG GCCGUUAGGC CGAA ICGGUCAG | 2152 |
| 284 | GACCGCUC U CCACAGCC | 378 | GGCUGUGG CUGAUGAG GCCGUUAGGC CGAA IAGCGGUC | 2153 |
| 286 | CCGCUCUC C ACAGCCCG | 379 | CGGGCUGU CUGAUGAG GCCGUUAGGC CGAA IAGAGCGG | 2154 |
| 287 | CGCUCUCC A CAGCCCGG | 380 | CCGGGCUG CUGAUGAG GCCGUUAGGC CGAA IGAGAGCG | 2155 |
| 289 | CUCUCCAC A GCCCGGAC | 381 | GUCCGGGC CUGAUGAG GCCGUUAGGC CGAA IUGGAGAG | 2156 |
| 292 | UCCACAGC C CGGACCCG | 382 | CGGGUCCG CUGAUGAG GCCGUUAGGC CGAA ICUGUGGA | 2157 |
| 293 | CCACAGCC C GGACCCGG | 383 | CCGGGUCC CUGAUGAG GCCGUUAGGC CGAA IGCUGUGG | 2158 |
| 298 | GCCCGGAC C CGGGGGCU | 384 | AGCCCCCG CUGAUGAG GCCGUUAGGC CGAA IUCCGGGC | 2159 |
| 299 | CCCGGACC C GGGGGCUG | 385 | CAGCCCCC CUGAUGAG GCCGUUAGGC CGAA IGUCCGGG | 2160 |
| 306 | CCGGGGGC U GGCCCAGG | 386 | CCUGGGCC CUGAUGAG GCCGUUAGGC CGAA ICCCCCGG | 2161 |
| 310 | GGGCUGGC C CAGGGCCC | 387 | GGGCCCUG CUGAUGAG GCCGUUAGGC CGAA ICCAGCCC | 2162 |
| 311 | GGCUGGCC C AGGGCCCU | 388 | AGGGCCCU CUGAUGAG GCCGUUAGGC CGAA IGCCAGCC | 2163 |
| 312 | GCUGGCCC A GGGCCCUG | 389 | CAGGGCCC CUGAUGAG GCCGUUAGGC CGAA IGGCCAGC | 2164 |
| 317 | CCCAGGGC C CUGCAGGC | 390 | GCCUGCAG CUGAUGAG GCCGUUAGGC CGAA ICCCUGGG | 2165 |
| 318 | CCAGGGCC C UGCAGGCC | 391 | GGCCUGCA CUGAUGAG GCCGUUAGGC CGAA IGCCCUGG | 2166 |
| 319 | CAGGGCCC U GCAGGCCC | 392 | GGGCCUGC CUGAUGAG GCCGUUAGGC CGAA IGGCCCUG | 2167 |
| 322 | GGCCCUGC A GGCCCUGG | 393 | CCAGGGCC CUGAUGAG GCCGUUAGGC CGAA ICAGGGCC | 2168 |
| 326 | CUGCAGGC C CUGGCGUC | 394 | GACGCCAG CUGAUGAG GCCGUUAGGC CGAA ICCUGCAG | 2169 |
| 327 | UGCAGGCC C UGGCGUCC | 395 | GGACGCCA CUGAUGAG GCCGUUAGGC CGAA IGCCUGCA | 2170 |
| 328 | GCAGGCCC U GGCGUCCU | 396 | AGGACGCC CUGAUGAG GCCGUUAGGC CGAA IGGCCUGC | 2171 |
| 335 | CUGGCGUC C UGAUGCCC | 397 | GGGCAUCA CUGAUGAG GCCGUUAGGC CGAA IACGCCAG | 2172 |
| 336 | UGGCGUCC U GAUGCCCC | 398 | GGGGCAUC CUGAUGAG GCCGUUAGGC CGAA IGACGCCA | 2173 |
| 342 | CCUGAUGC C CCCAAGCU | 399 | AGCUUGGG CUGAUGAG GCCGUUAGGC CGAA ICAUCAGG | 2174 |
| 343 | CUGAUGCC C CCAAGCUC | 400 | GAGCUUGG CUGAUGAG GCCGUUAGGC CGAA IGCAUCAG | 2175 |
| 344 | UGAUGCCC C CAAGCUCC | 401 | GGAGCUUG CUGAUGAG GCCGUUAGGC CGAA IGGCAUCA | 2176 |
| 345 | GAUGCCCC C AAGCUCCC | 402 | GGGAGCUU CUGAUGAG GCCGUUAGGC CGAA IGGGCAUC | 2177 |
| 346 | AUGCCCCC A AGCUCCCU | 403 | AGGGAGCU CUGAUGAG GCCGUUAGGC CGAA IGGGGCAU | 2178 |
| 350 | CCCCAAGC U CCCUCUCC | 404 | GGAGAGGG CUGAUGAG GCCGUUAGGC CGAA ICUUGGGG | 2179 |
| 352 | CCAAGCUC C CUCUCCUG | 405 | CAGGAGAG CUGAUGAG GCCGUUAGGC CGAA IAGCUUGG | 2180 |
| 353 | CAAGCUCC C UCUCCUGA | 406 | UCAGGAGA CUGAUGAG GCCGUUAGGC CGAA IGAGCUUG | 2181 |
| 354 | AAGCUCCC U CUCCUGAG | 407 | CUCAGGAG CUGAUGAG GCCGUUAGGC CGAA IGGAGCUU | 2182 |
| 356 | GCUCCCUC U CCUGAGAA | 408 | UUCUCAGG CUGAUGAG GCCGUUAGGC CGAA IAGGGAGC | 2183 |
| 358 | UCCCUCUC C UGAGAAGC | 409 | GCUUCUCA CUGAUGAG GCCGUUAGGC CGAA IAGAGGA | 2184 |
| 359 | CCCUCUCC U GAGAAGCC | 410 | GGCUUCUC CUGAUGAG GCCGUUAGGC CGAA IGAGAGGG | 2185 |
| 367 | UGAGAAGC C ACCAGCAC | 411 | GUGCUGGU CUGAUGAG GCCGUUAGGC CGAA ICUUCUCA | 2186 |
| 368 | GAGAAGCC A CCAGCACC | 412 | GGUGCUGG CUGAUGAG GCCGUUAGGC CGAA IGCUUCUC | 2187 |
| 370 | GAAGCCAC C AGCACCAC | 413 | GUGGUGCU CUGAUGAG GCCGUUAGGC CGAA IUGGCUUC | 2188 |
| 371 | AAGCCACC A GCACCACC | 414 | GGUGGUGC CUGAUGAG GCCGUUAGGC CGAA IGUGGCUU | 2189 |
| 374 | CCACCAGC A CCACCCAG | 415 | CUGGGUGG CUGAUGAG GCCGUUAGGC CGAA ICUGGUGG | 2190 |
| 376 | ACCAGCAC C ACCCAGAC | 416 | GUCUGGGU CUGAUGAG GCCGUUAGGC CGAA IUGCUGGU | 2191 |
| 377 | CCAGCACC A CCCAGACU | 417 | AGUCUGGG CUGAUGAG GCCGUUAGGC CGAA IGUGCUGG | 2192 |
| 379 | AGCACCAC C CAGACUUG | 418 | CAAGUCUG CUGAUGAG GCCGUUAGGC CGAA IUGGUGCU | 2193 |
| 380 | GCACCACC C AGACUUGG | 419 | CCAAGUCU CUGAUGAG GCCGUUAGGC CGAA IGUGGUGC | 2194 |
| 381 | CACCACCC A GACUUGGG | 420 | CCCAAGUC CUGAUGAG GCCGUUAGGC CGAA IGGUGGUG | 2195 |
| 385 | ACCCAGAC U UGGGGGCA | 421 | UGCCCCCA CUGAUGAG GCCGUUAGGC CGAA IUCUGGGU | 2196 |
| 393 | UUGGGGGC A GGCGCCAG | 422 | CUGGCGCC CUGAUGAG GCCGUUAGGC CGAA ICCCCCAA | 2197 |
| 399 | GCAGGCGC C AGGGACGG | 423 | CCGUCCCU CUGAUGAG GCCGUUAGGC CGAA ICGCCUGC | 2198 |
| 400 | CAGGCGCC A GGGACGGA | 424 | UCCGUCCC CUGAUGAG GCCGUUAGGC CGAA IGCGCCUG | 2199 |
| 416 | ACGUGGGC C AGUGCGAG | 425 | CUCGCACU CUGAUGAG GCCGUUAGGC CGAA ICCCACGU | 2200 |
| 417 | CGUGGGCC A GUGCGAGC | 426 | GCUCGCAC CUGAUGAG GCCGUUAGGC CGAA IGCCCACG | 2201 |
| 426 | GUGCGAGC C CAGAGGGC | 427 | GCCCUCUG CUGAUGAG GCCGUUAGGC CGAA ICUCGCAC | 2202 |
| 427 | UGCGAGCC C AGAGGGCC | 428 | GGCCCUCU CUGAUGAG GCCGUUAGGC CGAA IGCUCGCA | 2203 |
| 428 | GCGAGCCC A GAGGGCCC | 429 | GGGCCCUC CUGAUGAG GCCGUUAGGC CGAA IGGCUCGC | 2204 |
| 435 | CAGAGGGC C CGAAGGCC | 430 | GGCCUUCG CUGAUGAG GCCGUUAGGC CGAA ICCCUCUG | 2205 |
| 436 | AGAGGGCC C GAAGGCCG | 431 | CGGCCUUC CUGAUGAG GCCGUUAGGC CGAA IGCCCUCU | 2206 |
| 443 | CCGAAGGC C GGGGCCCA | 432 | UGGGCCCC CUGAUGAG GCCGUUAGGC CGAA ICCUUCGG | 2207 |
| 449 | GCCGGGGC C CACCAUGG | 433 | CCAUGGUG CUGAUGAG GCCGUUAGGC CGAA ICCCCGGC | 2208 |
| 450 | CCGGGGCC C ACCAUGGC | 434 | GCCAUGGU CUGAUGAG GCCGUUAGGC CGAA IGCCCCGG | 2209 |
| 451 | CGGGGCCC A CCAUGGCC | 435 | GGCCAUGG CUGAUGAG GCCGUUAGGC CGAA IGGCCCCG | 2210 |
| 453 | GGGCCCAC C AUGGCCCA | 436 | UGGGCCAU CUGAUGAG GCCGUUAGGC CGAA IUGGGCCC | 2211 |
| 454 | GGCCCACC A UGGCCCAA | 437 | UUGGGCCA CUGAUGAG GCCGUUAGGC CGAA IGUGGGCC | 2212 |
| 459 | ACCAUGGC C CAAGCCCU | 438 | AGGGCUUG CUGAUGAG GCCGUUAGGC CGAA ICCAUGGU | 2213 |
| 460 | CCAUGGCC C AAGCCCUG | 439 | CAGGGCUU CUGAUGAG GCCGUUAGGC CGAA IGCCAUGG | 2214 |
| 461 | CAUGGCCC A AGCCCUGC | 440 | GCAGGGCU CUGAUGAG GCCGUUAGGC CGAA IGGCCAUG | 2215 |
| 465 | GCCCAAGC C CUGCCCUG | 441 | CAGGGCAG CUGAUGAG GCCGUUAGGC CGAA ICUUGGGC | 2216 |
| 466 | CCCAAGCC C UGCCCUGG | 442 | CCAGGGCA CUGAUGAG GCCGUUAGGC CGAA IGCUUGGG | 2217 |
| 467 | CCAAGCCC U GCCCUGGC | 443 | GCCAGGGC CUGAUGAG GCCGUUAGGC CGAA IGGCUUGG | 2218 |
| 470 | AGCCCUGC C CUGGCUCC | 444 | GGAGCCAG CUGAUGAG GCCGUUAGGC CGAA ICAGGGCU | 2219 |
| 471 | GCCCUGCC C UGGCUCCU | 445 | AGGAGCCA CUGAUGAG GCCGUUAGGC CGAA IGCAGGGC | 2220 |
| 472 | CCCUGCCC U GGCUCCUG | 446 | CAGGAGCC CUGAUGAG GCCGUUAGGC CGAA IGGCAGGG | 2221 |
| 476 | GCCCUGGC U CCUGCUGU | 447 | ACAGCAGG CUGAUGAG GCCGUUAGGC CGAA ICCAGGGC | 2222 |
| 478 | CCUGGCUC C UGCUGUGG | 448 | CCACAGCA CUGAUGAG GCCGUUAGGC CGAA IAGCCAGG | 2223 |
| 479 | CUGGCUCC U GCUGUGGA | 449 | UCCACAGC CUGAUGAG GCCGUUAGGC CGAA IGAGCCAG | 2224 |
| 482 | GCUCCUGC U GUGGAUGG | 450 | CCAUCCAC CUGAUGAG GCCGUUAGGC CGAA ICAGGAGC | 2225 |
| 503 | GGGAGUGC U GCCUGCCC | 451 | GGGCAGGC CUGAUGAG GCCGUUAGGC CGAA ICACUCCC | 2226 |
| 506 | AGUGCUGC C UGCCCACG | 452 | CGUGGGCA CUGAUGAG GCCGUUAGGC CGAA ICAGCACU | 2227 |
| 507 | GUGCUGCC U GCCCACGG | 453 | CCGUGGGC CUGAUGAG GCCGUUAGGC CGAA IGCAGCAC | 2228 |
| 510 | CUGCCUGC C CACGGCAC | 454 | GUGCCGUG CUGAUGAG GCCGUUAGGC CGAA ICAGGCAG | 2229 |
| 511 | UGCCUGCC C ACGGCACC | 455 | GGUGCCGU CUGAUGAG GCCGUUAGGC CGAA IGCAGGCA | 2230 |
| 512 | GCCUGCCC A CGGCACCC | 456 | GGGUGCCG CUGAUGAG GCCGUUAGGC CGAA IGGCAGGC | 2231 |
| 517 | CCCACGGC A CCCAGCAC | 457 | GUGCUGGG CUGAUGAG GCCGUUAGGC CGAA ICCGUGGG | 2232 |
| 519 | CACGGCAC C CAGCACGG | 458 | CCGUGCUG CUCAUGAG GCCGUUAGGC CGAA IUGCCGUG | 2233 |
| 520 | ACGGCACC C AGCACGGC | 459 | GCCGUGCU CUGAUGAG GCCGUUAGGC CGAA IGUGCCGU | 2234 |
| 521 | CGGCACCC A GCACGGCA | 460 | UGCCGUGC CUGAUGAG GCCGUUAGGC CGAA IGGUGCCG | 2235 |
| 524 | CACCCAGC A CGGCAUCC | 461 | GGAUGCCG CUGAUGAG GCCGUUAGGC CGAA ICUGGGUG | 2236 |
| 529 | AGCACGGC A UCCGGCUG | 462 | CAGCCGGA CUGAUGAG GCCGUUAGGC CGAA ICCGUGCU | 2237 |
| 532 | ACGGCAUC C GGCUGCCC | 463 | GGGCAGCC CUGAUGAG GCCGUUAGGC CGAA IAUGCCGU | 2238 |
| 536 | CAUCCGGC U GCCCCUGC | 464 | GCAGGGGC CUGAUGAG GCCGUUAGGC CGAA ICCGGAUG | 2239 |
| 539 | CCGGCUGC C CCUGCGCA | 465 | UGCGCAGG CUGAUGAG GCCGUUAGGC CGAA ICAGCCGG | 2240 |
| 540 | CGGCUGCC C CUGCGCAG | 466 | CUGCGCAG CUGAUGAG GCCGUUAGGC CGAA IGCAGCCG | 2241 |
| 541 | GGCUGCCC C UGCGCAGC | 467 | GCUGCGCA CUGAUGAG GCCGUUAGGC CGAA IGGCAGCC | 2242 |
| 542 | GCUGCCCC U GCGCAGCG | 468 | CGCUGCGC CUGAUGAG GCCGUUAGGC CGAA IGGGCAGC | 2243 |
| 547 | CCCUGCGC A GCGGCCUG | 469 | CAGGCCGC CUGAUGAG GCCGUUAGGC CGAA ICGCAGGG | 2244 |
| 553 | GCAGCGGC C UGGGGGGC | 470 | GCCCCCCA CUGAUGAG GCCGUUAGGC CGAA ICCGCUGC | 2245 |
| 554 | CAGCGGCC U GGGGGGCG | 471 | CGCCCCCC CUGAUGAG GCCGUUAGGC CGAA IGCCGCUG | 2246 |
| 564 | GGGGGCGC C CCCCUGGG | 472 | CCCAGGGG CUGAUGAG GCCGUUAGGC CGAA ICGCCCCC | 2247 |
| 565 | GGGGCGCC C CCCUGGGG | 473 | CCCCAGGG CUGAUGAG GCCGUUAGGC CGAA IGCGCCCC | 2248 |
| 566 | GGGCGCCC C CCUGGGGC | 474 | GCCCCAGG CUGAUGAG GCCGUUAGGC CGAA IGGCGCCC | 2249 |
| 567 | GGCGCCCC C CUGGGGCU | 475 | AGCCCCAG CUGAUGAG GCCGUUAGGC CGAA IGGGCGCC | 2250 |
| 568 | GCGCCCCC C UGGGGCUG | 476 | CAGCCCCA CUGAUGAG GCCGUUAGGC CGAA IGGGGCGC | 2251 |
| 569 | CGCCCCCC U GGGGCUGC | 477 | GCAGCCCC CUGAUGAG GCCGUUAGGC CGAA IGGGGGCG | 2252 |
| 575 | CCUGGGGC U GCGGCUGC | 478 | GCAGCCGC CUGAUGAG GCCGUUAGGC CGAA ICCCCAGG | 2253 |
| 581 | GCUGCGGC U GCCCCGGG | 479 | CCCGGGGC CUGAUGAG GCCGUUAGGC CGAA ICCGCAGC | 2254 |
| 584 | GCGGCUGC C CCGGGAGA | 480 | UCUCCCGG CUGAUGAG GCCGUUAGGC CGAA ICAGCCGC | 2255 |
| 585 | CGGCUGCC C CGGGAGAC | 481 | GUCUCCCG CUGAUGAG GCCGUUAGGC CGAA IGCAGCCG | 2256 |
| 586 | GGCUGCCC C GGGAGACC | 482 | GGUCUCCC CUGAUGAG GCCGUUAGGC CGAA IGGCAGCC | 2257 |
| 594 | CGGGAGAC C GACGAAGA | 483 | UCUUCGUC CUGAUGAG GCCGUUAGGC CGAA IUCUCCCG | 2258 |
| 605 | CGAAGAGC C CGAGGAGC | 484 | GCUCCUCG CUGAUGAG GCCGUUAGGC CGAA ICUCUUCG | 2259 |
| 606 | GAAGAGCC C GAGGAGCC | 485 | GGCUCCUC CUGAUGAG GCCGUUAGGC CGAA IGCUCUUC | 2260 |
| 614 | CGAGGAGC C CGGCCGGA | 486 | UCCGGCCG CUGAUGAG GCCGUUAGGC CGAA ICUCCUCG | 2261 |
| 615 | GAGGAGCC C GGCCGGAG | 487 | CUCCGGCC CUGAUGAG GCCGUUAGGC CGAA IGCUCCUC | 2262 |
| 619 | AGCCCGGC C GGAGGGGC | 488 | GCCCCUCC CUGAUGAG GCCGUUAGGC CGAA ICCGGGCU | 2263 |
| 628 | GGAGGGGC A GCUUUGUG | 489 | CACAAAGC CUGAUGAG GCCGUUAGGC CGAA ICCCCUCC | 2264 |
| 631 | GGGGCAGC U UUGUGGAG | 490 | CUCCACAA CUGAUGAG GCCGUUAGGC CGAA ICUGCCCC | 2265 |
| 649 | UGGUGGAC A ACCUGAGG | 491 | CCUCAGGU CUGAUGAG GCCGUUAGGC CGAA IUCCACCA | 2266 |
| 652 | UGGACAAC C UGAGGGGC | 492 | GCCCCUCA CUGAUGAG GCCGUUAGGC CGAA IUUGUCCA | 2267 |
| 653 | GGACAACC U GAGGGGCA | 493 | UGCCCCUC CUGAUGAG GCCGUUAGGC CGAA IGUUGUCC | 2268 |
| 661 | UGAGGGGC A AGUCGGGG | 494 | CCCCGACU CUGAUGAG GCCGUUAGGC CGAA ICCCCUCA | 2269 |
| 671 | GUCGGGGC A GGGCUACU | 495 | AGUAGCCC CUGAUGAG GCCGUUAGGC CGAA ICCCCGAC | 2270 |
| 676 | GGCAGGGC U ACUACGUG | 496 | CACGUAGU CUGAUGAG GCCGUUAGGC CGAA ICCCUGCC | 2271 |
| 679 | AGGGCUAC U ACGUGGAG | 497 | CUCCACGU CUGAUGAG GCCGUUAGGC CGAA IUAGCCCU | 2272 |
| 693 | GAGAUGAC C GUGGGCAG | 498 | CUGCCCAC CUGAUGAG GCCGUUAGGC CGAA IUCAUCUC | 2273 |
| 700 | CCGUGGGC A GCCCCCCG | 499 | CGGGGGGC CUGAUGAG GCCGUUAGGC CGAA ICCCACGG | 2274 |
| 703 | UGGGCAGC C CCCCGCAG | 500 | CUGCGGGG CUGAUGAG GCCGUUAGGC CGAA ICUGCCCA | 2275 |
| 704 | GGGCAGCC C CCCGCAGA | 501 | UCUGCGGG CUGAUGAG GCCGUUAGGC CGAA IGCUGCCC | 2276 |
| 705 | GGCAGCCC C CCGCAGAC | 502 | GUCUGCGG CUGAUGAG GCCGUUAGGC CGAA IGGCUGCC | 2277 |
| 706 | GCAGCCCC C CGCAGACG | 503 | CGUCUGCG CUGAUGAG GCCGUUAGGC CGAA IGGGCUGC | 2278 |
| 707 | CAGCCCCC C GCAGACGC | 504 | GCGUCUGC CUGAUGAG GCCGUUAGGC CGAA IGGGGCUG | 2279 |
| 710 | CCCCCCGC A GACGCUCA | 505 | UGAGCGUC CUGAUGAG GCCGUUAGGC CGAA ICGGGGGG | 2280 |
| 716 | GCAGACGC U CAACAUCC | 506 | GGAUGUUG CUGAUGAG GCCGUUAGGC CGAA ICGUCUGC | 2281 |
| 718 | AGACGCUC A ACAUCCUG | 507 | CAGGAUGU CUGAUGAG GCCGUUAGGC CGAA IAGCGUCU | 2282 |
| 721 | CGCUCAAC A UCCUGGUG | 508 | CACCAGGA CUGAUGAG GCCGUUAGGC CGAA IUUGAGCG | 2283 |
| 724 | UCAACAUC C UGGUGGAU | 509 | AUCCACCA CUGAUGAG GCCGUUAGGC CGAA IAUGUUGA | 2284 |
| 725 | CAACAUCC U GGUGGAUA | 510 | UAUCCACC CUGAUGAG GCCGUUAGGC CGAA IGAUGUUG | 2285 |
| 735 | GUGGAUAC A GGCAGCAG | 511 | CUGCUGCC CUGAUGAG GCCGUUAGGC CGAA IUAUCCAC | 2286 |
| 739 | AUACAGGC A GCAGUAAC | 512 | GUUACUGC CUGAUGAG GCCGUUAGGC CGAA ICCUGUAU | 2287 |
| 742 | CAGGCAGC A GUAACUUU | 513 | AAAGUUAC CUGAUGAG GCCGUUAGGC CGAA ICUGCCUG | 2288 |
| 748 | GCAGUAAC U UUGCAGUG | 514 | CACUGCAA CUGAUGAG GCCGUUAGGC CGAA IUUACUGC | 2289 |
| 753 | AACUUUGC A GUGGGUGC | 515 | GCACCCAC CUGAUGAG GCCGUUAGGC CGAA ICAAAGUU | 2290 |
| 762 | GUGGGUGC U GCCCCCCA | 516 | UGGGGGGC CUGAUGAG GCCGUUAGGC CGAA ICACCCAC | 2291 |
| 765 | GGUGCUGC C CCCCACCC | 517 | GGGUGGGG CUGAUGAG GCCGUUAGGC CGAA ICAGCACC | 2292 |
| 766 | GUGCUGCC C CCCACCCC | 518 | GGGGUGGG CUGAUGAG GCCGUUAGGC CGAA IGCAGCAC | 2293 |
| 767 | UGCUGCCC C CCACCCCU | 519 | AGGGGUGG CUGAUGAG GCCGUUAGGC CGAA IGGCAGCA | 2294 |
| 768 | GCUGCCCC C CACCCCUU | 520 | AAGGGGUG CUGAUGAG GCCGUUAGGC CGAA IGGGCAGC | 2295 |
| 769 | CUGCCCCC C ACCCCUUC | 521 | GAAGGGGU CUGAUGAG GCCGUUAGGC CGAA IGGGGCAG | 2296 |
| 770 | UGCCCCCC A CCCCUUCC | 522 | GGAAGGGG CUGAUGAG GCCGUUAGGC CGAA IGGGGGCA | 2297 |
| 772 | CCCCCCAC C CCUUCCUG | 523 | CAGGAAGG CUGAUGAG GCCGUUAGGC CGAA IUGGGGGG | 2298 |
| 773 | CCCCCACC C CUUCCUGC | 524 | GCAGGAAG CUGAUGAG GCCGUUAGGC CGAA IGUGGGGG | 2299 |
| 774 | CCCCACCC C UUCCUGCA | 525 | UGCAGGAA CUGAUGAG GCCGUUAGGC CGAA IGGUGGGG | 2300 |
| 775 | CCCACCCC U UCCUGCAU | 526 | AUGCAGGA CUGAUGAG GCCGUUAGGC CGAA IGGGUGGG | 2301 |
| 778 | ACCCCUUC C UGCAUCGC | 527 | GCGAUGCA CUGAUGAG GCCGUUAGGC CGAA IAAGGGGU | 2302 |
| 779 | CCCCUUCC U GCAUCGCU | 528 | AGCGAUGC CUGAUGAG GCCGUUAGGC CGAA IGAAGGGG | 2303 |
| 782 | CUUCCUGC A UCGCUACU | 529 | AGUAGCGA CUGAUGAG GCCGUUAGGC CGAA ICAGGAAG | 2304 |
| 787 | UGCAUCGC U ACUACCAG | 530 | CUGGUAGU CUGAUGAG GCCGUUAGGC CGAA ICGAUGCA | 2305 |
| 790 | AUCGCUAC U ACCAGAGG | 531 | CCUCUGGU CUGAUGAG GCCGUUAGGC CGAA IUAGCGAU | 2306 |
| 793 | GCUACUAC C AGAGGCAG | 532 | CUGCCUCU CUGAUGAG GCCGUUAGGC CGAA IUAGUAGC | 2307 |
| 794 | CUACUACC A GAGGCAGC | 533 | GCUGCCUC CUGAUGAG GCCGUUAGGC CGAA IGUAGUAG | 2308 |
| 800 | CCAGAGGC A GCUGUCCA | 534 | UGGACAGC CUGAUGAG GCCGUUAGGC CGAA ICCUCUGG | 2309 |
| 803 | GAGGCAGC U GUCCAGCA | 535 | UGCUGGAC CUGAUGAG GCCGUUAGGC CGAA ICUGCCUC | 2310 |
| 807 | CAGCUGUC C AGCACAUA | 536 | UAUGUGCU CUGAUGAG GCCGUUAGGC CGAA IACAGCUG | 2311 |
| 808 | AGCUGUCC A GCACAGAC | 537 | GUAUGUGC CUGAUGAG GCCGUUAGGC CGAA IGACAGCU | 2312 |
| 811 | UGUCCAGC A CAUACCGG | 538 | CCGGUAUG CUGAUGAG GCCGUUAGGC CGAA ICUGGACA | 2313 |
| 813 | UCCAGCAC A UACCGGGA | 539 | UCCCGGUA CUGAUGAG GCCGUUAGGC CGAA IUGCUGGA | 2314 |
| 817 | GCACAUAC C GGGACCUC | 540 | GAGGUCCC CUGAUGAG GCCGUUAGGC CGAA IUAUGUGC | 2315 |
| 823 | ACCGGGAC C UCCGGAAG | 541 | CUUCCGGA CUGAUGAG GCCGUUAGGC CGAA IUCCCGGU | 2316 |
| 824 | CCGGGACC U CCGGAAGG | 542 | CCUUCCGG CUGAUGAG GCCGUUAGGC CGAA IGUCCCGG | 2317 |
| 826 | GGGACCUC C GGAAGGGU | 543 | ACCCUUCC CUGAUGAG GCCGUUAGGC CGAA IAGGUCCC | 2318 |
| 845 | GUAUGUGC C CUACACCC | 544 | GGGUGUAG CUGAUGAG GCCGUUAGGC CGAA ICACAUAC | 2319 |
| 846 | UAUGUGCC C UACACCCA | 545 | UGGGUGUA CUGAUGAG GCCGUUAGGC CGAA IGCACAUA | 2320 |
| 847 | AUGUGCCC U ACACCCAG | 546 | CUGGGUGU CUGAUGAG GCCGUUAGGC CGAA IGGCACAU | 2321 |
| 850 | UGCCCUAC A CCCAGGGC | 547 | GCCCUGGG CUGAUGAG GCCGUUAGGC CGAA IUAGGGCA | 2322 |
| 852 | CCCUACAC C CAGGGCAA | 548 | UUGCCCUG CUGAUGAG GCCGUUAGGC CGAA IUGUAGGG | 2323 |
| 853 | CCUACACC C AGGGCAAG | 549 | CUUGCCCU CUGAUGAG GCCGUUAGGC CGAA IGUGUAGG | 2324 |
| 854 | CUACACCC A GGGCAAGU | 550 | ACUUGCCC CUGAUGAG GCCGUUAGGC CGAA IGGUGUAG | 2325 |
| 859 | CCCAGGGC A AGUGGGAA | 551 | UUCCCACU CUGAUGAG GCCGUUAGGC CGAA ICCCUGGG | 2326 |
| 875 | AGGGGAGC U GGGCACCG | 552 | CGGUGCCC CUGAUGAG GCCGUUAGGC CGAA ICUCCCCU | 2327 |
| 880 | AGCUGGGC A CCGACCUG | 553 | CAGGUCGG CUGAUGAG GCCGUUAGGC CGAA ICCCAGCU | 2328 |
| 882 | CUGGGCAC C GACCUGGU | 554 | ACCAGGUC CUGAUGAG GCCGUUAGGC GCAA IUGCCCAG | 2329 |
| 886 | GCACCGAC C UGGUAAGC | 555 | GCUUACCA CUGAUGAG GCCGUUAGGC CGAA IUCGGUGC | 2330 |
| 887 | CACCGACC U GGUAAGCA | 556 | UGCUUACC CUGAUGAG GCCGUUAGGC CGAA IGUCGGUG | 2331 |
| 895 | UGGUAAGC A UCCCCCAU | 557 | AUGGGGGA CUGAUGAG GCCGUUAGGC CGAA ICUUACCA | 2332 |
| 898 | UAAGCAUC C CCCAUGGC | 558 | GCCAUGGG CUGAUGAG GCCGUUAGGC CGAA IAUGCUUA | 2333 |
| 899 | AAGCAUCC C CCAUGGCC | 559 | GGCCAUGG CUGAUGAG GCCGUUAGGC CGAA IGAUGCUU | 2334 |
| 900 | AGCAUCCC C CAUGGCCC | 560 | GGGCCAUG CUGAUGAG GCCGUUAGGC CGAA IGGAUGCU | 2335 |
| 901 | GCAUCCCC C AUGGCCCC | 561 | GGGGCCAU CUGAUGAG GCCGUUAGGC CGAA IGGGAUGC | 2336 |
| 902 | CAUCCCCC A UGGCCCCA | 562 | UGGGGCCA CUGAUGAG GCCGUUAGGC CGAA IGGGGAUG | 2337 |
| 907 | CCCAUGGC C CCAACGUC | 563 | GACGUUGG CUGAUGAG GCCGUUAGGC CGAA ICCAUGGG | 2338 |
| 908 | CCAUGGCC C CAACGUCA | 564 | UGACGUUG CUGAUGAG GCCGUUAGGC CGAA IGCCAUGG | 2339 |
| 909 | CAUGGCCC C AACGUCAC | 565 | GUGACGUU CUGAUGAG GCCGUUAGGC CGAA IGGCCAUG | 2340 |
| 910 | AUGGCCCC A ACGUCACU | 566 | AGUGACGU CUGAUGAG GCCGUUAGGC CGAA IGGGCCAU | 2341 |
| 916 | CCAACGUC A CUGUGCGU | 567 | ACGCACAG CUGAUGAG GCCGUUAGGC CGAA IACGUUGG | 2342 |
| 918 | AACGUCAC U GUGCGUGC | 568 | GCACGCAC CUGAUGAG GCCGUUAGGC CGAA IUGACGUU | 2343 |
| 927 | GUGCGUGC C AACAUUGC | 569 | GCAAUGUU CUGAUGAG GCCGUUAGGC CGAA ICACGCAC | 2344 |
| 928 | UGCGUGCC A ACAUUGCU | 570 | AGCAAUGU CUGAUGAG GCCGUUAGGC CGAA IGCACGCA | 2345 |
| 931 | GUGCCAAC A UUGCUGCC | 571 | GGCAGCAA CUGAUGAG GCCGUUAGGC CGAA IUUGGCAC | 2346 |
| 936 | AACAUUGC U GCCAUCAC | 572 | GUGAUGGC CUGAUGAG GCCGUUAGGC CGAA ICAAUGUU | 2347 |
| 939 | AUUGCUGC C AUCACUGA | 573 | UCAGUGAU CUGAUGAG GCCGUUAGGC CGAA ICAGCAAU | 2348 |
| 940 | UUGCUGCC A UCACUGAA | 574 | UUCAGUGA CUGAUGAG GCCGUUAGGC CGAA IGCAGCAA | 2349 |
| 943 | CUGCCAUC A CUGAAUCA | 575 | UGAUUCAG CUGAUGAG GCCGUUAGGC CGAA IAUGGCAG | 2350 |
| 945 | GCCAUCAC U GAAUCAGA | 576 | UCUGAUUC CUGAUGAG GCCGUUAGGC CGAA IUGAUGGC | 2351 |
| 951 | ACUGAAUC A GACAAGUU | 577 | AACUUGUC CUGAUGAG GCCGUUAGGC CGAA IAUUCAGU | 2352 |
| 955 | AAUCAGAC A AGUUCUUC | 578 | GAAGAACU CUGAUGAG GCCGUUAGGC CGAA IUCUGAUU | 2353 |
| 961 | ACAAGUUC U UCAUCAAC | 579 | GUUGAUGA CUGAUGAG GCCGUUAGGC CGAA IAACUUGU | 2354 |
| 964 | AGUUCUUC A UCAACGGC | 580 | GCCGUUGA CUGAUGAG GCCGUUAGGC CGAA IAAGAACU | 2355 |
| 967 | UCUUCAUC A ACGGCUCC | 581 | GGAGCCGU CUGAUGAG GCCGUUAGGC CGAA IAUGAAGA | 2356 |
| 973 | UCAACGGC U CCAACUGG | 582 | CCAGUUGG CUGAUGAG GCCGUUAGGC CGAA ICCGUUGA | 2357 |
| 975 | AACGGCUC C AACUGGGA | 583 | UCCCAGUU CUGAUGAG GCCGUUAGGC CGAA IAGCCGUU | 2358 |
| 976 | ACGGCUCC A ACUGGGAA | 584 | UUCCCAGU CUGAUGAG GCCGUUAGGC CGAA IGAGCCGU | 2359 |
| 979 | GCUCCAAC U GGGAAGGC | 585 | GCCUUCCC CUGAUGAG GCCGUUAGGC CGAA IUUGGAGC | 2360 |
| 988 | GGGAAGGC A UCCUGGGG | 586 | CCCCAGGA CUGAUGAG GCCGUUAGGC CGAA ICCUUCCC | 2361 |
| 991 | AAGGCAUC C UGGGGCUG | 587 | CAGCCCCA CUGAUGAG GCCGUUAGGC CGAA IAUGCCUU | 2362 |
| 992 | AGGCAUCC U GGGGCUGG | 588 | CCAGCCCC CUGAUGAG GCCGUUAGGC CGAA IGAUGCCU | 2363 |
| 998 | CCUGGGGC U GGCCUAUG | 589 | CAUAGGCC CUGAUGAG GCCGUUAGGC CGAA ICCCCAGG | 2364 |
| 1002 | GGGCUGGC C UAUGCUGA | 590 | UCAGCAUA CUGAUGAG GCCGUUAGGC CGAA ICCAGCCC | 2365 |
| 1003 | GGCUGGCC U AUGCUGAG | 591 | CUCAGCAU CUGAUGAG GCCGUUAGGC CGAA IGCCAGCC | 2366 |
| 1008 | GCCUAUGC U GAGAUUGC | 592 | GCAAUCUC CUGAUGAG GCCGUUAGGC CGAA ICAUAGGC | 2367 |
| 1017 | GAGAUUGC C AGGCCUGA | 593 | UCAGGCCU CUGAUGAG GCCGUUAGGC CGAA ICAAUCUC | 2368 |
| 1018 | AGAUUGCC A GGCCUGAC | 594 | GUCAGGCC CUGAUGAG GCCGUUAGGC CGAA IGCAAUCU | 2369 |
| 1022 | UGCCAGGC C UGACGACU | 595 | AGUCGUCA CUGAUGAG GCCGUUAGGC CGAA ICCUGGCA | 2370 |
| 1023 | GCCAGGCC U GACGACUC | 596 | GAGUCGUC CUGAUGAG GCCGUUAGGC CGAA IGCCUGGC | 2371 |
| 1030 | CUGACGAC U CCCUGGAG | 597 | CUCCAGGG CUGAUGAG GCCGUUAGGC CGAA IUCGUCAG | 2372 |
| 1032 | GACGACUC C CUGGAGCC | 598 | GGCUCCAG CUGAUGAG GCCGUUAGGC CGAA IAGUCGUC | 2373 |
| 1033 | ACGACUCC C UGGAGCCU | 599 | AGGCUCCA CUGAUGAG GCCGUUAGGC CGAA IGAGUCGU | 2374 |
| 1034 | CGACUCCC U GGAGCCUU | 600 | AAGGCUCC CUGAUGAG GCCGUUAGGC CGAA IGGAGUCG | 2375 |
| 1040 | CCUGGAGC C UUUCUUUG | 601 | CAAAGAAA CUGAUGAG GCCGUUAGGC CGAA ICUCCAGG | 2376 |
| 1041 | CUGGAGCC U UUCUUUGA | 602 | UCAAAGAA CUGAUGAG GCCGUUAGGC CGAA IGCUCCAG | 2377 |
| 1045 | AGCCUUUC U UUGACUCU | 603 | AGAGUCAA CUGAUGAG GCCGUUAGGC CGAA IAAAGGCU | 2378 |
| 1051 | UCUUUGAC U CUCUGGUA | 604 | UACCAGAG CUGAUGAG GCCGUUAGGC CGAA IUCAAAGA | 2379 |
| 1053 | UUUGACUC U CUGGUAAA | 605 | UUUACCAG CUGAUGAG GCCGUUAGGC CGAA IAGUCAAA | 2380 |
| 1055 | UGACUCUC U GGUAAAGC | 606 | GCUUUACC CUGAUGAG GCCGUUAGGC CGAA UAGAGUCA | 2381 |
| 1064 | GGUAAAGC A GACCCACG | 607 | CGUGGGUC CUGAUGAG GCCGUUAGGC CGAA ICUUUACC | 2382 |
| 1068 | AAGCAGAC C CACGUUCC | 608 | GGAACGUG CUGAUGAG GCCGUUAGGC CGAA IUCUGCUU | 2383 |
| 1069 | AGCAGACC C ACGUUCCC | 609 | GGGAACGU CUGAUGAG GCCGUUAGGC CGAA IGUCUGCU | 2384 |
| 1070 | GCAGACCC A CGUUCCCA | 610 | UGGGAACG CUGAUGAG GCCGUUAGGC CGAA IGGUCUGC | 2385 |
| 1076 | CCACGUUC C CAACCUCU | 611 | AGAGGUUG CUGAUGAG GCCGUUAGGC CGAA IAACGUGG | 2386 |
| 1077 | CACGUUCC C AACCUCUU | 612 | AAGAGGUU CUGAUGAG GCCGUUAGGC CGAA IGAACGUG | 2387 |
| 1078 | ACGUUCCC A ACCUCUUC | 613 | GAAGAGGU CUGAUGAG GCCGUUAGGC CGAA IGGAACGU | 2388 |
| 1081 | UUCCCAAC C UCUUCUCC | 614 | GGAGAAGA CUGAUGAG GCCGUUAGGC CGAA IUUGGGAA | 2389 |
| 1082 | UCCCAACC U CUUCUCCC | 615 | GGGAGAAG CUGAUGAG GCCGUUAGGC CGAA IGUUGGGA | 2390 |
| 1084 | CCAACCUC U UCUCCCUG | 616 | CAGGGAGA CUGAUGAG GCCGUUAGGC CGAA IAGGUUGG | 2391 |
| 1087 | ACCUCUUC U CCCUGCAG | 617 | CUGCAGGG CUGAUGAG GCCGUUAGGC CGAA IAAGAGGU | 2392 |
| 1089 | CUCUUCUC C CUGCAGCU | 618 | AGCUGCAG CUGAUGAG GCCGUUAGGC CGAA IAGAAGAG | 2393 |
| 1090 | UCUUCUCC C UGCAGCUU | 619 | AAGCUGCA CUGAUGAG GCCGUUAGGC CGAA IGAGAAGA | 2394 |
| 1091 | CUUCUCCC U GCAGCUUU | 620 | AAAGCUGC CUGAUGAG GCCGUUAGGC CGAA IGGAGAAG | 2395 |
| 1094 | CUCCCUGC A GCUUUGUG | 621 | CACAAAGC CUGAUGAG GCCGUUAGGC CGAA ICAGGGAG | 2396 |
| 1097 | CCUGCAGC U UUGUGGUG | 622 | CACCACAA CUGAUGAG GCCGUUAGGC CGAA ICUGCAGG | 2397 |
| 1107 | UGUGGUGC U GGCUUCCC | 623 | GGGAAGCC CUGAUGAG GCCGUUAGGC CGAA ICACCACA | 2398 |
| 1111 | GUGCUGGC U UCCCCCUC | 624 | GAGGGGGA CUGAUGAG GCCGUUAGGC CGAA ICCAGCAC | 2399 |
| 1114 | CUGGCUUC C CCCUCAAC | 625 | GUUGAGGG CUGAUGAG GCCGUUAGGC CGAA IAAGCCAG | 2400 |
| 1115 | UGGCUUCC C CCUCAACC | 626 | GGUUGAGG CUGAUGAG GCCGUUAGGC CAGG IGAAGCCA | 2401 |
| 1116 | GGCUUCCC C CUCAACCA | 627 | UGGUUGAG CUGAUGAG GCCGUUAGGC CGAA IGGAAGCC | 2402 |
| 1117 | GCUUCCCC C UCAACCAG | 628 | CUGGUUGA CUGAUGAG GCCGUUAGGC CGAA IGGGAAGC | 2403 |
| 1118 | CUUCCCCC U CAACCAGU | 629 | ACUGGUUG CUGAUGAG GCCGUUAGGC CGAA IGGGGAAG | 2404 |
| 1120 | UCCCCCUC A ACCAGUCU | 630 | AGACUGGU CUGAUGAG GCCGUUAGGC CGAA IAGGGGGA | 2405 |
| 1123 | CCCUCAAC C AGUCUGAA | 631 | UUCAGACU CUGAUGAG GCCGUUAGGC CGAA IUUGAGGG | 2406 |
| 1124 | CCUCAACC A GUCUGAAG | 632 | CUCCAGAC CUGAUGAG GCCGUUAGGC CGAA IGUUGAGG | 2407 |
| 1128 | AACCAGUC U GAAGUGCU | 633 | AGCACUUC CUGAUGAG GCCGUUAGGC CGAA IACUGGUU | 2408 |
| 1136 | UGAAGUGC U GGCCUCUG | 634 | CAGAGGCC CUGAUGAG GCCGUUAGGC CGAA ICACUUCA | 2409 |
| 1140 | GUGCUGGC C UCUGUCGG | 635 | CCGACAGA CUGAUGAG GCCGUUAGGC CGAA ICCAGCAC | 2410 |
| 1141 | UGCUGGCC U CUGUCGGA | 636 | UCCGACAG CUGAUGAG GCCGUUAGGC CGAA IGCCAGCA | 2411 |
| 1143 | CUGGCCUC U GUCGGAGG | 637 | CCUCCGAC CUGAUGAG GCCGUUAGGC CGAA IAGGCCAG | 2412 |
| 1156 | GAGGGAGC A UGAUCAUU | 638 | AAUGAUCA CUGAUGAG GCCGUUAGGC CGAA ICUCCCUC | 2413 |
| 1162 | GCAUGAUC A UUGGAGGU | 639 | ACCUCCAA CUGAUGAG GCCGUUAGGC CGAA IAUCAUGC | 2414 |
| 1177 | GUAUCGAC C ACUCGCUG | 640 | CAGCGAGU CUGAUGAG GCCGUUAGGC CGAA IUCGAUAC | 2415 |
| 1178 | UAUCGACC A CUCGCUGU | 641 | ACAGCGAG CUGAUGAG GCCGUUAGGC CGAA IGUCGAUA | 2416 |
| 1180 | UCGACCAC U CGCUGUAC | 642 | GUACAGCG CUGAUGAG GCCGUUAGGC CGAA IUGGUCGA | 2417 |
| 1184 | CCACUCGC U GUACACAG | 643 | CUGUGUAC CUGAUGAG GCCGUUAGGC CGAA ICGAGUGG | 2418 |
| 1189 | CGCUGUAC A CAGGCAGU | 644 | ACUGCCUG CUGAUGAG GCCGUUAGGC CGAA IUACAGCG | 2419 |
| 1191 | CUGUACAC A GGCAGUCU | 645 | AGACUGCC CUGAUGAG GCCGUUAGGC CGAA IUGUACAG | 2420 |
| 1195 | ACACAGGC A GUCUCUGG | 646 | CCAGAGAC CUGAUGAG GCCGUUAGGC CGAA ICCUGUGU | 2421 |
| 1199 | AGGCAGUC U CUGGUAUA | 647 | UAUACCAG CUGAUGAG GCCGUUAGGC CGAA IACUGCCU | 2422 |
| 1201 | GCAGUCUC U GGUAUACA | 648 | UGUAUACC CUGAUGAG GCCGUUAGGC CGAA IAGACUGC | 2423 |
| 1209 | UGGUAUAC A CCCAUCCG | 649 | CGGAUGGG CUGAUGAG GCCGUUAGGC CGAA IUAUACCA | 2424 |
| 1211 | GUAUACAC C CAUCCGGC | 650 | GCCGGAUG CUGAUGAG GCCGUUAGGC CGAA IUGUAUAC | 2425 |
| 1212 | UAUACACC C AUCCGGCG | 651 | CGCCGGAU CUGAUGAG GCCGUCAGGC CGAA IGUGUAUA | 2426 |
| 1213 | AUACACCC A UCCGGCGG | 652 | CCGCCGGA CUGAUGAG GCCGUUAGGC CGAA IGGUGUAU | 2427 |
| 1216 | CACCCAUC C GGCGGGAG | 653 | CUCCCGCC CUGAUGAG GCCGUUAGGC CGAA IAUGGGUG | 2428 |
| 1243 | AGGUGAUC A UUGUGCGG | 654 | CCGCACAA CUGAUGAG GCCGUUAGGC CGAA IAUCACCU | 2429 |
| 1261 | UGGAGAUC A AUGGACAG | 655 | CUGUCCAU CUGAUGAG GCCGUUAGGC CGAA IAUCUCCA | 2430 |
| 1268 | CAAUGGAC A GGAUCUGA | 656 | UCAGAUCC CUGAUGAG GCCGUUAGGC CGAA IUCCAUUG | 2431 |
| 1274 | ACAGGAUC U GAAAAUGG | 657 | CCAUUUUC CUGAUGAG GCCGUUAGGC CGAA IAUCCUGU | 2432 |
| 1285 | AAAUGGAC U CGAAGGAG | 658 | CUCCUUGC CUGAUGAG GCCGUUAGGC CGAA IUCCAUUU | 2433 |
| 1288 | UGGACUGC A AGGAGUAC | 659 | GUACUCCU CUGAUGAG GCCGUUAGGC CGAA ICAGUCCA | 2434 |
| 1297 | AGGAGUAC A ACUAUGAC | 660 | GUCAUAGU CUGAUGAG GCCGUUAGGC CGAA IUACUCCU | 2435 |
| 1300 | AGUACAAC U AUGACAAG | 661 | CUUGUCAU CUGAUGAG GCCGUUAGGC CGAA IUUGUACU | 2436 |
| 1306 | ACUAUGAC A AGAGCAUU | 662 | AAUGCUCU CUGAUGAG GCCGUUAGGC CGAA IUCAUAGU | 2437 |
| 1312 | ACAAGAGC A UUGUGGAC | 663 | GUCCACAA CUGAUGAG GCCGUUAGGC CGAA ICUCUUGU | 2438 |
| 1321 | UUGUGGAC A GUGGCACC | 664 | GGUGCCAC CUGAUGAG GCCGUUAGGC CGAA IUCCACAA | 2439 |
| 1327 | ACAGUGGC A CCACCAAC | 665 | GUUGGUGG CUGAUGAG GCCGUUAGGC CGAA ICCACUGU | 2440 |
| 1329 | AGUGGCAC C ACCAACCU | 666 | AGGUUGGU CUGAUGAG GCCGUUAGGC CGAA IUGCCACU | 2441 |
| 1330 | GUGGCACC A CCAACCUU | 667 | AAGGUUGG CUGAUGAG GCCGUUAGGC CGAA IGUGCCAC | 2442 |
| 1332 | GGCACCAC C AACCUUCG | 668 | CGAAGGUU CUGAUGAG GCCGUUAGGC CGAA IUGGUGCC | 2443 |
| 1333 | GCACCACC A ACCUUCGU | 669 | ACGAAGGU CUGAUGAG GCCGUUAGGC CGAA IGUGGUGC | 2444 |
| 1336 | CCACCAAC C UUCGUUUG | 670 | CAAACGAA CUGAUGAG GCCGUUAGGC CGAA IUUGGUGG | 2445 |
| 1337 | CACCAACC U UCGUUUGC | 671 | GCAAACGA CUGAUGAG GCCGUUAGGC CGAA IGUUGGUG | 2446 |
| 1346 | UCGUUUGC C CAAGAAAG | 672 | CUUUCUUG CUGAUGAG GCCGUUAGGC CGAA ICAAACGA | 2447 |
| 1347 | CGUUUGCC C AAGAAAGU | 673 | ACUUUCUU CUGAUGAG GCCGUUAGGC CGAA IGCAAACG | 2448 |
| 1348 | GUUUGCCC A AGAAAGUG | 674 | CACUUUCU CUGAUGAG GCCGUUAGGC CGAA IGGCAAAC | 2449 |
| 1365 | UUUGAAGC U GCAGUCAA | 675 | UUGACUGC CUGAUGAG GCCGUUAGGC CGAA ICUUCAAA | 2450 |
| 1368 | GAAGCUGC A GUCAAAUC | 676 | GAUUUGAC CUGAUGAG GCCGUUAGGC CGAA ICAGCUUC | 2451 |
| 1372 | CUGCAGUC A AAUCCAUC | 677 | GAUGGAUC CUGAUGAG GCCGUUAGGC CGAA IACUGCAG | 2452 |
| 1377 | GUCAAAUC C AUCAAGGC | 678 | GCCUUGAU CUGAUGAG GCCGUUAGGC CGAA IAUUUGAC | 2453 |
| 1378 | UCAAAUCC A UCAAGGCA | 679 | UGCCUUGA CUGAUGAG GCCGUUAGGC CGAA IGAUUUGA | 2454 |
| 1381 | AAUCCAUC A AGGCAGCC | 680 | GGCUGCCU CUGAUGAG GCCGUUAGGC CGAA IAUGGAUU | 2455 |
| 1386 | AUCAAGGC A GCCUCCUC | 681 | GAGGAGGC CUGAUGAG GCCGUUAGGC CGAA ICCUUGAU | 2456 |
| 1389 | AAGGCAGC C UCCUCCAC | 682 | GUGGAGGA CUGAUGAG GCCGUUAGGC CGAA ICUGCCUU | 2457 |
| 1390 | AGGCAGCC U CCUCCACG | 683 | CGUGGAGG CUGAUGAG GCCGUUAGGC CGAA IGCUGCCU | 2458 |
| 1392 | GCAGCCUC C UCCACGGA | 684 | UCCGUGGA CUGAUGAG GCCGUUAGGC CGAA IAGGCUGC | 2459 |
| 1393 | CAGCCUCC U CCACGGAG | 685 | CUCCGUGG CUGAUGAG GCCGUUAGGC CGAA IGAGGCUG | 2460 |
| 1395 | GCCUCCUC C ACGGAGAA | 686 | UUCUCCGU CUGAUGAG GCCGUUAGGC CGAA IAGGAGGC | 2461 |
| 1396 | CCUCCUCC A CGGAGAAG | 687 | CUUCUCCG CUGAUGAG GCCGUUAGGC CGAA IGAGGAGG | 2462 |
| 1408 | AGAAGUUC C CUGAUGGU | 688 | ACCAUCAG CUGAUGAG GCCGUUAGGC CGAA IAACUUCU | 2463 |
| 1409 | GAAGUUCC C UGAUGGUU | 689 | AACCAUCA CUGAUGAG GCCGUUAGGC CGAA IGAACUUC | 2464 |
| 1410 | AAGUUCCC U GAUGGUUU | 690 | AAACCAUC CUGAUGAG GCCGUUAGGC CGAA IGGAACUU | 2465 |
| 1420 | AUGGUUUC U GGCUAGGA | 691 | UCCUAGCC CUGAUGAG GCCGUUAGGC CGAA IAAACCAU | 2466 |
| 1424 | UUUCUGGC U AGGAGAGC | 692 | GCUCUCCU CUGAUGAG CGGCUUAGGC CGAA ICCAGAAA | 2467 |
| 1433 | AGGAGAGC A GCUGGUGU | 693 | ACACCAGC CUGAUGAG GCCGUUAGGC CGAA ICUCUCCU | 2468 |
| 1436 | AGAGCAGC U GGUGUGCU | 694 | AGCACACC CUGAUGAG GCCGUUAGGC CGAA ICUGCUCU | 2469 |
| 1444 | UGGUGUGC U GGCAAGCA | 695 | UGCUUGCC CUGAUGAG GCCGUUAGGC CGAA ICACACCA | 2470 |
| 1448 | GUGCUGGC A AGCAGGCA | 696 | UGCCUGCU CUGAUGAG GCCGUUAGGC CGAA ICCAGCAC | 2471 |
| 1452 | UGGCAAGC A GGCACCAC | 697 | GUGGUGCC CUGAUGAG GCCGUUAGGC CGAA ICUUGCCA | 2472 |
| 1456 | AAGCAGGC A CCACCCCU | 698 | AGGGGUGG CUGAUGAG GCCGUUAGGC CGAA ICCUGCUU | 2473 |
| 1458 | GCAGGCAC C ACCCCUUG | 699 | CAAGGGGU CUGAUGAG GCCGUUAGGC CGAA IUGCCUGC | 2474 |
| 1459 | CAGGCACC A CCCCUUGG | 700 | CCAAGGGG CUGAUGAG GCCGUUAGGC CGAA IGUGCCUG | 2475 |
| 1461 | GGCACCAC C CCUUGGAA | 701 | UUCCAAGG CUGAUGAG GCCGUUAGGC CGAA IUGGUGCC | 2476 |
| 1462 | GCACCACC C CUUGGAAC | 702 | GUUCCAAG CUGAUGAG GCCGUUAGGC CGAA IGUGGUGC | 2477 |
| 1463 | CACCACCC C UUGGAACA | 703 | UGUUCCAA CUGAUGAG GCCGUUAGGC CGAA IGGUGGUG | 2478 |
| 1464 | ACCACCCC U UGGAACAU | 704 | AUGUUCCA CUGAUGAG GCCGUUAGGC CGAA IGGGUGGU | 2479 |
| 1471 | CUUGGAAC A UUUUCCCA | 705 | UGGGAAAA CUGAUGAG GCCGUUAGGC CGAA IUUCCAAG | 2480 |
| 1477 | ACAUUUUC C CAGUCAUC | 706 | GAUGACUG CUGAUGAG GCCGUUAGGC CGAA IAAAAUGU | 2481 |
| 1478 | CAUUUUCC C AGUCAUCU | 707 | AGAUGACU CUGAUGAG GCCGUUAGGC CGAA IGAAAAUG | 2482 |
| 1479 | AUUUUCCC A GUCAUCUC | 708 | GAGAUGAC CUGAUGAG GCCGUUAGGC CGAA IGGAAAAU | 2483 |
| 1483 | UCCCAGUC A UCUCACUC | 709 | GAGUGAGA CUGAUGAG GCCGUUAGGC CGAA IACUGGGA | 2484 |
| 1486 | CAGUCAUC U CACUCUAC | 710 | GUAGAGUG CUGAUGAG GCCGUUAGGC CGAA IAUGACUG | 2485 |
| 1488 | GUCAUCUC A CUCUACCU | 711 | AGGUAGAG CUGAUGAG GCCGUUAGGC CGAA IAGAUGAC | 2486 |
| 1490 | CAUCUCAC U CUACCUAA | 712 | UUAGGUAG CUGAUGAG GCCGUUAGGC CGAA IUGAGAUG | 2487 |
| 1492 | UCUCACUC U ACCUAAUG | 713 | CAUUAGGU CUGAUGAG GCCGUUAGGC CGAA IAGUGAGA | 2488 |
| 1495 | CACUCUAC C UAAUGGGU | 714 | ACCCAUUA CUGAUGAG GCCGUUAGGC CGAA IUAGAGUG | 2489 |
| 1496 | ACUCUACC U AAUGGGUG | 715 | CACCCAUU CUGAUGAG GCCGUUAGGC CGAA IGUAGAGU | 2490 |
| 1512 | GAGGUUAC C AACCAGUC | 716 | GACUGGUU CUGAUGAG GCCGUUAGGC CGAA IUAACCUC | 2491 |
| 1513 | AGGUUACC A ACCAGUCC | 717 | GGACUGGU CUGAUGAG GCCGUUAGGC CGAA IGUAACCU | 2492 |
| 1516 | UUACCAAC C AGUCCUUC | 718 | GAAGGACU CUGAUGAG GCCGUUAGGC CGAA IUUGGUAA | 2493 |
| 1517 | UACCAACC A GUCCUUCC | 719 | GGAAGGAC CUGAUGAG GCCGUUAGGC CGAA IGUUGGUA | 2494 |
| 1521 | AACCAGUC C UUCCGCAU | 720 | AUGCGGAA CUGAUGAG GCCGUUAGGC CGAA IACUGGUU | 2495 |
| 1522 | ACCAGUCC U UCCGCAUC | 721 | GAUGCGGA CUGAUGAG GCCGUUAGGC CGAA IGACUGGU | 2496 |
| 1525 | AGUCCUUC C GCAUCACC | 722 | GGUGAUGC CUGAUGAG GCCGUUAGGC CGAA IAAGGACU | 2497 |
| 1528 | CCUUCCGC A UCACCAUC | 723 | GAUGGUGA CUGAUGAG GCCGUUAGGC CGAA ICGGAAGG | 2498 |
| 1531 | UCCGCAUC A CCAUCCUU | 724 | AAGGAUGG CUGAUGAG GCCGUUAGGC CGAA IAUGCGGA | 2499 |
| 1533 | CGCAUCAC C AUCCUUCC | 725 | GGAAGGAU CUGAUGAG GCCGUUAGGC CGAA IUGAUGCG | 2500 |
| 1534 | GCAUCACC A UCCUUCCG | 726 | CGGAAGGA CUGAUGAG GCCGUUAGGC CGAA IGUGAUGC | 2501 |
| 1537 | UCACCAUC C UUCCGCAG | 727 | CUGCGGAA CUGAUGAG GCCGUUAGGC CGAA IAUGGUGA | 2502 |
| 1538 | CACCAUCC U UCCGCAGC | 728 | GCUGCGGA CUGAUGAG GCCGUUAGGC CGAA IGAUGGUG | 2503 |
| 1541 | CAUCCUUC C GCAGCAAU | 729 | AUUGCUGC CUGAUGAG GCCGUUAGGC CGAA IAAGGAUG | 2504 |
| 1544 | CCUUCCGC A GCAAUACC | 730 | GGUAUUGC CUGAUGAG GCCGUUAGGC CGAA ICGGAAGG | 2505 |
| 1547 | UCCGCAGC A AUACCUGC | 731 | GCAGGUAU CUGAUGAG GCCGUUAGGC CGAA ICUGCGGA | 2506 |
| 1552 | AGCAAUAC C UGCGGCCA | 732 | UGGCCGCA CUGAUGAG GCCGUUAGGC CGAA IUAUUGCU | 2507 |
| 1553 | GCAAUACC U GCGGCCAG | 733 | CUGGCCGC CUGAUGAG GCCGUUAGGC CGAA IGUAUUGC | 2508 |
| 1559 | CCUGCGGC C AGUGGAAG | 734 | CUUCCACU CUGAUGAG GCCGUUAGGC CGAA ICCGCAGG | 2509 |
| 1560 | CUGCGGCC A GUGGAAGA | 735 | UCUUCCAC CUGAUGAG GCCGUUAGGC CGAA IGCCGCAG | 2510 |
| 1575 | GAUGUGGC C ACGUCCCA | 736 | UGGGACGU CUGAUGAG GCCGUUAGGC CGAA ICCACAUC | 2511 |
| 1576 | AUGUGGCC A CGUCCCAA | 737 | UUGGGACG CUGAUGAG GCCGUUAGGC CGAA IGCCACAU | 2512 |
| 1581 | GCCACGUC C CAAGACGA | 738 | UCGUCUUG CUGAUGAG GCCGUUAGGC CGAA IACGUGGC | 2513 |
| 1582 | CCACGUCC C AAGACGAC | 739 | GUCGUCUU CUGAUGAG GCCGUUAGGC CGAA IGACGUGG | 2514 |
| 1583 | CACGUCCC A AGACGACU | 740 | AGUCGUCU CUGAUGAG GCCGUUAGGC CGAA IGGACGUG | 2515 |
| 1591 | AAGACGAC U GUUACAAG | 741 | CUUGUAAC CUGAUGAG GCCGUUAGGC CGAA IUCGUCUU | 2516 |
| 1597 | ACUGUUAC A AGUUUGCC | 742 | GGCAAACU CUGAUGAG GCCGUUAGGC CGAA IUAACAGU | 2517 |
| 1605 | AAGUUUGC C AUCUCACA | 743 | UGUGAGAU CUGAUGAG GCCGUUAGGC CGAA ICAAACUU | 2518 |
| 1606 | AGUUUGCC A UCUCACAG | 744 | CUGUGAGA CUGAUGAG GCCGUUAGGC CGAA IGCAAACU | 2519 |
| 1609 | UUGCCAUC U CACAGUCA | 745 | UGACUGUG CUGAUGAG GCCGUUAGGC CGAA IAUGGCAA | 2520 |
| 1611 | GCCAUCUC A CAGUCAUC | 746 | GAUGACUG CUGAUGAG GCCGUUAGGC CGAA IAGAUGGC | 2521 |
| 1613 | CAUCUCAC A GUCAUCCA | 747 | UGGAUGAC CUGAUGAG GCCGUUAGGC CGAA IUGAGAUG | 2522 |
| 1617 | UCACAGUC A UCCACGGG | 748 | CCCGUGGA CUGAUGAG GCCGUUAGGC CGAA IACUGUGA | 2523 |
| 1620 | CAGUCAUC C ACGGGCAC | 749 | GUGCCCGU CUGAUGAG GCCGUUAGGC CGAA IAUGACUG | 2524 |
| 1621 | AGUCAUCC A CGGGCACU | 750 | AGUGCCCG CUGAUGAG GCCGUUAGGC CGAA IGAUGACU | 2525 |
| 1627 | CCACGGGC A CUGUUAUG | 751 | CAUAACAG CUGAUGAG GCCGUUAGGC CGAA ICCCGUGG | 2526 |
| 1629 | ACGGGCAC U GUUAUGGG | 752 | CCCAUAAC CUGAUGAG GCCGUUAGGC CGAA IUGCCCGU | 2527 |
| 1641 | AUGGGAGC U GUUAUCAU | 753 | AUGAUAAC CUGAUGAG GCCGUUAGGC CGAA ICUCCCAU | 2528 |
| 1648 | CUGUUAUC A UGGAGGGC | 754 | GCCCUCCA CUGAUGAG GCCGUUAGGC CGAA IAUAACAG | 2529 |
| 1657 | UGGAGGGC U UCUACGUU | 755 | AACGUAGA CUGAUGAG GCCGUUAGGC CGAA ICCCUCCA | 2530 |
| 1660 | AGGGCUUC U ACGUUGUC | 756 | GACAACGU CUGAUGAG GCCGUUAGGC CGAA IAAGCCCU | 2531 |
| 1669 | ACGUUGUC U UUGAUCGG | 757 | CCGAUCAA CUGAUGAG GCCGUUAGGC CGAA IACAACGU | 2532 |
| 1680 | GAUCGGGC C CGAAAACG | 758 | CGUUUUCG CUGAUGAG GCCGUUAGGC CGAA ICCCGAUC | 2533 |
| 1681 | AUCGGGCC C GAAAACGA | 759 | UCGUUUUC CUGAUGAG GCCGUUAGGC CGAA IGCCCGAU | 2534 |
| 1696 | GAAUUGGC U UUGCUGUC | 760 | GACAGCAA CUGAUGAG GCCGUUAGGC GCAA ICCAAUUC | 2535 |
| 1701 | GGCUUUGC U GUCAGCGC | 761 | GCGCUGAC CUGAUGAG GCCGUUAGGC CGAA ICAAAGCC | 2536 |
| 1705 | UUGCUGUC A GCGCUUGC | 762 | GCAAGCGC CUGAUGAG GCCGUUAGGC CGAA IACAGCAA | 2537 |
| 1710 | GUCAGCGC U UGCCAUGU | 763 | ACAUGGCA CUGAUGAG GCCGUUAGGC CGAA ICGCUGAC | 2538 |
| 1714 | GCGCUUGC C AUGUGCAC | 764 | GUGCACAU CUGAUGAG GCCGUUAGGC CGAA ICAAGCGC | 2539 |
| 1715 | CGCUUGCC A UGUGCACG | 765 | CGUGCACA CUGAUGAG GCCGUUAGGC CGAA IGCAAGCG | 2540 |
| 1721 | CCAUGUGC A CGAUGAGU | 766 | ACUCAUCG CUGAUGAG GCCGUUAGGC CGAA ICACAUGG | 2541 |
| 1732 | AUGAGUUC A GGACGGCA | 767 | UGCCGUCC CUGAUGAG GCCGUUAGGC CGAA IAACUCAU | 2542 |
| 1740 | AGGACGGC A GCGGUGGA | 768 | UCCACCGC CUGAUGAG GCCGUUAGGC CGAA ICCGUCCU | 2543 |
| 1753 | UGGAAGGC C CUUUUGUC | 769 | GACAAAAG CUGAUGAG GCCGUUAGGC CGAA ICCUUCCA | 2544 |
| 1754 | GGAAGGCC C UUUUGUCA | 770 | UGACAAAA CUGAUGAG GCCGUUAGGC CGAA IGCCUUCC | 2545 |
| 1755 | GAAGGCCC U UUUGUCAC | 771 | GUGACAAA CUGAUGAG GCCGUUAGGC CGAA IGGCCUUC | 2546 |
| 1762 | CUUUUGUC A CCUUGGAC | 772 | GUCCAAGG CUGAUGAG GCCGUUAGGC CGAA IACAAAAG | 2547 |
| 1764 | UUUGUCAC C UUGGACAU | 773 | AUGUCCAA CUGAUGAG GCCGUUAGGC CGAA IUGACAAA | 2548 |
| 1765 | UUGUCACC U UGGACAUG | 774 | CAUGUCCA CUGAUGAG GCCGUUAGGC CGAA IGUGACAA | 2549 |
| 1771 | CCUUGGAC A UGGAAGAC | 775 | GUCUUCCA CUGAUGAG GCCGUUAGGC CGAA IUCCAAGG | 2550 |
| 1780 | UGGAAGAC U GUGGCUAC | 776 | GUAGCCAC CUGAUGAG GCCGUUAGGC CGAA IUCUUCCA | 2551 |
| 1786 | ACUGUGGC U ACAACAUU | 777 | AAUGUUGU CUGAUGAG GCCGUUAGGC CGAA ICCACAGU | 2552 |
| 1789 | GUGGCUAC A ACAUUCCA | 778 | UGGAAUGU CUGAUGAG GCCGUUAGGC CGAA IUAGCCAC | 2553 |
| 1792 | GCUACAAC A UUCCACAG | 779 | CUGUGGAA CUGAUGAG GCCGUUAGGC CGAA IUUGUAGC | 2554 |
| 1796 | CAACAUUC C ACAGACAG | 780 | CUGUCUGU CUGAUGAG GCCGUUAGGC CGAA IAAUGUUG | 2555 |
| 1797 | AACAUUCC A CAGACAGA | 781 | UCUGUCUG CUGAUGAG GCCGUUAGGC CGAA IGAAUGUU | 2556 |
| 1799 | CAUUCCAC A GACAGAUG | 782 | CAUCUGUC CUGAUGAG GCCGUUAGGC CGAA IUGGAAUG | 2557 |
| 1803 | CCACAGAC A GAUGAGUC | 783 | GACUCAUC CUGAUGAG GCCGUUAGGC CGAA IUCUGUGG | 2558 |
| 1812 | GAUGAGUC A ACCCUCAU | 784 | AUGAGGGU CUGAUGAG GCCGUUAGGC CGAA IACUCAUC | 2559 |
| 1815 | GAGUCAAC C CUCAUGAC | 785 | GUCAUGAG CUGAUGAG GCCGUUAGGC CGAA IUUGACUC | 2560 |
| 1816 | AGUCAACC C UCAUGACC | 786 | GGUCAUGA CUGAUGAG GCCGUUAGGC CGAA IGUUGACU | 2561 |
| 1817 | GUCAACCC U CAUGACCA | 787 | UGGUCAUG CUGAUGAG GCCGUUAGGC CGAA IGGUUGAC | 2562 |
| 1819 | CAACCCUC A UGACCAUA | 788 | UAUGGUCA CUGAUGAG GCCGUUAGGC CGAA IAGGGUUG | 2563 |
| 1824 | CUCAUGAC C AUAGCCUA | 789 | UAGGCUAU CUGAUGAG GCCGUUAGGC CGAA IUCAUGAG | 2564 |
| 1825 | UCAUGACC A UAGCCUAU | 790 | AUAGGCUA CUGAUGAG GCCGUUAGGC CGAA IGUCAUGA | 2565 |
| 1830 | ACCAUAGC C UAUGUCAU | 791 | AUGACAUA CUGAUGAG GCCGUUAGGC CGAA ICUAUGGU | 2566 |
| 1831 | CCAUAGCC U AUGUCAUG | 792 | CAUGACAU CUGAUGAE GCCGUUAGGC CGAA IGCUAUGG | 2567 |
| 1837 | CCUAUGUC A UGGCUGCC | 793 | GGCAGCCA CUGAUGAG GCCGUUAGGC CGAA IACAUAGG | 2568 |
| 1842 | GUCAUGGC U CGGAUCUG | 794 | CAGAUGGC CUGAUGAG GCCGUUAGGC CGAA ICCAUGAC | 2569 |
| 1845 | AUGGCUGC C AUCUGCGC | 795 | GCGCAGAU CUGAUGAG GCCGUUAGGC CGAA ICAGCCAU | 2570 |
| 1846 | UGGCUGCC A UCUGCGCC | 796 | GGCGCAGA CUGAUGAG GCCGUUAGGC CGAA IGCAGCCA | 2571 |
| 1849 | CUGCCAUC U GCGCCCUC | 797 | GAGGGCGC CUGAUGAG GCCGUUAGGC CGAA IAUGGCAG | 2572 |
| 1854 | AUCUGCGC C CUCUUCAU | 798 | AUGAAGAG CUGAUGAG GCCGUUAGGC CGAA ICGCAGAU | 2573 |
| 1855 | UCUGCGCC C UCCUCAUG | 799 | CAUGAAGA CUGAUGAG GCCGUUAGGC CGAA IGCGCAGA | 2574 |
| 1856 | CUGCGCCC U CUUCAUGC | 800 | GCAUGAAG CUGAUGAG GCCGUUAGGC CGAA IGGCGCAG | 2575 |
| 1858 | GCGCCCUC U UCAUGCUG | 801 | CAGCAUGA CUGAUGAG GCCGUUAGGC CGAA IAGGGCGC | 2576 |
| 1861 | CCCUCUUC A UGCUGCCA | 802 | UGGCAGCA CUGAUGAG GCCGUUAGGC CGAA IAAGAGGG | 2577 |
| 1865 | CUUCAUGC U GCCACUCU | 803 | AGAGUGGC CUGAUGAG GCCGUUAGGC CGAA ICAUGAAG | 2578 |
| 1868 | CAUGCUGC C ACUCUGCC | 804 | GGCAGAGU CUGAUGAG GCCGUUAGGC CGAA ICAGCAUG | 2579 |
| 1869 | AUGCUGCC A CUCUGCCU | 805 | AGGCAGAG CUGAUGAG GCCGUUAGGC CGAA IGCAGCAU | 2580 |
| 1871 | GCUGCCAC U CUGCCUCA | 806 | UGAGGCAG CUGAUGAG GCCGUUAGGC CGAA IUGGCAGC | 2581 |
| 1873 | UGCCACUC U GCCUCAUG | 807 | CAUGAGGC CUGAUGAG GCCGUUAGGC CGAA IAGUGGCA | 2582 |
| 1876 | CACUCUGC C UCAUGGUG | 808 | CACCAUGA CUGAUGAG GCCGUUAGGC CGAA ICAGAGUG | 2583 |
| 1877 | ACUCUGCC U CAUGGUGU | 809 | ACACCAUG CUGAUGAG GCCGUUAGGC CGAA IGCAGAGU | 2584 |
| 1879 | UCUGCCUC A UGGUGUGU | 810 | ACACACCA CUGAUGAG GCCGUUAGGC CGAA IAGGCAGA | 2585 |
| 1889 | GGUGUGUC A GUGGCGCU | 811 | AGCGCCAC CUGAUGAG GCCGUUAGGC CGAA IACACACC | 2586 |
| 1897 | AGUGGCGC U GCCUCCGC | 812 | GCGGAGGC CUGAUGAG GCCGUUAGGC CGAA ICGCCACU | 2587 |
| 1900 | GGCGCUGC C UCCGCUGC | 813 | GCAGCGGA CUGAUGAG GCCGUUAGGC CGAA ICAGCGCC | 2588 |
| 1901 | GCGCUGCC U CCGCUGCC | 814 | GGCAGCGG CUGAUGAG GCCGUUAGGC CGAA IGCAGCGC | 2589 |
| 1903 | GCUGCCUC C GCUGCCUG | 815 | CAGGCAGC CUGAUGAG GCCGUUAGGC CGAA IAGGCAGC | 2590 |
| 1906 | GCCUCCGC U GCCUGCGC | 816 | GCGCAGGC CUGAUGAG GCCGUUAGGC CGAA ICGGAGGC | 2591 |
| 1909 | UCCGCUGC C UGCGCCAG | 817 | CUGGCGCA CUGAUGAG GCCGUUAGGC CGAA ICAGCGGA | 2592 |
| 1910 | CCGCUGCC U GCGCCAGC | 818 | GCUGGCGC CUGAUGAG GCCGUUAGGC CGAA IGCAGCGG | 2593 |
| 1915 | GCCUGCGC C AGCAGCAU | 819 | AUGCUGCU CUGAUGAG GCCGUUAGGC CGAA ICGCAGGC | 2594 |
| 1916 | CCUGCGCC A GCAGCAUG | 820 | CAUGCUGC CUGAUGAG GCCGUUAGGC CGAA IGCGCAGG | 2595 |
| 1919 | GCGCCAGC A GCAUGAUG | 821 | CAUCAUGC CUGAUGAG GCCGUUAGGC CGAA ICUGGCGC | 2596 |
| 1922 | CCAGCAGC A UGAUGACU | 822 | AGUCAUCA CUGAUGAG GCCGUUAGGC CGAA ICUGCUGG | 2597 |
| 1930 | AUGAUGAC U UUGCUGAU | 823 | AUCAGCAA CUGAUGAG GCCGUUAGGC CGAA IUCAUCAU | 2598 |
| 1935 | GACUUUGC U GAUGACAU | 824 | AUGUCAUC CUGAUGAG GCCGUUAGGC CGAA ICAAAGUC | 2599 |
| 1942 | CUGAUGAC A UCUCCCUG | 825 | CAGGGAGA CUGAUGAG GCCGUUAGGC CGAA IUCAUCAG | 2600 |
| 1945 | AUGACAUC U CCCUGCUG | 826 | CAGCAGGG CUGAUGAG GCCGUUAGGC CGAA IAUGUCAU | 2601 |
| 1947 | GACAUCUC C CUGCUGAA | 827 | UUCAGCAG CUGAUGAG GCCGUUAGGC CGAA IAGAUGUC | 2602 |
| 1948 | ACAUCUCC C UGCUGAAG | 828 | CUUCAGCA CUGAUGAG GCCGUUAGGC CGAA IGAGAUGU | 2603 |
| 1949 | CAUCUCCC U GCUGAAGU | 829 | ACUUCAGC CUGAUGAG GCCGUUAGGC CGAA IGGAGAUG | 2604 |
| 1952 | CUCCCUGC U GAAGUGAG | 830 | CUCACUUC CUGAUGAG GCCGUUAGGC CGAA ICAGGGAG | 2605 |
| 1966 | GAGGAGGC C CAUGGGCA | 831 | UGCCCAUG CUGAUGAG GCCGUUAGGC CGAA ICCUCCUC | 2606 |
| 1967 | AGGAGGCC C AUGGGCAG | 832 | CUGCCCAU CUGAUGAG GCCGUUAGGC CGAA IGCCUCCU | 2607 |
| 1968 | GGAGGCCC A UGGGCAGA | 833 | UCUGCCCA CUGAUGAG GCCGUUAGGC CGAA IGGCCUCC | 2608 |
| 1974 | CCAUGGGC A GAAGAUAC | 834 | CUAUCUUC CUGAUGAG GCCGUUAGGC CGAA ICCCAUGG | 2609 |
| 1989 | AGAGAUUC C CCUGGACC | 835 | GGUCCAGG CUGAUGAG GCCGUUAGGC CGAA IAAUCUCU | 2610 |
| 1990 | GAGAUUCC C CUGGACCA | 836 | UGGUCCAG CUGAUGAG GCCGUUAGGC CGAA IGAAUCUC | 2611 |
| 1991 | AGAUUCCC C UGGACCAC | 837 | GUGGUCCA CUGAUGAG GCCGUUAGGC CGAA IGGAAUCU | 2612 |
| 1992 | GAUUCCCC U GGACCACA | 838 | UGUGGUCC CUGAUGAG GCCGUUAGGC CGAA IGGGAAUC | 2613 |
| 1997 | CCCUGGAC C ACACCUCC | 839 | GGAGGUGU CUGAUGAG GCCGUUAGGC CGAA IUCCAGGG | 2614 |
| 1998 | CCUGGACC A CACCUCCG | 840 | CGGAGGUG CUGAUGAG GCCGUUAGGC CGAA IGUCCAGG | 2615 |
| 2000 | UGGACCAC A CCUCCGUG | 841 | CACGGAGG CUGAUGAG GCCGUUAGGC CGAA IUGGUCCA | 2616 |
| 2002 | GACCACAC C UCCGUGGU | 842 | ACCACGGA CUGAUGAG GCCGUUAGGC CGAA IUGUGGUC | 2617 |
| 2003 | ACCACACC U CCGUGGUU | 843 | AACCACGG CUGAUGAG GCCGUUAGGC CGAA IGUGUGGU | 2618 |
| 2005 | CACACCUC C GUGGUUCA | 844 | UGAACCAC CUGAUGAG GCCGUUAGGC CGAA IAGGUGUG | 2619 |
| 2013 | CGUGGUUC A CUUUGGUC | 845 | GACCAAAG CUGAUGAG GCCGUUAGGC CGAA IAACCACG | 2620 |
| 2015 | UGGUUCAC U UUGGUCAC | 846 | GUGACCAA CUGAUGAG GCCGUUAGGC CGAA IUGAACCA | 2621 |
| 2022 | CUUUGGUC A CAAGUAGG | 847 | CCUACUUG CUGAUGAG GCCGUUAGGC CGAA IACCAAAG | 2622 |
| 2024 | UUGGUCAC A AGUAGGAG | 848 | CUCCUACU CUGAUGAG GCCGUUAGGC CGAA IUGACCAA | 2623 |
| 2035 | UAGGAGAC A CAGAUGGC | 849 | GCCAUCUG CUGAUGAG GCCGUUAGGC CGAA IUCUCCUA | 2624 |
| 2037 | GGAGACAC A GAUGGCAC | 850 | GUGCCAUC CUGAUGAG GCCGUUAGGC CGAA IUGUCUCC | 2625 |
| 2044 | CAGAUGGC A CCUGUGGC | 851 | GCCACAGG CUGAUGAG GCCGUUAGGC CGAA ICCAUCUG | 2626 |
| 2046 | GAUGGCAC C UGUGGCCA | 852 | UGGCCACA CUGAUGAG GCCGUUAGGC CGAA IUGCCAUC | 2627 |
| 2047 | AUGGCACC U GUGGCCAG | 853 | CUGGCCAC CUGAUGAG GCCGUUAGGC CGAA IGUGCCAU | 2628 |
| 2053 | CCUGUGGC C AGAGCACC | 854 | GGUGCUCU CUGAUGAG GCCGUUAGGC CGAA IGGACAGG | 2629 |
| 2054 | CUGUGGCC A GAGCACCU | 855 | AGGUGCUC CUGAUGAG GCCGUUAGGC CGAA IGCCACAG | 2630 |
| 2059 | GCCAGAGC A CCUCAGGA | 856 | UCCUGAGG CUGAUGAG GCCGUUAGGC CGAA ICUCUGGC | 2631 |
| 2061 | CAGAGCAC C UCAGGACC | 857 | GGUCCUGA CUGAUGAG GCCGUUAGGC CGAA IUGCUCUG | 2632 |
| 2052 | AGAGCACC U CAGGACCC | 858 | GGGUCCUG CUGAUGAG GCCGUUAGGC CGAA IGUGCUCU | 2633 |
| 2064 | AGCACCUC A GGACCCUC | 859 | GAGGGUCC CUGAUGAG GCCGUUAGGC CGAA IAGGUGCU | 2634 |
| 2069 | CUCAGGAC C CUCCCCAC | 860 | GUGGGGAG CUGAUGAG GCCGUUAGGC CGAA IUCCUGAG | 2635 |
| 2070 | UCAGGACC C UCCCCACC | 861 | GGUGGGGA CUGAUGAG GCCGUUAGGC CGAA IGUCCUGA | 2636 |
| 2071 | CAGGACCC U CCCCACCC | 862 | GGGUGGGG CUGAUGAG GCCGUUAGGC CGAA IGGUCCUG | 2637 |
| 2073 | GGACCCUC C CCACCCAC | 863 | GUGGGUGG CUGAUGAG GCCGUUAGGC CGAA IAGGGUCC | 2638 |
| 2074 | GACCCUCC C CACCCACC | 864 | GGUGGGUG CUGAUGAG GCCGUUAGGC CGAA IGAGGGUC | 2639 |
| 2075 | ACCCUCCC C ACCCACCA | 865 | UGGUGGGU CUGAUGAG GCCGUUAGGC CGAA IGGAGGGU | 2640 |
| 2076 | CCCUCCCC A CCCACCAA | 866 | UUGGUGGG CUGAUGAG GCCGUUAGGC CGAA IGGGAGGG | 2641 |
| 2078 | CUCCCCAC C CACCAAAU | 867 | AUUUGGUG CUGAUGAG GCCGUUAGGC CGAA IUGGGGAG | 2642 |
| 2079 | UCCCCACC C ACCAAAUG | 868 | CAUUUGGU CUGAUGAG GCCGUUAGGC CGAA IGUGGGGA | 2643 |
| 2080 | CCCCACCC A CCAAAUGC | 869 | GCAUUUGG CUGAUGAG GCCGUUAGGC CGAA IGGUGGGG | 2644 |
| 2082 | CCACCCAC C AAAUGCCU | 870 | AGGCAUUU CUGAUGAG GCCGUUAGGC CGAA IUGGGUGG | 2645 |
| 2083 | CACCCACC A AAUGCCUC | 871 | GAGGCAUU CUGAUGAG GCCGUUAGGC CGAA IGUGGGUG | 2646 |
| 2089 | CCAAAUGC C UCUGCCUU | 872 | AAGGCAGA CUGAUGAG GCCGUUAGGC CGAA ICAUUUGG | 2647 |
| 2090 | CAAAUGCC U CUGCCUUG | 873 | CAAGGCAG CUGAUGAG GCCGUUAGGC CGAA IGCAUUUG | 2648 |
| 2092 | AAUGCCUC U GCCUUGAU | 874 | AUCAAGGC CUGAUGAG GCCGUUAGGC CGAA IAGGCAUU | 2649 |
| 2095 | GCCUCUGC C CUGAUGGA | 875 | UCCAUCAA CUGAUGAG GCCGUUAGGC CGAA ICAGAGGC | 2650 |
| 2096 | CCUCUGCC U UGAUGGAG | 876 | CUCCAUCA CUGAUGAG GCCGUUAGGC CGAA IGCAGAGG | 2651 |
| 2116 | GAAAAGGC U GGCAAGGU | 877 | ACCUUGCC CUGAUGAG GCCGUUAGGC CGAA ICCUUUUC | 2652 |
| 2120 | AGGCUGGC A AGGUGGGU | 878 | ACCCACCU CUGAUGAG GCCGUUAGGC CGAA ICCAGCCU | 2653 |
| 2131 | GUGGGUUC C AGGGACUG | 879 | CAGUCCCU CUGAUGAG GCCGUUAGGC CGAA IAACCCAC | 2654 |
| 2132 | UGGGUUCC A GGGACUGU | 880 | ACAGUCCC CUGAUGAG GCCGUUAGGC CGAA IGAACCCA | 2655 |
| 2138 | CCAGGGAC U GUACCUGU | 881 | ACAGGUAC CUGAUGAG GCCGUUAGGC CGAA IUCCCUGG | 2656 |
| 2143 | GACUGUAC C UGUAGGAA | 882 | UUCCUACA CUGAUGAG GCCGUUAGGC CGAA IUACAGUC | 2657 |
| 2144 | ACUGUACC U GUAGGAAA | 883 | UUUCCUAC CUGAUGAG GCCGUUAGGC CGAA IGUACAGU | 2658 |
| 2154 | UAGGAAAC A GAAAAGAG | 884 | CUCUUUUC CUGAUGAG GCCGUUAGGC CGAA IUUUCCUA | 2659 |
| 2174 | AAAGAAGC A CUCUGCUG | 885 | CAGCAGAG CUGAUGAG GCCGUUAGGC CGAA ICUUCUUU | 2660 |
| 2176 | AGAAGCAC U CUGCUGGC | 886 | GCCAGCAG CUGAUGAG GCCGUUAGGC CGAA IUGCUUCU | 2661 |
| 2178 | AAGCACUC U GCUGGCGG | 887 | CCGCCAGC CUGAUGAG GCCGUUAGGC CGAA IAGUGCUU | 2662 |
| 2181 | CACUCUGC U GGCGGGAA | 888 | UUCCCGCC CUGAUGAG GCCGUUAGGC CGAA ICAGAGUG | 2663 |
| 2193 | GGGAAUAC U CUUGGUCA | 889 | UGACCAAG CUGAUGAG GCCGUUAGGC CGAA IUAUUCCC | 2664 |
| 2195 | GAAUACUC U UGGUCACC | 890 | GGUGACCA CUGAUGAG GCCGUUAGGC CGAA IAGUAUUC | 2665 |
| 2201 | UCUUGGUC A CCUCAAAU | 891 | AUUUGAGG CUGAUGAG GCCGUUAGGC CGAA IACCAAGA | 2666 |
| 2203 | UUGGUCAC C UCAAAUUU | 892 | AAAUUUGA CUGAUGAG GCCGUUAGGC CGAA IUGACCAA | 2667 |
| 2204 | UGGUCACC U CAAAUUUA | 893 | UAAAUUUG CUGAUGAG GCCGUUAGGC CGAA IGUGACCA | 2668 |
| 2206 | GUCACCUC A AAUUUAAG | 894 | CUUAAAUU CUGAUGAG GCCGUUAGGC CGAA IAGGUGAC | 2669 |
| 2226 | GGAAAUUC U GCUGCUUG | 895 | CAAGCAGC CUGAUGAG GCCGUUAGGC CGAA IAAUUUCC | 2670 |
| 2229 | AAUUCUGC U GCUUGAAA | 896 | UUUCAAGC CUGAUGAG GCCGUUAGGC CGAA ICAGAAUU | 2671 |
| 2232 | UCUGCUGC U UGAAACUU | 897 | AAGUUUCA CUGAUGAG GCCGUUAGGC CGAA ICAGCAGA | 2672 |
| 2239 | CUUGAAAC U UCAGCCCU | 898 | AGGGCUGA CUGAUGAG GCCGUUAGGC CGAA IUUUCAAG | 2673 |
| 2242 | GAAACUUC A GCCCUGAA | 899 | UUCAGGGC CUGAUGAG GCCGUUAGGC CGAA IAAGUUUC | 2674 |
| 2245 | ACUUCAGC C CUGAACCU | 900 | AGGUUCAG CUGAUGAG GCCGUUAGGC CGAA ICUGAAGU | 2675 |
| 2246 | CUUCAGCC C UGAACCUU | 901 | AAGGUUCA CUGAUGAG GCCGUUAGGC CGAA IGCUGAAG | 2676 |
| 2247 | UUCAGCCC U GAACCUUU | 902 | AAAGGUUC CUGAUGAG GCCGUUAGGC CGAA IGGCUGAA | 2677 |
| 2252 | CCCUGAAC C UUUGUCCA | 902 | UGGACAAA CUGAUGAG GCCGUUAGGC CGAA IUUCAGGG | 2678 |
| 2253 | CCUGAACC U UUGUCCAC | 904 | GUGGACAA CUGAUGAG GCCGUUAGGC CGAA IGUUCAGG | 2679 |
| 2259 | CCUUUGUC C ACCAUUCC | 905 | GGAAUGGU CUGAUGAG GCCGUUAGGC CGAA IACAAAGG | 2680 |
| 2260 | CUUUGUCC A CCAUUCCU | 906 | AGGAAUGG CUGAUGAG GCCGUUAGGC CGAA IGACAAAG | 2681 |
| 2262 | UUGUCCAC C AUUCCUUU | 907 | AAAGGAAU CUGAUGAG GCCGUUAGGC CGAA IUGGACAA | 2682 |
| 2263 | UGUCCACC A UUCCUUUA | 908 | UAAAGGAA CUGAUGAG GCCGUUAGGC CGAA IGUGGACA | 2683 |
| 2267 | CACCAUUC C UUUAAAUU | 909 | AAUUUAAA CUGAUGAG GCCGUUAGGC CGAA IAAUGGUG | 2684 |
| 2268 | ACCAUUCC U UUAAAUUC | 910 | GAAUUUAA CUGAUGAG GCCGUUAGGC CGAA IGAAUGGU | 2685 |
| 2277 | UUAAAUUC U CCAACCCA | 911 | UGGGUUGG CUGAUGAG GCCGUUAGGC CGAA IAAUUUAA | 2686 |
| 2279 | AAAUUCUC C AACCCAAA | 912 | UUUGGGUU CUGAUGAG GCCGUUAGGC CGAA IAGAAUUU | 2687 |
| 2280 | AAUUCUCC A ACCCAAAG | 913 | CUUUGGGU CUGAUGAG GCCGUUAGGC CGAA IGAGAAUU | 2688 |
| 2283 | UCUCCAAC C CAAAGUAU | 914 | AUACUUUG CUGAUGAG GCCGUUAGGC CGAA IUUGGAGA | 2689 |
| 2284 | CUCCAACC C AAAGUAUU | 915 | AAUACUUU CUGAUGAG GCCGUUAGGC CGAA IGUUGGAG | 2690 |
| 2285 | UCCAACCC A AAGUAUUC | 916 | GAAUACUU CUGAUGAG GCCGUUAGGC CGAA IGGUUGGA | 2691 |
| 2294 | AAGUAUUC U UCUUUUCU | 917 | AGAAAAGA CUGAUGAG GCCGUUAGGC CGAA IAAUACUU | 2692 |
| 2297 | UAUUCUUC U UUUCUUAG | 918 | CUAAGAAA CUGAUGAG GCCGUUAGGC CGAA IAAGAAUA | 2693 |
| 2302 | UUCUUUUC U UAGUUUCA | 919 | UGAAACUA CUGAUGAG GCCGUUAGGC CGAA IAAAAGAA | 2694 |
| 2310 | UUAGUUUC A GAAGUACU | 920 | AGUACUUC CUGAUGAG GCCGUUAGGC CGAA IAAACUAA | 2695 |
| 2318 | AGAAGUAC U GGCAUCAC | 921 | GUGAUGCC CUGAUGAG GCCGUUAGGC CGAA IUACUUCU | 2696 |
| 2322 | GUACUGGC A UCACACGC | 922 | GCGUGUGA CUGAUGAG GCCGUUAGGC CGAA ICCAGUAC | 2697 |
| 2325 | CUGGCAUC A CACGCAGG | 923 | CCUGCGUG CUGAUGAG GCCGUUAGGC CGAA IAUGCCAG | 2698 |
| 2327 | GGCAUCAC A CGCAGGUU | 924 | AACCUGCG CUGAUGAG GCCGUUAGGC CGAA IUGAUGCC | 2699 |
| 2331 | UCACACGC A GGUUACCU | 925 | AGGUAACC CUGAUGAG GCCGUUAGGC CGAA ICGUGUGA | 2700 |
| 2338 | CAGGUUAC C UUGGCGUG | 926 | CACGCCAA CUGAUGAG GCCGUUAGGC CGAA IUAACCUG | 2701 |
| 2339 | AGGUUACC U UGGCGUGU | 927 | ACACGCCA CUGAUGAG GCCGUUAGGC CGAA IGUAACCU | 2702 |
| 2351 | CGUGUGUC C CUGUGGUA | 928 | UACCACAG CUGAUGAG GCCGUUAGGC CGAA IACACACG | 2703 |
| 2352 | GUGUGUCC C UGUGGUAC | 929 | GUACCACA CUGAUGAG GCCGUUAGGC CGAA IGACACAC | 2704 |
| 2353 | UGUGUCCC U GUGGUACC | 930 | GGUACCAC CUGAUGAG GCCGUUAGGC CGAA IGGACACA | 2705 |
| 2361 | UGUGGUAC C CUGGCAGA | 931 | UCUGCCAG CUGAUGAG GCCGUUAGGC CGAA IUACCACA | 2706 |
| 2362 | GUGGUACC C UGGCAGAG | 932 | CUCUGCCA CUGAUGAG GCCGUUAGGC CGAA IGUACCAC | 2707 |
| 2363 | UGGUACCC U GGCAGAGA | 933 | UCUCUGCC CUGAUGAG GCCGUUAGGC CGAA IGGUACCA | 2708 |
| 2367 | ACCCUGGC A GAGAAGAG | 934 | CUCUUCUC CUGAUGAG GCCGUUAGGC CGAA ICCAGGGU | 2709 |
| 2378 | GAAGAGAC C AAGCUUGU | 935 | ACAAGCUU CUGAUGAG GCCGUUAGGC CGAA IUCUCUUC | 2710 |
| 2379 | AAGAGACC A AGCUUGUU | 936 | AACAAGCU CUGAUGAG GCCGUUAGGC CGAA IGUCUCUU | 2711 |
| 2383 | GACCAAGC U UGUUUCCC | 937 | GGGAAACA CUGAUGAG GCCGUUAGGC CGAA ICUUGGUC | 2712 |
| 2390 | CUUGUUUC C CUGCUGGC | 938 | GCCAGCAG CUGAUGAG GCCGUUAGGC CGAA IAAACAAG | 2713 |
| 2391 | UUGUUUCC C UGCUGGCC | 939 | GGCCAGCA CUGAUGAG GCCGUUAGGC CGAA IGAAACAA | 2714 |
| 2392 | UGUUUCCC U GCUGGCCA | 940 | UGGCCAGC CUGAUGAG GCCGUUAGGC CGAA IGGAAACA | 2715 |
| 2395 | UUCCCUGC U GGCCAAAG | 941 | CUUUGGCC CUGAUGAG GCCGUUAGGC CGAA ICAGGGAA | 2716 |
| 2399 | CUGCUGGC C AAAGUCAG | 942 | CUGACUUU CUGAUGAG GCCGUUAGGC CGAA ICCAGCAG | 2717 |
| 2400 | UGCUGGCC A AAGUCAGU | 943 | ACUGACUU CUGAUGAG GCCGUUAGGC CGAA IGCCAGCA | 2718 |
| 2406 | CCAAAGUC A GUAGGAGA | 944 | UCUCCUAC CUGAUGAG GCCGUUAGGC CGAA IACUUUGG | 2719 |
| 2421 | GAGGAUGC A CAGUUUGC | 945 | GCAAACUG CUGAUGAG GCCGUUAGGC CGAA ICAUCCUC | 2720 |
| 2423 | GGAUGCAC A GUUUGCUA | 946 | UAGCAAAC CUGAUGAG GCCGUUAGGC CGAA IUGCAUCC | 2721 |
| 2430 | CAGUUUGC U AUUUGCUU | 947 | AAGCAAAU CUGAUGAG GCCGUUAGGC CGAA ICAAACUG | 2722 |
| 2437 | CUAUUUGC U UUAGAGAC | 948 | GUCUCUAA CUGAUGAG GCCGUUAGGC CGAA ICAAAUAG | 2723 |
| 2446 | UUAGAGAC A GGGACUGU | 949 | ACAGUCCC CUGAUGAG GCCGUUAGGC CGAA IUCUCUAA | 2724 |
| 2452 | ACAGGGAC U GUAUAAAC | 950 | GUUUAUAC CUGAUGAG GCCGUUAGGC CGAA IUCCCUGU | 2725 |
| 2461 | GUAUAAAC A AGCCUAAC | 951 | GUUAGGCU CUGAUGAG GCCGUUAGGC CGAA IUUUAUAC | 2726 |
| 2465 | AAACAAGC C UAACAUUG | 952 | CAAUGUUA CUGAUGAG GCCGUUAGGC CGAA ICUUGUUU | 2727 |
| 2466 | AACAAGCC U AACAUUGG | 953 | CCAAUGUU CUGAUGAG GCCGUUAGGC CGAA IGCUUGUU | 2728 |
| 2470 | AGCCUAAC A UUGGUGCA | 954 | UGCACCAA CUGAUGAG GCCGUUAGGC CGAA IUUAGGCU | 2729 |
| 2478 | AUUGGUGC A AAGAUUGC | 955 | GCAAUCUU CUGAUGAG GCCGUUAGGC CGAA ICACCAAU | 2730 |
| 2487 | AAGAUUGC C UCUUGAAU | 956 | AUUCAAGA CUGAUGAG GCCGUUAGGC CGAA ICAAUCUU | 2731 |
| 2488 | AGAUUGCC U CUUGAAUU | 957 | AAUUCAAG CUGAUGAG GCCGUUAGGC CGAA IGCAAUCA | 2732 |
| 2490 | AUUGCCUC U UGAAUUAA | 958 | UUAAUUCA CUGAUGAG GCCGUUAGGC CGAA IAGGCAAU | 2733 |
| 2509 | AAAAAAAC U AGAAAAAA | 959 | UUUUUUCU CUGAUGAG GCCGUUAGGC CGAA IUUUUUUU | 2734 |

Input Sequence = AF100308.
Cut Site = UH/.
Stem Length = 8.
Core Sequence = CUGAUGAG GCCGUUAGGC CGAA AF100308 (Hepatitis B virus strain 2-18, 3215 bp)
Underlined region can be any X sequence or linker, as described herein.
“I” standws for Inosine

TABLE V
Human BACE G-cleaver Ribozyme and Target Sequence

| Pos | Substrate | Seq ID | G-cleaver | Seq ID |
|---|---|---|---|---|
| 11 | ACGCGUCC G CAGCCCGC | 960 | GCGGGCUG UGAUG GCAUGCACUAUGC GCG GGACGCGU | 2735 |
| 18 | CGCAGCCC G CCCGGGAG | 961 | CUCCCGGG UGAUG GCAUGCACUAUGC GCG GGGCUGCG | 2736 |
| 29 | CGGGAGCU G CGAGCCGC | 962 | GCGGCUCG UGAUG GCAUGCACUAUGC GCG AGCUCCCG | 2737 |
| 31 | GGAGCUGC G AGCCGCGA | 963 | UCGCGGCU UGAUG GCAUGCACUAUGC GCG GCAGCUCC | 2738 |
| 36 | UGCGAGCC G CGAGCUGG | 964 | CCAGCUCG UGAUG GCAUGCACUAUGC GCG GGCUCGCA | 2739 |
| 38 | CGAGCCGC G AGCUGGAU | 965 | AUCCAGCU UGAUG GCAUGCACUAUGC GCG GCGGCUCG | 2740 |
| 58 | GGUGGCCU G AGCAGCCA | 966 | UGGCUGCU UGAUG GCAUGCACUAUGC GCG AGGCCACC | 2741 |
| 69 | CAGCCAAC G CAGCCGCA | 967 | UGCGGCUG UGAUG GCAUGCACUAUGC GCG GUUGGCUG | 2742 |
| 75 | ACGCAGCC G CAGGAGCC | 968 | GGCUCCUG UGAUG GCAUGCACUAUGC GCG GGCUGCGU | 2743 |
| 94 | GAGCCCUU G CCCCUGCC | 969 | GGCAGGGG UGAUG GCAUGCACUAUGC GCG AAGGGCUC | 2744 |
| 100 | UUGCCCCU G CCCGCGCC | 970 | GGCGCGGG UGAUG GCAUGCACUAUGC GCG AGGGGCAA | 2745 |
| 104 | CCCUGCCC G CGCCGCCG | 971 | CGGCGGCG UGAUG GCAUGCACUAUGC GCG GGGCAGGG | 2746 |
| 106 | CUGCCCGC G CCGCCGCC | 972 | GGCGGCGG UGAUG GCAUGCACUAUGC GCG GCGGGCAG | 2747 |
| 109 | CCCGCGCC G CCGCCCGC | 973 | GCGGGCGG UGAUG GCAUGCACUAUGC GCG GGCGCGGG | 2748 |
| 112 | GCGCCGCC G CCCGCCGG | 974 | CCGGCGGG UGAUG GCAUGCACUAUGC GCG GGCGGCGC | 2749 |
| 116 | CGCCGCCC G CCGGGGGG | 975 | CCCCCCGG UGAUG GCAUGCACUAUGC GCG GGGCGGCG | 2750 |
| 137 | GGGAAGCC G CCACCGGC | 976 | GCCGGUGG UGAUG GCAUGCACUAUGC GCG GGCUUCCC | 2751 |
| 148 | ACCGGCCC G CCAUGCCC | 977 | GGGCAUGG UGAUG GCAUGCACUAUGC GCG GGGCCGGU | 2752 |
| 153 | CCCGCCAU G CCCGCCCC | 978 | GGGGCGGG UGAUG GCAUGCACUAUGC GCG AUGGCGGG | 2753 |
| 157 | CCAUGCCC G CCCCUCCC | 979 | GGGAGGGG UGAUG GCAUGCACUAUGC GCG GGGCAUGG | 2754 |
| 172 | CCAGCCCC G CCGGGAGC | 980 | GCUCCCGG UGAUG GCAUCCACUAUGC GCG GGGGCUGG | 2755 |
| 183 | GGGAGCCC G CGCCCGCU | 981 | AGCGGGCG UGAUG GCAUGCACUAUGC GCG GGGCUCCC | 2756 |
| 185 | GAGCCCGC G CCCGCUGC | 982 | GCAGCGGG UGAUG GCAUGCACUAUGC GCG GCGGGCUC | 2757 |
| 189 | CCGCGCCC G CUGCCCAG | 983 | CUGGGCAG UGAUG GCAUGCACUAUGC GCG GGGCGCGG | 2758 |
| 192 | CGCCCGCU G CCCAGGCU | 984 | AGCCUGGG UGAUG GCAUGCACUAUGC GCG AGCGGGCG | 2759 |
| 205 | GGCUGGCC G CCGCCGUG | 985 | CACGGCGG UGAUG GCAUGCACUAUGC GCG GGCCAGCC | 2760 |
| 208 | UGGCCGCC G CCGUGCCG | 986 | CGGCACGG UGAUG GCAUGCACUAUGC GCG GGCGGCCA | 2761 |
| 213 | GCCGCCGU G CCGAUGUA | 987 | UACAUCGG UGAUG GCAUGCACUAUGC GCG ACGGCGGC | 2762 |
| 216 | GCCGUGCC G AUGUAGCG | 988 | CGCUACAU UGAUG GCAUGCACUAUGC GCG GGCACGGC | 2763 |
| 250 | UCUCCCCU G CUCCCGUG | 989 | CACGGGAG UGAUG GCAUGCACUAUGC GCG AGGGGAGA | 2764 |
| 258 | GCUCCCGU G CUCUGCGG | 990 | CCGCAGAG UGAUG GCAUGCACUAUGC GCG ACGGGAGC | 2765 |
| 263 | CGUGCUCU G CGGAUCUC | 991 | GAGAUCCG UGAUG GCAUGCACUAUGC GCG AGAGCACG | 2766 |
| 276 | UCUCCCCU G ACCGCUCU | 992 | AGAGCGGU UGAUG GCAUGCACUAUGC GCG AGGGGAGA | 2767 |
| 280 | CCCUGACC G CUCUCCAC | 993 | GUGGAGAG UGAUG GCAUGCACUAUGC GCG GGUCAGGG | 2768 |
| 320 | AGGGCCCU G CAGGCCCU | 994 | AGGGCCUG UGAUG GCAUGCACUAUGC GCG AGGGCCCU | 2769 |
| 337 | GGCGUCCU G AUGCCCCC | 995 | GGGGGCAU UGAUG GCAUGCACUAUGC GCG AGGACGCC | 2770 |
| 340 | GUCCUGAU G CCCCCAAG | 996 | CUUGGGGG UGAUG GCAUGCACUAUGC GCG AUCAGGAC | 2771 |
| 360 | CCUCUCCU G AGAAGCCA | 997 | UGGCUUCU UGAUG GCAUGCACUAUGC GCG AGGAGAGG | 2772 |
| 397 | GGGCAGGC G CCAGGGAC | 998 | GUCCCUGG UGAUG GCAUGCACUAUGC GCG GCCUGCCC | 2773 |
| 420 | GGGCCAGU G CGAGCCCA | 999 | UGGGCUCG UGAUG GCAUGCACUAUGC GCG ACUGGCCC | 2774 |
| 422 | GCCAGUGC G AGCCCAGA | 1000 | UCUGGGCU UGAUG GCAUGCACUAUGC GCG GCACUGGC | 2775 |
| 437 | GAGGGCCC G AAGGCCGG | 1001 | CCGGCCUU UGAUG GCAUGCACUAUGC GCG GGGCCCUC | 2776 |
| 468 | CAAGCCCU G CCCUGGCU | 1002 | AGCCAGGG UGAUG GCAUGCACUAUGC GCG AGGGCUUG | 2777 |
| 480 | UGGCUCCU G CUGUGGAU | 1003 | AUCCACAG UGAUG GCAUGCACUAUGC GCG AGGAGCCA | 2778 |
| 493 | GGAUGGGC G CGGGAGUG | 1004 | CACUCCCG UGAUG GCAUGCACUAUGC GCG GCCCAUCC | 2779 |
| 501 | GCGGGAGU G CUGCCUGC | 1005 | GCAGGCAG UGAUG GCAUGCACUAUGC GCG ACUCCCGC | 2780 |
| 504 | GGAGUGCU G CCUGCCCA | 1006 | UGGGCAGG UGAUG GCAUGCACUAUGC GCG AGCACUCC | 2781 |
| 508 | UGCUGCCU G CCCACGGC | 1007 | GCCGUGGG UGAUG GCAUGCACUAUGC GCG AGGCAGCA | 2782 |
| 537 | AUCCGGCU G CCCCUGCG | 1008 | CGCAGGGG UGAUG GCAUGCACUAUGC GCG AGCCGGAU | 2783 |
| 543 | CUGCCCCU G CGCAGCGG | 1009 | CCGCUGCG UGAUG GCAUGCACUAUGC GCG AGGGGCAG | 2784 |
| 545 | GCCCCUGC G CAGCGGCC | 1010 | GGCCGCUG UGAUG GCAUGCACUAUGC GCG GCAGGGGC | 2785 |
| 562 | UGGGGGGC G CCCCCCUG | 1011 | CAGGGGGG UGAUG GCAUGCACUAUGC GCG GCCCCCCA | 2786 |
| 576 | CUGGGGCU G CGGCUGCC | 1012 | GGCAGCCG UGAUG GCAUGCACUAUGC GCG AGCCCCAG | 2787 |
| 582 | CUGCGGCU G CCCCGGGA | 1013 | UCCCGGGG UGAUG GCAUGCACUAUGC GCG AGCCGCAG | 2788 |
| 595 | GGGAGACC G ACGAAGAG | 1014 | CUCUUCGU UGAUG GCAUGCACUAUGC GCG GGUCUCCC | 2789 |
| 598 | AGACCGAC G AAGAGCCC | 1015 | GGGCUCUU UGAUG GCAUGCACUAUGC GCG GUCGGUCU | 2790 |
| 607 | AAGAGCCC G AGGAGCCC | 1016 | GGGCUCCU UGAUG GCAUGCACUAUGC GCG GGGCUCUU | 2791 |
| 654 | GACAACCU G AGGGGCAA | 1017 | UUGCCCCU UGAUG GCAUGCACUAUGC GCG AGGUUGUC | 2792 |
| 690 | GUGGAGAU G ACCGUGGG | 1018 | CCCACGGU UGAUG GCAUGCACUAUGC GCG AUCUCCAC | 2793 |
| 708 | AGCCCCCC G CAGACGCU | 1019 | AGCGUCUG UGAUG GCAUGCACUAUGC GCG GGGGGGCU | 2794 |
| 714 | CCGCAGAC G CUCAACAU | 1020 | AUGUUGAG UGAUG GCAUGCACUAUGC GCG GUCUGCGG | 2795 |
| 751 | GUAACUUU G CAGUGGGU | 1021 | ACCCACUG UGAUG GCAUGCACUAUGC GCG AAAGUUAC | 2796 |
| 760 | CAGUGGGU G CUGCCCCC | 1022 | GGGGGCAG UGAUG GCAUGCACUAUGC GCG ACCCACUG | 2797 |
| 763 | UGGGUGCU G CCCCCCAC | 1023 | GUGGGGGG UGAUG GCAUGCACUAUGC GCG AGCACCCA | 2798 |
| 780 | CCCUUCCU G CAUCGCUA | 1024 | UAGCGAUG UGAUG GCAUGCACUAUGC GCG AGGAAGGG | 2799 |
| 785 | CCUGCAUC G CUACUACC | 1025 | GGUAGUAG UGAUG GCAUGCACUAUGC GCG GAUGCAGG | 2800 |
| 843 | GUGUAUGU G CCCUACAC | 1026 | GUGUAGGG UGAUG GCAUGCACUAUGC GCG ACAUACAC | 2801 |
| 883 | UGGGCACC G ACCUGGUA | 1027 | UACCAGGU UGAUG GCAUGCACUAUGC GCG GGUGCCCA | 2802 |
| 921 | GUCACUGU G CGUGCCAA | 1028 | UUGGCACG UGAUG GCAUGCACUAUGC GCG ACAGUGAC | 2803 |
| 925 | CUGUGCGU G CCAACAUU | 1029 | AAUGUUGG UGAUG GCAUGCACUAUGC GCG ACGCACAG | 2804 |
| 934 | CCAACAUU G CUGCCAUC | 1030 | GAUGGCAG UGAUG GCAUGCACUAUGC GCG AAUGUUGG | 2805 |
| 937 | ACAUUGCU G CCAUCACU | 1031 | AGUGAUGG UGAUG GCAUGCACUAUGC GCG AGCAAUGU | 2806 |
| 946 | CCAUCACU G AAUCAGAC | 1032 | GUCUGAUU UGAUG GCAUGCACUAUGC GCG AGUGAUGG | 2807 |
| 1006 | UGGCCUAU G CUGAGAUU | 1033 | AAUCUCAG UGAUG GCAUGCACUAUGC GCG AUAGGCCA | 2808 |
| 1009 | CCUAUGCU G AGAUUGCC | 1034 | GGCAAUCU UGAUG GCAUGCACUAUGC GCG AGCAUAGG | 2809 |
| 1015 | CUGAGAUU G CCAGGCCU | 1035 | AGGCCUGG UGAUG GCAUGCACUAUGC GCG AAUCUCAG | 2810 |
| 1024 | CCAGGCCU G ACGACUCC | 1036 | GGAGUCGU UGAUG GCAUGCACUAUGC GCG AGGCCUGG | 2811 |
| 1027 | GGCCUGAC G ACUCCCUG | 1037 | CAGGGAGU UGAUG GCAUGCACUAUGC GCG GUCAGGCC | 2812 |
| 1048 | CUUUCUUU G ACUCUCUG | 1038 | CAGAGAGU UGAUG GCAUGCACUAUGC GCG AAAGAAAG | 2813 |
| 1092 | UUCUCCCU G CAGCUUUG | 1039 | CAAAGCUG UGAUG GCAUGCACUAUGC GCG AGGGAGAA | 2814 |
| 1105 | UUUGUGGU G CUGGCUUC | 1040 | GAAGCCAG UGAUG GCAUGCACUAUGC GCG ACCACAAA | 2815 |
| 1129 | ACCAGUCU G AAGUGCUG | 1041 | CAGCACUU UGAUG GCAUGCACUAUGC GCG AGACUGGU | 2816 |
| 1134 | UCUGAAGU G CUGGCCUC | 1042 | GAGGCCAG UGAUG GCAUGCACUAUGC GCG ACUUCAGA | 2817 |
| 1158 | GGGAGCAU G AUCAUUGG | 1043 | CCAAUGAU UGAUG GCAUGCACUAUGC GCG AUGCUCCC | 2818 |
| 1174 | GAGGUAUC G ACCACUCG | 1044 | CGAGUGGU UGAUG GCAUGCACUAUGC GCG GAUACCUC | 2819 |
| 1182 | GACCACUC G CUGUACAC | 1045 | GUGUACAG UGAUG GCAUGCACUAUGC GCG GAGUGGUC | 2820 |
| 1234 | GGUAUUAU G AGGUGAUC | 1046 | GAUCACCU UGAUG GCAUGCACUAUGC GCG AUAAUACC | 2821 |
| 1239 | UAUGAGGU G AUCAUUGU | 1047 | ACAAUGAU UGAUG GCAUGCACUAUGC GCG ACCUCAUA | 2822 |
| 1248 | AUCAUUGU G CGGGUGGA | 1048 | UCCACCCG UGAUG GCAUGCACUAUGC GCG ACAAUGAU | 2823 |
| 1275 | CAGGAUCU G AAAAUGGA | 1049 | UCCAUUUU UGAUG GCAUGCACUAUGC GCG AGAUCCUG | 2824 |
| 1286 | AAUGGACU G CAAGGAGU | 1050 | ACUCCUUG UGAUG GCAUGCACUAUGC GCG AGUCCAUU | 2825 |
| 1303 | ACAACUAU G ACAAGAGC | 1051 | GCUCUUGU UGAUG GCAUGCACUAUGC GCG AUAGUUGU | 2826 |
| 1344 | CUUCGUUU G CCCAAGAA | 1052 | UUCUUGGG UGAUG GCAUGCACUAUGC GCG AAACGAAG | 2827 |
| 1360 | AAGUGUUU G AAGCUGCA | 1053 | UGCAGCUU UGAUG GCAUGCACUAUGC GCG AAACACUU | 2828 |
| 1366 | UUGAAGCU G CAGUCAAA | 1054 | UUUGACUG UGAUG GCAUGCACUAUGC GCG AGCUUCAA | 2829 |
| 1411 | AGUUCCCU G AUGGUUUC | 1055 | GAAACCAU UGAUG GCAUGCACUAUGC GCG AGGGAACU | 2830 |
| 1442 | GCUGGUGU G CUGGCAAG | 1056 | CUUGCCAG UGAUG GCAUGCACUAUGC GCG ACACCAGC | 2831 |
| 1504 | UAAUGGGU G AGGUUACC | 1057 | GGUAACCU UGAUG GCAUGCACUAUGC GCG ACCCAUUA | 2832 |
| 1526 | GUCCUUCC G CAUCACCA | 1058 | UGGUGAUG UGAUG GCAUGCACUAUGC GCG GGAAGGAC | 2833 |
| 1542 | AUCCUUCC G CAGCAAUA | 1059 | UAUUGCUG UGAUG GCAUGCACUAUGC GCG GGAAGGAU | 2834 |
| 1554 | CAAUACCU G CGGCCAGU | 1060 | ACUGGCCG UGAUG GCAUGCACUAUGC GCG AGGUAUUG | 2835 |
| 1588 | CCCAAGAC G ACUGUUAC | 1061 | GUAACAGU UGAUG GCAUGCACUAUGC GCG GUCUUGGG | 2836 |
| 1603 | ACAAGUUU G CCAUCUCA | 1062 | UGAGAUGG UGAUG GCAUGCACUAUGC GCG AAACUUGU | 2837 |
| 1672 | UUGUCUUU G AUCGGGCC | 1063 | GGCCCGAU UGAUG GCAUGCACUAUGC GCG AAAGACAA | 2838 |
| 1682 | UCGGGCCC G AAAACGAA | 1064 | UUCGUUUU UGAUG GCAUGCACUAUGC GCG GGGCCCGA | 2839 |
| 1688 | CCGAAAAC G AAUUGGCU | 1065 | AGCCAAUU UGAUG GCAUGCACUAUGC GCG GUUUUCGG | 2840 |
| 1699 | UUGGCUUU G CUGUCAGC | 1066 | GCUGACAG UGAUG GCAUGCACUAUGC GCG AAAGCCAA | 2841 |
| 1708 | CUGUCAGC G CUUGCCAU | 1067 | AUGGCAAG UGAUG GCAUGCACUAUGC GCG GCUGACAG | 2842 |
| 1712 | CAGCGCUU G CCAUGUGC | 1068 | GCACAUGG UGAUG GCAUGCACUAUGC GCG AAGCGCUG | 2843 |
| 1719 | UGCCAUGU G CACGAUGA | 1069 | UCAUCGUG UGAUG GCAUGCACUAUGC GCG ACAUGGCA | 2844 |
| 1723 | AUGUGCAC G AUGAGUUC | 1070 | GAACUCAU UGAUG GCAUGCACUAUGC GCG GUGCACAU | 2845 |
| 1726 | UGCACGAU G AGUUCAGG | 1071 | CCUGAACU UGAUG GCAUGCACUAUGC GCG AUCGUGCA | 2846 |
| 1807 | AGACAGAU G AGUCAACC | 1072 | GGUUGACU UGAUG GCAUGCACUAUGC GCG AUCUGUCU | 2847 |
| 1821 | ACCCUCAU G ACCAUAGC | 1073 | GCUAUGGU UGAUG GCAUGCACUAUGC GCG AUGAGGGU | 2848 |
| 1843 | UCAUGGCU G CCAUCUGC | 1074 | GCAGAUGG UGAUG GCAUGCACUAUGC GCG AGCCAUGA | 2849 |
| 1850 | UGCCAUCU G CGCCCUCU | 1075 | AGAGGGCG UGAUG GCAUGCACUAUGC GCG AGAUGGCA | 2850 |
| 1852 | CCAUCUGC G CCCUCUUC | 1076 | GAAGAGGG UGAUG GCAUGCACUAUGC GCG GCAGAUGG | 2851 |
| 1863 | CUCUUCAU G CUGCCACU | 1077 | AGUGGCAG UGAUG GCAUGCACUAUGC GCG AUGAAGAG | 2852 |
| 1866 | UUCAUGCU G CCACUCUG | 1078 | CAGAGUGG UGAUG GCAUGCACUAUGC GCG AGCAUGAA | 2853 |
| 1874 | GCCACUCU G CCUCAUGG | 1079 | CCAUGAGG UGAUG GCAUGCACUAUGC GCG AGAGUGGC | 2854 |
| 1895 | UCAGUGGC G CUGCCUCC | 1080 | GGAGGCAG UGAUG GCAUGCACUAUGC GCG GCCACUGA | 2855 |
| 1898 | GUGGCGCU G CCUCCGCU | 1081 | AGCGGAGG UGAUG GCAUGCACUAUGC GCG AGCGCCAC | 2856 |
| 1904 | CUGCCUCC G CUGCCUGC | 1082 | GCAGGCAG UGAUG GCAUGCACUAUGC GCG GGAGGCAG | 2857 |
| 1907 | CCUCCGCU G CCUGCGCC | 1083 | GGCGCAGG UGAUG GCAUGCACUAUGC GCG AGCGGAGG | 2858 |
| 1911 | CGCUGCCU G CGCCAGCA | 1084 | UGCUGGCG UGAUG GCAUGCACUAUGC GCG AGGCAGCG | 2859 |
| 1913 | CUGCCUGC G CCAGCAGC | 1085 | GCUGCUGG UGAUG GCAUGCACUAUGC GCG GCAGGCAG | 2860 |
| 1924 | AGCAGCAU G AUGACUUU | 1086 | AAAGUCAU UGAUG GCAUGCACUAUGC GCG AUGCUGCU | 2861 |
| 1927 | AGCAUGAU G ACUUUGCU | 1087 | AGCAAACU UGAUG GCAUGCACUAUGC GCG AUCAUGCU | 2862 |
| 1933 | AUGACUUU G CUGAUGAC | 1088 | GUCAUCAG UGAUG GCAUGCACUAUGC GCG AAAGUCAU | 2863 |
| 1936 | ACUUUGCU G AUGACAUC | 1089 | GAUGUCAU UGAUG GCAUGCACUAUGC GCG AGCAAAGU | 2864 |
| 1939 | UUGCUGAU G ACAUCUCC | 1090 | GGAGAUGU UGAUG GCAUGCACUAUGC GCG AUCAGCAA | 2865 |
| 1950 | AUCUCCCU G CUGAAGUG | 1091 | CACUUCAG UGAUG GCAUGCACUAUGC GCG AGGGAGAU | 2866 |
| 1953 | UCCCUGCU G AAGUGAGG | 1092 | CCUCACUU UGAUG GCAUGCACUAUGC GCG AGCAGGGA | 2867 |
| 1958 | GCUGAAGU G AGGAGGCC | 1093 | GGCCUCCU UGAUG GCAUGCACUAUGC GCG ACUUCAGC | 2868 |
| 2087 | CACCAAAU G CCUCUGCC | 1094 | GGCAGAGG UGAUG GCAUGCACUAUGC GCG AUUUGGUG | 2869 |
| 2093 | AUGCCUCU G CCUUGAUG | 1095 | CAUCAAGG UGAUG GCAUGCACUAUGC GCG AGAGGCAU | 2870 |
| 2098 | UCUGCCUU G AUGGAGAA | 1096 | UUCUCCAU UGAUG GCAUGCACUAUGC GCG AAGGCAGA | 2871 |
| 2179 | AGCACUCU G CUGGCGGG | 1097 | CCCGCCAG UGAUG GCAUGCACUAUGC GCG AGAGUGCU | 2872 |
| 2227 | GAAAUUCU G CUGCUUGA | 1098 | UCAAGCAG UGAUG GCAUGCACUAUGC GCG AGAAUUUC | 2873 |
| 2230 | AUUCUGCU G CUUGAAAC | 1099 | GUUUCAAG UGAUG GCAUGCACUAUGC GCG AGCAGAAU | 2874 |
| 2234 | UGCUGCUU G AAACUUCA | 1100 | UGAAGUUU UGAUG GCAUGCACUAUGC GCG AAGCAGCA | 2875 |
| 2248 | UCAGCCCU G AACCUUUG | 1101 | CAAAGGUU UGAUG GCAUGCACUAUGC GCG AGGGCUGA | 2876 |
| 2329 | CAUCACAC G CAGGUUAC | 1102 | GUAACCUG UGAUG GCAUGCACUAUGC GCG GUGUGAUG | 2877 |
| 2393 | GUUUCCCU G CUGGCCAA | 1103 | UUGGCCAG UGAUG GCAUGCACUAUGC GCG AGGGAAAC | 2878 |
| 2419 | GAGAGGAU G CACAGUUU | 1104 | AAACUGUG UGAUG GCAUGCACUAUGC GCG AUCCUCUC | 2879 |
| 2428 | CACAGUUU G CUAUUUGC | 1105 | GCAAAUAG UGAUG GCAUGCACUAUGC GCG AAACUGUG | 2880 |
| 2435 | UGCUAUUU G CUUUAGAG | 1106 | CUCUAAAG UGAUG GCAUGCACUAUGC GCG AAAUAGCA | 2881 |
| 2476 | ACAUUGGU G CAAAGAUU | 1107 | AAUCUUUG UGAUG GCAUGCACUAUGC GCG ACCAAUGU | 2882 |
| 2485 | CAAAGAUU G CCUCUUGA | 1108 | UCAAGAGG UGAUG GCAUGCACUAUGC GCG AAUCUUUG | 2883 |
| 2492 | UGCCUCUU G AAUUAAAA | 1109 | UUUUAAUU UGAUG GCAUGCACUAUGC GCG AAGAGGCA | 2884 |
| 219 | GUGCCGAU G UAGCGGGC | 1110 | GCCCGCUA UGAUG GCAUGCACUAUGC GCG AUCGGCAC | 2885 |
| 483 | CUCCUGCU G UGGAUGGG | 1111 | CCCAUCCA UGAUG GCAUGCACUAUGC GCG AGCAGGAG | 2886 |
| 634 | GCAGCUUU G UGGAGAUG | 1112 | CAUCUCCA UGAUG GCAUGCACUAUGC GCG AAAGCUGC | 2887 |
| 804 | AGGCAGCU G UCCAGCAC | 1113 | GUGCUGGA UGAUG GCAUGCACUAUGC GCG AGCUGCCU | 2888 |
| 835 | GGAAGGGU G UGUAUGUG | 1114 | CACAUACA UGAUG GCAUGCACUAUGC GCG ACCCUUCC | 2889 |
| 837 | AAGGGUGU G UAUGUGCC | 1115 | GGCACAUA UGAUG GCAUGCACUAUGC GCG ACACCCUU | 2890 |
| 841 | GUGUGUAU G UGCCCUAC | 1116 | GUAGGGCA UGAUG GCAUGCACUAUGC GCG AUACACAC | 2891 |
| 919 | ACGUCACU G UGCGUGCC | 1117 | GGCACGCA UGAUG GCAUGCACUAUGC GCG AGUGACGU | 2892 |
| 1100 | GCAGCUUU G UGGUGCUG | 1118 | CAGCACCA UGAUG GCAUGCACUAUGC GCG AAAGCUGC | 2893 |
| 1144 | UGGCCUCU G UCGGAGGG | 1119 | CCCUCCGA UGAUG GCAUGCACUAUGC GCG AGAGGCCA | 2894 |
| 1185 | CACUCGCU G UACACAGG | 1120 | CCUGUGUA UGAUG GCAUGCACUAUGC GCG AGCGAGUG | 2895 |
| 1246 | UGAUCAUU G UGCGGGUG | 1121 | CACCCGCA UGAUG GCAUGCACUAUGC GCG AAUGAUCA | 2896 |
| 1315 | AGAGCAUU G UCGACAGU | 1122 | ACUGUCCA UGAUG GCAUGCACUAUGC GCG AAUGCUCU | 2897 |
| 1356 | AAGAAAGU G UUUGAAGC | 1123 | GCUUCAAA UGAUG GCAUGCACUAUGC GCG ACUUUCUU | 2898 |
| 1440 | CAGCUGGU G UGCUGGCA | 1124 | UGCCAGCA UGAUG GCAUGCACUAUGC GCG ACCAGCUG | 2899 |
| 1570 | UGGAAGAU G UGGCCACG | 1125 | CGUGGCCA UGAUG GCAUGCACUAUGC GCG AUCUUCCA | 2900 |
| 1592 | AGACGACU G UUACAAGU | 1126 | ACUUGUAA UGAUG GCAUGCACUAUGC GCG AGUCGUCU | 2901 |
| 1630 | CGGGCACU G UUAUGGGA | 1127 | UCCCAUAA UGAUG GCAUGCACUAUGC GCG AGUGCCCG | 2902 |
| 1642 | UGGGAGCU G UUAUCAUG | 1128 | CAUGAUAA UGAUG GCAUGCACUAUGC GCG AGCUCCCA | 2903 |
| 1666 | UCUACGUU G UCUUUGAU | 1129 | AUCAAAGA UGAUG GCAUGCACUAUGC GCG AACGUAGA | 2904 |
| 1702 | GCUUUGCU G UCAGCGCU | 1130 | AGCGCUGA UGAUG GCAUGCACUAUGC GCG AGCAAAGC | 2905 |
| 1717 | CUUGCCAU G UGCACGAU | 1131 | AUCGUGCA UGAUG GCAUGCACUAUGC GCG AUGGCAAG | 2906 |
| 1759 | GCCCUUUU G UCACCUUG | 1132 | CAAGGUGA UGAUG GCAUGCACUAUGC GCG AAAAGGGC | 2907 |
| 1781 | GGAAGACU G UGGCUACA | 1133 | UGUAGCCA UGAUG GCAUGCACUAUGC GCG AGUCUUCC | 2908 |
| 1834 | UAGCCUAU G UCAUGGCU | 1134 | AGCCAUGA UGAUG GCAUGCACUAUGC GCG AUAGGCUA | 2909 |
| 1884 | CUCAUGGU G UGUCAGUG | 1135 | CACUGACA UGAUG GCAUGCACUAUGC GCG ACCAUGAG | 2910 |
| 1886 | CAUGGUGU G UCAGUGGC | 1136 | GCCACUGA UGAUG GCAUGCACUAUGC GCG ACACCAUG | 2911 |
| 2048 | UGGCACCU G UGGCCAGA | 1137 | UCUGGCCA UGAUG GCAUGCACUAUGC GCG AGGUGCCA | 2912 |
| 2139 | CAGGGACU G UACCUGUA | 1138 | UACAGGUA UGAUG GCAUGCACUAUGC GCG AGUCCCUG | 2913 |
| 2145 | CUGUACCU G UAGGAAAC | 1139 | GUUUCCUA UGAUG GCAUGCACUAUGC GCG AGGUACAG | 2914 |
| 2256 | GAACCUUU G UCCACCAU | 1140 | AUGGUGGA UGAUG GCAUGCACUAUGC GCG AAAGGUUC | 2915 |
| 2346 | CUUGGCGU G UGUCCCUG | 1141 | CAGGGACA UGAUG GCAUGCACUAUGC GCG ACGCCAAG | 2916 |
| 2348 | UGGCGUGU G UCCCUGUG | 1142 | CACAGGGA UGAUG GCAUGCACUAUGC GCG ACACGCCA | 2917 |
| 2354 | GUGUCCCU G UGGUACCC | 1143 | GGGUACCA UGAUG GCAUGCACUAUGC GCG AGGGACAC | 2918 |
| 2385 | CCAAGCUU G UUUCCCUG | 1144 | CAGGGAAA UGAUG GCAUGCACUAUGC GCG AAGCUUGG | 2919 |
| 2453 | CAGGGACU G UAUAAACA | 1145 | UGUUUAUA UGAUG GCAUGCACUAUGC GCG AGUCCCUG | 2920 |

Input Sequence = AF190725. Cut Site = G/.
Stem Length = 8. Core Sequence = UGAUG GCAUGCACUAUGC GCG
AF190725 (Homo sapiens beta-site APP cleaving enzyme (BACE) mRNA; 2526 bp)

TABLE VI
Human BACE Zinzyme Ribozyme and Target Sequence

| Pos | Substrate | Seq ID | Zinzyme | Seq ID |
|---|---|---|---|---|
| 11 | ACGCGUCC G CAGCCCGC | 960 | GCGGGCUG GCCGAAAGGCGAGUCAAGGUCU GGACGCGU | 2921 |
| 18 | CGCAGCCC G CCCGGGAG | 961 | CUCCCGGG GCCGAAAGGCGAGUCAAGGUCU GGGCUGCG | 2922 |
| 29 | CGGGAGCU G CGAGCCGC | 962 | GCGGCUCG GCCGAAAGGCGAGUCAAGGUCU AGCUCCCG | 2923 |
| 36 | UGCGAGCC G CGAGCUGG | 964 | CCAGCUCG GCCGAAAGGCGAGUCAAGGUCU GGCUCGCA | 2924 |
| 69 | CAGCCAAC G CAGCCGCA | 967 | UGCGGCUG GCCGAAAGGCGAGUCAAGGUCU GUUGGCUG | 2925 |
| 75 | ACGCAGCC G CAGGAGCC | 968 | GGCUCCUG GCCGAAAGGCGAGUCAAGGUCU GGCUGCGU | 2926 |
| 94 | GAGCCCUU G CCCCUGCC | 969 | GGCAGGGG GCCGAAAGGCGAGUCAAGGUCU AAGGGCUC | 2927 |
| 100 | UUGCCCCU G CCCGCGCC | 970 | GGCGCGGG GCCGAAAGGCGAGUCAAGGUCU AGGGGCAA | 2928 |
| 104 | CCCUGCCC G CGCCGCCG | 971 | CGGCGGCG GCCGAAAGGCGAGUCAAGGUCU GGGCAGGG | 2929 |
| 106 | CUGCCCGC G CCGCCGCC | 972 | GGCGGCGG GCCGAAAGGCGAGUCAAGGUCU GCGGGCAG | 2930 |
| 109 | CCCGCGCC G CCGCCCGC | 973 | GCGGGCGG GCCGAAAGGCGAGUCAAGGUCU GGCGCGGG | 2931 |
| 112 | GCGCCGCC G CCCGCCGG | 974 | CCGGCGGG GCCGAAAGGCGAGUCAAGGUCU GGCGGCGC | 2932 |
| 116 | CGCCGCCC G CCGGGGGG | 975 | CCCCCCGG GCCGAAAGGCGAGUCAAGGUCU GGGCGGCG | 2933 |
| 137 | GGGAAGCC G CCACCGGC | 976 | GCCGGUGG GCCGAAAGGCGAGUCAAGGUCU GGCUUCCC | 2934 |
| 148 | ACCGGCCC G CCAUGCCC | 977 | GGGCAUGG GCCGAAAGGCGAGUCAAGGUCU GGGCCGGU | 2935 |
| 153 | CCCGCCAU G CCCGCCCC | 978 | GGGGCGGG GCCGAAAGGCGAGUCAAGGUCU AUGGCGGG | 2936 |
| 157 | CCAUGCCC G CCCCUCCC | 979 | GGGAGGGG GCCGAAAGGCGAGUCAAGGUCU GGGCAUGG | 2937 |
| 172 | CCAGCCCC G CCGGGAGC | 980 | GCUCCCGG GCCGAAAGGCGAGUCAAGGUCU GGGGCUGC | 2938 |
| 183 | GGGAGCCC G CGCCCGCU | 981 | AGCGGGCG GCCGAAAGGCGAGUCAAGGUCU GGGCUCCC | 2939 |
| 185 | GAGCCCGC G CCCGCUGC | 982 | GCAGCGGG GCCGAAAGGCGAGUCAAGGUCU GCGGGCUC | 2940 |
| 189 | CCGCGCCC G CUGCCCAG | 983 | CUGGGCAG GCCGAAAGGCGAGUCAAGGUCU GGGCGCGG | 2941 |
| 192 | CGCCCGCU G CCCAGGCU | 984 | AGCCUGGG GCCGAAAGGCGAGUCAAGGUCU AGCGGGCG | 2942 |
| 205 | GGCUGGCC G CCGCCGUG | 985 | CACGGCGG GCCGAAAGGCGAGUCAAGGUCU GGCCAGCC | 2943 |
| 208 | UGGCCGCC G CCGUGCCG | 986 | CGGCACGG GCCGAAAGGCGAGUCAAGGUCU GGCGGCCA | 2944 |
| 213 | GCCGCCGU G CCGAUGUA | 987 | UACAUCGG GCCGAAAGGCGAGUCAAGGUCU ACGGCGGC | 2945 |
| 250 | UCUCCCCU G CUCCCGUG | 989 | CACGGGAG GCCGAAAGGCGAGUCAAGGUCU AGGGGAGA | 2946 |
| 258 | GCUCCCGU G CUCUGCGG | 990 | CCGCAGAG GCCCAAAGGCGAGUCAAGGUCU ACGGGAGC | 2947 |
| 263 | CGUGCUCU G CGGAUCUC | 991 | GAGAUCCG GCCGAAAGGCGAGUCAAGGUCU AGAGCACG | 2948 |
| 280 | CCCUGACC G CUCUCCAC | 993 | GUGGAGAG GCCGAAAGGCGAGUCAAGGUCU GGUCAGGG | 2949 |
| 320 | AGGGCCCU G CAGGCCCU | 994 | AGGGCCUG GCCGAAAGGCGAGUCAAGGUCU AGGGCCCU | 2950 |
| 340 | GUCCUGAU G CCCCCAAG | 996 | CUUGGGGG GCCGAAAGGCGAGUCAAGGUCU AUCAGGAC | 2951 |
| 397 | GGGCAGGC G CCAGGGAG | 998 | GUCCCUGG GCCGAAAGGCGAGUCAAGGUCU GCCUGCCC | 2952 |
| 420 | GGGCCAGU G CGAGCCCA | 999 | UGGGCUCG GCCGAAAGGCGAGUCAAGGUCU ACUGGCCC | 2953 |
| 468 | CAAGCCCU G CCCUGGCU | 1002 | AGCCAGGG GCCGAAAGGCGAGUCAAGGUCU AGGGCUUG | 2954 |
| 480 | UGGCUCCU G CUGUGGAU | 1003 | AUCCACAG GCCGAAAGGCGAGUCAAGGUCU AGGAGCCA | 2955 |
| 493 | GGAUGGGC G CGGGAGUG | 1004 | CACUCCCG GCCGAAAGGCGAGUCAAGGUCU GCCCAUCC | 2956 |
| 501 | GCGGGAGU G CUGCCUGC | 1005 | GCAGGCAG GCCGAAAGGCGAGUCAAGGUCU ACUCCCGC | 2957 |
| 504 | GGAGUGCU G CCUGCCCA | 1006 | UGGGCAGG GCCGAAAGGCGAGUCAAGGUCU AGCACUCC | 2958 |
| 508 | UGCUGCCU G CCCACGGC | 1007 | GCCGUGGG GCCGAAAGGCGAGUCAAGGUCU AGGCAGCA | 2959 |
| 537 | AUCCGGCU G CCCCUGCG | 1008 | CGCAGGGG GCCGAAAGGCGAGUCAAGGUCU AGCCGGAU | 2960 |
| 543 | CUGCCCCU G CGCAGCGG | 1009 | CCGCUGCG GCCGAAAGGCGAGUCAAGGUCU AGGGGCAG | 2961 |
| 545 | GCCCCUGC G CAGCGGCC | 1010 | GGCCGCUG GCCGAAAGGCGAGUCAAGGUCU GCAGCGGC | 2962 |
| 562 | UGGGGGGC G CCCCCCUG | 1011 | CAGGGGGG GCCGAAAGGCGAGUCAAGGUCU GCCCCCCA | 2963 |
| 576 | CUGGGGCU G CGGCUGCC | 1012 | GGCAGCCG GCCGAAAGGCGAGUCAAGGUCU AGCCCCAG | 2964 |
| 582 | CUGCGGCU G CCCCGGGA | 1013 | UCCCGGGG GCCGAAAGGCGAGUCAAGGUCU AGCCGCAG | 2965 |
| 708 | AGCCCCCC G CAGACGCU | 1019 | AGCGUCUG GCCGAAAGGCGAGUCAAGGUCU GGGGGGCU | 2966 |
| 714 | CCGCAGAC G CUCAACAU | 1020 | AUGUUGAG GCCGAAAGGCGAGUCAAGGUCU GUCUGCGG | 2967 |
| 751 | GUAACUUU G CAGUGGGU | 1021 | ACCCACUG GCCGAAAGGCGAGUCAAGGUCU AAAGUUAC | 2968 |
| 760 | CAGUGGGU G CUGCCCCC | 1022 | GGGGGCAG GCCGAAAGGCGAGUCAAGGUCU ACCCACUG | 2969 |
| 763 | UGGGUGCU G CCCCCCAC | 1023 | GUGGGGGG GCCGAAAGGCGAGUCAAGGUCU AGCACCCA | 2970 |
| 780 | CCCUUCCU G CAUCGCUA | 1024 | UAGCGAUG GCCGAAAGGCGAGUCAAGGUCU AGGAAGGG | 2971 |
| 785 | CCUGCAUC G CUACUACC | 1025 | GGUAGUAG GCCGAAAGGCGAGUCAAGGUCU GAUGCAGG | 2972 |
| 843 | GUGUAUGU G CCCUACAC | 1026 | GUGUAGGG GCCGAAAGGCGAGUCAAGGUCU ACAUACAC | 2973 |
| 921 | GUCACUGU G CGUGCCAA | 1028 | UUGGCACG GCCGAAAGGCGAGUCAAGGUCU ACAGUGAC | 2974 |
| 925 | CUGUGCGU G CCAACAUU | 1029 | AAUGUUGG GCCGAAAGGCGAGUCAAGGUCU ACGCACAG | 2975 |
| 934 | CCAACAUU G CUGCCAUC | 1030 | GAUGGCAG GCCGAAAGGCGAGUCAAGGUCU AAUGUUGG | 2976 |
| 937 | ACAUUGCU G CCAUCACU | 1031 | AGUGAUGG GCCGAAAGGCGAGUCAAGGUCU AGCAAUGU | 2977 |
| 1006 | UGGCCUAU G CUGAGAUU | 1033 | AAUCUCAG GCCGAAAGGCGAGUCAAGGUCU AUAGGCCA | 2978 |
| 1015 | CUGAGAUU G CCAGGCCU | 1035 | AGGCCUGG GCCGAAAGGCGAGUCAAGGUCU AAUCUCAG | 2979 |
| 1092 | UUCUCCCU G CAGCUUUG | 1039 | CAAAGCUG GCCGAAAGGCGAGUCAAGGUCU AGGGAGAA | 2980 |
| 1105 | UUUGUGGU G CUGGCUUC | 1040 | GAAGCCAG GCCGAAAGGCGAGUCAAGGUCU ACCACAAA | 2981 |
| 1134 | UCUGAAGU G CUGGCCUC | 1042 | GAGGCCAG GCCGAAAGGCGAGUCAAGGUCU ACUUCAGA | 2982 |
| 1182 | GACCACUC G CUGUACAC | 1045 | GUGUACAG GCCGAAAGGCGAGUCAAGGUCU GAGUGGUC | 2983 |
| 1248 | AUCAUUGU G CGGGUGGA | 1048 | UCCACCCG GCCGAAAGGCGAGUCAAGGUCU ACAAUGAU | 2984 |
| 1286 | AAUGGACU G CAAGGAGU | 1050 | ACUCCUUG GCCGAAAGGCGAGUCAAGGUCU AGUCCAUU | 2985 |
| 1344 | CUUCGUUU G CCCAAGAA | 1052 | UUCUUGGG GCCGAAAGGCGAGUCAAGGUCU AAACGAAG | 2986 |
| 1366 | UUGAAGCU G CAGUCAAA | 1054 | UUUGACUG GCCGAAAGGCGAGUCAAGGUCU AGCUUCAA | 2987 |
| 1442 | GCUGGUGU G CUGGCAAG | 1056 | CUUGCCAG GCCGAAAGGCGAGUCAAGGUCU ACACCAGC | 2988 |
| 1526 | GUCCUUCC G CAUCACCA | 1058 | UGGUGAUG GCCGAAAGGCGAGUCAAGGUCU GGAAGGAC | 2989 |
| 1542 | AUCCUUCC G CAGCAAUA | 1059 | UAUUGCUG GCCGAAAGGCGAGUCAAGGUCU GGAAGGAU | 2990 |
| 1554 | CAAUACCU G CGGCCAGU | 1060 | ACUGGCCG GCCGAAAGGCGAGUCAAGGUCU AGGUAUUG | 2991 |
| 1603 | ACAAGUUU G CCAUCUCA | 1062 | UGAGAUGG GCCGAAAGGCGAGUCAAGGUCU AAACUUGU | 2992 |
| 1699 | UUGGCUUU G CUGUCAGC | 1066 | GCUGACAG GCCGAAAGGCGAGUCAAGGUCU AAAGCCAA | 2993 |
| 1708 | CUGUCAGC G CUUGCCAU | 1067 | AUGGCAAG GCCGAAAGGCGAGUCAAGGUCU GCUGACAG | 2994 |
| 1712 | CAGCGCUU G CCAUGUGC | 1068 | GCACAUGG GCCGAAAGGCGAGUCAAGGUCU AAGCGCUG | 2995 |
| 1719 | UGCCAUGU G CACGAUGA | 1069 | UCAUCGUG GCCGAAAGGCGAGUCAAGGUCU ACAUGGCA | 2996 |
| 1843 | UCAUGGCU G CCAUCUGC | 1074 | GCAGAUGG GCCGAAAGGCGAGUCAAGGUCU AGCCAUGA | 2997 |
| 1850 | UGCCAUCU G CGCCCUCU | 1075 | AGAGGGCG GCCGAAAGGCGAGUCAAGGUCU AGAUGGCA | 2998 |
| 1852 | CCAUCUGC G CCCUCUUC | 1076 | GAACAGGG GCCGAAAGGCGAGUCAAGGUCU GCAGAUGG | 2999 |
| 1863 | CUCUUCAU G CUGCCACU | 1077 | AGUGGCAG GCCGAAAGGCGAGUCAAGGUCU AUGAAGAG | 3000 |
| 1866 | UUCAUGCU G CCACUCUG | 1078 | CAGAGUGG GCCGAAAGGCGAGUCAAGGUCU AGCAUGAA | 3001 |
| 1874 | GCCACUCU G CCUCAUGG | 1079 | CCAUGAGG GCCGAAAGGCGAGUCAAGGUCU AGAGUGGC | 3002 |
| 1895 | UCAGUGGC G CUGCCUCC | 1080 | GGAGGCAG GCCGAAAGGCGAGUCAAGGUCU GCCACUGA | 3003 |
| 1898 | GUGGCGCU G CCUCCGCU | 1081 | AGCGGAGG GCCGAAAGGCGAGUCAAGGUCU AGCGCCAC | 3004 |
| 1904 | CUGCCUCC G CUGCCUGC | 1082 | GCAGGCAG GCCGAAAGGCGAGUCAAGGUCU GGAGGCAG | 3005 |
| 1907 | CCUCCGCU G CCUGCGCC | 1083 | GGCGCAGG GCCGAAAGGCGAGUCAAGGUCU AGCGGAGG | 3006 |
| 1911 | CGCUGCCU G CGCCAGCA | 1084 | UGCUGGCG GCCGAAAGGCGAGUCAAGGUCU AGGCAGCG | 3007 |
| 1913 | CUGCCUGC G CCAGCAGC | 1085 | GCUGCUGG GCCGAAAGGCGAGUCAAGGUCU GCAGGCAG | 3008 |
| 1933 | AUGACUUU G CUGAUGAC | 1088 | GUCAUCAG GCCGAAAGGCGAGUCAAGGUCU AAAGUCAU | 3009 |
| 1950 | AUCUCCCU G CUGAAGUG | 1091 | CACUUCAG GCCGAAAGGCGAGUCAAGGUCU AGGGAGAU | 3010 |
| 2087 | CACCAAAU G CCUCUGCC | 1094 | GGCAGAGG GCCGAAAGGCGAGUCAAGGUCU AUUUGGUG | 3011 |
| 2093 | AUGCCUCU G CCUUGAUG | 1095 | CAUCAAGG GCCGAAAGGCGAGUCAAGGUCU AGAGGCAU | 3012 |
| 2179 | AGCACUCU G CUGGCGGG | 1097 | CCCGCCAG GCCGAAAGGCGAGUCAAGGUCU AGAGUGCU | 3013 |
| 2227 | GAAAUUCU G CUGCUUGA | 1098 | UCAAGCAG GCCGAAAGGCGAGUCAAGGUCU AGAAUUUC | 3014 |
| 2230 | AUUCUGCU G CUUGAAAC | 1099 | GUUUCAAG GCCGAAAGGCGAGUCAAGGUCU AGCAGAAU | 3015 |
| 2329 | CAUCACAC G CAGGUUAC | 1102 | GUAACCUG GCCGAAAGGCGAGUCAAGGUCU GUGUGAUG | 3016 |
| 2393 | GUUUCCCU G CUGGCCAA | 1103 | UUGGCCAG GCCGAAAGGCGAGUCAAGGUCU AGGGAAAC | 3017 |
| 2419 | GAGAGGAU G CACAGUUU | 1104 | AAACUGUG GCCGAAAGGCGAGUCAAGGUCU AUCCUCUC | 3018 |
| 2428 | CACAGUUU G CUAUUUGC | 1105 | GCAAAUAG GCCGAAAGGCGAGUCAAGGUCU AAACUGUG | 3019 |
| 2435 | UGCUAUUU G CUUUAGAG | 1106 | CUCUAAAG GCCGAAAGGCGAGUCAAGGUCU AAAUAGCA | 3020 |
| 2476 | ACAUUGGU G CAAAGAUU | 1107 | AAUCUUUG GCCGAAAGGCGAGUCAAGGUCU ACCAAUGU | 3021 |
| 2485 | CAAAGAUU G CCUCUUGA | 1108 | UCAAGAGG GCCGAAAGGCGAGUCAAGGUCU AAUCUUUG | 3022 |
| 219 | GUGCCGAU G UAGCGGGC | 1110 | GCCCGCUA GCCGAAAGGCGAGUCAAGGUCU AUCGGCAC | 3023 |
| 483 | CUCCUGCU G UGGAUGGG | 1111 | CCCAUCCA GCCGAAAGGCGAGUCAAGGUCU AGCAGGAG | 3024 |
| 634 | GCAGCUUU G UGGAGAUG | 1112 | CAUCUCCA GCCGAAAGGCGAGUCAAGGUCU AAAGCUGC | 3025 |
| 804 | AGGCAGCU G UCCAGCAC | 1113 | GUGCUGGA GCCGAAAGGCGAGUCAAGGUCU AGCUGCCU | 3026 |
| 835 | GGAAGGGU G UGUAUGUG | 1114 | CACAUACA GCCGAAAGGCGAGUCAAGGUCU ACCCUUCC | 3027 |
| 837 | AAGGGUGU G UAUGUGCC | 1115 | GGCACAUA GCCGAAAGGCGAGUCAAGGUCU ACACCCUU | 3028 |
| 841 | GUGUGUAU G UGCCCUAC | 1116 | GUAGGGCA GCCGAAAGGCGAGUCAAGGUCU AUACACAC | 3029 |
| 919 | ACGUCACU G UGCGUGCC | 1117 | GGCACGCA GCCGAAAGGCGAGUCAAGGUCU AGUGACGU | 3030 |
| 1100 | GCAGCUUU G UGGUGCUG | 1118 | CAGCACCA GCCGAAAGGCGAGUCAAGGUCU AAAGCUGC | 3031 |
| 1144 | UGGCCUCU G UCGGAGGG | 1119 | CCCUCCGA GCCGAAAGGCGAGUCAAGGUCU AGAGGCCA | 3032 |
| 1185 | CACUCGCU G UACACAGG | 1120 | CCUGUGUA GCCGAAAGGCGAGUCAAGGUCU AGCGAGUG | 3033 |
| 1246 | UGAUCAUU G UGCGGGUG | 1121 | CACCCGCA GCCGAAAGGCGAGUCAAGGUCU AAUGAUCA | 3034 |
| 1315 | AGAGCAUU G UGGACAGU | 1122 | ACUGUCCA GCCGAAAGGCGAGUCAAGGUCU AAUGCUCU | 3035 |
| 1356 | AAGAAAGU G UUUGAAGC | 1123 | GCUUCAAA GCCGAAAGGCGAGUCAAGGUCU ACUUUCUU | 3036 |
| 1440 | CAGCUGGU G UGCUGGCA | 1124 | UGCCAGCA GCCGAAAGGCGAGUCAAGGUCU ACCAGCUG | 3037 |
| 1570 | UGGAAGAU G UGGCCACG | 1125 | CGUGGCCA GCCGAAAGGCGAGUCAAGGUCU AUCUUCCA | 3038 |
| 1592 | AGACGACU G UUACAAGU | 1126 | ACUUGUAA GCCGAAAGGCGAGUCAAGGUCU AGUCGUCU | 3039 |
| 1630 | CGGGCACU G UUAUGGGA | 1127 | UCCCAUAA GCCGAAAGGCGAGUCAAGGUCU AGUGCCCG | 3040 |
| 1642 | UGGGAGCU G UUAUCAUG | 1128 | CAUGAUAA GCCGAAAGGCGAGUCAAGGUCU AGCUCCCA | 3041 |
| 1666 | UCUACGUU G UCUUUGAU | 1129 | AUCAAAGA GCCGAAAGGCGAGUCAAGGUCU AACGUAGA | 3042 |
| 1702 | GCUUUGCU G UCAGCGCU | 1130 | AGCGCUGA GCCGAAAGGCGAGUCAAGGUCU AGCAAAGC | 3043 |
| 1717 | CUUGCCAU G UGCACGAU | 1131 | AUCGUGCA GCCGAAAGGCGAGUCAAGGUCU AUGGCAAG | 3044 |
| 1759 | GCCCUUUU G UCACCUUG | 1132 | CAAGGUGA GCCGAAAGGCGAGUCAAGGUCU AAAAGGGC | 3045 |
| 1781 | GGAAGACU G UGGCUACA | 1133 | UGUAGCCA GCCGAAAGGCGAGUCAAGGUCU AGUCUUCC | 3046 |
| 1834 | UAGCCUAU G UCAUGGCU | 1134 | AGCCAUGA GCCGAAAGGCGAGUCAAGGUCU AUAGGCUA | 3047 |
| 1884 | CUCAUGGU G UGUCAGUG | 1135 | CACUGACA GCCGAAAGGCGAGUCAAGGUCU ACCAUGAG | 3048 |
| 1886 | CAUGGUGU G UCAGUGGC | 1136 | GCCACUGA GCCGAAAGGCGAGUCAAGGUCU ACACCAUG | 3049 |
| 2048 | UGGCACCU G UGGCCAGA | 1137 | UCUGGCCA GCCGAAAGGCGAGUCAAGGUCU AGGUGCCA | 3050 |
| 2139 | CAGGGACU G UACCUGUA | 1138 | UACAGGUA GCCGAAAGGCGAGUCAAGGUCU AGUCCCUG | 3051 |
| 2145 | CUGUACCU G UAGGAAAC | 1139 | GUUUCCUA GCCGAAAGGCGAGUCAAGGUCU AGGUACAG | 3052 |
| 2256 | GAACCUUU G UCCACCAU | 1140 | AUGGUGGA GCCGAAAGGCGAGUCAAGGUCU AAAGGUUC | 3053 |
| 2346 | CUUGGCGU G UGUCCCUG | 1141 | CAGGGACA GCCGAAAGGCGAGUCAAGGUCU ACGCCAAG | 3054 |
| 2348 | UGGCGUGU G UCCCUGUG | 1142 | CACAGGGA GCCGAAAGGCGAGUCAAGGUCU ACACGCCA | 3055 |
| 2354 | GUGUCCCU G UGGUACCC | 1143 | GGGUACCA GCCGAAAGGCGAGUCAAGGUCU AGGGACAC | 3056 |
| 2385 | CCAAGCUU G UUUCCCUG | 1144 | CAGGGAAA GCCGAAAGGCGAGUCAAGGUCU AAGCUUGG | 3057 |
| 2453 | CAGGGACU G UAUAAACA | 1145 | UGUUUAUA GCCGAAAGGCGAGUCAAGGUCU AGUCCCUG | 3058 |
| 14 | CGUCCGCA G CCCGCCCG | 1146 | CGGGCGGG GCCGAAAGGCGAGUCAAGGUCU UGCGGACG | 3059 |
| 26 | GCCCGGGA G CUGCGAGC | 1147 | GCUCGCAG GCCGAAAGGCGAGUCAAGGUCU UCCCGGGC | 3060 |
| 33 | AGCUGCGA G CCGCGAGC | 1148 | GCUCGCGG GCCGAAAGGCGAGUCAAGGUCU UCGCAGCU | 3061 |
| 40 | AGCCGCGA G CUGGAUUA | 1149 | UAAUCCAG GCCGAAAGGCGAGUCAAGGUCU UCGCGGCU | 3062 |
| 51 | GGAUUAUG G UGGCCUGA | 1150 | UCAGGCCA GCCGAAAGGCGAGUCAAGGUCU CAUAAUCC | 3063 |
| 54 | UUAUGGUG G CCUGAGCA | 1151 | UGCUCAGG GCCGAAAGGCGAGUCAAGGUCU CACCAUAA | 3064 |
| 60 | UGGCCUGA G CAGCCAAC | 1152 | GUUGGCUG GCCGAAAGGCGAGUCAAGGUCU UCAGGCCA | 3065 |
| 63 | CCUGAGCA G CCAACGCA | 1153 | UGCGUUGG GCCGAAAGGCGAGUCAAGGUCU UGCUCAGG | 3066 |
| 72 | CCAACGCA G CCGCAGGA | 1154 | UCCUGCGG GCCGAAAGGCGAGUCAAGGUCU UGCGUUGG | 3067 |
| 81 | CCGCAGGA G CCCGGAGC | 1155 | GCUCCGGG GCCGAAAGGCGAGUCAAGGUCU UCCUGCGG | 3068 |
| 88 | AGCCCGGA G CCCUUGCC | 1156 | GGCAAGGG GCCGAAAGGCGAGUCAAGGUCU UCCGGGCU | 3069 |
| 134 | CCAGGGAA G CCGCCACC | 1157 | GGUGGCGG GCCGAAAGGCGAGUCAAGGUCU UUCCCUGG | 3070 |
| 144 | CGCCACCG G CCCGCCAU | 1158 | AUGGCGGG GCCGAAAGGCGAGUCAAGGUCU CCGUGGCG | 3071 |
| 167 | CCCUCCCA G CCCCGCCG | 1159 | CGGCGGGG GCCGAAAGGCGAGUCAAGGUCU UGGGAGGG | 3072 |
| 179 | CGCCGGGA G CCCGCGCC | 1160 | GGCGCGGG GCCGAAAGGCGAGUCAAGGUCU UCCCGGCG | 3073 |
| 198 | CUGCCCAG G CUGGCCGC | 1161 | GCGGCCAG GCCGAAAGGCGAGUCAAGGUCU CUGGGCAG | 3074 |
| 202 | CCAGGCUG G CCGCCGCC | 1162 | GGCGGCGG GCCGAAAGGCGAGUCAAGGUCU CAGCCUGG | 3075 |
| 211 | CCGCCGCC G UGCCGAUG | 1163 | CAUCGGCA GCCGAAAGGCGAGUCAAGGUCU GGCGGCGG | 3076 |
| 222 | CCGAUGUA G CGGGCUCC | 1164 | GGAGCCCG GCCGAAAGGCGAGUCAAGGUCU UACAUCGG | 3077 |
| 226 | UGUAGCGG G CUCCGGAU | 1165 | AUCCGGAG GCCGAAAGGCGAGUCAAGGUCU CCGCUACA | 3078 |
| 239 | GGAUCCCA G CCUCUCCC | 1166 | GGGAGAGG GCCGAAAGGCGAGUCAAGGUCU UGGGAUCC | 3079 |
| 256 | CUGCUCCC G UGCUCUGC | 1167 | GCAGAGCA GCCGAAAGGCGAGUCAAGGUCU GGGAGCAG | 3080 |
| 290 | UCUCCACA G CCCGGACC | 1168 | GGUCCGGG GCCGAAAGGCGAGUCAAGGUCU UGUGGAGA | 3081 |
| 304 | ACCCGGGG G CUGGCCCA | 1169 | UGGGCCAG GCCGAAAGGCGAGUCAAGGUCU CCCCGGGU | 3082 |
| 308 | GGGGGCUG G CCCAGGGC | 1170 | GCCCUGGG GCCGAAAGGCGAGUCAAGGUCU CAGCCCCC | 3083 |
| 315 | GGCCCAGG G CCCUGCAG | 1171 | CUGCAGGG GCCGAAAGGCGAGUCAAGGUCU CCUGGGCC | 3084 |
| 324 | CCCUGCAG G CCCUGGCG | 1172 | CGCCAGGG GCCGAAAGGCGAGUCAAGGUCU CUGCAGGG | 3085 |
| 330 | AGGCCCUG G CGUCCUGA | 1173 | UCAGGACG GCCGAAAGGCGAGUCAAGGUCU CAGGGCCU | 3086 |
| 332 | GCCCUGGC G UCCUGAUG | 1174 | CAUCAGGA GCCGAAAGGCGAGUCAAGGUCU GCCAGGGC | 3087 |
| 348 | GCCCCCAA G CUCCCUCU | 1175 | AGAGGGAG GCCGAAAGGCGAGUCAAGGUCU UUGGGGGC | 3088 |
| 365 | CCUGAGAA G CCACCAGC | 1176 | GCUGGUGG GCCGAAAGGCGAGUCAAGGUCU UUCUCAGG | 3089 |
| 372 | AGCCACCA G CACCACCC | 1177 | GGGUGGUG GCCGAAAGGCGAGUCAAGGUCU UGGUGGCU | 3090 |
| 391 | ACUUGGGG G CAGGCGCC | 1178 | GGCGCCUG GCCGAAAGGCGAGUCAAGGUCU CCCCAAGU | 3091 |
| 395 | GGGGGCAG G CGCCAGGG | 1179 | CCCUGGCG GCCGAAAGGCGAGUCAAGGUCU CUGCCCCC | 3092 |
| 410 | GGACGGAC G UGGGCCAG | 1180 | CUGGCCCA GCCGAAAGGCGAGUCAAGGUCU GUCCGUCC | 3093 |
| 414 | GGACGUGG G CCAGUGCG | 1181 | CGCACUGG GCCGAAAGGCGAGUCAAGGUCU CCACGUCC | 3094 |
| 418 | GUGGGCCA G UGCGAGCC | 1182 | GGCUCGCA GCCGAAAGGCGAGUCAAGGUCU UGGCCCAC | 3095 |
| 424 | CAGUGCGA G CCCAGAGG | 1183 | CCUCUGGG GCCGAAAGGCGAGUCAAGGUCU UCGCACUG | 3096 |
| 433 | CCCAGAGG G CCCGAAGG | 1184 | CCUUCGGG GCCGAAAGGCGAGUCAAGGUCU CCUCUGGG | 3097 |
| 441 | GCCCGAAG G CCGGGGCC | 1185 | GGCCCCGG GCCGAAAGGCGAGUCAAGGUCU CUUCGGGC | 3098 |
| 447 | AGGCCGGG G CCCACCAU | 1186 | AUGGUGGG GCCGAAAGGCGAGUCAAGGUCU CCCGGCCU | 3099 |
| 457 | CCACCAUG G CCCAAGCC | 1187 | GGCUUGGG GCCGAAAGGCGAGUCAAGGUCU CAUGGUGG | 3100 |
| 463 | UGGCCCAA G CCCUGCCC | 1188 | GGGCAGGG GCCGAAAGGCGAGUCAAGGUCU UUGGGCCA | 3101 |
| 474 | CUGCCCUG G CUCCUGCU | 1189 | AGCAGGAG GCCGAAAGGCGAGUCAAGGUCU CAGGGCAG | 3102 |
| 491 | GUGGAUGG G CGCGGGAG | 1190 | CUCCCGCG GCCGAAAGGCGAGUCAAGGUCU CCAUCCAC | 3103 |
| 499 | GCGCGGGA G UGCUGCCU | 1191 | AGGCAGCA GCCGAAAGGCGAGUCAAGGUCU UCCCGCGC | 3104 |
| 515 | UGCCCACG G CACCCAGC | 1192 | GCUGGGUG GCCGAAAGGCGAGUCAAGGUCU CGUGGGCA | 3105 |
| 522 | GGCACCCA G CACGGCAU | 1193 | AUGCCGUG GCCGAAAGGCGAGUCAAGGUCU UGGGUGCC | 3106 |
| 527 | CCAGCACG G CAUCCGGC | 1194 | GCCGGAUG GCCGAAAGGCGAGUCAAGGUCU CGUGCUGG | 3107 |
| 534 | GGCAUCCG G CUGCCCCU | 1195 | AGGGGCAG GCCGAAAGGCGAGUCAAGGUCU CGGAUGCC | 3108 |
| 548 | CCUGCGCA G CGGCCUGG | 1196 | CCAGGCCG GCCGAAAGGCGAGUCAAGGUCU UGCGCAGG | 3109 |
| 551 | GCGCAGCG G CCUGGGGG | 1197 | CCCCCAGG GCCGAAAGGCGAGUCAAGGUCU CGCUGCGC | 3110 |
| 560 | CCUGGGGG G CGCCCCCC | 1198 | GGGGGGCG GCCGAAAGGCGAGUCAAGGUCU CCCCCAGG | 3111 |
| 573 | CCCCUGGG G CUGCGGCU | 1199 | AGCCGCAG GCCGAAAGGCGAGUCAAGGUCU CCCAGGGG | 3112 |
| 579 | GGGCUGCG G CUGCCCCG | 1200 | CGGGGCAG GCCGAAAGGCGAGUCAAGGUCU CGCAGCCC | 3113 |
| 603 | GACGAAGA G CCCGAGGA | 1201 | UCCUCGGG GCCGAAAGGCGAGUCAAGGUCU UCUUCGUC | 3114 |
| 612 | CCCGAGGA G CCCGGCCG | 1202 | CGGCCGGG GCCGAAAGGCGAGUCAAGGUCU UCCUCGGG | 3115 |
| 617 | GGAGCCCG G CCGGAGGG | 1203 | CCCUCCGG GCCGAAAGGCGAGUCAAGGUCU CGGGCUCC | 3116 |
| 626 | CCGGAGGG G CAGCUUUG | 1204 | CAAAGCUG GCCGAAAGGCGAGUCAAGGUCU CCCUCCGG | 3117 |
| 629 | GAGGGGCA G CUUUGUGG | 1205 | CCACAAAG GCCGAAAGGCGAGUCAAGGUCU UGCCCCUC | 3118 |
| 643 | UGGAGAUG G UGGACAAC | 1206 | GUUGUCCA GCCGAAAGGCGAGUCAAGGUCU CAUCUCCA | 3119 |
| 659 | CCUGAGGG G CAAGUCGG | 1207 | CCGACUUG GCCGAAAGGCGAGUCAAGGUCU CCCUCAGG | 3120 |
| 663 | AGGGGCAA G UCGGGGCA | 1208 | UGCCCCGA GCCGAAAGGCGAGUCAAGGUCU UUGCCCCU | 3121 |
| 669 | AAGUCGGG G CAGGGCUA | 1209 | UAGCCCUG GCCGAAAGGCGAGUCAAGGUCU CCCGACUU | 3122 |
| 674 | GGGGCAGG G CUACUACG | 1210 | CGUAGUAG GCCGAAAGGCGAGUCAAGGUCU CCUGCCCC | 3123 |
| 682 | GCUACUAC G UGGAGAUG | 1211 | CAUCUCCA GCCGAAAGGCGAGUCAAGGUCU GUAGUAGC | 3124 |
| 694 | AGAUGACC G UGGGCAGC | 1212 | GCUGCCCA GCCGAAAGGCGAGUCAAGGUCU GGUCAUCU | 3125 |
| 698 | GACCGUGG G CAGCCCCC | 1213 | GGGGGCUG GCCGAAAGGCGAGUCAAGGUCU CCACGGUC | 3126 |
| 701 | CGUGGGCA G CCCCCCGC | 1214 | GCGGGGGG GCCGAAAGGCGAGUCAAGGUCU UGCCCACG | 3127 |
| 727 | ACAUCCUG G UGGAUACA | 1215 | UGUAUCCA GCCGAAAGGCGAGUCAAGGUCU CAGGAUGU | 3128 |
| 737 | GGAUACAG G CAGCAGUA | 1216 | UACUGCUG GCCGAAAGGCGAGUCAAGGUCU CUGUAUCC | 3129 |
| 740 | UACAGGCA G CAGUAACU | 1217 | AGUUACUG GCCGAAAGGCGAGUCAAGGUCU UGCCUGUA | 3130 |
| 743 | AGGCAGCA G UAACUUUG | 1218 | CAAAGUUA GCCGAAAGGCGAGUCAAGGUCU UGCUGCCU | 3131 |
| 754 | ACUUUGCA G UGGGUGCU | 1219 | AGCACCCA GCCGAAAGGCGAGUCAAGGUCU UGCAAAGU | 3132 |
| 758 | UGCAGUGG G UGCUGCCC | 1220 | GGGCAGCA GCCGAAAGGCGAGUCAAGGUCU CCACUGCA | 3133 |
| 798 | UACCAGAG G CAGCUGUC | 1221 | GACAGCUG GCCGAAAGGCGAGUCAAGGUCU CUCUGGUA | 3134 |
| 801 | CAGAGGCA G CUGUCCAG | 1222 | CUGGACAG GCCGAAAGGCGAGUCAAGGUCU UGCCUCUG | 3135 |
| 809 | GCUGUCCA G CACAUACC | 1223 | GGUAUGUG GCCGAAAGGCGAGUCAAGGUCU UGGACAGC | 3136 |
| 833 | CCGGAAGG G UGUGUAUG | 1224 | CAUACACA GCCGAAAGGCGAGUCAAGGUCU CCUUCCGG | 3137 |
| 857 | CACCCAGG G CAAGUGGG | 1225 | CCCACUUG GCCGAAAGGCGAGUCAAGGUCU CCUGGGUG | 3138 |
| 861 | CAGGGCAA G UGGGAAGG | 1226 | CCUUCCCA GCCGAAAGGCGAGUCAAGGUCU UUGCCCUG | 3139 |
| 873 | GAAGGGGA G CUGGGCAC | 1227 | GUGCCCAG GCCGAAAGGCGAGUCAAGGUCU UCCCCUUC | 3140 |
| 878 | GGAGCUGG G CACCGACC | 1228 | GGUCGGUG GCCGAAAGGCGAGUCAAGGUCU CCAGCUCC | 3141 |
| 889 | CCGACCUG G UAAGCAUC | 1229 | GAUGCUUA GCCGAAAGGCGAGUCAAGGUCU CAGGUCGG | 3142 |
| 893 | CCUGGUAA G CAUCCCCC | 1230 | GGGGGAUG GCCGAAAGGCGAGUCAAGGUCU UUACCAGG | 3143 |
| 905 | CCCCCAUG G CCCCAACG | 1231 | CGUUGGGG GCCGAAAGGCGAGUCAAGGUCU CAUGGGGG | 3144 |
| 913 | GCCCCAAC G UCACUGUG | 1232 | CACAGUGA GCCGAAAGGCGAGUCAAGGUCU GUUGGGGC | 3145 |
| 923 | CACUGUGC G UGCCAACA | 1233 | UGUUGGCA GCCGAAAGGCGAGUCAAGGUCU UCACAGUG | 3146 |
| 957 | UCAGACAA G UUCUUCAU | 1234 | AUGAAGAA GCCGAAAGGCGAGUCAAGGUCU UUGUCUGA | 3147 |
| 971 | CAUCAACG G CUCCAACU | 1235 | AGUUGGAG GCCGAAAGGCGAGUCAAGGUCU CGUUGAUG | 3148 |
| 986 | CUGGGAAG G CAUCCUGG | 1236 | CCAGGAUG GCCGAAAGGCGAGUCAAGGUCU CUUCCCAG | 3149 |
| 996 | AUCCUGGG G CUGGCCUA | 1237 | UAGGCCAG GCCGAAAGGCGAGUCAAGGUCU CCCAGGAU | 3150 |
| 1000 | UGGGGCUG G CCUAUGCU | 1238 | AGCAUAGG GCCGAAAGGCGAGUCAAGGUCU CAGCCCCA | 3151 |
| 1020 | AUUGCCAG G CCUGACGA | 1239 | UCGUCAGG GCCGAAAGGCGAGUCAAGGUCU CUGGCAAU | 3152 |
| 1038 | UCCCUGGA G CCUUUCUU | 1240 | AAGAAAGG GCCGAAAGGCGAGUCAAGGUCU UCCAGGGA | 3153 |
| 1057 | ACUCUCUG G UAAAGCAG | 1241 | CUGCUUUA GCCGAAAGGCGAGUCAAGGUCU CAGAGAGU | 3154 |
| 1062 | CUGGUAAA G CAGACCCA | 1242 | UGGGUCUG GCCGAAAGGCGAGUCAAGGUCU UUUACCAG | 3155 |
| 1072 | AGACCCAC G UUCCCAAC | 1243 | GUUGGGAA GCCGAAAGGCGAGUCAAGGUCU GUGGGUCU | 3156 |
| 1095 | UCCCUGCA G CUUUGUGG | 1244 | CCACAAAG GCCGAAAGGCGAGUCAAGGUCU UGCAGGGA | 3157 |
| 1103 | GCUUUGUG G UGCUGGCU | 1245 | AGCCAGCA GCCGAAAGGCGAGUCAAGGUCU CACAAAGC | 3158 |
| 1109 | UGGUGCUG G CUUCCCCC | 1246 | GGGGGAAG GCCGAAAGGCGAGUCAAGGUCU CAGCACCA | 3159 |
| 1125 | CUCAACCA G UCUGAAGU | 1247 | ACUUCAGA GCCGAAAGGCGAGUCAAGGUCU UGGUUGAG | 3160 |
| 1132 | AGUCUGAA G UGCUGGCC | 1248 | GGCCAGCA GCCGAAAGGCGAGUCAAGGUCU UUCAGACU | 3161 |
| 1138 | AAGUGCUG G CCUCUGUC | 1249 | GACAGAGG GCCGAAAGGCGAGUCAAGGUCU CAGCACUU | 3162 |
| 1154 | CGGAGGGA G CAUGAUCA | 1250 | UGAUCAUG GCCGAAAGGCGAGUCAAGGUCU UCCCUCCG | 3163 |
| 1169 | CAUUGGAG G UAUCGACC | 1251 | GGUCGAUA GCCGAAAGGCGAGUCAAGGUCU CUCCAAUG | 3164 |
| 1193 | GUACACAG G CAGUCUCU | 1252 | AGAGACUG GCCGAAAGGCGAGUCAAGGUCU CUGUGUAC | 3165 |
| 1196 | CACAGGCA G UCUCUGGU | 1253 | ACCAGAGA GCCGAAAGGCGAGUCAAGGUCU UGCCUGUG | 3166 |
| 1203 | AGUCUCUG G UAUACACC | 1254 | GGUGUAUA GCCGAAAGGCGAGUCAAGGUCU CAGAGACU | 3167 |
| 1218 | CCCAUCCG G CGGGAGUG | 1255 | CACUCCCG GCCGAAAGGCGAGUCAAGGUCU CGGAUGGG | 3168 |
| 1224 | CGGCGGGA G UGGUAUUA | 1256 | UAAUACCA GCCGAAAGGCGAGUCAAGGUCU UCCCGCCG | 3169 |
| 1227 | CGGGAGUG G UAUUAUGA | 1257 | UCAUAAUA GCCGAAAGGCGAGUCAAGGUCU CACUCCCG | 3170 |
| 1237 | AUUAUGAG G UGAUCAUU | 1258 | AAUGAUCA GCCGAAAGGCGAGUCAAGGUCU CUCAUAAU | 3171 |
| 1252 | UUGUGCGG G UGGAGAUC | 1259 | GAUCUCCA GCCGAAAGGCGAGUCAAGGUCU CCGCACAA | 3172 |
| 1293 | UGCAAGGA G UACAACUA | 1260 | UAGUUGUA GCCGAAAGGCGAGUCAAGGUCU UCCUUGCA | 3173 |
| 1310 | UGACAAGA G CAUUGUGG | 1261 | CCACAAUG GCCGAAAGGCGAGUCAAGGUCU UCUUGUCA | 3174 |
| 1322 | UGUGGACA G UGGCACCA | 1262 | UGGUGCCA GCCGAAAGGCGAGUCAAGGUCU UGUCCACA | 3175 |
| 1325 | GGACAGUG G CACCACCA | 1263 | UGGUGGUG GCCGAAAGGCGAGUCAAGGUCU CACUGUCC | 3176 |
| 1340 | CAACCUUC G UUUGCCCA | 1264 | UGGGCAAA GCCGAAAGGCGAGUCAAGGUCU GAAGGUUG | 3177 |
| 1354 | CCAAGAAA G UGUUUGAA | 1265 | UUCAAACA GCCGAAAGGCGAGUCAAGGUCU UUUCUUGG | 3178 |
| 1363 | UGUUUGAA G CUGCAGUC | 1266 | GACUGCAG GCCGAAAGGCGAGUCAAGGUCU UUCAAACA | 3179 |
| 1369 | AAGCUGCA G UCAAAUCC | 1267 | GGAUUUGA GCCGAAAGGCGAGUCAAGGUCU UGCAGCUU | 3180 |
| 1384 | CCAUCAAG G CAGCCUCC | 1268 | GGAGGCUG GCCGAAAGGCGAGUCAAGGUCU CUUGAUGG | 3181 |
| 1387 | UCAAGGCA G CCUCCUCC | 1269 | GGAGGAGG GCCGAAAGGCGAGUCAAGGUCU UGCCUUGA | 3182 |
| 1404 | ACGGAGAA G UUCCCUGA | 1270 | UCAGGGAA GCCGAAAGGCGAGUCAAGGUCU UUCUCCGU | 3183 |
| 1415 | CCCUGAUG G UUUCUGGC | 1271 | GCCAGAAA GCCGAAAGGCGAGUCAAGGUCU CAUCAGGG | 3184 |
| 1422 | GGUUUCUG G CUAGGAGA | 1272 | UCUCCUAG GCCGAAAGGCGAGUCAAGGUCU CAGAAACC | 3185 |
| 1431 | CUAGGAGA G CAGCUGGU | 1273 | ACCAGCUG GCCGAAAGGCGAGUCAAGGUCU UCUCCUAG | 3186 |
| 1434 | GGAGAGCA G CUGGUGUG | 1274 | CACACCAG GCCGAAAGGCGAGUCAAGGUCU UGCUCUCC | 3187 |
| 1438 | AGCAGCUG G UGUGCUGG | 1275 | CCAGCACA GCCGAAAGGCGAGUCAAGGUCU CAGCUGCU | 3188 |
| 1446 | GUGUGCUG G CAAGCAGG | 1276 | CCUGCUUG GCCGAAAGGCGAGUCAAGGUCU CAGCACAC | 3189 |
| 1450 | GCUGGCAA G CAGGCACC | 1277 | GGUGCCUG GCCGAAAGGCGAGUCAAGGUCU UUGCCAGC | 3190 |
| 1454 | GCAAGCAG G CACCACCC | 1278 | GGGUGGUG GCCGAAAGGCGAGUCAAGGUCU CUGCUUGC | 3191 |
| 1480 | UUUUCCCA G UCAUCUCA | 1279 | UGAGAUGA GCCGAAAGGCGAGUCAAGGUCU UGGGAAAA | 3192 |
| 1502 | CCUAAUGG G UGAGGUUA | 1280 | UAACCUCA GCCGAAAGGCGAGUCAAGGUCU CCAUUAGG | 3193 |
| 1507 | UGGGUGAG G UUACCAAC | 1281 | GUUGGUAA GCCGAAAGGCGAGUCAAGGUCU CUCACCCA | 3194 |
| 1518 | ACCAACCA G UCCUUCCG | 1282 | CGGAAGGA GCCGAAAGGCGAGUCAAGGUCU UGGUUGGU | 3195 |
| 1545 | CUUCCGCA G CAAUACCU | 1283 | AGGUAUUG GCCGAAAGGCGAGUCAAGGUCU UGCGGAAG | 3196 |
| 1557 | UACCUGCG G CCAGUGGA | 1284 | UCCACUGG GCCGAAAGGCGAGUCAAGGUCU CGCAGGUA | 3197 |
| 1561 | UGCGGCCA G UGGAAGAU | 1285 | AUCUUCCA GCCGAAAGGCGAGUCAAGGUCU UGGCCGCA | 3198 |
| 1573 | AAGAUGUG G CCACGUCC | 1286 | GGACGUGG GCCGAAAGGCGAGUCAAGGUCU CACAUCUU | 3199 |
| 1578 | GUGGCCAC G UCCCAAGA | 1287 | UCUUGGGA GCCGAAAGGCGAGUCAAGGUCU GUGGCCAC | 3200 |
| 1599 | UGUUACAA G UUUGCCAU | 1288 | AUGGCAAA GCCGAAAGGCGAGUCAAGGUCU UUGUAACA | 3201 |
| 1614 | AUCUCACA G UCAUCCAC | 1289 | GUGGAUGA GCCGAAAGGCGAGUCAAGGUCU UGUGAGAU | 3202 |
| 1625 | AUCCACGG G CACUGUUA | 1290 | UAACAGUG GCCGAAAGGCGAGUCAAGGUCU CCGUGGAU | 3203 |
| 1639 | UUAUGGGA G CUGUUAUC | 1291 | GAUAACAG GCCGAAAGGCGAGUCAAGGUCU UCCCAUAA | 3204 |
| 1655 | CAUGGAGG G CUUCUACG | 1292 | CGUAGAAG GCCGAAAGGCGAGUCAAGGUCU CCUCCAUG | 3205 |
| 1663 | GCUUCUAC G UUGUCUUU | 1293 | AAAGACAA GCCGAAAGGCGAGUCAAGGUCU GUAGAAGC | 3206 |
| 1678 | UUGAUCGG G CCCGAAAA | 1294 | UUUUCGGG GCCGAAAGGCGAGUCAAGGUCU CCGAUCAA | 3207 |
| 1694 | ACGAAUUG G CUUUGCUG | 1295 | CAGCAAAG GCCGAAAGGCGAGUCAAGGUCU CAAUUCGU | 3208 |
| 1706 | UGCUGUCA G CGCUUGCC | 1296 | GGCAAGCG GCCGAAAGGCGAGUCAAGGUCU UGACAGCA | 3209 |
| 1728 | CACGAUGA G UCCAGGAC | 1297 | GUCCUGAA GCCGAAAGGCGAGUCAAGGUCU UCAUCGUG | 3210 |
| 1738 | UCAGGACG G CAGCGGUG | 1298 | CACCGCUG GCCGAAAGGCGAGUCAAGGUCU CGUCCUGA | 3211 |
| 1741 | GGACGGCA G CGGUGGAA | 1299 | UUCCACCG GCCGAAAGGCGAGUCAAGGUCU UGCCGUCC | 3212 |
| 1744 | CGGCAGCG G UGGAAGGC | 1300 | GCCUUCCA GCCGAAAGGCGAGUCAAGGUCU CGCUGCCG | 3213 |
| 1751 | GGUGGAAG G CCCUUUUG | 1301 | CAAAAGGG GCCGAAAGGCGAGUCAAGGUCU CUUCCACC | 3214 |
| 1784 | AGACUGUG G CUACAACA | 1302 | UGUUGUAG GCCGAAAGGCGAGUCAAGGUCU CACAGUCU | 3215 |
| 1809 | ACAGAUGA G UCAACCCU | 1303 | AGGGUUGA GCCGAAAGGCGAGUCAAGGUCU UCAUCUGU | 3216 |
| 1828 | UGACCAUA G CCUAUGUC | 1304 | GACAUAGG GCCGAAAGGCGAGUCAAGGUCU UAUGGUCA | 3217 |
| 1840 | AUGUCAUG G CUGCCAUC | 1305 | GAUGGCAG GCCGAAAGGCGAGUCAAGGUCU CAUGACAU | 3218 |
| 1882 | GCCUCAUG G UGUGUCAG | 1306 | CUGACACA GCCGAAAGGCGAGUCAAGGUCU CAUGAGGC | 3219 |
| 1890 | GUGUGUCA G UGGCGCUG | 1307 | CAGCGCCA GCCGAAAGGCGAGUCAAGGUCU UGACACAC | 3220 |
| 1893 | UGUCAGUG G CGCUGCCU | 1308 | AGGCAGCG GCCGAAAGGCGAGUCAAGGUCU CACUGACA | 3221 |
| 1917 | CUGCGCCA G CAGCAUGA | 1309 | UCAUGCUG GCCGAAAGGCGAGUCAAGGUCU UGGCGCAG | 3222 |
| 1920 | CGCCAGCA G CAUGAUGA | 1310 | UCAUCAUG GCCGAAAGGCGAGUCAAGGUCU UGCUGGCG | 3223 |
| 1956 | CUGCUGAA G UGAGGAGG | 1311 | CCUCCUCA GCCGAAAGGCGAGUCAAGGUCU UUCAGCAG | 3224 |
| 1964 | GUGAGGAG G CCCAUGGG | 1312 | CCCAUGGG GCCGAAAGGCGAGUCAAGGUCU CUCCUCAC | 3225 |
| 1972 | GCCCAUGG G CAGAAGAU | 1313 | AUCUUCUG GCCGAAAGGCGAGUCAAGGUCU CCAUGGGC | 3226 |
| 2006 | ACACCUCC G UGGUUCAC | 1314 | GUGAACCA GCCGAAAGGCGAGUCAAGGUCU GGAGGUGU | 3227 |
| 2009 | CCUCCGUG G UUCACUUU | 1315 | AAAGUGAA GCCGAAAGGCGAGUCAAGGUCU CACGGAGG | 3228 |
| 2019 | UCACUUUG G UCACAAGU | 1316 | ACUUGUGA GCCGAAAGGCGAGUCAAGGUCU CAAAGUGA | 3229 |
| 2026 | GGUCACAA G UAGGAGAC | 1317 | GUCUCCUA GCCGAAAGGCGAGUCAAGGUCU UUGUGACC | 3230 |
| 2042 | CACAGAUG G CACCUGUG | 1318 | CACAGGUG GCCGAAAGGCGAGUCAAGGUCU CAUCUGUG | 3231 |
| 2051 | CACCUGUG G CCAGAGCA | 1319 | UGCUCUGG GCCGAAAGGCGAGUCAAGGUCU CACAGGUG | 3232 |
| 2057 | UGGCCAGA G CACCUCAG | 1320 | CUGAGGUG GCCGAAAGGCGAGUCAAGGUCU UCUGGCCA | 3233 |
| 2114 | AGGAAAAG G CUGGCAAG | 1321 | CUUGCCAG GCCGAAAGGCGAGUCAAGGUCU CUUUUCCU | 3234 |
| 2118 | AAAGGCUG G CAAGGUGG | 1322 | CCACCUUG GCCGAAAGGCGAGUCAAGGUCU CAGCCUUU | 3235 |
| 2123 | CUGGCAAG G UGGGUUCC | 1323 | GGAACCCA GCCGAAAGGCGAGUCAAGGUCU CUUGCCAG | 3236 |
| 2127 | CAAGGUGG G UUCCAGGG | 1324 | CCCUGGAA GCCGAAAGGCGAGUCAAGGUCU CCACCUUG | 3237 |
| 2172 | AGAAAGAA G CACUCUGC | 1325 | GCAGAGUG GCCGAAAGGCGAGUCAAGGUCU UUCUUUCU | 3238 |
| 2183 | CUCUGCUG G CGGGAAUA | 1326 | UAUUCCCG GCCGAAAGGCGAGUCAAGGUCU CAGCAGAG | 3239 |
| 2198 | UACUCUUG G UCACCUCA | 1327 | UGAGGUGA GCCGAAAGGCGAGUCAAGGUCU CAAGAGUA | 3240 |
| 2214 | AAAUUUAA G UCGGGAAA | 1328 | UUUCCCGA GCCGAAAGGCGAGUCAAGGUCU UUAAAUUU | 3241 |
| 2243 | AAACUUCA G CCCUGAAC | 1329 | GUUCAGGG GCCGAAAGGCGAGUCAAGGUCU UGAAGUUU | 3242 |
| 2288 | AACCCAAA G UAUUCUUC | 1330 | GAAGAAUA GCCGAAAGGCGAGUCAAGGUCU UUUGGGUU | 3243 |
| 2305 | UUUUCUUA G UUUCAGAA | 1331 | UUCUGAAA GCCGAAAGGCGAGUCAAGGUCU UAAGAAAA | 3244 |
| 2314 | UUUCAGAA G UACUGGCA | 1332 | UGCCAGUA GCCGAAAGGCGAGUCAAGGUCU UUCUGAAA | 3245 |
| 2320 | AAGUACUG G CAUCACAC | 1333 | GUGUGAUG GCCGAAAGGCGAGUCAAGGUCU CAGUACUU | 3246 |
| 2333 | ACACGCAG G UUACCUUG | 1334 | CAAGGUAA GCCGAAAGGCGAGUCAAGGUCU CUGCGUGU | 3247 |
| 2342 | UUACCUUG G CGUGUGUC | 1335 | GACACACG GCCGAAAGGCGAGUCAAGGUCU CAAGGUAA | 3248 |
| 2344 | ACCUUGGC G UGUGUCCC | 1336 | GGGACACA GCCGAAAGGCGAGUCAAGGUCU GCCAAGGU | 3249 |
| 2357 | UCCCUGUG G UACCCUGG | 1337 | CCAGGGUA GCCGAAAGGCGAGUCAAGGUCU CACAGGGA | 3250 |
| 2365 | GUACCCUG G CAGAGAAG | 1338 | CUUCUCUG GCCGAAAGGCGAGUCAAGGUCU CAGGGUAC | 3251 |
| 2381 | GAGACCAA G CUUGUUUC | 1339 | GAAACAAG GCCGAAAGGCGAGUCAAGGUCU UUGGUCUC | 3252 |
| 2397 | CCCUGCUG G CCAAAGUC | 1340 | GACUUUGG GCCGAAAGGCGAGUCAAGGUCU CAGCAGGG | 3253 |
| 2403 | UGGCCAAA G UCAGUAGG | 1341 | CCUACUGA GCCGAAAGGCGAGUCAAGGUCU UUUGGCCA | 3254 |
| 2407 | CAAAGUCA G UAGGAGAG | 1342 | CUCUCCUA GCCGAAAGGCGAGUCAAGGUCU UGACUUUG | 3255 |
| 2424 | GAUGCACA G UUUGCUAU | 1343 | AUAGCAAA GCCGAAAGGCGAGUCAAGGUCU UGUGCAUC | 3256 |
| 2463 | AUAAACAA G CCUAACAU | 1344 | AUGUUAGG GCCGAAAGGCGAGUCAAGGUCU UUGUUUAU | 3257 |
| 2474 | UAACAUUG G UGCAAAGA | 1345 | UCUUUGCA GCCGAAAGGCGAGUCAAGGUCU CAAUGUUA | 3258 |

Input Sequence = AF190725. Cut Site = G/.
Stem Length = 8. Core Sequence = GCcgaaagGCGaGuCaaGGuCu
AF190725 (Homo sapiens beta-site APP cleaving enzyme (BACE) mRNA; 2526 bp)

TABLE VII
Human BACE DNAzyme and Target Sequence

| Pos | Substrate | Seq ID | DNAzyme | Seq ID |
|---|---|---|---|---|
| 48 | GCUGGAUU A UGGUGGCC | 3 | GGCCACCA GGCTAGCTACAACGA AATCCAGC | 3259 |
| 677 | GCAGGGCU A CUACGUGG | 27 | CCACGTAG GGCTAGCTACAACGA AGCCCTGC | 3260 |
| 680 | GGGCUACU A CGUGGAGA | 28 | TCTCCACG GGCTAGCTACAACGA AGTAGCCC | 3261 |
| 733 | UGGUGGAU A CAGGCAGC | 31 | GCTGCCTG GGCTAGCTACAACGA ATCCACCA | 3262 |
| 788 | GCAUCGCU A CUACCAGA | 38 | TCTGGTAG GGCTAGCTACAACGA AGCGATGC | 3263 |
| 791 | UCGCUACU A CCAGAGGC | 39 | GCCTCTGG GGCTAGCTACAACGA AGTAGCGA | 3264 |
| 815 | CAGCACAU A CCGGGACC | 41 | GGTCCCGG GGCTAGCTACAACGA ATGTGCTG | 3265 |
| 839 | GGGUGUGU A UGUGCCCU | 43 | AGGGCACA GGCTAGCTACAACGA ACACACCC | 3266 |
| 848 | UGUGCCCU A CACCCAGG | 44 | CCTGGGTG GGCTAGCTACAACGA AGGGCACA | 3267 |
| 1004 | GCUGGCCU A UGCUGAGA | 58 | TCTCAGCA GGCTAGCTACAACGA AGGCCAGC | 3268 |
| 1171 | UUGGAGGU A UCGACCAC | 85 | GTGGTCGA GGCTAGCTACAACGA ACCTCCAA | 3269 |
| 1187 | CUCGCUGU A CACAGGCA | 88 | TGCCTGTG GGCTAGCTACAACGA ACAGCGAG | 3270 |
| 1205 | UCUCUGGU A UACACCCA | 91 | TGGGTGTA GGCTAGCTACAACGA ACCAGAGA | 3271 |
| 1207 | UCUGGUAU A CACCCAUC | 92 | GATGGGTG GGCTAGCTACAACGA ATACCAGA | 3272 |
| 1229 | GGAGUGGU A UUAUGAGG | 94 | CCTCATAA GGCTAGCTACAACGA ACCACTCC | 3273 |
| 1232 | GUGGUAUU A UGAGGUGA | 96 | TCACCTCA GGCTAGCTACAACGA AATACCAC | 3274 |
| 1295 | CAAGGAGU A CAACUAUG | 101 | CATAGTTG GGCTAGCTACAACGA ACTCCTTG | 3275 |
| 1301 | GUACAACU A UGACAAGA | 102 | TCTTGTCA GGCTAGCTACAACGA AGTTGTAC | 3276 |
| 1493 | CUCACUCU A CCUAAUGG | 130 | CCATTAGG GGCTAGCTACAACGA AGAGTGAG | 3277 |
| 1510 | GUGAGGUU A CCAACCAG | 133 | CTGGTTGG GGCTAGCTACAACGA AACCTCAC | 3278 |
| 1550 | GCAGCAAU A CCUGCGGC | 141 | GCCGCAGG GGCTAGCTACAACGA ATTGCTGC | 3279 |
| 1595 | CGACUGUU A CAAGUUUG | 144 | CAAACTTG GGCTAGCTACAACGA AACAGTCG | 3280 |
| 1633 | GCACUGUU A UGGGAGCU | 152 | AGCTCCCA GGCTAGCTACAACGA AACAGTGC | 3281 |
| 1645 | GAGCUGUU A UCAUGGAG | 154 | CTCCATGA GGCTAGCTACAACGA AACAGCTC | 3282 |
| 1661 | GGGCUUCU A CGUUGUCU | 158 | AGACAACG GGCTAGCTACAACGA AGAAGCCC | 3283 |
| 1787 | CUGUGGCU A CAACAUUC | 176 | GAATGTTG GGCTAGCTACAACGA AGCCACAG | 3284 |
| 1832 | CAUAGCCU A UGUCAUGG | 182 | CCATGACA GGCTAGCTACAACGA AGGCTATG | 3285 |
| 2141 | GGGACUGU A CCUGUAGG | 212 | CCTACAGG GGCTAGCTACAACGA ACAGTCCC | 3286 |
| 2191 | GCGGGAAU A CUCUUGGU | 215 | ACCAAGAG GGCTAGCTACAACGA ATTCCCGC | 3287 |
| 2290 | CCCAAAGU A UUCUUCUU | 240 | AAGAAGAA GGCTAGCTACAACGA ACTTTGGG | 3288 |
| 2316 | UCAGAAGU A CUGGCAUC | 254 | GATGCCAG GGCTAGCTACAACGA ACTTCTGA | 3289 |
| 2336 | CGCAGGUU A CCUUGGCG | 257 | CGCCAAGG GGCTAGCTACAACGA AACCTGCG | 3290 |
| 2359 | CCUGUGGU A CCCUGGCA | 260 | TGCCAGGG GGCTAGCTACAACGA ACCACAGG | 3291 |
| 2431 | AGUUUGCU A UUUGCUUU | 269 | AAAGCAAA GGCTAGCTACAACGA AGCAAACT | 3292 |
| 2455 | GGGACUGU A UAAACAAG | 275 | CTTGTTTA GGCTAGCTACAACGA ACAGTCCC | 3293 |
| 140 | AAGCCGCC A CCGGCCCG | 322 | CGGGCCGG GGCTAGCTACAACGA GGCGGCTT | 3294 |
| 151 | GGCCCGCC A UGCCCGCC | 327 | GGCGGGCA GGCTAGCTACAACGA GGCGGGCC | 3295 |
| 287 | CGCUCUCC A CAGCCCGG | 380 | CCGGGCTG GGCTAGCTACAACGA GGAGAGCG | 3296 |
| 368 | GAGAAGCC A CCAGCACC | 412 | GGTGCTGG GGCTAGCTACAACGA GGCTTCTC | 3297 |
| 374 | CCACCAGC A CCACCCAG | 415 | CTGGGTGG GGCTAGCTACAACGA GCTGGTGG | 3298 |
| 377 | CCAGCACC A CCCAGACU | 417 | AGTCTGGG GGCTAGCTACAACGA GGTGCTGG | 3299 |
| 451 | CGGGGCCC A CCAUGGCC | 435 | GGCCATGG GGCTAGCTACAACGA GGGCCCCG | 3300 |
| 454 | GGCCCACC A UGGCCCAA | 437 | TTGGGCCA GGCTAGCTACAACGA GGTGGGCC | 3301 |
| 512 | GCCUGCCC A CGGCACCC | 456 | GGGTGCCG GGCTAGCTACAACGA GGGCAGGC | 3302 |
| 517 | CCCACGGC A CCCAGCAC | 457 | GTGCTGGG GGCTAGCTACAACGA GCCGTGGG | 3303 |
| 524 | CACCCAGC A CGGCAUCC | 461 | GGATGCCG GGCTAGCTACAACGA GCTGGGTG | 3304 |
| 529 | AGCACGGC A UCCGGCUG | 462 | CAGCCGGA GGCTAGCTACAACGA GCCGTGCT | 3305 |
| 721 | CGCUCAAC A UCCUGGUG | 508 | CACCAGGA GGCTAGCTACAACGA GTTGAGCG | 3306 |
| 770 | UGCCCCCC A CCCCUUCC | 522 | GGAAGGGG GGCTAGCTACAACGA GGGGGGCA | 3307 |
| 782 | CUUCCUGC A UCGCUACU | 529 | AGTAGCGA GGCTAGCTACAACGA GCAGGAAG | 3308 |
| 811 | UGUCCAGC A CAUACCGG | 538 | CCGGTATG GGCTAGCTACAACGA GCTGGACA | 3309 |
| 813 | UCCAGCAC A UACCGGGA | 539 | TCCCGGTA GGCTAGCTACAACGA GTGCTGGA | 3310 |
| 850 | UGCCCUAC A CCCAGGGC | 547 | GCCCTGGG GGCTAGCTACAACGA GTAGGGCA | 3311 |
| 880 | AGCUGGGC A CCGACCUG | 553 | CAGGTCGG GGCTAGCTACAACGA GCCCAGCT | 3312 |
| 895 | UGGUAAGC A UCCCCCAU | 557 | ATGGGGGA GGCTAGCTACAACGA GCTTACCA | 3313 |
| 902 | CAUCCCCC A UGGCCCCA | 562 | TGGGGCCA GGCTAGCTACAACGA GGGGGATG | 3314 |
| 916 | CCAACGUC A CUGUGCGU | 567 | ACGCACAG GGCTAGCTACAACGA GACGTTGG | 3315 |
| 931 | GUGCCAAC A UUGCUGCC | 571 | GGCAGCAA GGCTAGCTACAACGA GTTGGCAC | 3316 |
| 940 | UUGCUGCC A UCACUGAA | 574 | TTCAGTGA GGCTAGCTACAACGA GGCAGCAA | 3317 |
| 943 | CUGCCAUC A CUGAAUCA | 575 | TGATTCAG GGCTAGCTACAACGA GATGGCAG | 3318 |
| 964 | AGUUCUUC A UCAACGGC | 580 | GCCGTTGA GGCTAGCTACAACGA GAAGAACT | 3319 |
| 988 | GGGAAGGC A UCCUGGGG | 586 | CCCCAGGA GGCTAGCTACAACGA GCCTTCCC | 3320 |
| 1070 | GCAGACCC A CGUUCCCA | 610 | TGGGAACG GGCTAGCTACAACGA GGGTCTGC | 3321 |
| 1156 | GAGGGAGC A UGAUCAUU | 638 | AATGATCA GGCTAGCTACAACGA GCTCCCTC | 3322 |
| 1162 | GCAUGAUC A UUGGAGGU | 639 | ACCTCCAA GGCTAGCTACAACGA GATCATGC | 3323 |
| 1178 | UAUCGACC A CUCGCUGU | 641 | ACAGCGAG GGCTAGCTACAACGA GGTCGATA | 3324 |
| 1189 | CGCUGUAC A CAGGCAGU | 644 | ACTGCCTG GGCTAGCTACAACGA GTACAGCG | 3325 |
| 1209 | UGGUAUAC A CCCAUCCG | 649 | CGGATGGG GGCTAGCTACAACGA GTATACCA | 3326 |
| 1213 | AUACACCC A UCCGGCGG | 652 | CCGCCGGA GGCTAGCTACAACGA GGGTGTAT | 3327 |
| 1243 | AGGUGAUC A UUGUGCGG | 654 | CCGCACAA GGCTAGCTACAACGA GATCACCT | 3328 |
| 1312 | ACAAGAGC A UUGUGGAC | 663 | GTCCACAA GGCTAGCTACAACGA GCTCTTGT | 3329 |
| 1327 | ACAGUGGC A CCACCAAC | 665 | GTTGGTGG GGCTAGCTACAACGA GCCACTGT | 3330 |
| 1330 | GUGGCACC A CCAACCUU | 667 | AAGGTTGG GGCTAGCTACAACGA GGTGCCAC | 3331 |
| 1378 | UCAAAUCC A UCAAGGCA | 679 | TGCCTTGA GGCTAGCTACAACGA GGATTTGA | 3332 |
| 1396 | CCUCCUCC A CGGAGAAG | 687 | CTTCTCCG GGCTAGCTACAACGA GGAGGAGG | 3333 |
| 1456 | AAGCAGGC A CCACCCCU | 698 | AGGGGTGG GGCTAGCTACAACGA GCCTGCTT | 3334 |
| 1459 | CAGGCACC A CCCCUUGG | 700 | CCAAGGGG GGCTAGCTACAACGA GGTGCCTG | 3335 |
| 1471 | CUUGGAAC A UUUUCCCA | 705 | TGGGAAAA GGCTAGCTACAACGA GTTCCAAG | 3336 |
| 1483 | UCCCAGUC A UCUCACUC | 709 | GAGTGAGA GGCTAGCTACAACGA GACTGGGA | 3337 |
| 1488 | GUCAUCUC A CUCUACCU | 711 | AGGTAGAG GGCTAGCTACAACGA GAGATGAC | 3338 |
| 1528 | CCUUCCGC A UCACCAUC | 723 | GATGGTGA GGCTAGCTACAACGA GCGGAAGG | 3339 |
| 1531 | UCCGCAUC A CCAUCCUU | 724 | AAGGATGG GGCTAGCTACAACGA GATGCGGA | 3340 |
| 1534 | GCAUCACC A UCCUUCCG | 726 | CGGAAGGA GGCTAGCTACAACGA GGTGATGC | 3341 |
| 1576 | AUGUGGCC A CGUCCCAA | 737 | TTGGGACG GGCTAGCTACAACGA GGCCACAT | 3342 |
| 1606 | AGUUUGCC A UCUCACAG | 744 | CTGTGAGA GGCTAGCTACAACGA GGCAAACT | 3343 |
| 1611 | GCCAUCUC A CAGUCAUC | 746 | GATGACTG GGCTAGCTACAACGA GAGATGGC | 3344 |
| 1617 | UCACAGUC A UCCACGGG | 748 | CCCGTGGA GGCTAGCTACAACGA GACTGTGA | 3345 |
| 1621 | AGUCAUCC A CGGGCACU | 750 | AGTGCCCG GGCTAGCTACAACGA GGATGACT | 3346 |
| 1627 | CCACGGGC A CUGUUAUG | 751 | CATAACAG GGCTAGCTACAACGA GCCCGTGG | 3347 |
| 1648 | CUGUUAUC A UGGAGGGC | 754 | GCCCTCCA GGCTAGCTACAACGA GATAACAG | 3348 |
| 1715 | CGCUUGCC A UGUGCACG | 765 | CGTGCACA GGCTAGCTACAACGA GGCAAGCG | 3349 |
| 1721 | CCAUGUGC A CGAUGAGU | 766 | ACTCATCG GGCTAGCTACAACGA GCACATGG | 3350 |
| 1762 | CUUUUGUC A CCUUGGAC | 772 | GTCCAAGG GGCTAGCTACAACGA GACAAAAG | 3351 |
| 1771 | CCUUGGAC A UGGAAGAC | 775 | GTCTTCCA GGCTAGCTACAACGA GTCCAAGG | 3352 |
| 1792 | GCUACAAC A UUCCACAG | 779 | CTGTGGAA GGCTAGCTACAACGA GTTGTAGC | 3353 |
| 1797 | AACAUUCC A CAGACAGA | 781 | TCTGTCTG GGCTAGCTACAACGA GGAATGTT | 3354 |
| 1819 | CAACCCUC A UGACCAUA | 788 | TATGGTCA GGCTAGCTACAACGA GAGGGTTG | 3355 |
| 1825 | UCAUGACC A UAGCCUAU | 790 | ATAGGCTA GGCTAGCTACAACGA GGTCATGA | 3356 |
| 1837 | CCUAUGUC A UGGCUGCC | 793 | GGCAGCCA GGCTAGCTACAACGA GACATAGG | 3357 |
| 1846 | UGGCUGCC A UCUGCGCC | 796 | GGCGCAGA GGCTAGCTACAACGA GGCAGCCA | 3358 |
| 1861 | CCCUCUUC A UGCUGCCA | 802 | TGGCAGCA GGCTAGCTACAACGA GAAGAGGG | 3359 |
| 1869 | AUGCUGCC A CUCUGCCU | 805 | AGGCAGAG GGCTAGCTACAACGA GGCAGCAT | 3360 |
| 1879 | UCUGCCUC A UGGUGUGU | 810 | ACACACCA GGCTAGCTACAACGA GAGGCAGA | 3361 |
| 1922 | CCAGCAGC A UGAUGACU | 822 | AGTCATCA GGCTAGCTACAACGA GCTGCTGG | 3362 |
| 1942 | CUGAUGAC A UCUCCCUG | 825 | CAGGGAGA GGCTAGCTACAACGA GTCATCAG | 3363 |
| 1968 | GGAGGCCC A UGGGCAGA | 833 | TCTGCCCA GGCTAGCTACAACGA GGGCCTCC | 3364 |
| 1998 | CCUGGACC A CACCUCCG | 840 | CGGAGGTG GGCTAGCTACAACGA GGTCCAGG | 3365 |
| 2000 | UGGACCAC A CCUCCGUG | 841 | CACGGAGG GGCTAGCTACAACGA GTGGTCCA | 3366 |
| 2013 | CGUGGUUC A CUUUGGUC | 845 | GACCAAAG GGCTAGCTACAACGA GAACCACG | 3367 |
| 2022 | CUUUGGUC A CAAGUAGG | 847 | CCTACTTG GGCTAGCTACAACGA GACCAAAG | 3368 |
| 2035 | UAGGAGAC A CAGAUGGC | 849 | GCCATCTG GGCTAGCTACAACGA GTCTCCTA | 3369 |
| 2044 | CAGAUGGC A CCUGUGGC | 851 | GCCACAGG GGCTAGCTACAACGA GCCATCTG | 3370 |
| 2059 | GCCACAGC A CCUCAGGA | 856 | TCCTGAGG GGCTAGCTACAACGA GCTCTGGC | 3371 |
| 2076 | CCCUCCCC A CCCACCAA | 866 | TTGGTGGG GGCTAGCTACAACGA GGGGAGGG | 3372 |
| 2080 | CCCCACCC A CCAAAUGC | 869 | GCATTTGG GGCTAGCTACAACGA GGGTGGGG | 3373 |
| 2174 | AAAGAAGC A CUCUGCUG | 885 | CACCAGAG GGCTAGCTACAACGA GCTTCTTT | 3374 |
| 2201 | UCUUGGUC A CCUCAAAU | 891 | ATTTGAGG GGCTAGCTACAACGA GACCAAGA | 3375 |
| 2260 | CUUUGUCC A CCAUUCCU | 906 | AGGAATGG GGCTAGCTACAACGA GGACAAAG | 3376 |
| 2263 | UGUCCACC A UUCCUUUA | 908 | TAAAGGAA GGCTAGCTACAACGA GGTGGACA | 3377 |
| 2322 | GUACUGGC A UCACACGC | 922 | GCGTGTGA GGCTAGCTACAACGA GCCAGTAC | 3378 |
| 2325 | CUGGCAUC A CACGCAGG | 923 | CCTGCGTG GGCTAGCTACAACGA GATGCCAG | 3379 |
| 2327 | GGCAUCAC A CGCAGGUU | 924 | AACCTGCG GGCTAGCTACAACGA GTGATGCC | 3380 |
| 2421 | GAGGAUGC A CAGUUUGC | 945 | GCAAACTG GGCTAGCTACAACGA GCATCCTC | 3381 |
| 2470 | AGCCUAAC A UUGGUGCA | 954 | TGCACCAA GGCTAGCTACAACGA GTTAGGCT | 3382 |
| 11 | ACGCGUCC G CAGCCCGC | 960 | GCGGGCTG GGCTAGCTACAACGA GGACGCGT | 3383 |
| 18 | CGCAGCCC G CCCGGGAG | 961 | CTCCCGGG GGCTAGCTACAACGA GGGCTGCG | 3384 |
| 29 | CGGGAGCU G CGAGCCGC | 962 | GCGGCTCG GGCTAGCTACAACGA AGCTCCCG | 3385 |
| 36 | UGCGAGCC G CGAGCUGG | 964 | CCAGCTCG GGCTAGCTACAACGA GGCTCGCA | 3386 |
| 69 | CAGCCAAC G CAGCCGCA | 967 | TGCGGCTG GGCTAGCTACAACGA GTTGGCTG | 3387 |
| 75 | ACGCAGCC G CAGGAGCC | 968 | GGCTCCTG GGCTAGCTACAACGA GGCTGCGT | 3388 |
| 94 | GAGCCCUU G CCCCUGCC | 969 | GGCAGGGG GGCTAGCTACAACGA AAGGGCTC | 3389 |
| 100 | UUGCCCCU G CCCGCGCC | 970 | GGCGCGGG GGCTAGCTACAACGA AGGGGCAA | 3390 |
| 104 | CCCUGCCC G CGCCGCCG | 971 | CGGCGGCG GGCTAGCTACAACGA GGGCAGGG | 3391 |
| 106 | CUGCCCGC G CCGCCGCC | 972 | GGCGGCGG GGCTAGCTACAACGA GCGGGCAG | 3392 |
| 109 | CCCGCGCC G CCGCCCGC | 973 | GCGGGCGG GGCTAGCTACAACGA GGCGCGGG | 3393 |
| 112 | GCGCCGCC G CCCGCCGG | 974 | CCGGCGGG GGCTAGCTACAACGA GGCGGCGC | 3394 |
| 116 | CGCCGCCC G CCGGGGGG | 975 | CCCCCCGG GGCTAGCTACAACGA GGGCGGCG | 3395 |
| 137 | GGGAAGCC G CCACCGGC | 976 | GCCGGTGG GGCTAGCTACAACGA GGCTTCCC | 3396 |
| 148 | ACCGGCCC G CCAUGCCC | 977 | GGGCATGG GGCTAGCTACAACGA GGGCCGGT | 3397 |
| 153 | CCCGCCAU G CCCGCCCC | 978 | GGGGCGGG GGCTAGCTACAACGA ATGGCGGG | 3398 |
| 157 | CCAUGCCC G CCCCUCCC | 979 | GGGAGGGG GGCTAGCTACAACGA GGGCATGG | 3399 |
| 172 | CCAGCCCC G CCGGGAGC | 980 | GCTCCCGG GGCTAGCTACAACGA GGGGCTGG | 3400 |
| 183 | GGGAGCCC G CGCCCGCU | 981 | AGCGGGCG GGCTAGCTACAACGA GGGCTCCC | 3401 |
| 185 | GAGCCCGC G CCCGCUGC | 982 | GCAGCGGG GGCTAGCTACAACGA GCGGGCTC | 3402 |
| 189 | CCGCGCCC G CUGCCCAG | 983 | CTGGGCAG GGCTAGCTACAACGA GGGCGCGG | 3403 |
| 192 | CGCCCGCU G CCCAGGCU | 984 | AGCCTGGG GGCTAGCTACAACGA AGCGGGCG | 3404 |
| 205 | GGCUGGCC G CCGCCGUG | 985 | CACGGCGG GGCTAGCTACAACGA GGCCAGCC | 3405 |
| 208 | UGGCCGCC G CCGUGCCG | 986 | CGGCACGG GGCTAGCTACAACGA GGCGGCCA | 3406 |
| 213 | GCCGCCGU G CCGAUGUA | 987 | TACATCGG GGCTAGCTACAACGA ACGGCGGC | 3407 |
| 250 | UCUCCCCU G CUCCCGUG | 989 | CACGGGAG GGCTAGCTACAACGA AGGGGAGA | 3408 |
| 258 | GCUCCCGU G CUCUGCGG | 990 | CCGCAGAG GGCTAGCTACAACGA ACGGGAGC | 3409 |
| 263 | CGUGCUCU G CGGAUCUC | 991 | GAGATCCG GGCTAGCTACAACGA AGAGCACG | 3410 |
| 280 | CCCUGACC G CUCUCCAC | 993 | GTGGAGAG GGCTAGCTACAACGA GGTCAGGG | 3411 |
| 320 | AGGGCCCU G CAGGCCCU | 994 | AGGGCCTG GGCTAGCTACAACGA AGGGCCCT | 3412 |
| 340 | GUCCUGAU G CCCCCAAG | 996 | CTTGGGGG GGCTAGCTACAACGA ATCAGGAC | 3413 |
| 397 | GGGCAGGC G CCAGGGAC | 998 | GTCCCTGG GGCTAGCTACAACGA GCCTGCCC | 3414 |
| 420 | GGGCCAGU G CGAGCCCA | 999 | TGGGCTCG GGCTAGCTACAACGA ACTGGCCC | 3415 |
| 468 | CAAGCCCU G CCCUGGCU | 1002 | AGCCAGGG GGCTAGCTACAACGA AGGGCTTG | 3416 |
| 480 | UGGCUCCU G CUGUGGAU | 1003 | ATCCACAG GGCTAGCTACAACGA AGGAGCCA | 3417 |
| 493 | GGAUGGGC G CGGGAGUG | 1004 | CACTCCCG GGCTAGCTACAACGA GCCCATCC | 3418 |
| 501 | GCGGGAGU G CUGCCUGC | 1005 | GCAGGCAG GGCTAGCTACAACGA ACTCCCGC | 3419 |
| 504 | GGAGUGCU G CCUGCCCA | 1006 | TGGGCAGG GGCTAGCTACAACGA AGCACTCC | 3420 |
| 508 | UGCUGCCU G CCCACGGC | 1007 | GCCGTGGG GGCTAGCTACAACGA AGGCAGCA | 3421 |
| 537 | AUCCGGCU G CCCCUGCG | 1008 | CGCAGGGG GGCTAGCTACAACGA AGCCGGAT | 3422 |
| 543 | CUGCCCCU G CGCAGCGG | 1009 | CCGCTGCG GGCTAGCTACAACGA AGGGGCAG | 3423 |
| 545 | GCCCCUGC G CAGCGGCC | 1010 | GGCCGCTG GGCTAGCTACAACGA GCAGGGGC | 3424 |
| 562 | UGGGGGGC G CCCCCCUG | 1011 | CAGGGGGG GGCTAGCTACAACGA GCCCCCCA | 3425 |
| 576 | CUGGGGCU G CGGCUGCC | 1012 | GGCAGCCG GGCTAGCTACAACGA AGCCCCAG | 3426 |
| 582 | CUGCGGCU G CCCCGGGA | 1013 | TCCCGGGG GGCTAGCTACAACGA AGCCGCAG | 3427 |
| 708 | AGCCCCCC G CAGACGCU | 1019 | AGCGTCTG GGCTAGCTACAACGA GGGGGGCT | 3428 |
| 714 | CCGCAGAC G CUCAACAU | 1020 | ATGTTGAG GGCTAGCTACAACGA GTCTGCGG | 3429 |
| 751 | GUAACUUU G CAGUGGGU | 1021 | ACCCACTG GGCTAGCTACAACGA AAAGTTAC | 3430 |
| 760 | CAGUGGGU G CUGCCCCC | 1022 | GGGGGCAG GGCTAGCTACAACGA ACCCACTG | 3431 |
| 763 | UGGGUGCU G CCCCCCAC | 1023 | GTGGGGGG GGCTAGCTACAACGA AGCACCCA | 3432 |
| 780 | CCCUUCCU G CAUCGCUA | 1024 | TAGCGATG GGCTAGCTACAACGA AGGAAGGG | 3433 |
| 785 | CCUGCAUC G CUACUACC | 1025 | GGTAGTAG GGCTAGCTACAACGA GATGCAGG | 3434 |
| 843 | GUGUAUGU G CCCUACAC | 1026 | GTGTAGGG GGCTAGCTACAACGA ACATACAC | 3435 |
| 921 | GUCACUGU G CGUGCCAA | 1028 | TTGGCACG GGCTAGCTACAACGA ACAGTGAC | 3436 |
| 925 | CUGUGCGU G CCAACAUU | 1029 | AATGTTGG GGCTAGCTACAACGA ACGCACAG | 3437 |
| 934 | CCAACAUU G CUGCCAUC | 1030 | GATGGCAG GGCTAGCTACAACGA AATGTTGG | 3438 |
| 937 | ACAUUGCU G CCAUCACU | 1031 | AGTGATGG GGCTAGCTACAACGA AGCAATGT | 3439 |
| 1006 | UGGCCUAU G CUGAGAUU | 1033 | AATCTCAG GGCTAGCTACAACGA ATAGGCCA | 3440 |
| 1015 | CUGAGAUU G CCAGGCCU | 1035 | AGGCCTGG GGCTAGCTACAACGA AATCTCAG | 3441 |
| 1092 | UUCUCCCU G CAGCUUUG | 1039 | CAAAGCTG GGCTAGCTACAACGA AGGCAGAA | 3442 |
| 1105 | UUUGUGGU G CUGGCUUC | 1040 | GAAGCCAG GGCTAGCTACAACGA ACCACAAA | 3443 |
| 1134 | UCUGAAGU G CUGGCCUC | 1042 | GAGGCCAG GGCTAGCTACAACGA ACTTCAGA | 3444 |
| 1182 | GACCACUC G CUGUACAC | 1045 | GTGTACAG GGCTAGCTACAACGA GAGTGGTC | 3445 |
| 1248 | AUCAUUGU G CGGGUGGA | 1048 | TCCACCCG GGCTAGCTACAACGA ACAATGAT | 3446 |
| 1286 | AAUGGACU G CAAGGAGU | 1050 | ACTCCTTG GGCTAGCTACAACGA AGTCCATT | 3447 |
| 1344 | CUUCGUUU G CCCAAGAA | 1052 | TTCTTGGG GGCTAGCTACAACGA AAACGAAG | 3448 |
| 1366 | UUGAAGCU G CAGUCAAA | 1054 | TTTGACTG GGCTAGCTACAACGA AGCTTCAA | 3449 |
| 1442 | GCUGGUGU G CUGGCAAG | 1056 | CTTGCCAG GGCTAGCTACAACGA ACACCAGC | 3450 |
| 1526 | GUCCUUCC G CAUCACCA | 1058 | TGGTGATG GGCTAGCTACAACGA GGAAGGAC | 3451 |
| 1542 | AUCCUUCC G CAGCAAUA | 1059 | TATTGCTG GGCTAGCTACAACGA GGAAGGAT | 3452 |
| 1554 | CAAUACCU G CGGCCAGU | 1060 | ACTGGCCG GGCTAGCTACAACGA AGGTATTG | 3453 |
| 1603 | ACAAGUUU G CCAUCUCA | 1062 | TGAGATGG GGCTAGCTACAACGA AAACTTGT | 3454 |
| 1699 | UUGGCUUU G CUGUCAGC | 1066 | GCTGACAG GGCTAGCTACAACGA AAAGCCAA | 3455 |
| 1708 | CUGUCAGC G CUUGCCAU | 1067 | ATGGCAAG GGCTAGCTACAACGA GCTGACAG | 3456 |
| 1712 | CAGCGCUU G CCAUGUGC | 1068 | GCACATGG GGCTAGCTACAACGA AAGCGCTG | 3457 |
| 1719 | UGCCAUGU G CACGAUGA | 1069 | TCATCGTG GGCTAGCTACAACGA ACATGGCA | 3458 |
| 1843 | UCAUGGCU G CCAUCUGU | 1074 | GCAGATGG GGCTAGCTACAACGA AGCCATGA | 3459 |
| 1850 | UGCCAUCU G CGCCCUCU | 1075 | AGAGGGCG GGCTAGCTACAACGA AGATGGCA | 3460 |
| 1852 | CCAUCUGC G CCCUCUUC | 1076 | GAAGAGGG GGCTAGCTACAACGA GCAGATGG | 3461 |
| 1863 | CUCUUCAU G CUGCCACU | 1077 | AGTGGCAG GGCTAGCTACAACGA ATGAAGAG | 3462 |
| 1866 | UUCAUGCU G CCACUCUG | 1078 | CAGAGTGG GGCTAGCTACAACGA AGCATGAA | 3463 |
| 1874 | GCCACUCU G CCUCAUGG | 1079 | CCATGAGG GGCTAGCTACAACGA AGAGTGGC | 3464 |
| 1895 | UCAGUGGC G CUGCCUCC | 1080 | GGAGGCAG GGCTAGCTACAACGA GCCACTGA | 3465 |
| 1898 | GUGGCGCU G CCUCCGCU | 1081 | AGCGGAGG GGCTAGCTACAACGA AGCGCCAC | 3466 |
| 1904 | CUGCCUCC G CUGCCUGC | 1082 | GCAGGCAG GGCTAGCTACAACGA GGAGGCAG | 3467 |
| 1907 | CCUCCGCU G CCUGCGCC | 1083 | GGCGCAGG GGCTAGCTACAACGA AGCGGAGG | 3468 |
| 1911 | CGCUGCCU G CGCCAGCA | 1084 | TGCTGGCG GGCTAGCTACAACGA AGGCAGCG | 3469 |
| 1913 | CUGCCUGC G CCAGCAGC | 1085 | GCTGCTGG GGCTAGCTACAACGA GCAGGCAG | 3470 |
| 1933 | AUGACUUU G CUGAUGAC | 1088 | GTCATCAG GGCTAGCTACAACGA AAAGTCAT | 3471 |
| 1950 | AUCUCCCU G CUGAAGUG | 1091 | CACTTCAG GGCTAGCTACAACGA AGGGAGAT | 3472 |
| 2087 | CACCAAAU G CCUCUGCC | 1094 | GGCAGAGG GGCTAGCTACAACGA ATTTGGTG | 3473 |
| 2093 | AUGCCUCU G CCUUGAUG | 1095 | CATCAAGG GGCTAGCTACAACGA AGAGGCAT | 3474 |
| 2179 | AGCACUCU G CUGGCGGG | 1097 | CCCGCCAG GGCTAGCTACAACGA AGAGTGCT | 3475 |
| 2227 | GAAAUUCU G CUGCUUGA | 1098 | TCAAGCAG GGCTAGCTACAACGA AGAATTTC | 3476 |
| 2230 | AUUCUGCU G CUUGAAAC | 1099 | GTTTCAAG GGCTAGCTACAACGA AGCAGAAT | 3477 |
| 2329 | CAUCACAC G CAGGUUAC | 1102 | GTAACCTG GGCTAGCTACAACGA GTGTGATG | 3478 |
| 2393 | GUUUCCCU G CUGGCCAA | 1103 | TTGGCCAG GGCTAGCTACAACGA AGGGAAAC | 3479 |
| 2419 | GAGAGGAU G CACAGUUU | 1104 | AAACTGTG GGCTAGCTACAACGA ATCCTCTC | 3480 |
| 2428 | CACAGUUU G CUAUUUGC | 1105 | GCAAATAG GGCTAGCTACAACGA AAACTGTG | 3481 |
| 2435 | UGCUAUUU G CUUUAGAG | 1106 | CTCTAAAG GGCTAGCTACAACGA AAATAGCA | 3482 |
| 2476 | ACAUUGGU G CAAAGAUU | 1107 | AATCTTTG GGCTAGCTACAACGA ACCAATGT | 3483 |
| 2485 | CAAAGAUU G CCUCUUGA | 1108 | TCAAGAGG GGCTAGCTACAACGA AATCTTTG | 3484 |
| 219 | GUGCCGAU G UAGCGGGC | 1110 | GCCCGCTA GGCTAGCTACAACGA ATCGGCAC | 3485 |
| 483 | CUCCUGCU G UGGAUGGG | 1111 | CCCATCCA GGCTAGCTACAACGA AGCAGGAG | 3486 |
| 634 | GCAGCUUU G UGGAGAUG | 1112 | CATCTCCA GGCTAGCTACAACGA AAAGCTGC | 3487 |
| 804 | AGGCAGCU G UCCAGCAC | 1113 | GTGCTGGA GGCTAGCTACAACGA AGCTGCCT | 3488 |
| 835 | GGAAGGGU G UGUAUGUG | 1114 | CACATACA GGCTAGCTACAACGA ACCCTTCC | 3489 |
| 837 | AAGGGUGU G UAUGUGCC | 1115 | GGCACATA GGCTAGCTACAACGA ACACCCTT | 3490 |
| 841 | GUGUGUAU G UGCCCUAC | 1116 | GTAGGGCA GGCTAGCTACAACGA ATACACAC | 3491 |
| 919 | ACGUCACU G UGCGUGCC | 1117 | GGCACGCA GGCTAGCTACAACGA AGTCACGT | 3492 |
| 1100 | GCAGCUUU G UGGUGCUG | 1118 | CAGCACCA GGCTAGCTACAACGA AAAGCTGC | 3493 |
| 1144 | UGGCCUCU G UCGGAGGG | 1119 | CCCTCCGA GGCTAGCTACAACGA AGAGGCCA | 3494 |
| 1185 | CACUCGCU G UACACAGG | 1120 | CCTGTGTA GGCTAGCTACAACGA AGCGAGTG | 3495 |
| 1246 | UGAUCAUU G UGCGGGUG | 1121 | CACCCGCA GGCTAGCTACAACGA AATGATCA | 3496 |
| 1315 | AGAGCAUU G UGGACAGU | 1122 | ACTGTCCA GGCTAGCTACAACGA AATGCTCT | 3497 |
| 1356 | AAGAAAGU G UUUGAAGC | 1123 | GCTTCAAA GGCTAGCTACAACGA ACTTTCTT | 3498 |
| 1440 | CAGCUGGU G UGCUGGCA | 1124 | TGCCAGCA GGCTAGCTACAACGA ACCAGCTG | 3499 |
| 1570 | UGGAAGAU G UGGCCACG | 1125 | CGTGGCCA GGCTAGCTACAACGA ATCTTCCA | 3500 |
| 1592 | AGACGACU G UUACAAGU | 1126 | ACTTGTAA GGCTAGCTACAACGA AGTCGTCT | 3501 |
| 1630 | CGGGCACU G UUAUGGGA | 1127 | TCCCATAA GGCTAGCTACAACGA AGTGCCCG | 3502 |
| 1642 | UGGGAGCU G UUAUCAUG | 1128 | CATGATAA GGCTAGCTACAACGA AGCTCCCA | 3503 |
| 1666 | UCUACGUU G UCUUUGAU | 1129 | ATCAAAGA GGCTAGCTACAACGA AACGTAGA | 3504 |
| 1702 | GCUUUGCU G UCAGCGCU | 1130 | AGCGCTGA GGCTAGCTACAACGA AGCAAAGC | 3505 |
| 1717 | CUUGCCAU G UGCACGAU | 1131 | ATCGTGCA GGCTAGCTACAACGA ATGGCAAG | 3506 |
| 1759 | GCCCUUUU G UCACCUUG | 1132 | CAAGGTGA GGCTAGCTACAACGA AAAAGGGC | 3507 |
| 1781 | GGAAGACU G UGGCUACA | 1133 | TGTAGCCA GGCTAGCTACAACGA AGTCTTCC | 3508 |
| 1834 | UAGCCUAU G UCAUGGCU | 1134 | AGCCATGA GGCTAGCTACAACGA ATAGGCTA | 3509 |
| 1884 | CUCAUGGU G UGUCAGUG | 1135 | CACTGACA GGCTAGCTACAACGA ACCATGAG | 3510 |
| 1886 | CAUGGUGU G UCAGUGGC | 1136 | GCCACTGA GGCTAGCTACAACGA ACACCATG | 3511 |
| 2048 | UGGCACCU G UGGCCAGA | 1137 | TCTGGCCA GGCTAGCTACAACGA AGGTGCCA | 3512 |
| 2139 | CAGGGACU G UACCUGUA | 1138 | TACAGGTA GGCTAGCTACAACGA AGTCCCTG | 3513 |
| 2145 | CUGUACCU G UAGGAAAC | 1139 | GTTTCCTA GGCTAGCTACAACGA AGGTACAG | 3514 |
| 2256 | GAACCUUU G UCCACCAU | 1140 | ATGGTGGA GGCTAGCTACAACGA AAAGGTTC | 3515 |
| 2346 | CUUGGCGU G UGUCCCUG | 1141 | CAGGGACA GGCTAGCTACAACGA ACGCCAAG | 3516 |
| 2348 | UGGCGUGU G UCCCUGUG | 1142 | CACAGGGA GGCTAGCTACAACGA ACACGCCA | 3517 |
| 2354 | GUGUCCCU G UGGUACCC | 1143 | GGGTACCA GGCTAGCTACAACGA AGGGACAC | 3518 |
| 2385 | CCAAGCUU G UUUCCCUG | 1144 | CAGGGAAA GGCTAGCTACAACGA AAGCTTGG | 3519 |
| 2453 | CAGGGACU G UAUAAACA | 1145 | TGTTTATA GGCTAGCTACAACGA AGTCCCTG | 3520 |
| 14 | CGUCCGCA G CCCGCCCG | 1146 | CGGGCGGG GGCTAGCTACAACGA TGCGGACG | 3521 |
| 26 | GCCCGGGA G CUGCGAGC | 1147 | GCTCGCAG GGCTAGCTACAACGA TCCCGGGC | 3522 |
| 33 | AGCUGCGA G CCGCGAGC | 1148 | GCTCGCGG GGCTAGCTACAACGA TCGCAGCT | 3523 |
| 40 | AGCCGCGA G CUGGAUUA | 1149 | TAATCCAG GGCTAGCTACAACGA TCGCGGCT | 3524 |
| 51 | GGAUUAUG G UGGCCUGA | 1150 | TCAGGCCA GGCTAGCTACAACGA CATAATCC | 3525 |
| 54 | UUAUGGUG G CCUGAGCA | 1151 | TGCTCAGG GGCTAGCTACAACGA CACCATAA | 3526 |
| 60 | UGGCCUGA G CAGCCAAC | 1152 | GTTGGCTG GGCTAGCTACAACGA TCAGGCCA | 3527 |
| 63 | CCUGAGCA G CCAACGCA | 1153 | TGCGTTGG GGCTAGCTACAACGA TGCTCAGG | 3528 |
| 72 | CCAACGCA G CCGCAGGA | 1154 | TCCTGCGG GGCTAGCTACAACGA TGCGTTGG | 3529 |
| 81 | CCGCAGGA G CCCGGAGC | 1155 | GCTCCGGG GGCTAGCTACAACGA TCCTGCGG | 3530 |
| 88 | AGCCCGGA G CCCUUGCC | 1156 | GGCAAGGG GGCTAGCTACAACGA TCCGGGCT | 3531 |
| 134 | CCAGGGAA G CCGCCACC | 1157 | GGTGGCGG GGCTAGCTACAACGA TTCCCTGG | 3532 |
| 144 | CGCCACCG G CCCGCCAU | 1158 | ATGGCGGG GGCTAGCTACAACGA CGGTGGCG | 3533 |
| 167 | CCCUCCCA G CCCCGCCG | 1159 | CGGCGGGG GGCTAGCTACAACGA TGGGAGGG | 3534 |
| 179 | CGCCGGGA G CCCGCGCC | 1160 | GGCGCGGG GGCTAGCTACAACGA TCCCGGCG | 3535 |
| 198 | CUGCCCAG G CUGGCCGC | 1161 | GCGGCCAG GGCTAGCTACAACGA CTGGGCAG | 3536 |
| 202 | CCAGGCUG G CCGCCGCC | 1162 | GGCGGCGG GGCTAGCTACAACGA CAGCCTGG | 3537 |
| 211 | CCGCCGCC G UGCCGAUG | 1163 | CATCGGCA GGCTAGCTACAACGA GGCGGCGG | 3538 |
| 222 | CCGAUGUA G CGGGCUCC | 1164 | GGAGCCCG GGCTAGCTACAACGA TACATCGG | 3539 |
| 226 | UGUAGCGG G CUCCGGAU | 1165 | ATCCGGAG GGCTAGCTACAACGA CCGCTACA | 3540 |
| 239 | GGAUCCCA G CCUCUCCC | 1166 | GGGAGAGG GGCTAGCTACAACGA TGGGATCC | 3541 |
| 256 | CUGCUCCC G UGCUCUGC | 1167 | GCAGAGCA GGCTAGCTACAACGA GGGAGCAG | 3542 |
| 290 | UCUCCACA G CCCGGACC | 1168 | GGTCCGGG GGCTAGCTACAACGA TGTGGAGA | 3543 |
| 304 | ACCCGGGG G CUGGCCCA | 1169 | TGGGCCAG GGCTAGCTACAACGA CCCCGGGT | 3544 |
| 308 | GGGGGCUG G CCCAGGGC | 1170 | GCCCTGGG GGCTAGCTACAACGA CAGCCCCC | 3545 |
| 315 | GGCCCAGG G CCCUGCAG | 1171 | CTGCAGGG GGCTAGCTACAACGA CCTGGGCC | 3546 |
| 324 | CCCUGCAG G CCCUGGCG | 1172 | CGCCAGGG GGCTAGCTACAACGA CTGCAGGG | 3547 |
| 330 | AGGCCCUG G CGUCCUGA | 1173 | TCAGGACG GGCTAGCTACAACGA CAGGGCCT | 3548 |
| 332 | GCCCUGGC G UCCUGAUG | 1174 | CATCAGGA GGCTAGCTACAACGA GCCAGGGC | 3549 |
| 348 | GCCCCCAA G CUCCCUCU | 1175 | AGAGGGAG GGCTAGCTACAACGA TTGGGGGC | 3550 |
| 365 | CCUGAGAA G CCACCAGC | 1176 | GCTGGTGG GGCTAGCTACAACGA TTCTCAGG | 3551 |
| 372 | AGCCACCA G CACCACCC | 1177 | GGGTGGTG GGCTAGCTACAACGA TGGTGGCT | 3552 |
| 391 | ACUUGGGG G CAGGCGCC | 1178 | GGCGCCTG GGCTAGCTACAACGA CCCCAAGT | 3553 |
| 395 | GGGGGCAG G CGCCAGGG | 1179 | CCCTGGCG GGCTAGCTACAACGA CTGCCCCC | 3554 |
| 410 | GGACGGAC G UGGGCCAG | 1180 | CTGGCCCA GGCTAGCTACAACGA GTCCGTCC | 3555 |
| 414 | GGACGUGG G CCAGUGCG | 1181 | CGCACTGG GGCTAGCTACAACGA CCACGTCC | 3556 |
| 418 | GUGGGCCA G UGCGAGCC | 1182 | GGCTCGCA GGCTAGCTACAACGA TGGCCCAC | 3557 |
| 424 | CAGUGCGA G CCCAGAGG | 1183 | CCTCTGGG GGCTAGCTACAACGA TCGCACTG | 3558 |
| 433 | CCCAGAGG G CCCGAAGG | 1184 | CCTTCGGG GGCTAGCTACAACGA CCTCTGGG | 3559 |
| 441 | GCCCGAAG G CCGGGGCC | 1185 | GGCCCCGG GGCTAGCTACAACGA CTTCGGGC | 3560 |
| 447 | AGGCCGGG G CCCACCAU | 1186 | ATGGTGGG GGCTAGCTACAACGA CCCGGCCT | 3561 |
| 457 | CCACCAUG G CCCAAGCC | 1187 | GGCTTGGG GGCTAGCTACAACGA CATGGTGG | 3562 |
| 463 | UGGCCCAA G CCCUGCCC | 1188 | GGGCAGGG GGCTAGCTACAACGA TTGGGCCA | 3563 |
| 474 | CUGCCCUG G CUCCUGCU | 1189 | AGCAGGAG GGCTAGCTACAACGA CAGGGCAG | 3564 |
| 491 | GUGGAUGG G CGCGGGAG | 1190 | CTCCCGCG GGCTAGCTACAACGA CCATCCAC | 3565 |
| 499 | GCGCGGGA G UGCUGCCU | 1191 | AGGCAGCA GGCTAGCTACAACGA TCCCGCGC | 3566 |
| 515 | UGCCCACG G CACCCAGC | 1192 | GCTGGGTG GGCTAGCTACAACGA CGTGGGCA | 3567 |
| 522 | GGCACCCA G CACGGCAU | 1193 | ATGCCGTG GGCTAGCTACAACGA TGGGTGCC | 3568 |
| 527 | CCAGCACG G CAUCCGGC | 1194 | GCCGGATG GGCTAGCTACAACGA CGTGCTGG | 3569 |
| 534 | GGCAUCCG G CUGCCCCU | 1195 | AGGGGCAG GGCTAGCTACAACGA CGGATGCC | 3570 |
| 548 | CCUGCGCA G CGGCCUGG | 1196 | CCAGGCCG GGCTAGCTACAACGA TGCGCAGG | 3571 |
| 551 | GCGCAGCG G CCUGGGGG | 1197 | CCCCCAGG GGCTAGCTACAACGA CGCTGCGC | 3572 |
| 560 | CCUGGGGG G CGCCCCCC | 1198 | GGGGGGCG GGCTAGCTACAACGA CCCCCAGG | 3573 |
| 573 | CCCCUGGG G CUGCGGCU | 1199 | AGCCGCAG GGCTAGCTACAACGA CCCAGGGG | 3574 |
| 579 | GGGCUGCG G CUGCCCCG | 1200 | CGGGGCAG GGCTAGCTACAACGA CGCAGCCC | 3575 |
| 603 | GACGAAGA G CCCGAGGA | 1201 | TCCTCGGG GGCTAGCTACAACGA TCTTCGTC | 3576 |
| 612 | CCCGAGGA G CCCGGCCG | 1202 | CGGCCGGG GGCTAGCTACAACGA TCCTCGGG | 3577 |
| 617 | GGAGCCCG G CCGGAGGG | 1203 | CCCTCCGG GGCTAGCTACAACGA CGGGCTCC | 3578 |
| 626 | CCGGAGGG G CAGCUUUG | 1204 | CAAAGCTG GGCTAGCTACAACGA CCCTCCGG | 3579 |
| 629 | GAGGGGCA G CUUUGUGG | 1205 | CCACAAAG GGCTAGCTACAACGA TGCCCCTC | 3580 |
| 643 | UGGAGAUG G UGGACAAC | 1206 | GTTGTCCA GGCTAGCTACAACGA CATCTCCA | 3581 |
| 659 | CCUGAGGG G CAAGUCGG | 1207 | CCGACTTG GGCTAGCTACAACGA CCCTCAGG | 3582 |
| 663 | AGGGGCAA G UCGGGGCA | 1208 | TGCCCCGA GGCTAGCTACAACGA TTGCCCCT | 3583 |
| 669 | AAGUCGGG G CAGGGCUA | 1209 | TAGCCCTG GGCTAGCTACAACGA CCCGACTT | 3584 |
| 674 | GGGGCAGG G CUACUACG | 1210 | CGTAGTAG GGCTAGCTACAACGA CCTGCCCC | 3585 |
| 682 | GCUACUAC G UGGAGAUG | 1211 | CATCTCCA GGCTAGCTACAACGA GTAGTAGC | 3586 |
| 694 | AGAUGACC G UGGGCAGC | 1212 | GCTGCCCA GGCTAGCTACAACGA GGTCATCT | 3587 |
| 698 | GACCGUGG G CAGCCCCC | 1213 | GGGGGCTG GGCTAGCTACAACGA CCACGGTC | 3588 |
| 701 | CGUGGGCA G CCCCCCGC | 1214 | GCGGGGGG GGCTAGCTACAACGA TGCCCACG | 3589 |
| 727 | ACAUCCUG G UGGAUACA | 1215 | TGTATCCA GGCTAGCTACAACGA CAGGATGT | 3590 |
| 737 | GGAUACAG G CAGCAGUA | 1216 | TACTGCTG GGCTAGCTACAACGA CTGTATCC | 3591 |
| 740 | UACAGGCA G CAGUAACU | 1217 | AGTTACTG GGCTAGCTACAACGA TGCCTGTA | 3592 |
| 743 | AGGCAGCA G UAACUUUG | 1218 | CAAAGTTA GGCTAGCTACAACGA TGCTGCCT | 3593 |
| 754 | ACUUUGCA G UGGGUGCU | 1219 | AGCACCCA GGCTAGCTACAACGA TGCAAAGT | 3594 |
| 758 | UGCAGUGG G UGCUGCCC | 1220 | GGGCAGCA GGCTAGCTACAACGA CCACTGCA | 3595 |
| 798 | UACCAGAG G CAGCUGUC | 1221 | GACAGCTG GGCTAGCTACAACGA CTCTGGTA | 3596 |
| 801 | CAGAGGCA G CUGUCCAG | 1222 | CTGGACAG GGCTAGCTACAACGA TGCCTCTG | 3597 |
| 809 | GCUGUCCA G CACAUACC | 1223 | GGTATGTG GGCTAGCTACAACGA TGGACAGC | 3598 |
| 833 | CCGGAAGG G UGUGUAUG | 1224 | CATACACA GGCTAGCTACAACGA CCTTCCGG | 3599 |
| 857 | CACCCAGG G CAAGUGGG | 1225 | CCCACTTG GGCTAGCTACAACGA CCTGGGTG | 3600 |
| 861 | CAGGGCAA G UGGGAAGG | 1226 | CCTTCCCA GGCTAGCTACAACGA TTGCCCTG | 3601 |
| 873 | GAAGGGGA G CUGGGCAC | 1227 | GTGCCCAG GGCTAGCTACAACGA TCCCCTTC | 3602 |
| 878 | GGAGCUGG G CACCGACC | 1228 | GGTCGGTG GGCTAGCTACAACGA CCAGCTCC | 3603 |
| 889 | CCGACCUG G UAAGCAUG | 1229 | GATGCTTA GGCTAGCTACAACGA CAGGTCGG | 3604 |
| 893 | CCUGGUAA G CAUCCCCC | 1230 | GGGGGATG GGCTAGCTACAACGA TTACCAGG | 3605 |
| 905 | CCCCCAUG G CCCCAACG | 1231 | CGTTGGGG GGCTAGCTACAACGA CATGGGGG | 3606 |
| 913 | GCCCCAAC G UCACUGUG | 1232 | CACAGTGA GGCTAGCTACAACGA GTTGGGGC | 3607 |
| 923 | CACUGUGC G UGCCAACA | 1233 | TGTTGGCA GGCTAGCTACAACGA GCACAGTG | 3608 |
| 957 | UCAGACAA G UUCUUCAU | 1234 | ATGAAGAA GGCTAGCTACAACGA TTGTCTGA | 3609 |
| 971 | CAUCAACG G CUCCAACU | 1235 | AGTTGGAG GGCTAGCTACAACGA CGTTGATG | 3610 |
| 986 | CUGGGAAG G CAUCCUGG | 1236 | CCAGGATG GGCTAGCTACAACGA CTTCCCAG | 3611 |
| 996 | AUCCUGGG G CUGGCCUA | 1237 | TAGGCCAG GGCTAGCTACAACGA CCCAGGAT | 3612 |
| 1000 | UGGGGCUG G CCUAUGCU | 1238 | AGCATAGG GGCTAGCTACAACGA CAGCCCCA | 3613 |
| 1020 | AUUGCCAG G CCUGACGA | 1239 | TCGTCAGG GGCTAGCTACAACGA CTGGCAAT | 3614 |
| 1038 | UCCCUGGA G CCUUUCUU | 1240 | AAGAAAGG GGCTAGCTACAACGA TCCAGGGA | 3615 |
| 1057 | ACUCUCUG G UAAAGCAG | 1241 | CTGCTTTA GGCTAGCTACAACGA CAGAGAGT | 3616 |
| 1062 | CUGGUAAA G CAGACCCA | 1242 | TGGGTCTG GGCTAGCTACAACGA TTTACCAG | 3617 |
| 1072 | AGACCCAC G UUCCCAAC | 1243 | GTTGGGAA GGCTAGCTACAACGA GTGGGTCT | 3618 |
| 1095 | UCCCUGCA G CUUUGUGG | 1244 | CCACAAAG GGCTAGCTACAACGA TGCAGGGA | 3619 |
| 1103 | GCUUUGUG G UGCUGGCU | 1245 | AGCCAGCA GGCTAGCTACAACGA CACAAAGC | 3620 |
| 1109 | UGGUGCUG G CUUCCCCC | 1246 | GGGGGAAG GGCTAGCTACAACGA CAGCACCA | 3621 |
| 1125 | CUCAACCA G UCUGAAGU | 1247 | ACTTCAGA GGCTAGCTACAACGA TGGTTGAG | 3622 |
| 1132 | AGUCUGAA G UGCUGGCC | 1248 | GGCCAGCA GGCTAGCTACAACGA TTCAGACT | 3623 |
| 1138 | AAGUGCUG G CCUCUGUC | 1249 | GACAGAGG GGCTAGCTACAACGA CAGCACTT | 3624 |
| 1154 | CGGAGGGA G CAUGAUCA | 1250 | TGATCATG GGCTAGCTACAACGA TCCCTCCG | 3625 |
| 1169 | CAUUGGAG G UAUCGACC | 1251 | GGTCGATA GGCTAGCTACAACGA CTCCAATG | 3626 |
| 1193 | GUACACAG G CAGUCUCU | 1252 | AGAGACTG GGCTAGCTACAACGA CTGTGTAC | 3627 |
| 1196 | CACAGGCA G UCUCUGGU | 1253 | ACCAGAGA GGCTAGCTACAACGA TGCCTGTG | 3628 |
| 1203 | AGUCUCUG G UAUACACC | 1254 | GGTGTATA GGCTAGCTACAACGA CAGAGACT | 3629 |
| 1218 | CCCAUCCG G CGGGAGUG | 1255 | CACTCCCG GGCTAGCTACAACGA CGGATGGG | 3630 |
| 1224 | CGGCGGGA G UGGUAUUA | 1256 | TAATACCA GGCTAGCTACAACGA TCCCGCCG | 3631 |
| 1227 | CGGGAGUG G UAUUAUGA | 1257 | TCATAATA GGCTAGCTACAACGA CACTCCCG | 3632 |
| 1237 | AUUAUGAG G UGAUCAUU | 1258 | AATGATCA GGCTAGCTACAACGA CTCATAAT | 3633 |
| 1252 | UUGUGCGG G UGGAGAUC | 1259 | GATCTCCA GGCTAGCTACAACGA CCGCACAA | 3634 |
| 1293 | UGCAAGGA G UACAACUA | 1260 | TAGTTGTA GGCTAGCTACAACGA TCCTTGCA | 3635 |
| 1310 | UGACAAGA G CAUUGUGG | 1261 | CCACAATG GGCTAGCTACAACGA TCTTGTCA | 3636 |
| 1322 | UGUGGACA G UGGCACCA | 1262 | TGGTGCCA GGCTAGCTACAACGA TGTCCACA | 3637 |
| 1325 | GGACAGUG G CACCACCA | 1263 | TGGTGGTG GGCTAGCTACAACGA CACTGTCC | 3638 |
| 1340 | CAACCUUC G UUUGCCCA | 1264 | TGGGCAAA GGCTAGCTACAACGA GAAGGTTG | 3639 |
| 1354 | CCAAGAAA G UGUUUGAA | 1265 | TTCAAACA GGCTAGCTACAACGA TTTCTTGG | 3640 |
| 1363 | UGUUUGAA G CUGCAGUC | 1266 | GACTGCAG GGCTAGCTACAACGA TTCAAACA | 3641 |
| 1369 | AAGCUGCA G UCAAAUCC | 1267 | GGATTTGA GGCTAGCTACAACGA TGCAGCTT | 3642 |
| 1384 | CCAUCAAG G CAGCCUCC | 1268 | GGAGGCTG GGCTAGCTACAACGA CTTGATGG | 3643 |
| 1387 | UCAAGGCA G CCUCCUCC | 1269 | GGAGGAGG GGCTAGCTACAACGA TGCCTTGA | 3644 |
| 1404 | ACGGAGAA G UUCCCUGA | 1270 | TCAGGGAA GGCTAGCTACAACGA TTCTCCGT | 3645 |
| 1415 | CCCUGAUG G UUUCUGGC | 1271 | GCCAGAAA GGCTAGCTACAACGA CATCAGGG | 3646 |
| 1422 | GGUUUCUG G CUAGGAGA | 1272 | TCTCCTAG GGCTAGCTACAACGA CAGAAACC | 3647 |
| 1431 | CUAGGAGA G CAGCUGGU | 1273 | ACCAGCTG GGCTAGCTACAACGA TCTCCTAG | 3648 |
| 1434 | GGAGAGCA G CUGGUGUG | 1274 | CACACCAG GGCTAGCTACAACGA TGCTCTCC | 3649 |
| 1438 | AGCAGCUG G UGUGCUGG | 1275 | CCAGCACA GGCTAGCTACAACGA CAGCTGCT | 3650 |
| 1446 | GUGUGCUG G CAAGCAGG | 1276 | CCTGCTTG GGCTAGCTACAACGA CAGCACAC | 3651 |
| 1450 | GCUGGCAA G CAGGCACC | 1277 | GGTGCCTG GGCTAGCTACAACGA TTGCCAGC | 3652 |
| 1454 | GCAAGCAG G CACCACCC | 1278 | GGGTGGTG GGCTAGCTACAACGA CTGCTTGC | 3653 |
| 1480 | UUUUCCCA G UCAUCUCA | 1279 | TGAGATGA GGCTAGCTACAACGA TGGGAAAA | 3654 |
| 1502 | CCUAAUGG G UGAGGUUA | 1280 | TAACCTCA GGCTAGCTACAACGA CCATTAGG | 3655 |
| 1507 | UGGGUGAG G UUACCAAC | 1281 | GTTGGTAA GGCTAGCTACAACGA CTCACCCA | 3656 |
| 1518 | ACCAACCA G UCCUUCCG | 1282 | CGGAAGGA GGCTAGCTACAACGA TGGTTGGT | 3657 |
| 1545 | CUUCCGCA G CAAUACCU | 1283 | AGGTATTG GGCTAGCTACAACGA TGCGGAAG | 3658 |
| 1557 | UACCUGCG G CCAGUGGA | 1284 | TCCACTGG GGCTAGCTACAACGA CGCAGGTA | 3659 |
| 1561 | UGCGGCCA G UGGAAGAU | 1285 | ATCTTCCA GGCTAGCTACAACGA TGGCCGCA | 3660 |
| 1573 | AAGAUGUG G CCACGUCC | 1286 | GGACGTGG GGCTAGCTACAACGA CACATCTT | 3661 |
| 1578 | GUGGCCAC G UCCCAAGA | 1287 | TCTTGGGA GGCTAGCTACAACGA GTGGCCAC | 3662 |
| 1599 | UGUUACAA G UUUGCCAU | 1288 | ATGGCAAA GGCTAGCTACAACGA TTGTAACA | 3663 |
| 1614 | AUCUCACA G UCAUCCAC | 1289 | GTGGATGA GGCTAGCTACAACGA TGTGAGAT | 3664 |
| 1625 | AUCCACGG G CACUGUUA | 1290 | TAACAGTG GGCTAGCTACAACGA CCGTGGAT | 3665 |
| 1639 | UUAUGGGA G CUGUUAUC | 1291 | GATAACAG GGCTAGCTACAACGA TCCCATAA | 3666 |
| 1655 | CAUGGAGG G CUUCUACG | 1292 | CGTAGAAG GGCTAGCTACAACGA CCTCCATG | 3667 |
| 1663 | GCUUCUAC G UUGUCUUU | 1293 | AAAGACAA GGCTAGCTACAACGA GTAGAAGC | 3668 |
| 1678 | UUGAUCGG G CCCGAAAA | 1294 | TTTTCGGG GGCTAGCTACAACGA CCGATCAA | 3669 |
| 1694 | ACGAAUUG G CUUUGCUG | 1295 | CAGCAAAG GGCTAGCTACAACGA CAATTCGT | 3670 |
| 1706 | UGCUGUCA G CGCUUGCC | 1296 | GGCAAGCG GGCTAGCTACAACGA TGACAGCA | 3671 |
| 1728 | CACGAUGA G UUCAGGAC | 1297 | GTCCTGAA GGCTAGCTACAACGA TCATCGTG | 3672 |
| 1738 | UCAGGACG G CAGCGGUG | 1298 | CACCGCTG GGCTAGCTACAACGA CGTCCTGA | 3673 |
| 1741 | GGACGGCA G CGGUGGAA | 1299 | TTCCACCG GGCTAGCTACAACGA TGCCGTCC | 3674 |
| 1744 | CGGCAGCG G UGGAAGGC | 1300 | GCCTTCCA GGCTAGCTACAACGA CGCTGCCG | 3675 |
| 1751 | GGUGGAAG G CCCUUUUG | 1301 | CAAAAGGG GGCTAGCTACAACGA CTTCCACC | 3676 |
| 1784 | AGACUGUG G CUACAACA | 1302 | TGTTGTAG GGCTAGCTACAACGA CACAGTCT | 3677 |
| 1809 | ACAGAUGA G UCAACCCU | 1303 | AGGGTTGA GGCTAGCTACAACGA TCATCTGT | 3678 |
| 1828 | UGACCAUA G CCUAUGUC | 1304 | GACATAGG GGCTAGCTACAACGA TATGGTCA | 3679 |
| 1840 | AUGUCAUG G CUGCCAUC | 1305 | GATGGCAG GGCTAGCTACAACGA CATGACAT | 3680 |
| 1882 | GCCUCAUG G UGUGUCAG | 1306 | CTGACACA GGCTAGCTACAACGA CATGAGGC | 3681 |
| 1890 | GUGUGUCA G UGGCGCUG | 1307 | CAGCGCCA GGCTAGCTACAACGA TGACACAC | 3682 |
| 1893 | UGUCAGUG G CGCUGCCU | 1308 | AGGCAGCG GGCTAGCTACAACGA CACTGACA | 3683 |
| 1917 | CUGCGCCA G CAGCAUGA | 1309 | TCATGCTG GGCTAGCTACAACGA TGGCGCAG | 3684 |
| 1920 | CGCCAGCA G CAUGAUGA | 1310 | TCATCATG GGCTAGCTACAACGA TGCTGGCG | 3685 |
| 1956 | CUGCUGAA G UGAGGAGG | 1311 | CCTCCTCA GGCTAGCTACAACGA TTCAGCAG | 3686 |
| 1964 | GUGAGGAG G CCCAUGGG | 1312 | CCCATGGG GGCTAGCTACAACGA CTCCTCAC | 3687 |
| 1972 | GCCCAUGG G CAGAAGAU | 1313 | ATCTTCTG GGCTAGCTACAACGA CCATGGGC | 3688 |
| 2006 | ACACCUCC G UGGUUCAC | 1314 | GTGAACCA GGCTAGCTACAACGA GGAGGTGT | 3689 |
| 2009 | CCUCCGUG G UUCACUUU | 1315 | AAAGTGAA GGCTAGCTACAACGA CACGGAGG | 3690 |
| 2019 | UCACUUUG G UCACAAGU | 1316 | ACTTGTGA GGCTAGCTACAACGA CAAAGTGA | 3691 |
| 2026 | GGUCACAA G UAGGAGAC | 1317 | GTCTCCTA GGCTAGCTACAACGA TTGTGACC | 3692 |
| 2042 | CACAGAUG G CACCUGUG | 1318 | CACAGGTG GGCTAGCTACAACGA CATCTGTG | 3693 |
| 2051 | CACCUGUG G CCAGAGCA | 1319 | TGCTCTGG GGCTAGCTACAACGA CACAGGTG | 3694 |
| 2057 | UGGCCAGA G CACCUCAG | 1320 | CTGAGGTG GGCTAGCTACAACGA TCTGGCCA | 3695 |
| 2114 | AGGAAAAG G CUGGCAAG | 1321 | CTTGCCAG GGCTAGCTACAACGA CTTTTCCT | 3696 |
| 2118 | AAAGGCUG G CAAGGUGG | 1322 | CCACCTTG GGCTAGCTACAACGA CAGCCTTT | 3697 |
| 2123 | CUGGCAAG G UGGGUUCC | 1323 | GGAACCCA GGCTAGCTACAACGA CTTGCCAG | 3698 |
| 2127 | CAAGGUGG G UUCCAGGG | 1324 | CCCTGGAA GGCTAGCTACAACGA CCACCTTG | 3699 |
| 2172 | AGAAAGAA G CACUCUGC | 1325 | GCAGAGTG GGCTAGCTACAACGA TTCTTTCT | 3700 |
| 2183 | CUCUGCUG G CGGGAAUA | 1326 | TATTCCCG GGCTAGCTACAACGA CAGCAGAG | 3701 |
| 2198 | UACUCUUG G UCACCUCA | 1327 | TGAGGTGA GGCTAGCTACAACGA CAAGAGTA | 3702 |
| 2214 | AAAUUUAA G UCGGGAAA | 1328 | TTTCCCGA GGCTAGCTACAACGA TTAAATTT | 3703 |
| 2243 | AAACUUCA G CCCUGAAC | 1329 | GTTCAGGG GGCTAGCTACAACGA TGAAGTTT | 3704 |
| 2288 | AACCCAAA G UAUUCUUC | 1330 | GAAGAATA GGCTAGCTACAACGA TTTGGGTT | 3705 |
| 2305 | UUUUCUUA G UUUCAGAA | 1331 | TTCTGAAA GGCTAGCTACAACGA TAAGAAAA | 3706 |
| 2314 | UUUCAGAA G UACUGGCA | 1332 | TGCCAGTA GGCTAGCTACAACGA TTCTGAAA | 3707 |
| 2320 | AAGUACUG G CAUCACAC | 1333 | GTGTGATG GGCTAGCTACAACGA CAGTACTT | 3708 |
| 2333 | ACACGCAG G UUACCUUG | 1334 | CAAGGTAA GGCTAGCTACAACGA CTGCGTGT | 3709 |
| 2342 | UUACCUUG G CGUGUGUC | 1335 | GACACACG GGCTAGCTACAACGA CAAGGTAA | 3710 |
| 2344 | ACCUUGGC G UGUGUCCC | 1336 | GGGACACA GGCTAGCTACAACGA GCCAAGGT | 3711 |
| 2357 | UCCCUGUG G UACCCUGG | 1337 | CCAGGGTA GGCTAGCTACAACGA CACAGGGA | 3712 |
| 2365 | GUACCCUG G CAGAGAAG | 1338 | CTTCTCTG GGCTAGCTACAACGA CAGGGTAC | 3713 |
| 2381 | GAGACCAA G CUUGUUUC | 1339 | GAAACAAG GGCTAGCTACAACGA TTGGTCTC | 3714 |
| 2397 | CCCUGCUG G CCAAAGUC | 1340 | GACTTTGG GGCTAGCTACAACGA CAGCAGGG | 3715 |
| 2403 | UGGCCAAA G UCAGUAGG | 1341 | CCTACTGA GGCTAGCTACAACGA TTTGGCCA | 3716 |
| 2407 | CAAAGUCA G UAGGAGAG | 1342 | CTCTCCTA GGCTAGCTACAACGA TGACTTTG | 3717 |
| 2424 | GAUGCACA G UUUGCUAU | 1343 | ATAGCAAA GGCTAGCTACAACGA TGTGCATC | 3718 |
| 2463 | AUAAACAA G CCUAACAU | 1344 | ATGTTAGG GGCTAGCTACAACGA TTGTTTAT | 3719 |
| 2474 | UAACAUUG G UGCAAAGA | 1345 | TCTTTGCA GGCTAGCTACAACGA CAATGTTA | 3720 |
| 45 | CGAGCUGG A UCAUGGUG | 1346 | CACCATAA GGCTAGCTACAACGA CCAGCTCG | 3721 |
| 67 | AGCAGCCA A CGCAGCCG | 1347 | CGGCTGCG GGCTAGCTACAACGA TGGCTGCT | 3722 |
| 125 | CCGGGGGG A CCAGGGAA | 1348 | TTCCCTGG GGCTAGCTACAACGA CCCCCCGG | 3723 |
| 217 | CCGUGCCG A UGUAGCGG | 1349 | CCGCTACA GGCTAGCTACAACGA CGGCACGG | 3724 |
| 233 | GGCUCCGG A UCCCAGCC | 1350 | GGCTGGGA GGCTAGCTACAACGA CCGGAGCC | 3725 |
| 267 | CUCUGCGG A UCUCCCCU | 1351 | AGGGCAGA GGCTAGCTACAACGA CCGCAGAG | 3726 |
| 277 | CUCCCCUG A CCGCUCUC | 1352 | GAGAGCGG GGCTAGCTACAACGA CAGGGGAG | 3727 |
| 296 | CAGCCCGG A CCCGGGGG | 1353 | CCCCCGGG GGCTAGCTACAACGA CCGGGCTG | 3728 |
| 338 | GCGUCCUG A UGCCCCCA | 1354 | TGGGGGCA GGCTAGCTACAACGA CAGGACGC | 3729 |
| 383 | CCACCCAG A CUUGGGGG | 1355 | CCCCCAAG GGCTAGCTACAACGA CTGGGTGG | 3730 |
| 404 | CGCCAGGG A CGGACGUG | 1356 | CACGTCCG GGCTAGCTACAACGA CCCTGGCG | 3731 |
| 408 | AGGGACGG A CGUGGGCC | 1357 | GGCCCACG GGCTAGCTACAACGA CCGTCCCT | 3732 |
| 487 | UGCUGUGG A UGGGCGCG | 1358 | CGCGCCCA GGCTAGCTACAACGA CCACAGCA | 3733 |
| 592 | CCCGGGAG A CCGACGAA | 1359 | TTCGTCGG GGCTAGCTACAACGA CTCCCGGG | 3734 |
| 596 | GGAGACCG A CGAAGAGC | 1360 | GCTCTTCG GGCTAGCTACAACGA CGGTCTCC | 3735 |
| 640 | UUGUGGAG A UGGUGGAC | 1361 | GTCCACCA GGCTAGCTACAACGA CTCCACAA | 3736 |
| 647 | GAUGGUGG A CAACCUGA | 1362 | TCAGGTTG GGCTAGCTACAACGA CCACCATC | 3737 |
| 650 | GGUGGACA A CCUGAGGG | 1363 | CCCTCAGG GGCTAGCTACAACGA TGTCCACC | 3738 |
| 688 | ACGUGGAG A UGACCGUG | 1364 | CACGGTCA GGCTAGCTACAACGA CTCCACGT | 3739 |
| 691 | UGGAGAUG A CCGUGGGC | 1365 | GCCCACGG GGCTAGCTACAACGA CATCTCCA | 3740 |
| 712 | CCCCGCAG A CGCUCAAC | 1366 | GTTGAGCG GGCTAGCTACAACGA CTGCGGGG | 3741 |
| 719 | GACGCUCA A CAUCCUGG | 1367 | CCAGGATG GGCTAGCTACAACGA TGAGCGTC | 3742 |
| 731 | CCUGGUGG A UACAGGCA | 1368 | TGCCTGTA GGCTAGCTACAACGA CCACCAGG | 3743 |
| 746 | CAGCAGUA A CUUUGCAG | 1369 | CTGCAAAG GGCTAGCTACAACGA TACTGCTG | 3744 |
| 821 | AUACCGGG A CCUCCGGA | 1370 | TCCGGAGG GGCTAGCTACAACGA CCCGGTAT | 3745 |
| 884 | GGGCACCG A CCUGGUAA | 1371 | TTACCAGG GGCTAGCTACAACGA CGGTGCCC | 3746 |
| 911 | UGGCCCCA A CGUCACUG | 1372 | CAGTGACG GGCTAGCTACAACGA TGGGGCCA | 3747 |
| 929 | GCGUGCCA A CAUUGCUG | 1373 | CAGCAATG GGCTAGCTACAACGA TGGCACGC | 3748 |
| 948 | AUCACUGA A UCAGACAA | 1374 | TTGTCTGA GGCTAGCTACAACGA TCAGTGAT | 3749 |
| 953 | UGAAUCAG A CAAGUUCU | 1375 | AGAACTTG GGCTAGCTACAACGA CTGATTCA | 3750 |
| 968 | CUUCAUCA A CGGCUCCA | 1376 | TGGAGCCG GGCTAGCTACAACGA TGATGAAG | 3751 |
| 977 | CGGCUCCA A CUGGGAAG | 1377 | CTTCCCAG GGCTAGCTACAACGA TGGAGCCG | 3752 |
| 1012 | AUGCUGAG A UUGCCAGG | 1378 | CCTGGCAA GGCTAGCTACAACGA CTCAGCAT | 3753 |
| 1025 | CAGGCCUG A CGACUCCC | 1379 | GGGAGTCG GGCTAGCTACAACGA CAGGCCTG | 3754 |
| 1028 | GCCUGACG A CUCCCUGG | 1380 | CCAGGGAG GGCTAGCTACAACGA CGTCAGGC | 3755 |
| 1049 | UUUCUUUG A CUCUCUGG | 1381 | CCAGAGAG GGCTAGCTACAACGA CAAAGAAA | 3756 |
| 1066 | UAAAGCAG A CCCACGUU | 1382 | AACGTGGG GGCTAGCTACAACGA CTGCTTTA | 3757 |
| 1079 | CGUUCCCA A CCUCUUCU | 1383 | AGAAGAGG GGCTAGCTACAACGA TGGGAACG | 3758 |
| 1121 | CCCCCUCA A CCAGUCUG | 1384 | CAGACTGG GGCTAGCTACAACGA TGAGGGGG | 3759 |
| 1159 | GGAGCAUG A UCAUUGGA | 1385 | TCCAATGA GGCTAGCTACAACGA CATGCTCC | 3760 |
| 1175 | AGGUAUCG A CCACUCGC | 1386 | GCGAGTGG GGCTAGCTACAACGA CGATACCT | 3761 |
| 1240 | AUGAGGUG A UCAUUGUG | 1387 | CACAATGA GGCTAGCTACAACGA CACCTCAT | 3762 |
| 1258 | GGGUGGAG A UCAAUGGA | 1388 | TCCATTGA GGCTAGCTACAACGA CTCCACCC | 3763 |
| 1262 | GGAGAUCA A UGGACAGG | 1389 | CCTGTCCA GGCTAGCTACAACGA TGATCTCC | 3764 |
| 1266 | AUCAAUGG A CAGGAUCU | 1390 | AGATCCTG GGCTAGCTACAACGA CCATTGAT | 3765 |
| 1271 | UGGACAGG A UCUGAAAA | 1391 | TTTTCAGA GGCTAGCTACAACGA CCTGTCCA | 3766 |
| 1279 | AUCUGAAA A UGGACUGC | 1392 | GCAGTCCA GGCTAGCTACAACGA TTTCAGAT | 3767 |
| 1283 | GAAAAUGG A CUGCAAGG | 1393 | CCTTGCAG GGCTAGCTACAACGA CCATTTTC | 3768 |
| 1298 | GGAGUACA A CUAUGACA | 1394 | TGTCATAG GGCTAGCTACAACGA TGTACTCC | 3769 |
| 1304 | CAACUAUG A CAAGAGCA | 1395 | TGCTCTTG GGCTAGCTACAACGA CATAGTTG | 3770 |
| 1319 | CAUUGUGG A CAGUGGCA | 1396 | TGCCACTG GGCTAGCTACAACGA CCACAATG | 3771 |
| 1334 | CACCACCA A CCUUCGUU | 1397 | AACGAAGG GGCTAGCTACAACGA TGGTGGTG | 3772 |
| 1374 | GCAGUCAA A UCCAUCAA | 1398 | TTGATGGA GGCTAGCTACAACGA TTGACTGC | 3773 |
| 1412 | GUUCCCUG A UGGUUUCU | 1399 | AGAAACCA GGCTAGCTACAACGA CAGGGAAC | 3774 |
| 1469 | CCCUUGGA A CAUUUUCC | 1400 | GGAAAATG GGCTAGCTACAACGA TCCAAGGG | 3775 |
| 1498 | UCUACCUA A UGGGUGAG | 1401 | CTCACCCA GGCTAGCTACAACGA TAGGTAGA | 3776 |
| 1514 | GGUUACCA A CCAGUCCU | 1402 | AGGACTGG GGCTAGCTACAACGA TGGTAACC | 3777 |
| 1548 | CCGCAGCA A UACCUGCG | 1403 | CGCAGGTA GGCTAGCTACAACGA TGCTGCGG | 3778 |
| 1568 | AGUGGAAG A UGUGGCCA | 1404 | TGGCCACA GGCTAGCTACAACGA CTTCCACT | 3779 |
| 1586 | GUCCCAAG A CGACUGUU | 1405 | AACAGTCG GGCTAGCTACAACGA CTTGGGAC | 3780 |
| 1589 | CCAAGACG A CUGUUACA | 1406 | TGTAACAG GGCTAGCTACAACGA CGTCTTGG | 3781 |
| 1673 | UGUCUUUG A UCGGGCCC | 1407 | GGGCCCGA GGCTAGCTACAACGA CAAAGACA | 3782 |
| 1686 | GCCCGAAA A CGAAUUGG | 1408 | CCAATTCG GGCTAGCTACAACGA TTTCGGGC | 3783 |
| 1690 | GAAAACGA A UUGGCUUU | 1409 | AAAGCCAA GGCTAGCTACAACGA TCGTTTTC | 3784 |
| 1724 | UGUGCACG A UGAGUUCA | 1410 | TGAACTCA GGCTAGCTACAACGA CGTGCACA | 3785 |
| 1735 | AGUUCAGG A CGGCAGCG | 1411 | CGCTGCCG GGCTAGCTACAACGA CCTGAACT | 3786 |
| 1769 | CACCUUGG A CAUGGAAG | 1412 | CTTCCATG GGCTAGCTACAACGA CCAAGGTG | 3787 |
| 1778 | CAUGGAAG A CUGUGGCU | 1413 | AGCCACAG GGCTAGCTACAACGA CTTCCATG | 3788 |
| 1790 | UGGCUACA A CAUUCCAC | 1414 | GTGGAATG GGCTAGCTACAACGA TGTAGCCA | 3789 |
| 1801 | UUCCACAG A CAGAUGAG | 1415 | CTCATCTG GGCTAGCTACAACGA CTGTGGAA | 3790 |
| 1805 | ACAGACAG A UGAGUCAA | 1416 | TTGACTCA GGCTAGCTACAACGA CTGTCTGT | 3791 |
| 1813 | AUGAGUCA A CCCUCAUG | 1417 | CATGAGGG GGCTAGCTACAACGA TGACTCAT | 3792 |
| 1822 | CCCUCAUG A CCAUAGCC | 1418 | GGCTATGG GGCTAGCTACAACGA CATGAGGG | 3793 |
| 1925 | GCAGCAUG A UGACUUUG | 1419 | CAAAGTCA GGCTAGCTACAACGA CATGCTGC | 3794 |
| 1928 | GCAUGAUG A CUUUGCUG | 1420 | CAGCAAAG GGCTAGCTACAACGA CATCATGC | 3795 |
| 1937 | CUUUGCUG A UGACAUCU | 1421 | AGATGTCA GGCTAGCTACAACGA CAGCAAAG | 3796 |
| 1940 | UGCUGAUG A CAUCUCCC | 1422 | GGGAGATG GGCTAGCTACAACGA CATCAGCA | 3797 |
| 1979 | GGCAGAAG A UAGAGAUU | 1423 | AATCTCTA GGCTAGCTACAACGA CTTCTGCC | 3798 |
| 1985 | AGAUAGAG A UUCCCCUG | 1424 | CAGGGGAA GGCTAGCTACAACGA CTCTATCT | 3799 |
| 1995 | UCCCCUGG A CCACACCU | 1425 | AGGTGTGG GGCTAGCTACAACGA CCAGGGGA | 3800 |
| 2033 | AGUAGGAG A CACAGAUG | 1426 | CATCTGTG GGCTAGCTACAACGA CTCCTACT | 3801 |
| 2039 | AGACACAG A UGGCACCU | 1427 | AGGTGCCA GGCTAGCTACAACGA CTGTGTCT | 3802 |
| 2067 | ACCUCAGG A CCCUCCCC | 1428 | GGGGAGGG GGCTAGCTACAACGA CCTGAGGT | 3803 |
| 2085 | CCCACCAA A UGCCUCUG | 1429 | CAGAGGCA GGCTAGCTACAACGA TTGGTGGG | 3804 |
| 2099 | CUGCCUUG A UGGAGAAG | 1430 | CTTCTCCA GGCTAGCTACAACGA CAAGGCAG | 3805 |
| 2136 | UUCCAGGG A CUGUACCU | 1431 | AGGTACAG GGCTAGCTACAACGA CCCTGGAA | 3806 |
| 2152 | UGUAGGAA A CAGAAAAG | 1432 | CTTTTCTG GGCTAGCTACAACGA TTCCTACA | 3807 |
| 2189 | UGGCGGGA A UACUCUUG | 1433 | CAAGAGTA GGCTAGCTACAACGA TCCCGCCA | 3808 |
| 2208 | CACCUCAA A UUUAAGUC | 1434 | GACTTAAA GGCTAGCTACAACGA TTGAGGTG | 3809 |
| 2222 | GUCGGGAA A UUCUCCUG | 1435 | CAGCAGAA GGCTAGCTACAACGA TTCCCGAC | 3810 |
| 2237 | UGCUUGAA A CUUCAGCC | 1436 | GGCTGAAG GGCTAGCTACAACGA TTCAAGCA | 3811 |
| 2250 | AGCCCUGA A CCUUUGUC | 1437 | GACAAAGG GGCTAGCTACAACGA TCAGGGCT | 3812 |
| 2273 | UCCUUUAA A UUCUCCAA | 1438 | TTGGAGAA GGCTAGCTACAACGA TTAAAGGA | 3813 |
| 2281 | AUUCUCCA A CCCAAAGU | 1439 | ACTTTGGG GGCTAGCTACAACGA TGGAGAAT | 3814 |
| 2376 | GAGAAGAG A CCAAGCUU | 1440 | AAGCTTGG GGCTAGCTACAACGA CTCTTCTC | 3815 |
| 2417 | AGGAGAGG A UGCACAGU | 1441 | ACTGTGCA GGCTAGCTACAACGA CCTCTCCT | 3816 |
| 2444 | CUUUAGAG A CAGGGACU | 1442 | AGTCCCTG GGCTAGCTACAACGA CTCTAAAG | 3817 |
| 2450 | AGACAGGG A CUGUAUAA | 1443 | TTATACAG GGCTAGCTACAACGA CCCTGTCT | 3818 |
| 2459 | CUGUAUAA A CAAGCCUA | 1444 | TAGGCTTG GGCTAGCTACAACGA TTATACAG | 3819 |
| 2468 | CAAGCCUA A CAUUGGUG | 1445 | CACCAATG GGCTAGCTACAACGA TAGGCTTG | 3820 |
| 2482 | GUGCAAAG A UUGCCUCU | 1446 | AGAGGCAA GGCTAGCTACAACGA CTTTGCAC | 3821 |
| 2494 | CCUCUUGA A UUAAAAAA | 1447 | TTTTTTAA GGCTAGCTACAACGA TCAAGAGG | 3822 |
| 2507 | AAAAAAAA A CUAGAAAA | 1448 | TTTTCTAG GGCTAGCTACAACGA TTTTTTTT | 3823 |

Input Sequence = AF190725. Cut Site = G/.
Stem Length = 8. Core Sequence = GGCTAGCTACAACGA
AF190725 (Homo sapiens beta-site APP cleaving enzyme (BACE) mRNA; 2526 bp)

TABLE VIII

Human BACE Amberzyme Ribozyme and Target Sequences

| | | | | |
|---|---|---|---|---|
| Pos | Substrate | Seq ID | Amberzyme | Seq ID |

| | | | | |
|---|---|---|---|---|
| 11 | ACGCGUCC G CAGCCCGC | 960 | GCGGGCUG GGAGGAAACUCC CU UCAAGGACAUCGUCCGGG GGACGCGU | 3824 |
| 18 | CGCAGCCC G CCCGGGAG | 961 | CUCCCGGG GGAGGAAACUCC CU UCAAGGACAUCGUCCGGG GGGCUGCG | 3825 |
| 29 | CGGGAGCU G CGAGCCGC | 962 | GCGGCUCG GGAGGAAACUCC CU UCAAGGACAUCGUCCGGG AGCUCCCG | 3826 |
| 31 | GGAGCUGC G AGCCGCGA | 963 | UCGCGGCU GGAGGAAACUCC CU UCAAGGACAUCGUCCGGG GCAGCUCC | 3827 |
| 36 | UGCGAGCC G CGAGCUGG | 964 | CCAGCUCG GGAGGAAACUCC CU UCAAGGACAUCGUCCGGG GGCUCGCA | 3828 |
| 38 | CGAGCCGC G AGCUGGAU | 965 | AUCCAGCU GGAGGAAACUCC CU UCAAGGACAUCGUCCGGG GCGGCUCG | 3829 |
| 58 | GGUGGCCU G AGCAGCCA | 966 | UGGCUGCU GGAGGAAACUCC CU UCAAGGACAUCGUCCGGG AGGCCACC | 3830 |
| 69 | CAGCCAAC G CAGCCGCA | 967 | UCCGGCUC GGAGGAAACUCC CU UCAAGGACAUCGUCCGGG GUUGGCUG | 3831 |
| 75 | ACGCAGCC G CAGGAGCC | 968 | GGCUCCUG GGAGGAAACUCC CU UCAAGGACAUCGUCCGGG GGCUGCGU | 3832 |
| 94 | GAGCCCUU G CCCCUGCC | 969 | GUCAGGGG GGAGGAAACUCC CU UCAAGGACAUCGUCCGGG AAGGGCUC | 3833 |
| 100 | UUGCCCCU G CCCGCGCC | 970 | GGCGCGGG GGAGGAAACUCC CU UCAAGGACAUCGUCCGGG AGGGGCAA | 3834 |
| 104 | CCCUGCCC G CGCCGCCG | 971 | CGGCGGCG GGAGGAAACUCC CU UCAAGGACAUCGUCCGGG GGGCAGGG | 3835 |
| 106 | CUGCCCGC G CCGCCGCC | 972 | GGCGGCGG GGAGGAAACUCC CU UCAAGGACAUCGUCCGGG GCGGGCAG | 3836 |
| 109 | CCCGCGCC G CCGCCCGC | 973 | GCGGGCGG GGAGGAAACUCC CU UCAAGGACAUCGUCCGGG GGCGCGGG | 3837 |
| 112 | GCGCCGCC G CCCGCCGG | 974 | CCGGCGGG GGAGGAAACUCC CU UCAAGGACAUCGUCCGGG GGCGGCGC | 3838 |
| 116 | CGCCGCCC G CCGGGGGG | 975 | CCCCCCGG GGAGGAAACUCC CU UCAAGGACAUCGUCCGGG GGGCGGCG | 3839 |
| 137 | GGGAAGCC G CCACCGGC | 976 | GCCGGUGG GGAGGAAACUCC CU UCAAGGACAUCGUCCGGG GGCUUCCC | 3840 |
| 148 | ACCGGCCC G CCAUGCCC | 977 | GGGCAUGG GGAGGAAACUCC CU UCAAGGACAUCGUCCGGG GGGCCGGU | 3841 |
| 153 | CCCGCCAU G CCCGCCCC | 978 | GGGGCGGG GGAGGAAACUCC CU UCAAGGACAUCGUCCGGG AUGGCGGG | 3842 |
| 157 | CCAUGCCC G CCCCUCCC | 979 | GGGAGGGG GGAGGAAACUCC CU UCAAGGACAUCGUCCGGG GGGCAUGG | 3843 |
| 172 | CCAGCCCC G CCGGGAGC | 980 | GCUCCCGG GGAGGAAACUCC CU UCAAGGACAUCGUCCGGG GGGGCUGG | 3844 |
| 183 | GGGAGCCC G CGCCCGCU | 981 | AGCGGGCG GGAGGAAACUCC CU UCAAGGACAUCGUCCGGG GGGCUCCC | 3845 |
| 185 | GAGCCCGC G CCCGCUGC | 982 | GCAGCGGG GGAGGAAACUCC CU UCAAGGACAUCGUCCGGG GCGGGCUC | 3846 |
| 189 | CCGCGCCC G CUGCCCAG | 983 | CUGGGCAG GGAGGAAACUCC CU UCAAGGACAUCGUCCGGG GGGCGCGG | 3847 |
| 192 | CGCCCGCU G CCCAGGCU | 984 | AGCCUGGG GGAGGAAACUCC CU UCAAGGACAUCGUCCGGG AGCGGGCG | 3848 |
| 205 | GGCUGGCC G CCGCCGUG | 985 | CACGGCGG GGAGGAAACUCC CU UCAAGGACAUCGUCCGGG GGCCAGCC | 3849 |
| 208 | UGGCCGCC G CCGUGCCG | 986 | CGGCACGG GGAGGAAACUCC CU UCAAGGACAUCGUCCGGG GGCGGCCA | 3850 |
| 213 | GCCGCCGU G CCGAUGUA | 987 | UACAUCGG GGAGGAAACUCC CU UCAAGGACAUCGUCCGGG ACGGCGGC | 3851 |
| 216 | GCCGUGCC G AUGUAGCG | 988 | CGCUACAU GGAGGAAACUCC CU UCAAGGACAUCGUCCGGG GGCACGGC | 3852 |
| 250 | UCUCCCCU G CUCCCGUG | 989 | CACGGGAG GGAGGAAACUCC CU UCAAGGACAUCGUCCGGG AGGGGAGA | 3853 |
| 258 | GCUCCCGU G CUCUGCGG | 990 | CCGCAGAG GGAGGAAACUCC CU UCAAGGACAUCGUCCGGG ACGGGAGC | 3854 |
| 263 | CGUGCUCU G CGGAUCUC | 991 | GAGAUCCG GGAGGAAACUCC CU UCAAGGACAUCGUCCGGG AGAGCACG | 3855 |
| 276 | UCUCCCCU G ACCGCUCU | 992 | AGAGCGGU GGAGGAAACUCC CU UCAAGGACAUCGUCCGGG AGGGGAGA | 3856 |
| 280 | CCCUGACC G CUCUCCAC | 993 | GUGGAGAG GGAGGAAACUCC CU UCAAGGACAUCGUCCGGG GGUCAGGG | 3857 |
| 320 | AGGGCCCU G CAGGCCCU | 994 | AGGGCCUG GGAGGAAACUCC CU UCAAGGACAUCGUCCGGG AGGGCCCU | 3858 |
| 337 | GGCGUCCU G AUGCCCCC | 995 | GGGGGCAU GGAGGAAACUCC CU UCAAGGACAUCGUCCGGG AGGACGCC | 3859 |
| 340 | GUCCUGAU G CCCCCAAG | 996 | CUUGGGGG GGAGGAAACUCC CU UCAAGGACAUCGUCCGGG AUCAGGAC | 3860 |
| 360 | CCUCUCCU G AGAAGCCA | 997 | UGGCUUCU GGAGGAAACUCC CU UCAAGGACAUCGUCCGGG AGGAGAGG | 3861 |
| 397 | GGGCAGGC G CCAGGGAC | 998 | GUCCCUGG GGAGGAAACUCC CU UCAAGGACAUCGUCCGGG GCCUGCCC | 3862 |
| 420 | GGGCCAGU G CGAGCCCA | 999 | UGGGCUCG GGAGGAAACUCC CU UCAAGGACAUCGUCCGGG ACUGGCCC | 3863 |
| 422 | GCCAGUGC G AGCCCAGA | 1000 | UCUGGGCU GGAGGAAACUCC CU UCAAGGACAUCGUCCGGG GCACUGGC | 3864 |
| 437 | GAGGGCCC G AAGGCCGG | 1001 | CCGGCCUU GGAGGAAACUCC CU UCAAGGACAUCGUCCGGG GGGCCCUC | 3865 |
| 468 | CAAGCCCU G CCCUGGCU | 1002 | AGCCAGGG GGAGGAAACUCC CU UCAAGGACAUCGUCCGGG AGGGCUUG | 3866 |
| 480 | UGGCUCCU G CUGUGGAU | 1003 | AUCCACAG GGAGGAAACUCC CU UCAAGGACAUCGUCCGGG AGGAGCCA | 3867 |
| 493 | GGAUGGGC G CGGGAGUG | 1004 | CACUCCCG GGAGGAAACUCC CU UCAAGGACAUCGUCCGGG GCCCAUCC | 3868 |
| 501 | GCGGGAGU G CUGCCUGC | 1005 | GCAGGCAG GGAGGAAACUCC CU UCAAGGACAUCGUCCGGG ACUCCCGC | 3869 |
| 504 | GGAGUGCU G CCUGCCCA | 1006 | UGGGCAGG GGAGGAAACUCC CU UCAAGGACAUCGUCCGGG AGCACUCC | 3870 |
| 508 | UGCUGCCU G CCCACGGC | 1007 | GCCGUGGG GGAGGAAACUCC CU UCAAGGACAUCGUCCGGG AGGCAGCA | 3871 |
| 537 | AUCCGGCU G CCCCUGCG | 1008 | CGCAGGGG GGAGGAAACUCC CU UCAAGGACAUCGUCCGGG AGCCGGAU | 3872 |
| 543 | CUGCCCCU G CGCAGCGG | 1009 | CCGCUGCG GGAGGAAACUCC CU UCAAGGACAUCGUCCGGG AGGGGCAG | 3873 |
| 545 | GCCCCUGC G CAGCGGCC | 1010 | GGCCGCUG GGAGGAAACUCC CU UCAAGGACAUCGUCCGGG GCAGGGGC | 3874 |
| 562 | UGGGGGGC G CCCCCCUG | 1011 | CAGGGGGG GGAGGAAACUCC CU UCAAGGACAUCGUCCGGG GCCCCCCA | 3875 |
| 576 | CUGGGGCU G CGGCUGCC | 1012 | GGCAGCCG GGAGGAAACUCC CU UCAAGGACAUCGUCCGGG AGCCCCAG | 3876 |
| 582 | CUGCGGCU G CCCCGGGA | 1013 | UCCCGGGG GGAGGAAACUCC CU UCAAGGACAUCGUCCGGG AGCCGCAG | 3877 |
| 595 | GGGAGACC G ACGAAGAG | 1014 | CUCUUCGU GGAGGAAACUCC CU UCAAGGACAUCGUCCGGG GGUCUCCC | 3878 |
| 598 | AGACCGAC G AAGAGCCC | 1015 | GGGCUCUU GGAGGAAACUCC CU UCAAGGACAUCGUCCGGG GUCGGUCU | 3879 |
| 607 | AAGAGCCC G AGGAGCCC | 1016 | GGGCUCCU GGAGGAAACUCC CU UCAAGGACAUCGUCCGGG GGGCUCUU | 3880 |
| 654 | GACAACCU G AGGGGCAA | 1017 | UUGCCCCU GGAGGAAACUCC CU UCAAGGACAUCGUCCGGG AGGUUGUC | 3881 |
| 690 | GUGGAGAU G ACCGUGGG | 1018 | CCCACGGU GGAGGAAACUCC CU UCAAGGACAUCGUCCGGG AUCUCCAC | 3882 |
| 708 | AGCCCCCC G CAGACGCU | 1019 | AGCGUCUG GGAGGAAACUCC CU UCAAGGACAUCGUCCGGG GGGGGGCU | 3883 |
| 714 | CCGCAGAC G CUCAACAU | 1020 | AUGUUGAG GGAGGAAACUCC CU UCAAGGACAUCGUCCGGG GUCUGCGG | 3884 |
| 751 | GUAACUUU G CAGUGGGU | 1021 | ACCCACUG GGAGGAAACUCC CU UCAAGGACAUCGUCCGGG AAACUUAC | 3885 |
| 760 | CAGUGGGU G CUGCCCCC | 1022 | GGGGGCAG GGAGGAAACUCC CU UCAAGGACAUCGUCCGGG ACCCACUG | 3886 |
| 763 | UGGGUGCU G CCCCCCAC | 1023 | GUGGGGGG GGAGGAAACUCC CU UCAAGGACAUCGUCCGGG AGCACCCA | 3887 |
| 780 | CCCUUCCU G CAUCGCUA | 1024 | UAGCGAUG GGAGGAAACUCC CU UCAAGGACAUCGUCCGGG AGGAAGGG | 3888 |
| 785 | CCUGCAUC G CUACUACC | 1025 | GGUAGUAG GGAGGAAACUCC CU UCAAGGACAUCGUCCGGG GAUGCAGG | 3889 |
| 843 | GUGUAUGU G CCCUACAC | 1026 | GUGUAGGG GGAGGAAACUCC CU UCAAGGACAUCGUCCGGG ACAUACAC | 3890 |
| 883 | UGGGCACC G ACCUGGUA | 1027 | UACCAGGU GGAGGAAACUCC CU UCAAGGACAUCGUCCGGG GGUGCCCA | 3891 |
| 921 | GUCACUGU G CGUGCCAA | 1028 | UUGGCACG GGAGGAAACUCC CU UCAAGGACAUCGUCCGGG ACAGUGAC | 3892 |
| 925 | CUGUGCGU G CCAACAUU | 1029 | AAUGUUGG GGAGGAAACUCC CU UCAAGGACAUCGUCCGGG ACGCACAG | 3893 |
| 934 | CCAACAUU G CUGCCAUC | 1030 | GAUGGCAG GGAGGAAACUCC CU UCAAGGACAUCGUCCGGG AAUGUUGG | 3894 |
| 937 | ACAUUGCU G CCAUCACU | 1031 | AGUGAUGG GGAGGAAACUCC CU UCAAGGACAUCGUCCGGG AGCAAUGU | 3895 |
| 946 | CCAUCACU G AAUCAGAC | 1032 | GUCUGAUU GGAGGAAACUCC CU UCAAGGACAUCGUCCGGG AGUGAUGG | 3896 |
| 1006 | UGGCCUAU G CUGAGAUU | 1033 | AAUCUCAG GGAGGAAACUCC CU UCAAGGACAUCGUCCGGG AUAGGCCA | 3897 |
| 1009 | CCUAUGCU G AGAUUGCC | 1034 | GGCAAUCU GGAGGAAACUCC CU UCAAGGACAUCGUCCGGG AGCAUAGG | 3898 |
| 1015 | CUGAGAUU G CCAGGCCU | 1035 | AGGCCUGG GGAGGAAACUCC CU UCAAGGACAUCGUCCGGG AAUCUCAG | 3899 |
| 1024 | CCAGGCCU G ACGACUCC | 1036 | GGAGUCGU GGAGGAAACUCC CU UCAAGGACAUCGUCCGGG AGGCCUGG | 3990 |
| 1027 | GGCCUGAC G ACUCCCUG | 1037 | CAGGGAGU GGAGGAAACUCC CU UCAAGGACAUCGUCCGGG GUCAGGCC | 3901 |
| 1048 | CUUUCUUU G ACUCUCUG | 1038 | CAGAGAGU GGAGGAAACUCC CU UCAAGGACAUCGUCCGGG AAAGAAAG | 3902 |
| 1092 | UUCUCCCU G CAGCUUUG | 1039 | CAAAGCUG GGAGGAAACUCC CU UCAAGGACAUCGUCCGGG AGGGAGAA | 3903 |
| 1105 | UUUGUGGU G CUGGCUUC | 1040 | GAAGCCAG GGAGGAAACUCC CU UCAAGGACAUCGUCCGGG ACCACAAA | 3904 |
| 1129 | ACCAGUCU G AAGUGCUG | 1041 | CAGCACUU GGAGGAAACUCC CU UCAAGGACAUCGUCCGGG AGACUGGU | 3905 |
| 1134 | UCUGAAGU G CUGGCCUC | 1042 | GAGGCCAG GGAGGAAACUCC CU UCAAGGACAUCGUCCGGG ACUUCAGA | 3906 |
| 1158 | GGGAGCAU G AUCAUUGG | 1043 | CCAAUGAU GGAGGAAACUCC CU UCAAGGACAUCGUCCGGG AUGCUCCC | 3907 |
| 1174 | GAGGUAUC G ACCACUCG | 1044 | CGAGUGGU GGAGGAAACUCC CU UCAAGGACAUCGUCCGGG GAUACCUC | 3908 |
| 1182 | GACCACUC G CUGUACAC | 1045 | GUGUACAG GGAGGAAACUCC CU UCAAGGACAUCGUCCGGG GAGUGGUC | 3909 |
| 1234 | GGUAUUAU G AGGUGAUC | 1046 | GAUCACCU GGAGGAAACUCC CU UCAAGGACAUCGUCCGGG AUAAUACC | 3910 |
| 1239 | UAUGAGGU G AUCAUUGU | 1047 | ACAAUGAU GGAGGAAACUCC CU UCAAGGACAUCGUCCGGG ACCUCAUA | 3911 |
| 1248 | AUCAUUGU G CGGGUGGA | 1048 | UCCACCCG GGAGGAAACUCC CU UCAAGGACAUCGUCCGGG ACAAUGAU | 3912 |
| 1275 | CAGGAUCU G AAAAUGGA | 1049 | UCCAUUUU GGAGGAAACUCC CU UCAAGGACAUCGUCCGGG AGAUCCUG | 3913 |
| 1286 | AAUGGACU G CAAGGAGU | 1050 | ACUCCUUG GGAGGAAACUCC CU UCAAGGACAUCGUCCGGG AGUCCAUU | 3914 |
| 1303 | ACAACUAU G ACAAGAGC | 1051 | GCUCUUGU GGAGGAAACUCC CU UCAAGGACAUCGUCCGGG AUAGUUGU | 3915 |
| 1344 | CUUCGUUU G CCCAAGAA | 1052 | UUCUUGGG GGAGGAAACUCC CU UCAAGGACAUCGUCCGGG AAACGAAG | 3916 |
| 1360 | AAGUGUUU G AAGCUGCA | 1053 | UGCAGCUU GGAGGAAACUCC CU UCAAGGACAUCGUCCGGG AAACACUU | 3917 |
| 1366 | UUGAAGCU G CAGUCAAA | 1054 | UUUGACUG GGAGGAAACUCC CU UCAAGGACAUCGUCCGGG AGCUUCAA | 3918 |
| 1411 | AGUUCCCU G AUGGUUUC | 1055 | GAAACCAU GGAGGAAACUCC CU UCAAGGACAUCGUCCGGG AGGGAACU | 3919 |
| 1442 | GCUGGUGU G CUGGCAAG | 1056 | CUUGCCAG GGAGGAAACUCC CU UCAAGGACAUCGUCCGGG ACACCAGC | 3920 |
| 1504 | UAAUGGGU G AGGUUACC | 1057 | GGUAACCU GGAGGAAACUCC CU UCAAGGACAUCGUCCGGG ACCCAUUA | 3921 |
| 1526 | GUCCUUCC G CAUCACCA | 1058 | UGGUGAUG GGAGGAAACUCC CU UCAAGGACAUCGUCCGGG GGAAGGAC | 3922 |
| 1542 | AUCCUUCC G CAGCAAUA | 1059 | UAUUGCUG GGAGGAAACUCC CU UCAAGGACAUCGUCCGGG GGAAGGAU | 3923 |
| 1554 | CAAUACCU G CGGCCAGU | 1060 | ACUGGCCG GGAGGAAACUCC CU UCAAGGACAUCGUCCGGG AGGUAUUG | 3924 |
| 1588 | CCCAAGAC G ACUGUUAC | 1061 | GUAACAGU GGAGGAAACUCC CU UCAAGGACAUCGUCCGGG GUCUUGGG | 3925 |
| 1603 | ACAAGUUU G CCAUCUCA | 1062 | UGAGAUGG GGAGGAAACUCC CU UCAAGGACAUCGUCCGGG AAACUUGU | 3926 |
| 1672 | UUGUCUUU G AUCGGGCC | 1063 | GGCCCGAU GGAGGAAACUCC CU UCAAGGACAUCGUCCGGG AAAGACAA | 3927 |
| 1682 | UCGGGCCC G AAAACGAA | 1064 | UUCGUUUU GGAGGAAACUCC CU UCAAGGACAUCGUCCGGG GGGCCCGA | 3928 |
| 1688 | CCGAAAAC G AAUUGGCU | 1065 | AGCCAAUU GGAGGAAACUCC CU UCAAGGACAUCGUCCGGG GUUUUCGG | 3929 |
| 1699 | UUGGCUUU G CUGUCAGC | 1066 | GCUGACAG GGAGGAAACUCC CU UCAAGGACAUCGUCCGGG AAAGCCAA | 3930 |
| 1708 | CUGUCAGC G CUUGCCAU | 1067 | AUGGCAAG GGAGGAAACUCC CU UCAAGGACAUCGUCCGGG GCUGACAG | 3931 |
| 1712 | CAGCGCUU G CCAUGUGC | 1068 | GCACAUGG GGAGGAAACUCC CU UCAAGGACAUCGUCCGGG AAGCGCUG | 3932 |
| 1719 | UGCCAUGU G CACCAUGA | 1069 | UCAUCGUG GGAGGAAACUCC CU UCAAGGACAUCGUCCGGG ACAUGGCA | 3933 |
| 1723 | AUGUGCAC G AUGAGUUC | 1070 | GAACUCAU GGAGGAAACUCC CU UCAAGGACAUCGUCCGGG GUGCACAU | 3934 |
| 1726 | UGCACGAU G AGUUCAGG | 1071 | CCUGAACU GGAGGAAACUCC CU UCAAGGACAUCGUCCGGG AUCGUGCA | 3935 |
| 1807 | AGACAGAU G AGUCAACC | 1072 | GGUUGACU GGAGGAAACUCC CU UCAAGGACAUCGUCCGGG AUCUGUCU | 3936 |
| 1821 | ACCCUCAU G ACCAUAGC | 1073 | GCUAUGGU GGAGGAAACUCC CU UCAAGGACAUCGUCCGGG AUGAGGGU | 3937 |
| 1843 | UCAUGGCU G CCAUCUGC | 1074 | GCAGAUGG GGAGGAAACUCC CU UCAAGGACAUCGUCCGGG AGCCAUGA | 3938 |
| 1850 | UGCCAUCU G CGCCCUCU | 1075 | AGAGGGCG GGAGGAAACUCC CU UCAAGGACAUCGUCCGGG AGAUGGCA | 3939 |
| 1852 | CCAUCUGC G CCCUCUUC | 1076 | GAAGAGGG GGAGGAAACUCC CU UCAAGGACAUCGUCCGGG GCAGAUGG | 3940 |
| 1863 | CUCUUCAU G CUGCCACU | 1077 | AGUGGCAG GGAGGAAACUCC CU UCAAGGACAUCGUCCGGG AUGAAGAG | 3941 |
| 1866 | UUCAUGCU G CCACUCUG | 1078 | CAGAGUGG GGAGGAAACUCC CU UCAAGGACAUCGUCCGGG AGCAUGAA | 3942 |
| 1874 | GCCACUCU G CCUCAUGG | 1079 | CCAUGAGG GGAGGAAACUCC CU UCAAGGACAUCGUCCGGG AGAGUGGC | 3943 |
| 1895 | UCAGUGGC G CUGCCUCC | 1080 | GGAGGCAG GGAGGAAACUCC CU UCAAGGACAUCGUCCGGG GCCACUGA | 3944 |
| 1898 | CUGGCGCU G CCUCCGCU | 1081 | AGCGGAGG GGAGGAAACUCC CU UCAAGGACAUCGUCCGGG AGCGCCAC | 3945 |
| 1904 | CUGCCUCC G CUGCCUGC | 1082 | GCAGGCAG GGAGGAAACUCC CU UCAAGGACAUCGUCCGGG GGAGGCAG | 3946 |
| 1907 | CCUCCGCU G CCUGCGCC | 1083 | GGCCCAGG GGAGGAAACUCC CU UCAAGGACAUCGUCCGGG AGCGGAGG | 3947 |
| 1911 | CGCUGCCU G CGCCAGCA | 1084 | UGCUGGCG GGAGGAAACUCC CU UCAAGGACAUCGUCCGGG AGGCAGCG | 3948 |
| 1913 | CUGCCUGC G CCAGCAGC | 1085 | GCUGCUGG GGAGGAAACUCC CU UCAAGGACAUCGUCCGGG GCAGGCAG | 3949 |
| 1924 | AGCAGCAU G AUGACUUU | 1086 | AAACUCAU GGAGGAAACUCC CU UCAAGGACAUCGUCCGGG AUGCUGCU | 3950 |
| 1927 | AGCAUGAU G ACUUUGCU | 1087 | AGCAAAGU GGAGGAAACUCC CU UCAAGGACAUCGUCCGGG AUCAUGCU | 3951 |
| 1933 | AUGACUUU G CUGAUGAC | 1088 | GUCAUCAG GGAGGAAACUCC CU UCAAGGACAUCGUCCGGG AAAGUCAU | 3952 |
| 1936 | ACUUUGCU G AUGACAUC | 1089 | GAUGUCAU GGAGGAAACUCC CU UCAAGGACAUCGUCCGGG AGCAAAGU | 3953 |
| 1939 | UUGCUGAU G ACAUCUCC | 1090 | GGAGAUGU GGAGGAAACUCC CU UCAAGGACAUCGUCCGGG AUCAGCAA | 3954 |
| 1950 | AUCUCCCU G CUCAAGUG | 1091 | CACUUCAG GGAGGAAACUCC CU UCAAGGACAUCGUCCGGG AGGGAGAU | 3955 |
| 1953 | UCCCUGCU G AAGUGAGG | 1092 | CCUCACUU GGAGGAAACUCC CU UCAAGGACAUCGUCCGGG AGCAGGGA | 3956 |
| 1958 | GCUGAAGU G AGGAGGCC | 1093 | GGCCUCCU GGAGGAAACUCC CU UCAAGGACAUCGUCCGGG ACUUCAGC | 3957 |
| 2087 | CACCAAAU G CCUCUGCC | 1094 | GGCAGAGG GGAGGAAACUCC CU UCAAGGACAUCGUCCGGG AUUUGGUG | 3958 |
| 2093 | AUGCCUCU G CCUUGAUG | 1095 | CAUCAAGG GGAGGAAACUCC CU UCAAGGACAUCGUCCGGG AGAGGCAU | 3959 |
| 2098 | UCUGCCUU G AUGGAGAA | 1096 | UUCUCCAU GGAGGAAACUCC CU UCAAGGACAUCGUCCGGG AAGGCAGA | 3960 |
| 2179 | AGCACUCU G CUGGCGGG | 1097 | CCCGCCAG GGAGGAAACUCC CU UCAAGGACAUCGUCCGGG AGAGUGCU | 3961 |
| 2227 | GAAAUUCU G CUGCUUGA | 1098 | UCAAGCAG GGAGGAAACUCC CU UCAAGGACAUCGUCCGGG AGAAUUUC | 3962 |
| 2230 | AUUCUGCU C CUUGAAAC | 1099 | GUUUCAAG GGAGGAAACUCC CU UCAAGGACAUCGUCCGGG AGCAGAAU | 3963 |
| 2234 | UGCUGCUU G AAACUUCA | 1100 | UGAAGUUU GGAGGAAACUCC CU UCAAGGACAUCGUCCGGG AAGCAGCA | 3964 |
| 2248 | UCAGCCCU G AACCUUUG | 1101 | CAAAGGUU GGAGGAAACUCC CU UCAAGGACAUCGUCCGGG AGGGCUGA | 3965 |
| 2329 | CAUCACAC G CAGGUUAC | 1102 | GUAACCUG GGAGGAAACUCC CU UCAAGGACAUCGUCCGGG GUGUGAUG | 3966 |
| 2393 | GUUUCCCU G CUGGCCAA | 1103 | UUGGCCAG GGAGGAAACUCC CU UCAAGGACAUCGUCCGGG AGGGAAAC | 3967 |
| 2419 | GAGAGGAU G CACAGUUU | 1104 | AAACUGUG GGAGGAAACUCC CU UCAAGGACAUCGUCCGGG AUCCUCUC | 3968 |
| 2428 | CACAGUUU G CUAUUUGC | 1105 | GCAAAUAG GGAGGAAACUCC CU UCAAGGACAUCGUCCGGG AAACUGUG | 3969 |
| 2435 | UGCUAUUU G CUUUAGAG | 1106 | CUCUAAAG GGAGGAAACUCC CU UCAAGGACAUCGUCCGGG AAAUAGCA | 3970 |
| 2476 | ACAUUGGU G CAAAGAUU | 1107 | AAUCUUUG GGAGGAAACUCC CU UCAAGGACAUCGUCCGGG ACCAAUGU | 3971 |
| 2485 | CAAAGAUU G CCUCUUGA | 1108 | UCAAGAGG GGAGGAAACUCC CU UCAAGGACAUCGUCCGGG AAUCUUUG | 3972 |
| 2492 | UGCCUCUU G AAUUAAAA | 1109 | UUUUAAUU GGAGGAAACUCC CU UCAAGGACAUCGUCCGGG AAGAGGCA | 3973 |
| 219 | GUGCCGAU G UAGCGGGC | 1110 | GCCCGCUA GGAGGAAACUCC CU UCAAGGACAUCGUCCGGG AUCGGCAC | 3974 |
| 483 | CUCCUGCU G UGGAUGGG | 1111 | CCCAUCCA GGAGGAAACUCC CU UCAAGGACAUCGUCCGGG AGCAGGAG | 3975 |
| 634 | GCAGCUUU G UGGAGAUG | 1112 | CAUCUCCA GGAGGAAACUCC CU UCAAGGACAUCGUCCGGG AAAGCUGC | 3976 |
| 804 | AGGCAGCU G UCCAGCAC | 1113 | GUGCUGGA GGAGGAAACUCC CU UCAAGGACAUCGUCCGGG AGCUGCCU | 3977 |
| 835 | GGAAGGGU G UGUAUGUG | 1114 | CACAUACA GGAGGAAACUCC CU UCAAGGACAUCGUCCGGG ACCCUUCC | 3978 |
| 837 | AACGGUGU G UAUGUGCC | 1115 | GGCACAUA GGAGGAAACUCC CU UCAAGGACAUCGUCCGGG ACACCCUU | 3979 |
| 841 | GUGUGUAU G UGCCCUAC | 1116 | GUAGCGCA GGAGGAAACUCC CU UCAAGGACAUCGUCCGGG AUACACAC | 3980 |
| 919 | ACGUCACU G UGCGUGCC | 1117 | GGCACGCA GGAGGAAACUCC CU UCAAGGACAUCGUCCGGG AGUGACGU | 3981 |
| 1100 | GCAGCUUU G UGGUGCUG | 1118 | CAGCACCA GGAGGAAACUCC CU UCAAGGACAUCGUCCGGG AAAGCUGC | 3982 |
| 1144 | UGGCCUCU G UCGGAGGG | 1119 | CCCUCCGA GGAGGAAACUCC CU UCAAGGACAUCGUCCGGG AGAGGCCA | 3983 |
| 1185 | CACUCGCU G UACACAGG | 1120 | CCUGUGUA GGAGGAAACUCC CU UCAAGGACAUCGUCCGGG AGCGAGUG | 3984 |
| 1246 | UGAUCAUU G UGCGGGUG | 1121 | CACCCGCA GGAGGAAACUCC CU UCAAGGACAUCGUCCGGG AAUGAUCA | 3985 |
| 1315 | AGAGCAUU G UGGACACU | 1122 | ACUGUCCA GGAGGAAACUCC CU UCAAGGACAUCGUCCGGG AAUGCUCU | 3986 |
| 1356 | AAGAAAGU G UUUGAAGC | 1123 | GCUUCAAA GGAGGAAACUCC CU UCAAGGACAUCGUCCGGG ACUUUCUU | 3987 |
| 1440 | CAGCUGGU G UGCUGGCA | 1124 | UGCCAGCA GGAGGAAACUCC CU UCAAGGACAUCGUCCGGG ACCAGCUG | 3988 |
| 1570 | UGGAAGAU G UGGCCACG | 1125 | CGUGGCCA GGAGGAAACUCC CU UCAAGGACAUCGUCCGGG AUCUUCCA | 3989 |
| 1592 | AGACGACU G UUACAAGU | 1126 | ACUUGUAA GGAGGAAACUCC CU UCAAGGACAUCGUCCGGG AGUCGUCU | 3990 |
| 1630 | CGGGCACU G UUAUGGGA | 1127 | UCCCAUAA GGAGGAAACUCC CU UCAAGGACAUCGUCCGGG AGUGCCCG | 3991 |
| 1642 | UGGGAGCU G UUAUCAUG | 1128 | CAUGAUAA GGAGGAAACUCC CU UCAAGGACAUCGUCCGGG AGCUCCCA | 3992 |
| 1666 | UCUACGUU G UCUUUGAU | 1129 | AUCAAAGA GGAGGAAACUCC CU UCAAGGACAUCGUCCGGG AACGUAGA | 3993 |
| 1702 | GCUUUGCU G UCAGCGCU | 1130 | AGCGCUGA GGAGGAAACUCC CU UCAAGGACAUCGUCCGGG AGCAAAGC | 3994 |
| 1717 | CUUGCCAU G UGCACGAU | 1131 | AUCGUGCA GGAGGAAACUCC CU UCAAGGACAUCGUCCGGG AUGGCAAG | 3995 |
| 1759 | GCCCUUUU G UCACCUUG | 1132 | CAAGGUGA GGAGGAAACUCC CU UCAAGGACAUCGUCCGGG AAAAGGGC | 3996 |
| 1781 | GGAAGACU G UGGCUACA | 1133 | UGUAGCCA GGAGGAAACUCC CU UCAAGGACAUCGUCCGGG AGUCUUCC | 3997 |
| 1834 | UAGCCUAU G UCAUGGCU | 1134 | AGCCAUGA GGAGGAAACUCC CU UCAAGGACAUCGUCCGGG AUAGGCUA | 3998 |
| 1884 | CUCAUGGU G UGUCAGUG | 1135 | CACUGACA GGAGGAAACUCC CU UCAAGGACAUCGUCCGGG ACCAUGAG | 3999 |
| 1886 | CAUGGUGU G UCAGUGGC | 1136 | GCCACUGA GGAGGAAACUCC CU UCAAGGACAUCGUCCGGG ACACCAUG | 4000 |
| 2048 | UGGCACCU G UGGCCAGA | 1137 | UCUGGCCA GGAGGAAACUCC CU UCAAGGACAUCGUCCGGG AGGUGCCA | 4001 |
| 2139 | CAGGGACU G UACCUGUA | 1138 | UACAGGUA GGAGGAAACUCC CU UCAAGGACAUCGUCCGGG AGUCCCUG | 4002 |
| 2145 | CUGUACCU G UAGGAAAC | 1139 | GUUUCCUA GGAGGAAACUCC CU UCAAGGACAUCGUCCGGG AGGUACAG | 4003 |
| 2256 | GAACCUUU G UCCACCAU | 1140 | AUGGUGGA GGAGGAAACUCC CU UCAAGGACAUCGUCCGGG AAAGGUUC | 4004 |
| 2346 | CUUGGCGU G UGUCCCUG | 1141 | CAGGGACA GGAGGAAACUCC CU UCAAGGACAUCGUCCGGG ACGCCAAG | 4005 |
| 2348 | UGGCGUGU G UCCCUGUG | 1142 | CACAGGGA GGAGGAAACUCC CU UCAAGGACAUCGUCCGGG ACACGCCA | 4006 |
| 2354 | GUGUCCCU G UGGUACCC | 1143 | GGGUACCA GGAGGAAACUCC CU UCAAGGACAUCGUCCGGG AGGGACAC | 4007 |
| 2385 | CCAAGCUU G UUUCCCUG | 1144 | CAGGGAAA GGAGGAAACUCC CU UCAAGGACAUCGUCCGGG AAGCUUGG | 4008 |
| 2453 | CAGGGACU G UAUAAACA | 1145 | UGUUUAUA GGAGGAAACUCC CU UCAAGGACAUCGUCCGGG AGUCCCUG | 4009 |
| 14 | CGUCCGCA G CCCGCCCG | 1146 | CGGGCGGG GGAGGAAACUCC CU UCAAGGACAUCGUCCGGG UGCGGACG | 4010 |
| 26 | GCCCGGGA G CUGCGAGC | 1147 | GCUCGCAG GGAGGAAACUCC CU UCAAGGACAUCGUCCGGG UCCCGGGC | 4011 |
| 33 | AGCUGCGA G CCGCGAGC | 1148 | GCUCGCGG GGAGGAAACUCC CU UCAAGGACAUCGUCCGGG UCGCAGCU | 4012 |
| 40 | AGCCGCGA G CUGGAUUA | 1149 | UAAUCCAG GGAGGAAACUCC CU UCAAGGACAUCGUCCGGG UCGCGGCU | 4013 |
| 51 | GGAUUAUG G UGGCCUGA | 1150 | UCAGGCCA GGAGGAAACUCC CU UCAAGGACAUCGUCCGGG CAUAAUCC | 4014 |
| 54 | UUAUGGUG G CCUGAGCA | 1151 | UGCUCAGG GGAGGAAACUCC CU UCAAGGACAUCGUCCGGG CACCAUAA | 4015 |
| 60 | UGGCCUGA G CAGCCAAC | 1152 | GUUGGCUG GGAGGAAACUCC CU UCAAGGACAUCGUCCGGG UCAGGCCA | 4016 |
| 63 | CCUGAGCA G CCAACGCA | 1153 | UGCGUUGG GGAGGAAACUCC CU UCAAGGACAUCGUCCGGG UGCUCAGG | 4017 |
| 72 | CCAACGCA G CCGCAGGA | 1154 | UCCUGCGG GGAGGAAACUCC CU UCAAGGACAUCGUCCGGG UGCGUUGG | 4018 |
| 81 | CCGCAGGA G CCCGGAGC | 1155 | GCUCCGGG GGAGGAAACUCC CU UCAAGGACAUCGUCCGGG UCCUGCGG | 4019 |
| 88 | AGCCCGGA G CCCUUGCC | 1156 | GGCAAGGG GGAGGAAACUCC CU UCAAGGACAUCGUCCGGG UCCGGGCU | 4020 |
| 134 | CCAGGGAA G CCGCCACC | 1157 | GGUGGCGG GGAGGAAACUCC CU UCAAGGACAUCGUCCGGG UUCCCUGG | 4021 |
| 144 | CGCCACCG G CCCGCCAU | 1158 | AUGGCGGG GGAGGAAACUCC CU UCAAGGACAUCGUCCGGG CGGUGGCG | 4022 |
| 167 | CCCUCCCA G CCCCGCCG | 1159 | CGGCGGGG GGAGGAAACUCC CU UCAAGGACAUCGUCCGGG UGGGAGGG | 4023 |
| 179 | CGCCGGGA G CCCGCGCC | 1160 | GGCGCGGG GGAGGAAACUCC CU UCAAGGACAUCGUCCGGG UCCCGGCG | 4024 |
| 198 | CUGCCCAG G CUGGCCGC | 1161 | GCGGCCAG GGAGGAAACUCC CU UCAAGGACAUCGUCCGGG CUGGGCAG | 4025 |
| 202 | CCAGGCUG G CCGCCGCC | 1162 | GGCGGCGG GGAGGAAACUCC CU UCAAGGACAUCGUCCGGG CAGCCUGG | 4026 |
| 211 | CCGCCGCC G UGCCGAUG | 1163 | CAUCGGCA GGAGGAAACUCC CU UCAAGGACAUCGUCCGGG GGCGGCGG | 4027 |
| 222 | CCGAUGUA G CGGGCUCC | 1164 | GGAGCCCG GGAGGAAACUCC CU UCAAGGACAUCGUCCGGG UACAUCGG | 4028 |
| 226 | UGUAGCGG G CUCCGGAU | 1165 | AUCCGGAG GGAGGAAACUCC CU UCAAGGACAUCGUCCGGG CCGCUACA | 4029 |
| 239 | GGAUCCCA G CCUCUCCC | 1166 | GGGAGAGG GGAGGAAACUCC CU UCAAGGACAUCGUCCGGG UGGGAUCC | 4030 |
| 256 | CUGCUCCC G UGCUCUGC | 1167 | GCAGAGCA GGAGGAAACUCC CU UCAAGGACAUCGUCCGGG GGGAGCAG | 4031 |
| 290 | UCUCCACA G CCCGGACC | 1168 | GGUCCGGG GGAGGAAACUCC CU UCAAGGACAUCGUCCGGG UGUGGAGA | 4032 |
| 304 | ACCCGGGG G CUGGCCCA | 1169 | UGGGCCAG GGAGGAAACUCC CU UCAAGGACAUCGUCCGGG CCCCGGGU | 4033 |
| 308 | GGGGGCUG G CCCAGGGC | 1170 | GCCCUGGG GGAGGAAACUCC CU UCAAGGACAUCGUCCGGG CAGCCCCC | 4034 |
| 315 | GGCCCAGG G CCCUGCAG | 1171 | CUGCAGGG GGAGGAAACUCC CU UCAAGGACAUCGUCCGGG CCUGGGCC | 4035 |
| 324 | CCCUGCAG G CCCUGGCG | 1172 | CGCCAGGG GGAGGAAACUCC CU UCAAGGACAUCGUCCGGG CUGCAGGG | 4036 |
| 330 | AGGCCCUG G CGUCCUGA | 1173 | UCAGGACG GGAGGAAACUCC CU UCAAGGACAUCGUCCGGG CAGGGCCU | 4037 |
| 332 | GCCCUGGC G UCCUGAUG | 1174 | CAUCAGGA GGAGGAAACUCC CU UCAAGGACAUCGUCCGGG GCCAGGGC | 4038 |
| 348 | GCCCCCAA G CUCCCUCU | 1175 | AGAGGGAC GGAGGAAACUCC CU UCAAGGACAUCGUCCGGG UUGGGGGC | 4039 |
| 365 | CCUGAGAA G CCACCAGC | 1176 | GCUGGUGG GGAGGAAACUCC CU UCAAGGACAUCGUCCGGG UUCUCAGG | 4040 |
| 372 | AGCCACCA G CACCACCC | 1177 | GGGUGGUG GGAGGAAACUCC CU UCAAGGACAUCGUCCGGG UGGUGGCU | 4041 |
| 391 | ACUUGGGG G CAGGCGCC | 1178 | GGCGCCUG GGAGGAAACUCC CU UCAAGGACAUCGUCCGGG CCCCAAGU | 4042 |
| 395 | GGGGGCAG G CGCCAGGG | 1179 | CCCUGGCG GGAGGAAACUCC CU UCAAGGACAUCGUCCGGG CUGCCCCC | 4043 |
| 410 | GGACGGAC G UGGGCCAG | 1180 | CUGGCCCA GGAGGAAACUCC CU UCAAGGACAUCGUCCGGG GUCCGUCC | 4044 |
| 414 | GGACGUGG G CCAGUGCG | 1181 | CGCACUGG GGAGGAAACUCC CU UCAAGGACAUCGUCCGGG CCACGUCC | 4045 |
| 418 | GUGGGCCA G UGCGAGCC | 1182 | GGCUCGCA GGAGGAAACUCC CU UCAAGGACAUCGUCCGGG UGGCCCAC | 4046 |
| 424 | CAGUGCGA G CCCAGAGG | 1183 | CCUCUGGG GGAGGAAACUCC CU UCAAGGACAUCGUCCGGG UCGCACUG | 4047 |
| 433 | CCCAGAGG G CCCGAAGG | 1184 | CCUUCGGG GGAGGAAACUCC CU UCAAGGACAUCGUCCGGG CCUCUGGG | 4048 |
| 441 | GCCCGAAG G CCGGGGCC | 1185 | GGCCCCGG GGAGGAAACUCC CU UCAAGGACAUCGUCCGGG CUUCGGGC | 4049 |
| 447 | AGGCCGGG G CCCACCAU | 1186 | AUGGUGGG GGAGGAAACUCC CU UCAAGGACAUCGUCCGGG CCCGGCCU | 4050 |
| 457 | CCACCAUG G CCCAAGCC | 1187 | GGCUUGGG GGAGGAAACUCC CU UCAAGGACAUCGUCCGGG CAUGGUGG | 4051 |
| 463 | UGGCCCAA G CCCUGCCC | 1188 | GGGCAGGG GGAGGAAACUCC CU UCAAGGACAUCGUCCGGG UUGGGCCA | 4052 |
| 474 | CUGCCCUG G CUCCUGCU | 1189 | AGCAGGAG GGAGGAAACUCC CU UCAAGGACAUCGUCCGGG CAGGGCAG | 4053 |
| 491 | GUGGAUGG G CGCGGGAG | 1190 | CUCCCGCG GGAGGAAACUCC CU UCAAGGACAUCGUCCGGG CCAUCCAC | 4054 |
| 499 | GCGCGGGA G UGCUGCCU | 1191 | AGGCAGCA GGAGGAAACUCC CU UCAAGGACAUCGUCCGGG UCCCGCGC | 4055 |
| 515 | UGCCCACG G CACCCAGC | 1192 | GCUGGGUG GGAGGAAACUCC CU UCAAGGACAUCGUCCGGG CGUGGGCA | 4056 |
| 522 | GGCACCCA G CACGGCAU | 1193 | AUGCCGUG GGAGGAAACUCC CU UCAAGGACAUCGUCCGGG UGGGUGCC | 4057 |
| 527 | CCAGCACG G CAUCCGGC | 1194 | GCCGGAUG GGAGGAAACUCC CU UCAAGGACAUCGUCCGGG CGUGCUGG | 4058 |
| 534 | GGCAUCCG G CUGCCCCU | 1195 | AGGGGCAG GGAGGAAACUCC CU UCAAGGACAUCGUCCGGG CGGAUGCC | 4059 |
| 548 | CCUGCGCA G CGGCCUGG | 1196 | CCAGGCCG GGAGGAAACUCC CU UCAAGGACAUCGUCCGGG UGCGCAGG | 4060 |
| 551 | GCGCAGCG G CCUGGGGG | 1197 | CCCCCAGG GGAGGAAACUCC CU UCAAGGACAUCGUCCGGG CGCUGCGC | 4061 |
| 560 | CCUGGGGG G CGCCCCCC | 1198 | GGGGGGCG GGAGGAAACUCC CU UCAAGGACAUCGUCCGGG CCCCCAGG | 4062 |
| 573 | CCCCUGGG G CUGCGGCU | 1199 | AGCCGCAG GGAGGAAACUCC CU UCAAGGACAUCGUCCGGG CCCAGGGG | 4063 |
| 579 | GGGCUGCG G CUGCCCCG | 1200 | CGGGGCAG GGAGGAAACUCC CU UCAAGGACAUCGUCCGGG CGCAGCCC | 4064 |
| 603 | GACGAAGA G CCCGAGGA | 1201 | UCCUCGGG GGAGGAAACUCC CU UCAAGGACAUCGUCCGGG UCUUCGUC | 4065 |
| 612 | CCCGAGGA G CCCGGCCG | 1202 | CGGCCGGG GGAGGAAACUCC CU UCAAGGACAUCGUCCGGG UCCUCGGG | 4066 |
| 617 | GGAGGCCG G CCGGAGGG | 1203 | CCCUCCGG GGAGGAAACUCC CU UCAAGGACAUCGUCCGGG CGGGCUCC | 4067 |
| 626 | CCGGAGGG G CAGCUUUG | 1204 | CAAAGCUG GGAGGAAACUCC CU UCAAGGACAUCGUCCGGG CCCUCCGG | 4068 |
| 629 | GAGGGGCA G CUUUGUGG | 1205 | CCACAAAG GGAGGAAACUCC CU UCAAGGACAUCGUCCGGG UGCCCCUC | 4069 |
| 643 | UGGAGAUG G UGGACAAC | 1206 | GUUGUCCA GGAGGAAACUCC CU UCAAGGACAUCGUCCGGG CAUCUCCA | 4070 |
| 659 | CCUGAGGG G CAAGUCGG | 1207 | CCGACUUG GGAGGAAACUCC CU UCAAGGACAUCGUCCGGG CCCUCAGG | 4071 |
| 663 | AGGGGCAA G UCGGGGCA | 1208 | UGCCCCGA GGAGGAAACUCC CU UCAAGGACAUCGUCCGGG UUGCCCCU | 4072 |
| 669 | AAGUCGGG G CAGGGCUA | 1209 | UAGCCCUG GGAGGAAACUCC CU UCAAGGACAUCGUCCGGG CCCGACUU | 4073 |
| 674 | GGGGCAGG G CUACUACG | 1210 | CGUAGUAG GGAGGAAACUCC CU UCAAGGACAUCGUCCGGG CCUGCCCC | 4074 |
| 682 | GCUACUAC G UGGAGAUG | 1211 | CAUCUCCA GGAGGAAACUCC CU UCAAGGACAUCGUCCGGG GUAGUAGC | 4075 |
| 694 | AGAUGACC G UGGGCAGC | 1212 | GCUGCCCA GGAGGAAACUCC CU UCAAGGACAUCGUCCGGG GGUCAUCU | 4076 |
| 698 | GACCGUGG G CAGCCCCC | 1213 | GGGGGCUG GGAGGAAACUCC CU UCAAGGACAUCGUCCGGG CCACGGUC | 4077 |
| 701 | CGUGGGCA G CCCCCCGC | 1214 | GCGGGGGG GGAGGAAACUCC CU UCAAGGACAUCGUCCGGG UGCCCACG | 4078 |
| 727 | ACAUCCUG G UGGAUACA | 1215 | UGUAUCCA GGAGGAAACUCC CU UCAAGGACAUCGUCCGGG CAGGAUGU | 4079 |
| 737 | GGAUACAG G CAGCAGUA | 1216 | UACUGCUG GGAGGAAACUCC CU UCAAGGACAUCGUCCGGG CUGUAUCC | 4080 |
| 740 | UACAGGCA G CAGUAACU | 1217 | AGUUACUG GGAGGAAACUCC CU UCAAGGACAUCGUCCGGG UGCCUGUA | 4081 |
| 743 | AGGCAGCA G UAACUUUG | 1218 | CAAAGUUA GGAGGAAACUCC CU UCAAGGACAUCGUCCGGG UGCUGCCU | 4082 |
| 754 | ACUUUGCA G UGGGUGCU | 1219 | AGCACCCA GGAGGAAACUCC CU UCAAGGACAUCGUCCGGG UGCAAAGU | 4083 |
| 758 | UGCAGUGG G UGCUGCCC | 1220 | GGGCAGCA GGAGGAAACUCC CU UCAAGGACAUCGUCCGGG CCACUGCA | 4084 |
| 798 | UACCAGAG G CAGCUGUC | 1221 | GACAGCUG GGAGGAAACUCC CU UCAAGGACAUCGUCCGGG CUCUGGUA | 4085 |
| 801 | CAGAGGCA G CUGUCCAG | 1222 | CUGGACAG GGAGGAAACUCC CU UCAAGGACAUCGUCCGGG UGCCUCUG | 4086 |
| 809 | GCUGUCCA G CACAUACC | 1223 | GGUAUGUG GGAGGAAACUCC CU UCAAGGACAUCGUCCGGG UGGACAGC | 4087 |
| 833 | CCGGAAGG G UGUGUAUG | 1224 | CAUACACA GGAGGAAACUCC CU UCAAGGACAUCGUCCGGG CCUUCCGG | 4088 |
| 857 | CACCCAGG G CAAGUGGG | 1225 | CCCACUUG GGAGGAAACUCC CU UCAAGGACAUCGUCCGGG CCUGGGUG | 4089 |
| 861 | CAGGGCAA G UGGGAAGG | 1226 | CCUUCCCA GGAGGAAACUCC CU UCAAGGACAUCGUCCGGG UUGCCCUG | 4090 |
| 873 | GAAGGGGA G CUGGGCAC | 1227 | CUGCCCAG GGAGGAAACUCC CU UCAAGGACAUCGUCCGGG UCCCCUUC | 4091 |
| 878 | GGAGCUGG G CACCGACC | 1228 | GGUCGGUG GGAGGAAACUCC CU UCAAGGACAUCGUCCGGG CCAGCUCC | 4092 |
| 889 | CCGACCUG G UAAGCAUC | 1229 | GAUGCUUA GGAGGAAACUCC CU UCAAGGACAUCGUCCGGG CAGGUCGG | 4093 |
| 893 | CCUGGUAA G CAUCCCCC | 1230 | GGGGGAUG GGAGGAAACUCC CU UCAAGGACAUCGUCCGGG UUACCAGG | 4094 |
| 905 | CCCCCAUG G CCCCAACG | 1231 | CGUUGGGG GGAGGAAACUCC CU UCAAGGACAUCGUCCGGG CAUGGGGG | 4095 |
| 913 | GCCCCAAC G UCACUGUG | 1232 | CACAGUGA GGAGGAAACUCC CU UCAAGGACAUCGUCCGGG GUUGGGGC | 4096 |
| 923 | CACUGUGC G UGCCAACA | 1233 | UGUUGGCA GGAGGAAACUCC CU UCAAGGACAUCGUCCGGG GCACAGUG | 4097 |
| 957 | UCAGACAA G UUCUUCAU | 1234 | AUGAAGAA GGAGGAAACUCC CU UCAAGGACAUCGUCCGGG UUGUCUGA | 4098 |
| 971 | CAUCAACG G CUCCAACU | 1235 | AGUUGGAG GGAGGAAACUCC CU UCAAGGACAUCGUCCGGG CGUUGAUG | 4099 |
| 986 | CUGGGAAG G CAUCCUGG | 1236 | CCAGGAUG GGAGGAAACUCC CU UCAAGGACAUCGUCCGGG CUUCCCAG | 4100 |
| 996 | AUCCUGGG G CUGGCCUA | 1237 | UAGGCCAG GGAGGAAACUCC CU UCAAGGACAUCGUCCGGG CCCAGGAU | 4101 |
| 1000 | UGGGGCUG G CCUAUGCU | 1238 | AGCAUAGG GGAGGAAACUCC CU UCAAGGACAUCGUCCGGG CAGCCCCA | 4102 |
| 1020 | AUUGCCAG G CCUGACGA | 1239 | UCGUCAGG GGAGGAAACUCC CU UCAAGGACAUCGUCCGGG CUGGCAAU | 4103 |
| 1038 | UCCCUGGA G CCUUUCUU | 1240 | AAGAAAGG GGAGGAAACUCC CU UCAAGGACAUCGUCCGGG UCCAGGGA | 4104 |
| 1057 | ACUCUCUG G UAAAGCAG | 1241 | CUGCUUUA GGAGGAAACUCC CU UCAAGGACAUCGUCCGGG CAGAGAGU | 4105 |
| 1062 | CUGGUAAA G CAGACCCA | 1242 | UGGGUCUG GGAGGAAACUCC CU UCAAGGACAUCGUCCGGG UUUACCAG | 4106 |
| 1072 | AGACCCAC G UUCCCAAC | 1243 | GUUGGGAA GGAGGAAACUCC CU UCAAGGACAUCGUCCGGG GUGGGUCU | 4107 |
| 1095 | UCCCUGCA G CUUUGUGG | 1244 | CCACAAAG GGAGGAAACUCC CU UCAAGGACAUCGUCCGGG UGCAGGGA | 4108 |
| 1103 | GCUUUGUG G UGCUGGCU | 1245 | AGCCAGCA GGAGGAAACUCC CU UCAAGGACAUCGUCCGGG CACAAAGC | 4109 |
| 1109 | UGGUGCUG G CUUCCCCC | 1246 | GGGGGAAG GGAGGAAACUCC CU UCAAGGACAUCGUCCGGG CAGCACCA | 4110 |
| 1125 | CUCAACCA G UCUGAAGU | 1247 | ACUUCAGA GGAGGAAACUCC CU UCAAGGACAUCGUCCGGG UGGUUGAG | 4111 |
| 1132 | AGUCUGAA G UGCUGGCC | 1248 | GGCCAGCA GGAGGAAACUCC CU UCAAGGACAUCGUCCGGG UUCAGACU | 4112 |
| 1138 | AAGUGCUG G CCUCUGUC | 1249 | GACAGAGG GGAGGAAACUCC CU UCAAGGACAUCGUCCGGG CAGCACUU | 4113 |
| 1154 | CGGAGGGA G CAUGAUCA | 1250 | UGAUCAUG GGAGGAAACUCC CU UCAAGGACAUCGUCCGGG UCCCUCCG | 4114 |
| 1169 | CAUUGGAG G UAUCGACC | 1251 | GGUCGAUA GGAGGAAACUCC CU UCAAGGACAUCGUCCGGG CUCCAAUG | 4115 |
| 1193 | GUACACAG G CAGUCUCU | 1252 | AGAGACUG GGAGGAAACUCC CU UCAAGGACAUCGUCCGGG CUGUGUAC | 4116 |
| 1196 | CACAGGCA G UCUCUGGU | 1253 | ACCAGAGA GGAGGAAACUCC CU UCAAGGACAUCGUCCGGG UGCCUGUG | 4117 |
| 1203 | AGUCUCUG G UAUACACC | 1254 | GGUGUAUA GGAGGAAACUCC CU UCAAGGACAUCGUCCGGG CAGAGACU | 4118 |
| 1218 | CCCAUCCG G CGGGAGUG | 1255 | CACUCCCG GGAGGAAACUCC CU UCAAGGACAUCGUCCGGG CGGAUGGG | 4119 |
| 1224 | CGGCGGGA G UGGUAUUA | 1256 | UAAUACCA GGAGGAAACUCC CU UCAAGGACAUCGUCCGGG UCCCGCCG | 4120 |
| 1227 | CGGGAGUG G UAUUAUGA | 1257 | UCAUAAUA GGAGGAAACUCC CU UCAAGGACAUCGUCCGGG CACUCCCG | 4121 |
| 1237 | AUUAUGAG G UGAUCAUU | 1258 | AAUGAUCA GGAGGAAACUCC CU UCAAGGACAUCGUCCGGG CUCAUAAU | 4122 |
| 1252 | UUGUGCGG G UGGAGAUC | 1259 | GAUCUCCA GGAGGAAACUCC CU UCAAGGACAUCGUCCGGG CCGCACAA | 4123 |
| 1293 | UGCAAGGA G UACAACUA | 1260 | UAGUUGUA GGAGGAAACUCC CU UCAAGGACAUCGUCCGGG UCCUUGCA | 4124 |
| 1310 | UGACAAGA G CAUUGUGG | 1261 | CCACAAUG GGAGGAAACUCC CU UCAAGGACAUCGUCCGGG UCUUGUCA | 4125 |
| 1322 | UGUGGACA G UGGCACCA | 1262 | UGGUGCCA GGAGGAAACUCC CU UCAAGGACAUCGUCCGGG UGUCCACA | 4126 |
| 1325 | GGACAGUG G CACCACCA | 1263 | UGGUGGUG GGAGGAAACUCC CU UCAAGGACAUCGUCCGGG CACUGUCC | 4127 |
| 1340 | CAACCUUC G UUUGCCCA | 1264 | UGGGCAAA GGAGGAAACUCC CU UCAAGGACAUCGUCCGGG GAAGGUUG | 4128 |
| 1354 | CCAAGAAA G UGUUUGAA | 1265 | UUCAAACA GGAGGAAACUCC CU UCAAGGACAUCGUCCGGG UUUCUUGG | 4129 |
| 1363 | UGUUUGAA G CUGCAGUC | 1266 | GACUGCAG GGAGGAAACUCC CU UCAAGGACAUCGUCCGGG UUCAAACA | 4130 |
| 1369 | AAGCUGCA G UCAAAUCC | 1267 | GGAUUUGA GGAGGAAACUCC CU UCAAGGACAUCGUCCGGG UGCAGCUU | 4131 |
| 1384 | CCAUCAAG G CAGCCUCC | 1268 | GGAGGCUG GGAGGAAACUCC CU UCAAGGACAUCGUCCGGG CUUGAUGG | 4132 |
| 1387 | UCAAGGCA G CCUCCUCC | 1269 | GGAGGAGG GGAGGAAACUCC CU UCAAGGACAUCGUCCGGG UGCCUUGA | 4133 |
| 1404 | ACGGAGAA G UUCCCUGA | 1270 | UCAGGGAA GGAGGAAACUCC CU UCAAGGACAUCGUCCGGG UUCUCCGU | 4134 |
| 1415 | CCCUGAUG G UUUCUGGC | 1271 | GCCAGAAA GGAGGAAACUCC CU UCAAGGACAUCGUCCGGG CAUCAGGG | 4135 |
| 1422 | GGUUUCUG G CUAGGAGA | 1272 | UCUCCUAG GGAGGAAACUCC CU UCAAGGACAUCGUCCGGG CAGAAACC | 4136 |
| 1431 | CUAGGAGA G CAGCUGGU | 1273 | ACCAGCUG GGAGGAAACUCC CU UCAAGGACAUCGUCCGGG UCUCCUAG | 4137 |
| 1434 | GGAGAGCA G CUGGUGUG | 1274 | CACACCAG GGAGGAAACUCC CU UCAAGGACAUCGUCCGGG UGCUCUCC | 4138 |
| 1438 | AGCAGCUG G UGUGCUGG | 1275 | CCAGCACA GGAGGAAACUCC CU UCAAGGACAUCGUCCGGG CAGCUGCU | 4139 |
| 1446 | GUGUGCUG G CAAGCAGG | 1276 | CCUGCUUG GGAGGAAACUCC CU UCAAGGACAUCGUCCGGG CAGCACAC | 4140 |
| 1450 | GCUGGCAA G CAGGCACC | 1277 | GGUGCCUG GGAGGAAACUCC CU UCAAGGACAUCGUCCGGG UUGCCAGC | 4141 |
| 1454 | GCAAGCAG G CACCACCC | 1278 | GGGUGGUG GGAGGAAACUCC CU UCAAGGACAUCGUCCGGG CUGCUUGC | 4142 |
| 1480 | UUUUCCCA G UCAUCUCA | 1279 | UGAGAUGA GGAGGAAACUCC CU UCAAGGACAUCGUCCGGG UGGGAAAA | 4143 |
| 1502 | CCUAAUGG G UGAGGUUA | 1280 | UAACCUCA GGAGGAAACUCC CU UCAAGGACAUCGUCCGGG CCAUUAGG | 4144 |
| 1507 | UGGGUGAG G UUACCAAC | 1281 | GUUGGUAA GGAGGAAACUCC CU UCAAGGACAUCGUCCGGG CUCACCCA | 4145 |
| 1518 | ACCAACCA G UCCUUCCG | 1282 | CGGAAGGA GGAGGAAACUCC CU UCAAGGACAUCGUCCGGG UGGUUGGU | 4146 |
| 1545 | CUUCCGCA G CAAUACCU | 1283 | AGGUAUUG GGAGGAAACUCC CU UCAAGGACAUCGUCCGGG UGCGGAAG | 4147 |
| 1557 | UACCUGCG G CCAGUGGA | 1284 | UCCACUGG GGAGGAAACUCC CU UCAAGGACAUCGUCCGGG CGCAGGUA | 4148 |
| 1561 | UGCGGCCA G UGGAAGAU | 1285 | AUCUUCCA GGAGGAAACUCC CU UCAAGGACAUCGUCCGGG UGGCCGCA | 4149 |
| 1573 | AAGAUGUG G CCACGUCC | 1286 | GGACGUGG GGAGGAAACUCC CU UCAAGGACAUCGUCCGGG CACAUCUU | 4150 |
| 1578 | GUGGCCAC G UCCCAAGA | 1287 | UCUUGGGA GGAGGAAACUCC CU UCAAGGACAUCGUCCGGG GUGGCCAC | 4151 |
| 1599 | UGUUACAA G UUUGCCAU | 1288 | AUGGCAAA GGAGGAAACUCC CU UCAAGGACAUCGUCCGGG UUGUAACA | 4152 |
| 1614 | AUCUCACA G UCAUCCAC | 1289 | GUGGAUGA GGAGGAAACUCC CU UCAAGGACAUCGUCCGGG UGUGAGAU | 4153 |
| 1625 | AUCCACGG G CACUGUUA | 1290 | UAACAGUG GGAGGAAACUCC CU UCAAGGACAUCGUCCGGG CCGUGGAU | 4154 |
| 1639 | UUAUGGGA G CUGUUAUC | 1291 | GAUAACAG GGAGGAAACUCC CU UCAAGGACAUCGUCCGGG UCCCAUAA | 4155 |
| 1655 | CAUGGAGG G CUUCUACG | 1292 | CGUAGAAG GGAGGAAACUCC CU UCAAGGACAUCGUCCGGG CCUCCAUG | 4156 |
| 1663 | GCUUCUAC G UUGUCUUU | 1293 | AAAGACAA GGAGGAAACUCC CU UCAAGGACAUCGUCCGGG GUAGAAGC | 4157 |
| 1678 | UUGAUCGG G CCCGAAAA | 1294 | UUUUCGGG GGAGGAAACUCC CU UCAAGGACAUCGUCCGGG CCGAUCAA | 4158 |
| 1694 | ACGAAUUG G CUUUGCUG | 1295 | CAGCAAAG GGAGGAAACUCC CU UCAAGGACAUCGUCCGGG CAAUUCGU | 4159 |
| 1706 | UGCUGUCA G CGCUUGCC | 1296 | GGCAAGCG GGAGGAAACUCC CU UCAAGGACAUCGUCCGGG UGACAGCA | 4160 |
| 1728 | CACGAUGA G UUCAGGAC | 1297 | GUCCUGAA GGAGGAAACUCC CU UCAAGGACAUCGUCCGGG UCAUCGUG | 4161 |
| 1738 | UCAGGACG G CAGCGGUG | 1298 | CACCGCUG GGAGGAAACUCC CU UCAAGGACAUCGUCCGGG CGUCCUGA | 4162 |
| 1741 | GGACGGCA G CGGUGGAA | 1299 | UUCCACCG GGAGGAAACUCC CU UCAAGGACAUCGUCCGGG UGCCGUCC | 4163 |
| 1744 | CGGCAGCG G UGGAAGGC | 1300 | GCCUUCCA GGAGGAAACUCC CU UCAAGGACAUCGUCCGGG CGCUGCCG | 4164 |
| 1751 | GGUGGAAG G CCCUUUUG | 1301 | CAAAAGGG GGAGGAAACUCC CU UCAAGGACAUCGUCCGGG CUUCCACC | 4165 |
| 1784 | AGACUGUG G CUACAACA | 1302 | UGUUGUAG GGAGGAAACUCC CU UCAAGGACAUCGUCCGGG CACAGUCU | 4166 |
| 1809 | ACAGAUGA G UCAACCCU | 1303 | AGGGUUGA GGAGGAAACUCC CU UCAAGGACAUCGUCCGGG UCAUCUGU | 4167 |
| 1828 | UGACCAUA G CCUAUGUC | 1304 | GACAUAGG GGAGGAAACUCC CU UCAAGGACAUCGUCCGGG UAUGGUCA | 4168 |
| 1840 | AUGUCAUG G CUGCCAUC | 1305 | GAUGGCAG GGAGGAAACUCC CU UCAAGGACAUCGUCCGGG CAUGACAU | 4169 |
| 1882 | GCCUCAUG G UGUGUCAG | 1306 | CUGACACA GGAGGAAACUCC CU UCAAGGACAUCGUCCGGG CAUGAGGC | 4170 |
| 1890 | GUGUGUCA G UGGCGCUG | 1307 | CAGCGCCA GGAGGAAACUCC CU UCAAGGACAUCGUCCGGG UGACACAC | 4171 |
| 1893 | UGUCAGUG G CGCUGCCU | 1308 | AGGCAGCG GGAGGAAACUCC CU UCAAGGACAUCGUCCGGG CACUGACA | 4172 |
| 1917 | CUGCGCCA G CAGCAUGA | 1309 | UCAUCCUG GGAGGAAACUCC CU UCAAGGACAUCGUCCGGG UGGCGCAG | 4173 |
| 1920 | CGCCAGCA G CAUGAUGA | 1310 | UCAUCAUG GGAGGAAACUCC CU UCAAGGACAUCGUCCGGG UGCUGGCG | 4174 |
| 1956 | CUGCUGAA G UGAGGAGG | 1311 | CCUCCUCA GGAGGAAACUCC CU UCAAGGACAUCGUCCGGG UUCAGCAG | 4175 |
| 1964 | GUGAGGAG G CCCAUGGG | 1312 | CCCAUGGG GGAGGAAACUCC CU UCAAGGACAUCGUCCGGG CUCCUCAC | 4176 |
| 1972 | GCCCAUGG G CAGAAGAU | 1313 | AUCUUCUG GGAGGAAACUCC CU UCAAGGACAUCGUCCGGG CCAUGGGC | 4177 |
| 2006 | ACACCUCC G UGGUUCAC | 1314 | GUGAACCA GGAGGAAACUCC CU UCAAGGACAUCGUCCGGG GGAGGUGU | 4178 |
| 2009 | CCUCCGUG G UUCACUUU | 1315 | AAAGUGAA GGAGGAAACUCC CU UCAAGGACAUCGUCCGGG CACGGAGG | 4179 |
| 2019 | UCACUUUG G UCACAAGU | 1316 | ACUUGUGA GGAGGAAACUCC CU UCAAGGACAUCGUCCGGG CAAAGUGA | 4180 |
| 2026 | GGUCACAA G UAGGAGAC | 1317 | GUCUCCUA GGAGGAAACUCC CU UCAAGGACAUCGUCCGGG UUGUGACC | 4181 |
| 2042 | CACAGAUG G CACCUGUG | 1318 | CACAGGUG GGAGGAAACUCC CU UCAAGGACAUCGUCCGGG CAUCUGUG | 4182 |
| 2051 | CACCUGUG G CCAGAGCA | 1319 | UGCUCUGG GGAGGAAACUCC CU UCAAGGACAUCGUCCGGG CACAGGUG | 4183 |
| 2057 | UGGCCAGA G CACCUCAG | 1320 | CUGAGGUG GGAGGAAACUCC CU UCAAGGACAUCGUCCGGG UCUGGCCA | 4184 |
| 2114 | AGGAAAAG G CUGGCAAG | 1321 | CUUGCCAG GGAGGAAACUCC CU UCAAGGACAUCGUCCGGG CUUUUCCU | 4185 |
| 2118 | AAAGGCUG G CAAGGUGG | 1322 | CCACCUUG GGAGGAAACUCC CU UCAAGGACAUCGUCCGGG CAGCCUUU | 4186 |
| 2123 | CUGGCAAG G UGGGUUCC | 1323 | GGAACCCA GGAGGAAACUCC CU UCAAGGACAUCGUCCGGG CUUGCCAG | 4187 |
| 2127 | CAAGGUGG G UUCCAGGG | 1324 | CCCUGGAA GGAGGAAACUCC CU UCAAGGACAUCGUCCGGG CCACCUUG | 4188 |
| 2172 | AGAAAGAA G CACUCUGC | 1325 | GCAGAGUG GGAGGAAACUCC CU UCAAGGACAUCGUCCGGG UUCUUUCU | 4189 |
| 2183 | CUCUGCUG G CGGGAAUA | 1326 | UAUUCCCG GGAGGAAACUCC CU UCAAGGACAUCGUCCGGG CAGCAGAG | 4190 |
| 2198 | UACUCUUG G UCACCUCA | 1327 | UCAGGUGA GGAGGAAACUCC CU UCAAGGACAUCGUCCGGG CAAGAGUA | 4191 |
| 2214 | AAAUUUAA G UCGGGAAA | 1328 | UUUCCCGA GGAGGAAACUCC CU UCAAGGACAUCGUCCGGG UUAAAUUU | 4192 |
| 2243 | AAACUUCA G CCCUGAAC | 1329 | GUUCAGGG GGAGGAAACUCC CU UCAAGGACAUCGUCCGGG UGAAGUUU | 4193 |
| 2288 | AACCCAAA G UAUUCUUC | 1330 | GAAGAAUA GGAGGAAACUCC CU UCAAGGACAUCGUCCGGG UUUGGGUU | 4194 |
| 2305 | UUUUCUUA G UUUCAGAA | 1331 | UUCUGAAA GGAGGAAACUCC CU UCAAGGACAUCGUCCGGG UAAGAAAA | 4195 |
| 2314 | UUUCAGAA G UACUGGCA | 1332 | UGCCAGUA GGAGGAAACUCC CU UCAAGGACAUCGUCCGGG UUCUGAAA | 4196 |
| 2320 | AAGUACUG G CAUCACAC | 1333 | GUGUGAUG GGAGGAAACUCC CU UCAAGGACAUCGUCCGGG CAGUACUU | 4197 |
| 2333 | ACACGCAG G UUACCUUG | 1334 | CAAGGUAA GGAGGAAACUCC CU UCAAGGACAUCGUCCGGG CUGCGUGU | 4198 |
| 2342 | UUACCUUG G CGUGUGUC | 1335 | GACACACG GGAGGAAACUCC CU UCAAGGACAUCGUCCGGG CAAGGUAA | 4199 |
| 2344 | ACCUUGGC G UGUGUCCC | 1336 | GGGACACA GGAGGAAACUCC CU UCAAGGACAUCGUCCGGG GCCAAGGU | 4200 |
| 2357 | UCCCUGUG G UACCCUGG | 1337 | CCAGGGUA GGAGGAAACUCC CU UCAAGGACAUCGUCCGGG CACAGGGA | 4201 |
| 2365 | GUACCCUG G CAGAGAAG | 1338 | CUUCUCUG GGAGGAAACUCC CU UCAAGGACAUCGUCCGGG CAGGGUAC | 4202 |
| 2381 | GAGACCAA G CUUGUUUC | 1339 | GAAACAAG GGAGGAAACUCC CU UCAAGGACAUCGUCCGGG UUGGUCUC | 4203 |
| 2397 | CCCUGCUG G CCAAAGUC | 1340 | GACUUUGG GGAGGAAACUCC CU UCAAGGACAUCGUCCGGG CAGCAGGG | 4204 |
| 2403 | UGGCCAAA G UCAGUAGG | 1341 | CCUACUGA GGAGGAAACUCC CU UCAAGGACAUCGUCCGGG UUUGGCCA | 4205 |
| 2407 | CAAAGUCA G UAGGAGAG | 1342 | CUCUCCUA GGAGGAAACUCC CU UCAAGGACAUCGUCCGGG UGACUUUG | 4206 |
| 2424 | GAUGCACA G UUUGCUAU | 1343 | AUAGCAAA GGAGGAAACUCC CU UCAAGGACAUCGUCCGGG UGUGCAUC | 4207 |
| 2463 | AUAAACAA G CCUAACAU | 1344 | AUGUUAGG GGAGGAAACUCC CU UCAAGGACAUCGUCCGGG UUGUUUAU | 4208 |
| 2474 | UAACAUUG G UGCAAAGA | 1345 | UCUUUGCA GGAGGAAACUCC CU UCAAGGACAUCGUCCGGG CAAUGUUA | 4209 |
| 22 | GCCCGCCC G GGAGCUGC | 1449 | GCAGCUCC GGAGGAAACUCC CU UCAAGGACAUCGUCCGGG GGGCGGGC | 4210 |
| 23 | CCCGCCCG G GAGCUGCG | 1450 | CGCAGCUC GGAGGAAACUCC CU UCAAGGACAUCGUCCGGG CGGGCGGG | 4211 |
| 24 | CCGCCCGG G AGCUGCGA | 1451 | UCGCAGCU GGAGGAAACUCC CU UCAAGGACAUCGUCCGGG CCGGGCGG | 4212 |
| 43 | CGCGAGCU G GAUUAUGG | 1452 | CCAUAAUC GGAGGAAACUCC CU UCAAGGACAUCGUCCGGG AGCUCGCG | 4213 |
| 44 | GCGAGCUG G AUUAUGGU | 1453 | ACCAUAAU GGAGGAAACUCC CU UCAAGGACAUCGUCCGGG CAGCUCGC | 4214 |
| 50 | UGGAUUAU G GUGGCCUG | 1454 | CAGGCCAC GGAGGAAACUCC CU UCAAGGACAUCGUCCGGG AUAAUCCA | 4215 |
| 53 | AUUAUGGU G GCCUGAGC | 1455 | GCUCAGGC GGAGGAAACUCC CU UCAAGGACAUCGUCCGGG ACCAUAAU | 4216 |
| 78 | CAGCCGCA G GAGCCCGG | 1456 | CCGGGCUC GGAGGAAACUCC CU UCAAGGACAUCGUCCGGG UGCGGCUG | 4217 |
| 79 | AGCCGCAG G AGCCCGGA | 1457 | UCCGGGCU GGAGGAAACUCC CU UCAAGGACAUCGUCCGGG CUGCGGCU | 4218 |
| 85 | AGGAGCCC G GAGCCCUU | 1458 | AAGGGCUC GGAGGAAACUCC CU UCAAGGACAUCGUCCGGG GGGCUCCU | 4219 |
| 86 | GGAGCCCG G AGCCCUUG | 1459 | CAAGGGCU GGAGGAAACUCC CU UCAAGGACAUCGUCCGGG CGGGCUCC | 4220 |
| 119 | CGCCCGCC G GGGGGACC | 1460 | GGUCCCCC GGAGGAAACUCC CU UCAAGGACAUCGUCCGGG GGCGGGCG | 4221 |
| 120 | GCCCGCCG G GGGGACCA | 1461 | UGGUCCCC GGAGGAAACUCC CU UCAAGGACAUCGUCCGGG CGGCGGGC | 4222 |
| 121 | CCCGCCGG G GGGACCAG | 1462 | CUGGUCCC GGAGGAAACUCC CU UCAAGGACAUCGUCCGGG CCGGCGGG | 4223 |
| 122 | CCGCCGGG G GGACCAGG | 1463 | CCUGGUCC GGAGGAAACUCC CU UCAAGGACAUCGUCCGGG CCCGGCGG | 4224 |
| 123 | CGCCGGGG G GACCAGGG | 1464 | CCCUGGUC GGAGGAAACUCC CU UCAAGGACAUCGUCCGGG CCCCGGCG | 4225 |
| 124 | GCCGGGGG G ACCAGGGA | 1465 | UCCCUGGU GGAGGAAACUCC CU UCAAGGACAUCGUCCGGG CCCCCGGC | 4226 |
| 129 | GGGGACCA G GGAAGCCG | 1466 | CGGCUUCC GGAGGAAACUCC CU UCAAGGACAUCGUCCGGG UGGUCCCC | 4227 |
| 130 | GGGACCAG G GAAGCCGC | 1467 | GCGGCUUC GGAGGAAACUCC CU UCAAGGACAUCGUCCGGG CUGGUCCC | 4228 |
| 131 | GGACCAGG G AAGCCGCC | 1468 | GGCGGCUU GGAGGAAACUCC CU UCAAGGACAUCGUCCGGG CCUGGUCC | 4229 |
| 143 | CCGCCACC G GCCCGCCA | 1469 | UGGCGGGC GGAGGAAACUCC CU UCAAGGACAUCGUCCGGG GGUGGCGG | 4230 |
| 175 | GCCCCGCC G GGAGCCCG | 1470 | CGGGCUCC GGAGGAAACUCC CU UCAAGGACAUCGUCCGGG GGCGGGGC | 4231 |
| 176 | CCCCGCCG G GAGCCCGC | 1471 | GCGGGCUC GGAGGAAACUCC CU UCAAGGACAUCGUCCGGG CGGCGGGG | 4232 |
| 177 | CCCGCCGG G AGCCCGCG | 1472 | CGCGGGCU GGAGGAAACUCC CU UCAAGGACAUCGUCCGGG CCGGCGGG | 4233 |
| 197 | GCUGCCCA G GCUGGCCG | 1473 | CGGCCAGC GGAGGAAACUCC CU UCAAGGACAUCGUCCGGG UGGGCAGC | 4234 |
| 201 | CCCAGGCU G GCCGCCGC | 1474 | GCGGCGGC GGAGGAAACUCC CU UCAAGGACAUCGUCCGGG AGCCUGGG | 4235 |
| 224 | GAUGUAGC G GGCUCCGG | 1475 | CCGGAGCC GGAGGAAACUCC CU UCAAGGACAUCGUCCGGG GCUACAUC | 4236 |
| 225 | AUGUAGCG G GCUCCGGA | 1476 | UCCGGAGC GGAGGAAACUCC CU UCAAGGACAUCGUCCGGG CGCUACAU | 4237 |
| 231 | CGGGCUCC G GAUCCCAG | 1477 | CUGGGAUC GGAGGAAACUCC CU UCAAGGACAUCGUCCGGG GGAGCCCG | 4238 |
| 232 | GGGCUCCG G AUCCCAGC | 1478 | GCUGGGAU GGAGGAAACUCC CU UCAAGGACAUCGUCCGGG CGGAGCCC | 4239 |
| 265 | UGCUCUGC G GAUCUCCC | 1479 | GGGAGAUC GGAGGAAACUCC CU UCAAGGACAUCGUCCGGG GCAGAGCA | 4240 |
| 266 | GCUCUGCG G AUCUCCCC | 1480 | GGGGAGAU GGAGGAAACUCC CU UCAAGGACAUCGUCCGGG CGCAGAGC | 4241 |
| 294 | CACAGCCC G GACCCGGG | 1481 | CCCGGGUC GGAGGAAACUCC CU UCAAGGACAUCGUCCGGG GGGCUGUG | 4242 |
| 295 | ACAGCCCG G ACCCGGGG | 1482 | CCCCGGGU GGAGGAAACUCC CU UCAAGGACAUCGUCCGGG CGGGCUGU | 4243 |
| 300 | CCGGACCC G GGGGCUGG | 1483 | CCAGCCCC GGAGGAAACUCC CU UCAAGGACAUCGUCCGGG GGGUCCGG | 4244 |
| 301 | CGGACCCG G GGGCUGCC | 1484 | GCCAGCCC GGAGGAAACUCC CU UCAAGGACAUCGUCCGGG CGGGUCCG | 4245 |
| 302 | GCACCCGG G GGCUGGCC | 1485 | GGCCAGCC GGAGGAAACUCC CU UCAAGGACAUCGUCCGGG CCGGGUCC | 4246 |
| 303 | GACCCGGG G GCUGGCCC | 1486 | GGGCCAGC GGAGGAAACUCC CU UCAAGGACAUCGUCCGGG CCCGGGUC | 4247 |
| 307 | CGGGGGCU G GCCCAGGG | 1487 | CCCUGGGC GGAGGAAACUCC CU UCAAGGACAUCGUCCGGG AGCCCCCG | 4248 |
| 313 | CUGGCCCA G GGCCCUGC | 1488 | GCAGGGCC GGAGGAAACUCC CU UCAAGGACAUCGUCCGGG UGGGCCAG | 4249 |
| 314 | UGGCCCAG G CCCCUGCA | 1489 | UGCAGGGC GGAGGAAACUCC CU UCAAGGACAUCGUCCGGG CUGGGCCA | 4250 |
| 323 | GCCCUGCA G GCCCUGGC | 1490 | GCCAGGGC GGAGGAAACUCC CU UCAAGGACAUCGUCCGGG UGCAGGGC | 4251 |
| 329 | CAGGCCCU G GCGUCCUG | 1491 | CAGGACGC GGAGGAAACUCC CU UCAAGGACAUCGUCCGGG AGGGCCUG | 4252 |
| 362 | UCUCCUGA G AAGCCACC | 1492 | GGUGGCUU GGAGGAAACUCC CU UCAAGGACAUCGUCCGGG UCAGGAGA | 4253 |
| 382 | ACCACCCA G ACUUGGGG | 1493 | CCCCAAGU GGAGGAAACUCC CU UCAAGGACAUCGUCCGGG UGGGUGGU | 4254 |
| 387 | CCAGACUU G GGGGCAGG | 1494 | CCUGCCCC GGAGGAAACUCC CU UCAAGGACAUCGUCCGGG AAGUCUGG | 4255 |
| 388 | CAGACUUG G GGGCAGGC | 1495 | GCCUGCCC GGAGGAAACUCC CU UCAAGGACAUCGUCCGGG CAAGUCUG | 4256 |
| 389 | AGACUUGG G GGCAGGCG | 1496 | CGCCUGCC GGAGGAAACUCC CU UCAAGGACAUCGUCCGGG CCAAGUCU | 4257 |
| 390 | GACUUGGG G GCAGGCGC | 1497 | GCGCCUGC GGAGGAAACUCC CU UCAAGGACAUCGUCCGGG CCCAAGUC | 4258 |
| 394 | UGGGGGCA G GCGCCAGG | 1498 | CCUGGCGC GGAGGAAACUCC CU UCAAGGACAUCGUCCGGG UGCCCCCA | 4259 |
| 401 | AGGCGCCA G GGACGGAC | 1499 | GUCCGUCC GGAGGAAACUCC CU UCAAGGACAUCGUCCGGG UGGCGCCU | 4260 |
| 402 | GGCGCCAG G GACGGACG | 1500 | CGUCCGUC GGAGGAAACUCC CU UCAAGGACAUCGUCCGGG CUGGCGCC | 4261 |
| 403 | GCGCCAGG G ACGGACGU | 1501 | ACGUCCGU GGAGGAAACUCC CU UCAAGGACAUCGUCCGGG CCUGGCGC | 4262 |
| 406 | CCAGGGAC G GACGUGGG | 1502 | CCCACGUC GGAGGAAACUCC CU UCAAGGACAUCGUCCGGG GUCCCUGG | 4263 |
| 407 | CAGGGACG G ACGUGGGC | 1503 | GCCCACGU GGAGGAAACUCC CU UCAAGGACAUCGUCCGGG CGUCCCUG | 4264 |
| 412 | ACGGACGU G GGCCAGUG | 1504 | CACUGGCC GGAGGAAACUCC CU UCAAGGACAUCGUCCGGG ACGUCCGU | 4265 |
| 413 | CGGACGUG G GCCAGUGC | 1505 | GCACUGGC GGAGGAAACUCC CU UCAAGGACAUCGUCCGGG CACGUCCG | 4266 |
| 429 | CGAGCCCA G AGGGCCCG | 1506 | CGGGCCCU GGAGGAAACUCC CU UCAAGGACAUCGUCCGGG UGGGCUCG | 4267 |
| 431 | AGCCCAGA G GGCCCGAA | 1507 | UUCGGGCC GGAGGAAACUCC CU UCAAGGACAUCGUCCGGG UCUGGGCU | 4268 |
| 432 | GCCCAGAG G GCCCGAAG | 1508 | CUUCGGGC GGAGGAAACUCC CU UCAAGGACAUCGUCCGGG CUCUGGGC | 4269 |
| 440 | GGCCCGAA G GCCGGGGC | 1509 | GCCCCGGC GGAGGAAACUCC CU UCAAGGACAUCGUCCGGG UUCGGGCC | 4270 |
| 444 | CGAAGGCC G GGGCCCAC | 1510 | GUGGGCCC GGAGGAAACUCC CU UCAAGGACAUCGUCCGGG GGCCUUCG | 4271 |
| 445 | GAAGGCCG G GGCCCACC | 1511 | GGUGGGCC GGAGGAAACUCC CU UCAAGGACAUCGUCCGGG CGGCCUUC | 4272 |
| 446 | AAGGCCGG G GCCCACCA | 1512 | UGGUGGGC GGAGGAAACUCC CU UCAAGGACAUCGUCCGGG CCGGCCUU | 4273 |
| 456 | CCCACCAU G GCCCAAGC | 1513 | GCUUGGGC GGAGGAAACUCC CU UCAAGGACAUCGUCCGGG AUGGUGGG | 4274 |
| 473 | CCUGCCCU G GCUCCUGC | 1514 | GCAGGAGC GGAGGAAACUCC CU UCAAGGACAUCGUCCGGG AGGGCAGG | 4275 |
| 485 | CCUGCUGU G GAUGGGCG | 1515 | CGCCCAUC GGAGGAAACUCC CU UCAAGGACAUCGUCCGGG ACAGCAGG | 4276 |
| 486 | CUGCUGUG G AUGGGCGC | 1516 | GCGCCCAU GGAGGAAACUCC CU UCAAGGACAUCGUCCGGG CACAGCAG | 4277 |
| 489 | CUGUGGAU G GGCGCGGG | 1517 | CCCGCGCC GGAGGAAACUCC CU UCAAGGACAUCGUCCGGG AUCCACAG | 4278 |
| 490 | UGUGGAUG G GCGCGGGA | 1518 | UCCCGCGC GGAGGAAACUCC CU UCAAGGACAUCGUCCGGG CAUCCACA | 4279 |
| 495 | AUGGGCGC G GGAGUGCU | 1519 | AGCACUCC GGAGGAAACUCC CU UCAAGGACAUCGUCCGGG GCGCCCAU | 4280 |
| 496 | UGGGCGCG G GAGUGCUG | 1520 | CAGCACUC GGAGGAAACUCC CU UCAAGGACAUCGUCCGGG CGCGCCCA | 4281 |
| 497 | GGGCGCGG G AGUGCUGC | 1521 | GCAGCACU GGAGGAAACUCC CU UCAAGGACAUCGUCCGGG CCGCGCCC | 4282 |
| 514 | CUGCCCAC G GCACCCAG | 1522 | CUGGGUGC GGAGGAAACUCC CU UCAAGGACAUCGUCCGGG GUGGGCAG | 4283 |
| 526 | CCCAGCAC G GCAUCCGG | 1523 | CCGGAUGC GGAGGAAACUCC CU UCAAGGACAUCGUCCGGG GUGCUGGG | 4284 |
| 533 | CGGCAUCC G GCUGCCCC | 1524 | GGGGCAGC GGAGGAAACUCC CU UCAAGGACAUCGUCCGGG GGAUGCCG | 4285 |
| 550 | UGCGCAGC G GCCUGGGG | 1525 | CCCCAGGC GGAGGAAACUCC CU UCAAGGACAUCGUCCGGG GCUGCGCA | 4286 |
| 555 | AGCGGCCU G GGGGGCGC | 1526 | GCGCCCCC GGAGGAAACUCC CU UCAAGGACAUCGUCCGGG AGGCCGCU | 4287 |
| 556 | GCGGCCUG G GGGGCGCC | 1527 | GGCGCCCC GGAGGAAACUCC CU UCAAGGACAUCGUCCGGG CAGGCCGC | 4288 |
| 557 | CGGCCUGG G GGGCGCCC | 1528 | GGGCGCCC GGAGGAAACUCC CU UCAAGGACAUCGUCCGGG CCAGGCCG | 4289 |
| 558 | GGCCUGGG G CGCGCCCC | 1529 | GGGGCGCC GGAGGAAACUCC CU UCAAGGACAUCGUCCGGG CCCAGGCC | 4290 |
| 559 | GCCUGGGG G GCGCCCCC | 1530 | GGGGGCGC GGAGGAAACUCC CU UCAAGGACAUCGUCCGGG CCCCAGGC | 4291 |
| 570 | GCCCCCCU G GGGCUGCG | 1531 | CGCAGCCC GGAGGAAACUCC CU UCAAGGACAUCGUCCGGG AGGGGGGC | 4292 |
| 571 | CCCCCCUG G GGCUGCGG | 1532 | CCGCAGCC GGAGGAAACUCC CU UCAAGGACAUCGUCCGGG CAGGGGGG | 4293 |
| 572 | CCCCCUGG G GCUGCGGC | 1533 | GCCGCAGC GGAGGAAACUCC CU UCAAGGACAUCGUCCGGG CCAGGGGG | 4294 |
| 578 | GGGGCUGC G GCUGCCCC | 1534 | GGGGCAGC GGAGGAAACUCC CU UCAAGGACAUCGUCCGGG GCAGCCCC | 4295 |
| 587 | GCUGCCCC G GGAGACCG | 1535 | CGGUCUCC GGAGGAAACUCC CU UCAAGGACAUCGUCCGGG GGGGCAGC | 4296 |
| 588 | CUGCCCCG G GAGACCGA | 1536 | UCGGUCUC GGAGGAAACUCC CU UCAAGGACAUCGUCCGGG CGGGGCAG | 4297 |
| 589 | UGCCCCGG G AGACCGAC | 1537 | GUCGGUCU GGAGGAAACUCC CU UCAAGGACAUCGUCCGGG CCGGGGCA | 4298 |
| 591 | CCCCGGGA G ACCGACGA | 1538 | UCGUCGGU GGAGGAAACUCC CU UCAAGGACAUCGUCCGGG UCCCGGGG | 4299 |
| 601 | CCGACGAA G AGCCCGAG | 1539 | CUCGGGCU GGAGGAAACUCC CU UCAAGGACAUCGUCCGGG UUCGUCGG | 4300 |
| 609 | GAGCCCGA G GAGCCCGG | 1540 | CCGGGCUC GGAGGAAACUCC CU UCAAGGACAUCGUCCGGG UCGGGCUC | 4301 |
| 610 | AGCCCGAG G AGCCCGGC | 1541 | GCCGGGCU GGAGGAAACUCC CU UCAAGGACAUCGUCCGGG CUCGGGCU | 4302 |
| 616 | AGGAGCCC G GCCGGAGG | 1542 | CCUCCGGC GGAGGAAACUCC CU UCAAGGACAUCGUCCGGG GGGCUCCU | 4303 |
| 620 | GCCCGGCC G GAGGGGCA | 1543 | UGCCCCUC GGAGGAAACUCC CU UCAAGGACAUCGUCCGGG GGCCGGGC | 4304 |
| 621 | CCCGGCCG G AGGGGCAG | 1544 | CUGCCCCU GGAGGAAACUCC CU UCAAGGACAUCGUCCGGG CGGCCGGG | 4305 |
| 623 | CGGCCGGA G GGGCAGCU | 1545 | AGCUGCCC GGAGGAAACUCC CU UCAAGGACAUCGUCCGGG UCCGGCCG | 4306 |
| 624 | GGCCGGAG G GGCAGCUU | 1546 | AAGCUGCC GGAGGAAACUCC CU UCAAGGACAUCGUCCGGG CUCCGGCC | 4307 |
| 625 | GCCGGAGG G GCAGCUUU | 1547 | AAAGCUGC GGAGGAAACUCC CU UCAAGGACAUCGUCCGGG CCUCCGGC | 4308 |
| 636 | AGCUUUGU G GAGAUGGU | 1548 | ACCAUCUC GGAGGAAACUCC CU UCAAGGACAUCGUCCGGG ACAAAGCU | 4309 |
| 637 | GCUUUGUG G AGAUGGUG | 1549 | CACCAUCU GGAGGAAACUCC CU UCAAGGACAUCGUCCGGG CACAAAGC | 4310 |
| 639 | UUUGUGGA G AUGGUGGA | 1550 | UCCACCAU GGAGGAAACUCC CU UCAAGGACAUCGUCCGGG UCCACAAA | 4311 |
| 642 | GUGGAGAU G GUGGACAA | 1551 | UUGUCCAC GGAGGAAACUCC CU UCAAGGACAUCGUCCGGG AUCUCCAC | 4312 |
| 645 | GAGAUGGU G GACAACCU | 1552 | AGGUUGUC GGAGGAAACUCC CU UCAAGGACAUCGUCCGGG ACCAUCUC | 4313 |
| 646 | AGAUGGUG G ACAACCUG | 1553 | CAGGUUGU GGAGGAAACUCC CU UCAAGGACAUCGUCCGGG CACCAUCU | 4314 |
| 656 | CAACCUGA G GGGCAAGU | 1554 | ACUUGCCC GGAGGAAACUCC CU UCAAGGACAUCGUCCGGG UCAGGUUG | 4315 |
| 657 | AACCUGAG G GGCAAGUC | 1555 | GACUUGCC GGAGGAAACUCC CU UCAAGGACAUCGUCCGGG CUCAGGUU | 4316 |
| 658 | ACCUGAGG G GCAAGUCG | 1556 | CGACUUGC GGAGGAAACUCC CU UCAAGGACAUCGUCCGGG CCUCAGGU | 4317 |
| 666 | GGCAAGUC G GGGCAGGG | 1557 | CCCUGCCC GGAGGAAACUCC CU UCAAGGACAUCGUCCGGG GACUUGCC | 4318 |
| 667 | GCAAGUCG G CGCAGGGC | 1558 | GCCCUGCC GGAGGAAACUCC CU UCAAGGACAUCGUCCGGG CGACUUGC | 4319 |
| 668 | CAAGUCGG G GCAGGGCU | 1559 | AGCCCUGC GGAGGAAACUCC CU UCAAGGACAUCGUCCGGG CCGACUUG | 4320 |
| 672 | UCGGGGCA G GGCUACUA | 1560 | UAGUAGCC GGAGGAAACUCC CU UCAAGGACAUCGUCCGGG UGCCCCGA | 4321 |
| 673 | CGGGGCAG G GCUACUAC | 1561 | GUAGUAGC GGAGGAAACUCC CU UCAAGGACAUCGUCCGGG CUGCCCCG | 4322 |
| 684 | UACUACGU G GAGAUGAC | 1562 | GUCAUCUC GGAGGAAACUCC CU UCAAGGACAUCGUCCGGG ACGUAGUA | 4323 |
| 685 | ACUACGUG G AGAUGACC | 1563 | GGUCAUCU GGAGGAAACUCC CU UCAAGGACAUCGUCCGGG CACGUAGU | 4324 |
| 687 | UACGUGGA G AUGACCGU | 1564 | ACGGUCAU GGAGGAAACUCC CU UCAAGGACAUCGUCCGGG UCCACGUA | 4325 |
| 696 | AUGACCGU G GGCAGCCC | 1565 | GGGCUGCC GGAGGAAACUCC CU UCAAGGACAUCGUCCGGG ACGGUCAU | 4326 |
| 697 | UGACCGUG G GCAGCCCC | 1566 | GGGGCUGC GGAGGAAACUCC CU UCAAGGACAUCGUCCGGG CACGGUCA | 4327 |
| 711 | CCCCCGCA G ACGCUCAA | 1567 | UUGAGCGU GGAGGAAACUCC CU UCAAGGACAUCGUCCGGG UGCGGGGG | 4328 |
| 726 | AACAUCCU G GUGGAUAC | 1568 | GUAUCCAC GGAGGAAACUCC CU UCAAGGACAUCGUCCGGG AGGAUGUU | 4329 |
| 729 | AUCCUGGU G GAUACAGG | 1569 | CCUGUAUC GGAGGAAACUCC CU UCAAGGACAUCGUCCGGG ACCAGGAU | 4330 |
| 730 | UCCUGGUG G AUACAGGC | 1570 | GCCUGUAU GGAGGAAACUCC CU UCAAGGACAUCGUCCGGG CACCAGGA | 4331 |
| 736 | UGGAUACA G GCAGCAGU | 1571 | ACUGCUGC GGAGGAAACUCC CU UCAAGGACAUCGUCCGGG UGUAUCCA | 4332 |
| 756 | UUUGCAGU G GGUGCUGC | 1572 | GCAGCACC GGAGGAAACUCC CU UCAAGGACAUCGUCCGGG ACUGCAAA | 4333 |
| 757 | UUGCAGUG G GUGCUGCC | 1573 | GGCAGCAC GGAGGAAACUCC CU UCAAGGACAUCGUCCGGG CACUGCAA | 4334 |
| 795 | UACUACCA G AGGCAGCU | 1574 | AGCUGCCU GGAGGAAACUCC CU UCAAGGACAUCGUCCGGG UGGUAGUA | 4335 |
| 797 | CUACCAGA G GCAGCUGU | 1575 | ACAGCUGC GGAGGAAACUCC CU UCAAGGACAUCGUCCGGG UCUGGUAG | 4336 |
| 818 | CACAUACC G GGACCUCC | 1576 | GGAGGUCC GGAGGAAACUCC CU UCAAGGACAUCGUCCGGG GGUAUGUG | 4337 |
| 819 | ACAUACCG G GACCUCCG | 1577 | CGGAGGUC GGAGGAAACUCC CU UCAAGGACAUCGUCCGGG CGGUAUGU | 4338 |
| 820 | CAUACCGG G ACCUCCGG | 1578 | CCGGAGGU GGAGGAAACUCC CU UCAAGGACAUCGUCCGGG CCGGUAUG | 4339 |
| 827 | GGACCUCC G GAAGGGUG | 1579 | CACCCUUC GGAGGAAACUCC CU UCAAGGACAUCGUCCGGG GGAGGUCC | 4340 |
| 828 | GACCUCCG G AAGGGUGU | 1580 | ACACCCUU GGAGGAAACUCC CU UCAAGGACAUCGUCCGGG CGGAGGUC | 4341 |
| 831 | CUCCGGAA G GGUGUGUA | 1581 | UACACACC GGAGGAAACUCC CU UCAAGGACAUCGUCCGGG UUCCGGAG | 4342 |
| 832 | UCCGGAAG G GUGUGUAU | 1582 | AUACACAC GGAGGAAACUCC CU UCAAGGACAUCGUCCGGG CUUCCGGA | 4343 |
| 855 | UACACCCA G GGCAAGUG | 1583 | CACUUGCC GGAGGAAACUCC CU UCAAGGACAUCGUCCGGG UGGGUGUA | 4344 |
| 856 | ACACCCAG G GCAAGUGG | 1584 | CCACUUGC GGAGGAAACUCC CU UCAAGGACAUCGUCCGGG CUGGGUGU | 4345 |
| 863 | GGGCAAGU G GGAAGGGG | 1585 | CCCCUUCC GGAGGAAACUCC CU UCAAGGACAUCGUCCGGG ACUUGCCC | 4346 |
| 864 | GGCAAGUG G GAAGGGGA | 1586 | UCCCCUUC GGAGGAAACUCC CU UCAAGGACAUCGUCCGGG CACUUGCC | 4347 |
| 865 | GCAAGUGG G AAGGGGAG | 1587 | CUCCCCUU GGAGGAAACUCC CU UCAAGGACAUCGUCCGGG CCACUUGC | 4348 |
| 868 | AGUGGGAA G GGGAGCUG | 1588 | CAGCUCCC GGAGGAAACUCC CU UCAAGGACAUCGUCCGGG UUCCCACU | 4349 |
| 869 | GUGGGAAG G GGAGCUGG | 1589 | CCAGCUCC GGAGGAAACUCC CU UCAAGGACAUCGUCCGGG CUUCCCAC | 4350 |
| 870 | UGGGAAGG G GAGCUGGG | 1590 | CCCAGCUC GGAGGAAACUCC CU UCAAGGACAUCGUCCGGG CCUUCCCA | 4351 |
| 871 | GGGAAGGG G AGCUGGGC | 1591 | GCCCAGCU GGAGGAAACUCC CU UCAAGGACAUCGUCCGGG CCCUUCCC | 4352 |
| 876 | GGGGAGCU G GGCACCGA | 1592 | UCGGUGCC GGAGGAAACUCC CU UCAAGGACAUCGUCCGGG AGCUCCCC | 4353 |
| 877 | GGGAGCUG G GCACCGAC | 1593 | GUCGGUGC GGAGGAAACUCC CU UCAAGGACAUCGUCCGGG CAGCUCCC | 4354 |
| 888 | ACCGACCU G GUAAGCAU | 1594 | AUGCUUAC GGAGGAAACUCC CU UCAAGGACAUCGUCCGGG AGGUCGGU | 4355 |
| 904 | UCCCCCAU G GCCCCAAC | 1595 | GUUGGGGC GGAGGAAACUCC CU UCAAGGACAUCGUCCGGG AUGGGGGA | 4356 |
| 952 | CUGAAUCA G ACAAGUUC | 1596 | GAACUUGU GGAGGAAACUCC CU UCAAGGACAUCGUCCGGG UGAUUCAG | 4357 |
| 970 | UCAUCAAC G GCUCCAAC | 1597 | GUUGGAGC GGAGGAAACUCC CU UCAAGGACAUCGUCCGGG GUUGAUGA | 4358 |
| 980 | CUCCAACU G GGAAGGCA | 1598 | UGCCUUCC GGAGGAAACUCC CU UCAAGGACAUCGUCCGGG AGUUGGAG | 4359 |
| 981 | UCCAACUG G GAAGGCAU | 1599 | AUGCCUUC GGAGGAAACUCC CU UCAAGGACAUCGUCCGGG CAGUUGGA | 4360 |
| 982 | CCAACUGG G AAGGCAUC | 1600 | GAUGCCUU GGAGGAAACUCC CU UCAAGGACAUCGUCCGGG CCAGUUGG | 4361 |
| 985 | ACUGGGAA G GCAUCCUG | 1601 | CAGGAUGC GGAGGAAACUCC CU UCAAGGACAUCGUCCGGG UUCCCAGU | 4362 |
| 993 | GGCAUCCU G GGGCUGGC | 1602 | GCCAGCCC GGAGGAAACUCC CU UCAAGGACAUCGUCCGGG AGGAUGCC | 4363 |
| 994 | GCAUCCUG G GGCUGGCC | 1603 | GGCCAGCC GGAGGAAACUCC CU UCAAGGACAUCGUCCGGG CAGGAUGC | 4364 |
| 995 | CAUCCUGG G GCUGGCCU | 1604 | AGGCCAGC GGAGGAAACUCC CU UCAAGGACAUCGUCCGGG CCAGGAUG | 4365 |
| 999 | CUGGGGCU G GCCUAUGC | 1605 | GCAUAGGC GGAGGAAACUCC CU UCAAGGACAUCGUCCGGG AGCCCCAG | 4366 |
| 1011 | UAUGCUGA G AUUGCCAG | 1606 | CUGGCAAU GGAGGAAACUCC CU UCAAGGACAUCGUCCGGG UCAGCAUA | 4367 |
| 1019 | GAUUGCCA G GCCUGACG | 1607 | CGUCAGGC GGAGGAAACUCC CU UCAAGGACAUCGUCCGGG UGGCAAUC | 4368 |
| 1035 | GACUCCCU G GAGCCUUU | 1608 | AAAGGCUC GGAGGAAACUCC CU UCAAGGACAUCGUCCGGG AGGGAGUC | 4369 |
| 1036 | ACUCCCUG G AGCCUUUC | 1609 | GAAAGGCU GGAGGAAACUCC CU UCAAGGACAUCGUCCGGG CAGGGAGU | 4370 |
| 1056 | GACUCUCU G GUAAAGCA | 1610 | UGCUUUAC GGAGGAAACUCC CU UCAAGGACAUCGUCCGGG AGAGAGUC | 4371 |
| 1065 | GUAAAGCA G ACCCACGU | 1611 | ACGUGGGU GGAGGAAACUCC CU UCAAGGACAUCGUCCGGG UGCUUUAC | 4372 |
| 1102 | AGCUUUGU G GUGCUGGC | 1612 | GCCAGCAC GGAGGAAACUCC CU UCAAGGACAUCGUCCGGG ACAAAGCU | 4373 |
| 1108 | GUGGUGCU G GCUUCCCC | 1613 | GGGGAAGC GGAGGAAACUCC CU UCAAGGACAUCGUCCGGG AGCACCAC | 4374 |
| 1137 | GAAGUGCU G GCCUCUGU | 1614 | ACAGAGGC GGAGGAAACUCC CU UCAAGGACAUCGUCCGGG AGCACUUC | 4375 |
| 1147 | CCUCUGUC G GAGGCAGC | 1615 | GCUCCCUC GGAGGAAACUCC CU UCAAGGACAUCGUCCGGG GACAGAGG | 4376 |
| 1148 | CUCUGUCG G AGGGAGCA | 1616 | UGCUCCCU GGAGGAAACUCC CU UCAAGGACAUCGUCCGGG CGACAGAG | 4377 |
| 1150 | CUGUCGGA G GGAGCAUG | 1617 | CAUGCUCC GGAGGAAACUCC CU UCAAGGACAUCGUCCGGG UCCGACAG | 4378 |
| 1151 | UGUCGGAG G GAGCAUGA | 1618 | UCAUGCUC GGAGGAAACUCC CU UCAAGGACAUCGUCCGGG CUCCGACA | 4379 |
| 1152 | GUCGGAGG G AGCAUGAU | 1619 | AUCAUGCU GGAGGAAACUCC CU UCAAGGACAUCGUCCGGG CCUCCGAC | 4380 |
| 1165 | UGAUCAUU G GAGGUAUC | 1620 | GAUACCUC GGAGGAAACUCC CU UCAAGGACAUCGUCCGGG AAUGAUCA | 4381 |
| 1166 | GAUCAUUG G AGGUAUCG | 1621 | CGAUACCU GGAGGAAACUCC CU UCAAGGACAUCGUCCGGG CAAUGAUC | 4382 |
| 1168 | UCAUUGGA G GUAUCGAC | 1622 | GUCGAUAC GGAGGAAACUCC CU UCAAGGACAUCGUCCGGG UCCAAUGA | 4383 |
| 1192 | UGUACACA G GCAGUCUC | 1623 | GAGACUGC GGAGGAAACUCC CU UCAAGGACAUCGUCCGGG UGUGUACA | 4384 |
| 1202 | CAGUCUCU G GUAUACAC | 1624 | GUGUAUAC GGAGGAAACUCC CU UCAAGGACAUCGUCCGGG AGAGACUG | 4385 |
| 1217 | ACCCAUCC G GCGGGAGU | 1625 | ACUCCCGC GGAGGAAACUCC CU UCAAGGACAUCGUCCGGG GGAUGGGU | 4386 |
| 1220 | CAUCCGGC G GGAGUGGU | 1626 | ACCACUCC GGAGGAAACUCC CU UCAAGGACAUCGUCCGGG GCCGGAUG | 4387 |
| 1221 | AUCCGGCG G GAGUGGUA | 1627 | UACCACUC GGAGGAAACUCC CU UCAAGGACAUCGUCCGGG CGCCGGAU | 4388 |
| 1222 | UCCGGCGG G AGUGGUAU | 1628 | AUACCACU GGAGGAAACUCC CU UCAAGGACAUCGUCCGGG CCGCCGGA | 4389 |
| 1226 | GCGGGAGU G GUAUUAUG | 1629 | CAUAAUAC GGAGGAAACUCC CU UCAAGGACAUCGUCCGGG ACUCCCGC | 4390 |
| 1236 | UAUUAUGA G GUGAUCAU | 1630 | AUGAUCAC GGAGGAAACUCC CU UCAAGGACAUCGUCCGGG UCAUAAUA | 4391 |
| 1250 | CAUUGUGC G GGUGGAGA | 1631 | UCUCCACC GGAGGAAACUCC CU UCAAGGACAUCGUCCGGG GCACAAUG | 4392 |
| 1251 | AUUGUGCG G GUGGAGAU | 1632 | AUCUCCAC GGAGGAAACUCC CU UCAAGGACAUCGUCCGGG CGCACAAU | 4393 |
| 1254 | GUGCGGGU G GAGAUCAA | 1633 | UUGAUCUC GGAGGAAACUCC CU UCAAGGACAUCGUCCGGG ACCCGCAC | 4394 |
| 1255 | UGCGGGUG G AGAUCAAU | 1634 | AUUGAUCU GGAGGAAACUCC CU UCAAGGACAUCGUCCGGG CACCCGCA | 4395 |
| 1257 | CGGGUGGA G AUCAAUGG | 1635 | CCAUUGAU GGAGGAAACUCC CU UCAAGGACAUCGUCCGGG UCCACCCG | 4396 |
| 1264 | ACAUCAAU G GACAGGAU | 1636 | AUCCUGUC GGAGGAAACUCC CU UCAAGGACAUCGUCCGGG AUUGAUCU | 4397 |
| 1265 | GAUCAAUG G ACAGGAUC | 1637 | GAUCCUGU GGAGGAAACUCC CU UCAAGGACAUCGUCCGGG CAUUGAUC | 4398 |
| 1269 | AAUGGACA G GAUCUGAA | 1638 | UUCAGAUC GGAGGAAACUCC CU UCAAGGACAUCGUCCGGG UGUCCAUU | 4399 |
| 1270 | AUGGACAG G AUCUGAAA | 1639 | UUUCAGAU GGAGGAAACUCC CU UCAAGGACAUCGUCCGGG CUGUCCAU | 4400 |
| 1281 | CUGAAAAU G GACUGCAA | 1640 | UUGCAGUC GGAGGAAACUCC CU UCAAGGACAUCGUCCGGG AUUUUCAG | 4401 |
| 1282 | UGAAAAUG G ACUGCAAG | 1641 | CUUGCAGU GGAGGAAACUCC CU UCAAGGACAUCGUCCGGG CAUUUUCA | 4402 |
| 1290 | GACUGCAA G GAGUACAA | 1642 | UUGUACUC GGAGGAAACUCC CU UCAAGGACAUCGUCCGGG UUGCAGUC | 4403 |
| 1291 | ACUGCAAG G AGUACAAC | 1643 | GUUGUACU GGAGGAAACUCC CU UCAAGGACAUCGUCCGGG CUUGCAGU | 4404 |
| 1308 | UAUGACAA G AGCAUUGU | 1644 | ACAAUGCU GGAGGAAACUCC CU UCAAGGACAUCGUCCGGG UUGUCAUA | 4405 |
| 1317 | AGCAUUGU G GACAGUGG | 1645 | CCACUGUC GGAGGAAACUCC CU UCAAGGACAUCGUCCGGG ACAAUGCU | 4406 |
| 1318 | GCAUUGUG G ACAGUGGC | 1646 | GCCACUGU GGAGGAAACUCC CU UCAAGGACAUCGUCCGGG CACAAUGC | 4407 |
| 1324 | UGGACAGU G GCACCACC | 1647 | GGUGGUGC GGAGGAAACUCC CU UCAAGGACAUCGUCCGGG ACUGUCCA | 4408 |
| 1350 | UUGCCCAA G AAAGUGUU | 1648 | AACACUUU GGAGGAAACUCC CU UCAAGGACAUCGUCCGGG UUGGGCAA | 4409 |
| 1383 | UCCAUCAA G GCAGCCUC | 1649 | GAGGCUGC GGAGGAAACUCC CU UCAAGGACAUCGUCCGGG UUGAUGGA | 4410 |
| 1398 | UCCUCCAC G GAGAAGUU | 1650 | AACUUCUC GGAGGAAACUCC CU UCAAGGACAUCGUCCGGG GUGGAGGA | 4411 |
| 1399 | CCUCCACG G AGAAGUUC | 1651 | GAACUUCU GGAGGAAACUCC CU UCAAGGACAUCGUCCGGG CGUGGAGG | 4412 |
| 1401 | UCCACGGA G AAGUUCCC | 1652 | GGGAACUU GGAGGAAACUCC CU UCAAGGACAUCGUCCGGG UCCGUGGA | 4413 |
| 1414 | UCCCUGAU G GUUUCUGG | 1653 | CCAGAAAC GGAGGAAACUCC CU UCAAGGACAUCGUCCGGG AUCAGGGA | 4414 |
| 1421 | UGGUUUCU G GCUAGGAG | 1654 | CUCCUAGC GGAGGAAACUCC CU UCAAGGACAUCGUCCGGG AGAAACCA | 4415 |
| 1426 | UCUGGCUA G GAGAGCAG | 1655 | CUGCUCUC GGAGGAAACUCC CU UCAAGGACAUCGUCCGGG UAGCCAGA | 4416 |
| 1427 | CUGGCUAG G AGAGCAGC | 1656 | GCUGCUCU GGAGGAAACUCC CU UCAAGGACAUCGUCCGGG CUAGCCAG | 4417 |
| 1429 | GGCUAGGA G ACCAGCUG | 1657 | CAGCUGCU GGAGGAAACUCC CU UCAAGGACAUCGUCCGGG UCCUAGCC | 4418 |
| 1437 | GAGCAGCU G GUGUGCUG | 1658 | CAGCACAC GGAGGAAACUCC CU UCAAGGACAUCGUCCGGG AGCUGCUC | 4419 |
| 1445 | GGUGUGCU G GCAAGCAG | 1659 | CUGCUUGC GGAGGAAACUCC CU UCAAGGACAUCGUCCGGG AGCACACC | 4420 |
| 1453 | GGCAAGCA G GCACCACC | 1660 | GGUGGUGC GGAGGAAACUCC CU UCAAGGACAUCGUCCGGG UGCUUGCC | 4421 |
| 1466 | CACCCCUU G GAACAUUU | 1661 | AAAUGUUC GGAGGAAACUCC CU UCAAGGACAUCGUCCGGG AAGGGGUG | 4422 |
| 1467 | ACCCCUUG G AACAUUUU | 1662 | AAAAUGUU GGAGGAAACUCC CU UCAAGGACAUCGUCCGGG CAAGGGGU | 4423 |
| 1500 | UACCUAAU G GGUGAGGU | 1663 | ACCUCACC GGAGGAAACUCC CU UCAAGGACAUCGUCCGGG AUUAGGUA | 4424 |
| 1501 | ACCUAAUG G GUGAGGUU | 1664 | AACCUCAC GGAGGAAACUCC CU UCAAGGACAUCGUCCGGG CAUUAGGU | 4425 |
| 1506 | AUGGGUGA G GUUACCAA | 1665 | UUGGUAAC GGAGGAAACUCC CU UCAAGGACAUCGUCCGGG UCACCCAU | 4426 |
| 1556 | AUACCUGC G GCCAGUGG | 1666 | CCACUGGC GGAGGAAACUCC CU UCAAGGACAUCGUCCGGG GCAGGUAU | 4427 |
| 1563 | CGGCCAGU G GAAGAUGU | 1667 | ACAUCUUC GGAGGAAACUCC CU UCAAGGACAUCGUCCGGG ACUGGCCG | 4428 |
| 1564 | GGCCAGUG G AAGAUGUG | 1668 | CACAUCUU GGAGGAAACUCC CU UCAAGGACAUCGUCCGGG CACUGGCC | 4429 |
| 1567 | CAGUGGAA G AUGUGGCC | 1669 | GGCCACAU GGAGGAAACUCC CU UCAAGGACAUCGUCCGGG UUCCACUG | 4430 |
| 1572 | GAAGAUGU G GCCACGUC | 1670 | GACGUGGC GGAGGAAACUCC CU UCAAGGACAUCGUCCGGG ACAUCUUC | 4431 |
| 1585 | CGUCCCAA G ACGACUGU | 1671 | ACAGUCGU GGAGGAAACUCC CU UCAAGGACAUCGUCCGGG UUGGGACG | 4432 |
| 1623 | UCAUCCAC G GGCACUGU | 1672 | ACAGUGCC GGAGGAAACUCC CU UCAAGGACAUCGUCCGGG GUGGAUGA | 4433 |
| 1624 | CAUCCACG G GCACUGUU | 1673 | AACAGUGC GGAGGAAACUCC CU UCAAGGACAUCGUCCGGG CGUGGAUG | 4434 |
| 1635 | ACUGUUAU G GGAGCUGU | 1674 | ACAGCUCC GGAGGAAACUCC CU UCAAGGACAUCGUCCGGG AUAACAGU | 4435 |
| 1636 | CUGUUAUG G GAGCUGUU | 1675 | AACAGCUC GGAGGAAACUCC CU UCAAGGACAUCGUCCGGG CAUAACAG | 4436 |
| 1637 | UGUUAUGG G AGCUGUUA | 1676 | UAACAGCU GGAGGAAACUCC CU UCAAGGACAUCGUCCGGG CCAUAACA | 4437 |
| 1650 | GUUAUCAU G GAGGGCUU | 1677 | AAGCCCUC GGAGGAAACUCC CU UCAAGGACAUCGUCCGGG AUGAUAAC | 4438 |
| 1651 | UUAUCAUG G AGGGCUUC | 1678 | GAAGCCCU GGAGGAAACUCC CU UCAAGGACAUCGUCCGGG CAUGAUAA | 4439 |
| 1653 | AUCAUGGA G GGCUUCUA | 1679 | UAGAAGCC GGAGGAAACUCC CU UCAAGGACAUCGUCCGGG UCCAUGAU | 4440 |
| 1654 | UCAUGGAG G GCUUCUAC | 1680 | GUAGAAGC GGAGGAAACUCC CU UCAAGGACAUCGUCCGGG CUCCAUGA | 4441 |
| 1676 | CUUUGAUC G GGCCCGAA | 1681 | UUCGGGCC GGAGGAAACUCC CU UCAAGGACAUCGUCCGGG GAUCAAAG | 4442 |
| 1677 | UUUGAUCG G GCCCGAAA | 1682 | UUUCGGGC GGAGGAAACUCC CU UCAAGGACAUCGUCCGGG CGAUCAAA | 4443 |
| 1693 | AACGAAUU G GCUUUGCU | 1683 | AGCAAAGC GGAGGAAACUCC CU UCAAGGACAUCGUCCGGG AAUUCGUU | 4444 |
| 1733 | UGAGUUCA G GACGGCAG | 1684 | CUGCCGUC GGAGGAAACUCC CU UCAAGGACAUCGUCCGGG UGAACUCA | 4445 |
| 1734 | GAGUUCAG G ACGGCAGC | 1685 | GCUGCCGU GGAGGAAACUCC CU UCAAGGACAUCGUCCGGG CUGAACUC | 4446 |
| 1737 | UUCAGGAC G GCAGCGGU | 1686 | ACCGCUGC GGAGGAAACUCC CU UCAAGGACAUCGUCCGGG GUCCUGAA | 4447 |
| 1743 | ACGGCAGC G GUGGAAGG | 1687 | CCUUCCAC GGAGGAAACUCC CU UCAAGGACAUCGUCCGGG GCUGCCGU | 4448 |
| 1746 | GCAGCGGU G GAAGGCCC | 1688 | GGGCCUUC GGAGGAAACUCC CU UCAAGGACAUCGUCCGGG ACCGCUGC | 4449 |
| 1747 | CAGCGGUG G AAGGCCCU | 1689 | AGGGCCUU GGAGGAAACUCC CU UCAAGGACAUCGUCCGGG CACCGCUG | 4450 |
| 1750 | CGGUGGAA G GCCCUUUU | 1690 | AAAAGGGC GGAGGAAACUCC CU UCAAGGACAUCGUCCGGG UUCCACCG | 4451 |
| 1767 | GUCACCUU G GACAUGGA | 1691 | UCCAUGUC GGAGGAAACUCC CU UCAAGGACAUCGUCCGGG AAGGUGAC | 4452 |
| 1768 | UCACCUUG G ACAUGGAA | 1692 | UUCCAUGU GGAGGAAACUCC CU UCAAGGACAUCGUCCGGG CAAGGUGA | 4453 |
| 1773 | UUGGACAU G GAAGACUG | 1693 | CAGUCUUC GGAGGAAACUCC CU UCAAGGACAUCGUCCGGG AUGUCCAA | 4454 |
| 1774 | UGGACAUG G AAGACUGU | 1694 | ACAGUCUU GGAGGAAACUCC CU UCAAGGACAUCGUCCGGG CAUGUCCA | 4455 |
| 1777 | ACAUGGAA G ACUGUGGC | 1695 | GCCACAGU GGAGGAAACUCC CU UCAAGGACAUCGUCCGGG UUCCAUGU | 4456 |
| 1783 | AAGACUGU G GCUACAAC | 1696 | GUUGUAGC GGAGGAAACUCC CU UCAAGGACAUCGUCCGGG ACAGUCUU | 4457 |
| 1800 | AUUCCACA G ACAGAUGA | 1697 | UCAUCUGU GGAGGAAACUCC CU UCAAGGACAUCGUCCGGG UGUGGAAU | 4458 |
| 1804 | CACAGACA G AUGAGUCA | 1698 | UGACUCAU GGAGGAAACUCC CU UCAAGGACAUCGUCCGGG UGUCUGUG | 4459 |
| 1839 | UAUGUCAU G GCUGCCAU | 1699 | AUGGCAGC GGAGGAAACUCC CU UCAAGGACAUCGUCCGGG AUGACAUA | 4460 |
| 1881 | UGCCUCAU G GUGUGUCA | 1700 | UGACACAC GGAGGAAACUCC CU UCAAGGACAUCGUCCGGG AUGAGGCA | 4461 |
| 1892 | GUGUCAGU G GCGCUGCC | 1701 | GGCAGCGC GGAGGAAACUCC CU UCAAGGACAUCGUCCGGG ACUGACAC | 4462 |
| 1960 | UGAAGUGA G GAGGCCCA | 1702 | UGGGCCUC GGAGGAAACUCC CU UCAAGGACAUCGUCCGGG UCACUUCA | 4463 |
| 1961 | GAAGUGAG G AGGCCCAU | 1703 | AUGGGCCU GGAGGAAACUCC CU UCAAGGACAUCGUCCGGG CUCACUUC | 4464 |
| 1963 | AGUGAGGA G GCCCAUGG | 1704 | CCAUGGGC GGAGGAAACUCC CU UCAAGGACAUCGUCCGGG UCCUCACU | 4465 |
| 1970 | AGGCCCAU G GGCAGAAG | 1705 | CUUCUGCC GGAGGAAACUCC CU UCAAGGACAUCGUCCGGG AUGGGCCU | 4466 |
| 1971 | GGCCCAUG G GCAGAAGA | 1706 | UCUUCUGC GGAGGAAACUCC CU UCAAGGACAUCGUCCGGG CAUGGGCC | 4467 |
| 1975 | CAUGGGCA G AAGAUAGA | 1707 | UCUAUCUU GGAGGAAACUCC CU UCAAGGACAUCGUCCGGG UGCCCAUG | 4468 |
| 1978 | GGGCAGAA G AUAGAGAU | 1708 | AUCUCUAU GGAGGAAACUCC CU UCAAGGACAUCGUCCGGG UUCUGCCC | 4469 |
| 1982 | AGAAGAUA G AGAUUCCC | 1709 | GGGAAUCU GGAGGAAACUCC CU UCAAGGACAUCGUCCGGG UAUCUUCU | 4470 |
| 1984 | AAGAUAGA G AUUCCCCU | 1710 | AGGGGAAU GGAGGAAACUCC CU UCAAGGACAUCGUCCGGG UCUAUCUU | 4471 |
| 1993 | AUUCCCCU G GACCACAC | 1711 | GUGUGGUC GGAGGAAACUCC CU UCAAGGACAUCGUCCGGG AGGGGAAU | 4472 |
| 1994 | UUCCCCUG G ACCACACC | 1712 | GGUGUGGU GGAGGAAACUCC CU UCAAGGACAUCGUCCGGG CAGGGGAA | 4473 |
| 2008 | ACCUCCGU G GUUCACUU | 1713 | AAGUGAAC GGAGGAAACUCC CU UCAAGGACAUCGUCCGGG ACGGAGGU | 4474 |
| 2018 | UUCACUUU G GUCACAAG | 1714 | CUUGUGAC GGAGGAAACUCC CU UCAAGGACAUCGUCCGGG AAAGUGAA | 4475 |
| 2029 | CACAAGUA G GAGACACA | 1715 | UGUGUCUC GGAGGAAACUCC CU UCAAGGACAUCGUCCGGG UACUUGUG | 4476 |
| 2030 | ACAAGUAG G AGACACAG | 1716 | CUGUGUCU GGAGGAAACUCC CU UCAAGGACAUCGUCCGGG CUACUUGU | 4477 |
| 2032 | AAGUAGGA G ACACAGAU | 1717 | AUCUGUGU GGAGGAAACUCC CU UCAAGGACAUCGUCCGGG UCCUACUU | 4478 |
| 2038 | GAGACACA G AUGGCACC | 1718 | GGUGCCAU GGAGGAAACUCC CU UCAAGGACAUCGUCCGGG UGUGUCUC | 4479 |
| 2041 | ACACAGAU G GCACCUGU | 1719 | ACAGGUGC GGAGGAAACUCC CU UCAAGGACAUCGUCCGGG AUCUGUGU | 4480 |
| 2050 | GCACCUGU G GCCAGAGC | 1720 | GCUCUGGC GGAGGAAACUCC CU UCAAGGACAUCGUCCGGG ACAGGUGC | 4481 |
| 2055 | UGUGGCCA G AGCACCUC | 1721 | GAGGUGCU GGAGGAAACUCC CU UCAAGGACAUCGUCCGGG UGGCCACA | 4482 |
| 2065 | GCACCUCA G GACCCUCC | 1722 | GGAGGGUC GGAGGAAACUCC CU UCAAGGACAUCGUCCGGG UGAGGUGC | 4483 |
| 2066 | CACCUCAG G ACCCUCCC | 1723 | GGGAGGGU GGAGGAAACUCC CU UCAAGGACAUCGUCCGGG CUGAGGUG | 4484 |
| 2101 | GCCUUGAU G GAGAAGGA | 1724 | UCCUUCUC GGAGGAAACUCC CU UCAAGGACAUCGUCCGGG AUCAAGGC | 4485 |
| 2102 | CCUUGAUG G AGAAGGAA | 1725 | UUCCUUCU GGAGGAAACUCC CU UCAAGGACAUCGUCCGGG CAUCAAGG | 4486 |
| 2104 | UUGAUGGA G AAGGAAAA | 1726 | UUUUCCUU GGAGGAAACUCC CU UCAAGGACAUCGUCCGGG UCCAUCAA | 4487 |
| 2107 | AUGGAGAA G GAAAAGGC | 1727 | GCCUUUUC GGAGGAAACUCC CU UCAAGGACAUCGUCCGGG UUCUCCAU | 4488 |
| 2108 | UGGAGAAG G AAAAGGCU | 1728 | AGCCUUUU GGAGGAAACUCC CU UCAAGGACAUCGUCCGGG CUUCUCCA | 4489 |
| 2113 | AAGGAAAA G GCUGGCAA | 1729 | UUGCCAGC GGAGGAAACUCC CU UCAAGGACAUCGUCCGGG UUUUCCUU | 4490 |
| 2117 | AAAAGGCU G GCAAGGUG | 1730 | CACCUUGC GGAGGAAACUCC CU UCAAGGACAUCGUCCGGG AGCCUUUU | 4491 |
| 2122 | GCUGGCAA G GUGGGUUC | 1731 | GAACCCAC GGAGGAAACUCC CU UCAAGGACAUCGUCCGGG UUGCCAGC | 4492 |
| 2125 | GGCAAGGU G GGUUCCAG | 1732 | CUGGAACC GGAGGAAACUCC CU UCAAGGACAUCGUCCGGG ACCUUGCC | 4493 |
| 2126 | GCAAGGUG G GUUCCAGG | 1733 | CCUGGAAC GGAGGAAACUCC CU UCAAGGACAUCGUCCGGG CACCUUGC | 4494 |
| 2133 | GGGUUCCA G GGACUGUA | 1734 | UACAGUCC GGAGGAAACUCC CU UCAAGGACAUCGUCCGGG UGGAACCC | 4495 |
| 2134 | GGUUCCAG G GACUGUAC | 1735 | GUACAGUC GGAGGAAACUCC CU UCAAGGACAUCGUCCGGG CUGGAACC | 4496 |
| 2135 | GUUCCAGG G ACUGUACC | 1736 | GGUACAGU GGAGGAAACUCC CU UCAAGGACAUCGUCCGGG CCUGGAAC | 4497 |
| 2148 | UACCUGUA G GAAACAGA | 1737 | UCUGUUUC GGAGGAAACUCC CU UCAAGGACAUCGUCCGGG UACAGGUA | 4498 |
| 2149 | ACCUGUAG G AAACAGAA | 1738 | UUCUGUUU GGAGGAAACUCC CU UCAAGGACAUCGUCCGGG CUACAGGU | 4499 |
| 2155 | AGGAAACA G AAAAGAGA | 1739 | UCUCUUUU GGAGGAAACUCC CU UCAAGGACAUCGUCCGGG UGUUUCCU | 4500 |
| 2160 | ACAGAAAA G AGAAGAAA | 1740 | UUUCUUCU GGAGGAAACUCC CU UCAAGGACAUCGUCCGGG UUUUCUGU | 4501 |
| 2162 | AGAAAAGA G AAGAAAGA | 1741 | UCUUUCUU GGAGGAAACUCC CU UCAAGGACAUCGUCCGGG UCUUUUCU | 4502 |
| 2165 | AAAGAGAA G AAAGAAGC | 1742 | GCUUCUUU GGAGGAAACUCC CU UCAAGGACAUCGUCCGGG UUCUCUUU | 4503 |
| 2169 | AGAAGAAA G AAGCACUC | 1743 | GAGUGCUU GGAGGAAACUCC CU UCAAGGACAUCGUCCGGG UUUCUUCU | 4504 |
| 2182 | ACUCUGCU G GCGGGAAU | 1744 | AUUCCCGC GGAGGAAACUCC CU UCAAGGACAUCGUCCGGG AGCAGAGU | 4505 |
| 2185 | CUGCUGGC G GGAAUACU | 1745 | AGUAUUCC GGAGGAAACUCC CU UCAAGGACAUCGUCCGGG GCCAGCAG | 4506 |
| 2186 | UGCUGGCG G GAAUACUC | 1746 | CAGUAUUC GGAGGAAACUCC CU UCAAGGACAUCGUCCGGG CGCCAGCA | 4507 |
| 2187 | GCUGGCGG G AAUACUCU | 1747 | ACAGUAUU GGAGGAAACUCC CU UCAAGGACAUCGUCCGGG CCGCCAGC | 4508 |
| 2197 | AUACUCUU G GUCACCUC | 1748 | GAGGUGAC GGAGGAAACUCC CU UCAAGGACAUCGUCCGGG AAGAGUAU | 4509 |
| 2217 | UUUAAGUC G GGAAAUUC | 1749 | GAAUUUCC GGAGGAAACUCC CU UCAAGGACAUCGUCCGGG GACUUAAA | 4510 |
| 2218 | UUAAGUCG G GAAAUUCU | 1750 | AGAAUUUC GGAGGAAACUCC CU UCAAGGACAUCGUCCGGG CGACUUAA | 4511 |
| 2219 | UAAGUCGG G AAAUUCUG | 1751 | CAGAAUUU GGAGGAAACUCC CU UCAAGGACAUCGUCCGGG CCGACUUA | 4512 |
| 2311 | UAGUUUCA G AAGUACUG | 1752 | CAGUACUU GGAGGAAACUCC CU UCAAGGACAUCGUCCGGG UGAAACUA | 4513 |
| 2319 | GAAGUACU G GCAUCACA | 1753 | UGUGAUGC GGAGGAAACUCC CU UCAAGGACAUCGUCCGGG AGUACUUC | 4514 |
| 2332 | CACACGCA G GUUACCUU | 1754 | AAGGUAAC GGAGGAAACUCC CU UCAAGGACAUCGUCCGGG UGCGUGUG | 4515 |
| 2341 | GUUACCUU G GCGUGUGU | 1755 | ACACACGC GGAGGAAACUCC CU UCAAGGACAUCGUCCGGG AAGGUAAC | 4516 |
| 2356 | GUCCCUGU G GUACCCUG | 1756 | CAGGGUAC GGAGGAAACUCC CU UCAAGGACAUCGUCCGGG ACAGGGAC | 4517 |
| 2364 | GGUACCCU G GCAGAGAA | 1757 | UUCUCUGC GGAGGAAACUCC CU UCAAGGACAUCGUCCGGG AGGGUACC | 4518 |
| 2368 | CCCUGGCA G AGAAGAGA | 1758 | UCUCUUCU GGAGGAAACUCC CU UCAAGGACAUCGUCCGGG UGCCAGGG | 4519 |
| 2370 | CUGGCAGA G AAGAGACC | 1759 | GGUCUCUU GGAGGAAACUCC CU UCAAGGACAUCGUCCGGG UCUGCCAG | 4520 |
| 2373 | GCAGAGAA G AGACCAAG | 1760 | CUUGGUCU GGAGGAAACUCC CU UCAAGGACAUCGUCCGGG UUCUCUGC | 4521 |
| 2375 | AGAGAAGA G ACCAAGCU | 1761 | AGCUUGGU GGAGGAAACUCC CU UCAAGGACAUCGUCCGGG UCUUCUCU | 4522 |
| 2396 | UCCCUGCU G GCCAAAGU | 1762 | ACUUUGGC GGAGGAAACUCC CU UCAAGGACAUCGUCCGGG AGCAGGGA | 4523 |
| 2410 | AGUCAGUA G GAGAGGAU | 1763 | AUCCUCUC GGAGGAAACUCC CU UCAAGGACAUCGUCCGGG UACUGACU | 4524 |
| 2411 | GUCAGUAG G AGAGGAUG | 1764 | CAUCCUCU GGAGGAAACUCC CU UCAAGGACAUCGUCCGGG CUACUGAC | 4525 |
| 2413 | CAGUAGGA G AGGAUGCA | 1765 | UGCAUCCU GGAGGAAACUCC CU UCAAGGACAUCGUCCGGG UCCUACUG | 4526 |
| 2415 | GUAGGAGA G GAUGCACA | 1766 | UGUGCAUC GGAGGAAACUCC CU UCAAGGACAUCGUCCGGG UCUCCUAC | 4527 |
| 2416 | UAGGAGAG G AUGCACAG | 1767 | CUGUGCAU GGAGGAAACUCC CU UCAAGGACAUCGUCCGGG CUCUCCUA | 4528 |
| 2441 | UUGCUUUA G AGACAGGG | 1768 | CCCUGUCU GGAGGAAACUCC CU UCAAGGACAUCGUCCGGG UAAAGCAA | 4529 |
| 2443 | GCUUUAGA G ACAGGGAC | 1769 | GUCCCUGU GGAGGAAACUCC CU UCAAGGACAUCGUCCGGG UCUAAAGC | 4530 |
| 2447 | UAGAGACA G GGACUGUA | 1770 | UACAGUCC GGAGGAAACUCC CU UCAAGGACAUCGUCCGGG UGUCUCUA | 4531 |
| 2448 | AGAGACAG G GACUGUAU | 1771 | AUACAGUC GGAGGAAACUCC CU UCAAGGACAUCGUCCGGG CUGUCUCU | 4532 |
| 2449 | GAGACAGG G ACUGUAUA | 1772 | UAUACAGU GGAGGAAACUCC CU UCAAGGACAUCGUCCGGG CCUGUCUC | 4533 |
| 2473 | CUAACAUU G GUGCAAAG | 1773 | CUUUGCAC GGAGGAAACUCC CU UCAAGGACAUCGUCCGGG AAUGUUAG | 4534 |
| 2481 | GGUGCAAA G AUUGCCUC | 1774 | GAGGCAAU GGAGGAAACUCC CU UCAAGGACAUCGUCCGGG UUUGCACC | 4535 |
| 2511 | AAAAACUA G AAAAAAAA | 1775 | UUUUUUUU GGAGGAAACUCC CU UCAAGGACAUCGUCCGGG UAGUUUUU | 4536 |

Input Sequence = AF190725.
Cut Site = G/.
Stem Length = 8.
Core Sequence = GGAGGAAACUCC CU UCAAGGACAUCGUCCGGG
AF190725 (Homo sapiens beta-site APP cleaving enzyme (BACE) mRNA; 2526 bp)

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of Markush group or other group.

Other embodiments are within the following claims.

What we claim is:

1. A nucleic acid sensor molecule which modulates expression of a beta site APP-cleaving enzyme (BACE) gene.

2. A nucleic acid sensor molecule which modulates expression of a presenilin (ps-2) gene.

3. A nucleic acid sensor molecule which modulates expression of an amyloid precursor protein (APP) gene.

4. The nucleic acid sensor molecule of any of claims 1-3, wherein said nucleic acid sensor molecule is adapted for use to treat Alzheimer's disease.

5. The nucleic acid sensor molecule of claim 1, wherein said nucleic acid sensor molecule has an endonuclease activity to cleave RNA encoded by said BACE gene.

6. The nucleic acid sensor molecule of claim 2, wherein said nucleic acid sensor molecule has an endonuclease activity to cleave RNA encoded by said ps-2 gene.

7. The nucleic acid sensor molecule of claim 3, wherein said nucleic acid sensor molecule has an endonuclease activity to cleave RNA encoded by said APP gene.

8. The nucleic acid sensor molecule of claim 1, wherein a binding arm of the nucleic acid sensor molecule comprise sequences complementary to any of sequences having SEQ ID NOs: 1-1775.

9. The nucleic acid sensor molecule of claim 1, wherein an enzymatic nucleic acid portion of the nucleic acid sensor molecule comprises any of sequences having SEQ ID NOs: 1776-3972.

10. The nucleic acid sensor molecule of any of claims 1-3, wherein said nucleic acid sensor molecule comprises a hammerhead enzymatic nucleic acid motif.

11. The nucleic acid sensor molecule of any of claims 1-3, wherein said nucleic acid sensor molecule comprises an Inozyme enzymatic nucleic acid motif.

12. The nucleic acid sensor molecule of any of claims 1-3, wherein said nucleic acid sensor molecule comprises a Zinzyme enzymatic nucleic acid motif.

13. The nucleic acid sensor molecule of any of claims 1-3, wherein said nucleic acid sensor molecule comprises an Amberzyme enzymatic nucleic acid motif.

14. The nucleic acid sensor molecule of any of claims 1-3, wherein said nucleic acid sensor molecule comprises a G-cleaver enzymatic nucleic acid motif.

15. The nucleic acid sensor molecule of any of claims 1-3, wherein said nucleic acid sensor molecule comprises a hairpin enzymatic nucleic acid motif.

16. The nucleic acid sensor molecule of any of claims 1-3, wherein said nucleic acid sensor molecule comprises a DNAzyme.

17. The nucleic acid sensor molecule of claim 1, wherein said nucleic acid sensor molecule comprises between 12 and 100 bases complementary to RNA of a BACE gene.

18. The nucleic acid sensor molecule of claim 1, wherein said nucleic acid sensor molecule comprises between 14 and 24 bases complementary toRNA of a BACE gene.

19. The nucleic acid sensor molecule of claim 2, wherein said nucleic acid sensor molecule comprises between 12 and 100 bases complementary to RNA of a ps-2 gene.

20. The nucleic acid sensor molecule of claim 2, wherein said nucleic acid sensor molecule comprises between 14 and 24 bases complementary to RNA of a ps-2 gene.

21. The nucleic acid sensor molecule of claim 3, wherein said nucleic acid sensor molecule comprises between 12 and 100 bases complementary to RNA of a APP gene.

22. The nucleic acid sensor molecule of claim 3, wherein said nucleic acid sensor molecule comprises between 14 and 24 bases complementary to RNA of an APP gene.

23. The nucleic acid sensor molecule of any of claims 1-3, wherein said nucleic acid sensor molecule is chemically synthesized.

24. The nucleic acid sensor molecule of any of claims 1-3, wherein said nucleic acid sensor molecule comprises at least one 2′-sugar modification.

25. The nucleic acid sensor molecule of any of claims 1-3, wherein said nucleic acid sensor molecule comprises at least one nucleic acid base modification.

26. The nucleic acid sensor molecule of any of claims 1-3, wherein said nucleic acid sensor molecule comprises at least one phosphate backbone modification.

27. A mammalian cell including the nucleic acid sensor molecule of any of claims 1-3.

28. The mammalian cell of claim 27, wherein said mammalian cell is a human cell.

29. A method of reducing BACE activity in a cell, comprising contacting said cell with the nucleic acid sensor molecule of claim 1, under conditions suitable for said inhibition.

30. A method of treatment of a patient having a condition associated with the level of BACE, comprising contacting cells of said patient with the nucleic acid sensor molecule of claim 1, under conditions suitable for said treatment.

31. The method of claim 30 further comprising the use of one or more drug therapies under conditions suitable for said treatment.

32. A method of cleaving RNA of a BACE gene, comprising, contacting the nucleic acid sensor molecule of claim 1, with said RNA under conditions suitable for the cleavage of said RNA.

33. The method of claim 32, wherein said cleavage is carried out in the presence of a divalent cation.

34. The method of claim 33, wherein said divalent cation is Mg²⁺.

35. The nucleic acid sensor molecule of any of claims 1-3, wherein said nucleic acid sensor molecule comprises a cap structure, wherein the cap structure is at the 5′-end or 3′-end or both the 5′-end and the 3′-end.

36. A method for treatment of Alzheimer's disease comprising administering to a patient the nucleic acid sensor molecule of any of claims 1-3 under conditions suitable for said treatment.

37. The method of claim 36, wherein said method further comprises administering to said patient the enzymatic nucleic acid molecule in conjunction with one or more of other therapies.

38. A nucleic acid sensor molecule capable of modulating the expression of BACE in the presence of beta-amyloid protein.

39. A nucleic acid sensor molecule capable of modulating the expression of presenilin-2 in the presence of beta-amyloid protein.

40. A nucleic acid sensor molecule capable of modulating the expression of amyloid precursor protein in the presence of beta-amyloid protein.

41. A nucleic acid sensor molecule capable of modulating the expression of BACE in the presence of amyloid precursor protein.

42. A nucleic acid sensor molecule capable of modulating the expression of presenilin-2 in the presence of amyloid precursor protein.

43. A nucleic acid sensor molecule capable of modulating the expression of amyloid precursor protein in the presence of amyloid precursor protein.

44. A nucleic acid sensor molecule capable of modulating the expression of BACE in the presence of BACE RNA.

45. A nucleic acid sensor molecule capable of modulating the expression of presenilin-2 in the presence of BACE RNA.

46. A nucleic acid sensor molecule capable of modulating the expression of amyloid precursor protein in the presence of BACE RNA.

47. A nucleic acid sensor molecule capable of modulating the expression of BACE, in the presence of presenilin-2 RNA.

48. A nucleic acid sensor molecule capable of modulating the expression of presenilin-2 in the presence of presenilin-2 RNA.

49. A nucleic acid sensor molecule capable of modulating the expression of amyloid precursor protein in the presence of presenilin-2 RNA.

50. A nucleic acid sensor molecule capable of modulating the expression of BACE in the presence of amyloid precursor protein RNA.

51. A nucleic acid sensor molecule capable of modulating the expression of presenilin-2 in the presence of amyloid precursor protein RNA.

52. A nucleic acid sensor molecule capable of modulating the expression of amyloid precursor protein in the presence of amyloid precursor protein RNA.

53. The nucleic acid sensor molecule of any of claims 1-3, wherein said modulation is inhibition.

54. The nucleic acid sensor molecule of any of claims 38-53, wherein said modulation is inhibition.
